(19) United States
(12) Patent Application Publication
(10) Pub. No.: US 2004/0014083 A1
(43) Pub. Date: Jan. 22, 2004

(54) Detection of heteroduplex polynucleotides using mutant nucleic acid repair enzymes with attenuated catalytic activity

(76) Inventors: Chong-Sheng Yuan, San Diego, CA (US); Abhijit Datta, Carlsbad, CA (US)

Correspondence Address: Peng Chen, Morrison & Foerster LLP, Suite 500, 3811 Valley Centre Drive, San Diego, CA, 92130-2332, US

(21) Appl. No.: 10/373,238

(22) Filed: Feb. 24, 2003

Related U.S. Application Data
(63) Continuation-in-part of application No. 09/514,016, filed on Feb. 25, 2000.

(51) Int. Cl. C12Q 1/68

(52) U.S. Cl. 435/6

(57) ABSTRACT

Methods for detecting, localizing and removing abnormal base-pairing in a nucleic acid duplex are provided. These methods can be used for prognosis and diagnosis of diseases, disorders, pathogenic infections and nucleic acid polymorphisms. Combinations, kits and articles of manufacture for use in these methods are also provided.

### RELATED APPLICATIONS

This application is a continuation-in-part application of U.S. patent application Ser. No. 09/514,016, filed Feb. 25, 2000, now pending. This application is also related to U.S. application Ser. No. 09/347,878, filed Jul. 6, 1999, entitled “COMPOSITIONS AND METHODS FOR ASSAYING ANALYTES” and U.S. application Ser. No. 09/457,205, filed Dec. 6, 1999, entitled “COMPOSITIONS AND METHODS FOR ASSAYING ANALYTES.” U.S. application Ser. No. 09/457,205 is a continuation-in-part application of U.S. patent application Ser. No. 09/347,878, filed Jul. 6, 1999, now U.S. Pat. No. 6,376,210 B 1. The contents of each of these applications is incorporated herein in its entirety.

### FIELD OF THE INVENTION

Methods for detecting nucleic acids that contain any abnormal base-pairing in a nucleic acid duplex are provided. The methods are particularly useful for prognosis and diagnosis of diseases, disorders and pathogenic infections and for detection of nucleic acid polymorphisms. Also provided are mutant nucleic acid binding enzymes, particularly repair enzymes, that retain binding specificity and affinity, but lack catalytic activity. Combinations, kits and articles of manufactures that contain these mutant enzymes are also provided.

### BACKGROUND OF THE INVENTION

In the wake of the human genome project, future medical practice will use more and more human genetic information for disease prognosis, diagnosis and prevention. The need for rapid and accurate methods of genetic variation detection are escalating. It is these nucleic acid mutation detection technologies that will ultimately help to reveal the relation between human genetic makeup and diseases. Although methods are available for detecting DNA mutations/polymorphisms, none is suitable for use in a high throughput format for detecting large numbers of mutations/polymorphisms simultaneously in a single assay format.

This lack of suitability derives from the requisite use of specific probes for detecting mutations in the target nucleic acids. For example, PCR-restriction fragment length polymorphism (PCR-RFLP) (see, e.g., Bashiruddin, Methods Mol. Biol., 104:167-78 (1998); Hyland et al., Transfus. Med. Rev., 9(4):289-301 (1995); Gasser and Chilton, Acta Trop., 59(1):31-40 (1995); and Pourzand and Cerutti, Mutat. Res., 288(1):113-21 (1993)), not only requires the design of target-specific probes, but also involves a gel-electrophoresis step to analyze the DNA digestion patterns in comparison with the wild type gene. It is a time consuming and expensive procedure. Similar problem exists with other methods such as single-strand conformation polymorphism (PCR-SSCP) detection, which also requires specific probes and gel-electrophoresis (Hayashi and Yandell, Hum. Mutat., 2(5):338-46 (1993); Hayashi, Genet. Anal. Tech. Appl., 9(3):73-9 (1992); and Hayashi, PCR Methods Appl., 1(1):34-8 (1991)). Methods, such as the Invader™ assay (Third Wave Technologies, Inc.) for detection of polymorphism based on the use of Cleavase enzymes to cleave a complex formed by hybridization of overlapping oligonucleotide probes (Marshall et al., J. Clin. Microbiol., 35(12):3156-62 (1997)) eliminates the gel-electrophoresis step, but the method requires more probes specific for the genes to be tested. Moreover, the Invader™ assay method works only when the exact mutation and mutation position are known. Therefore, it is difficult to automate this method for detecting large number of genes in a single format.

Therefore, a need to develop nucleic acid detection and mapping methods amenable to high throughput formats. Thus, it is an object herein to provide a nucleic acid mutation detecting method that requires neither specific probes nor gel-electrophoresis. It is another object herein to provide a nucleic acid mutation detecting method that is amendable to automation for simultaneous detection of large numbers of nucleic acid mutations.

### SUMMARY

Provided herein are nucleic acid mutation detecting methods that meet the above-noted objectives. These methods have wide application in various areas such as prognosis and diagnosis of diseases, disorders or pathological infections, and selectively binding, such as for removal or purification, nucleic acid duplexes that include abnormal base-pairings in a population of nucleic acid duplexes.

The nucleic acid mutation detecting methods provided herein use mutant nucleic acid binding enzymes, such as mutant repair enzymes, and other enzymes that specifically bind to abnormal base pairs, such as base-pair mismatch, a base insertion, a base deletion and a pyrimidine dimer. The mutant enzymes substantially retain the specific binding affinities for abnormal base-pairings of the wild-type enzymes but have reduced or lack the catalytic activities. The mutant enzymes thus act like an antibody (herein designated a pseudo-antibody) and specifically bind to abnormal base-pairings in a duplex. The mutant enzymes are enzymes, such as repair enzymes, particularly DNA repair enzymes, that typically bind to a abnormally matched base pairs, such as base-pair mismatches, base insertions, a base deletions and pyrimidine dimers, and then catalytically repair the duplex. Methods of detection, diagnosis and other methods that rely on the affinity of the mutant enzymes for duplexes with abnormal base pairings, such as mismatches, are provided.

Among the methods provided, are methods for identifying and quantifying mutations. These methods are based upon the specificity of the mutant enzyme for a particularly abnormal base pairing. Hybridizing perfectly matched nucleic acid strands forms a nucleic acid duplex without any abnormal base-pairings and hybridizing imperfectly matched nucleic acid strands forms a nucleic acid duplex with one or more abnormal base-pairings. By contacting the formed nucleic acid duplex with one or more mutant repair enzyme(s), the duplex containing abnormal base-pairing(s) binds to the mutant repair enzyme. Detection and quantitation of the complex formed between the nucleic acid duplex with the one or more abnormal base-pairings and the mutant DNA repair enzyme leads to identification and quantitation of nucleic acid mutations.

Hence, provided herein is a method for detecting abnormal base-pairing in a nucleic acid duplex by contacting a nucleic acid duplex having or suspected of having an abnormal base-pairing with a mutant DNA repair enzyme or complex thereof that has binding affinity for the abnormal base-pairing in the duplex but has attenuated catalytic activity; and then detecting binding between the nucleic acid duplex and the mutant DNA repair enzyme or complex thereof. The amount of mutant enzyme bound is used to assess the presence or quantity of the abnormal base-pairing in the duplex.

The nucleic acid duplex that is assayed includes DNA:DNA, DNA:RNA and RNA:RNA duplexes. Preferably, the nucleic acid duplex to be assayed is a DNA:DNA duplex.

The abnormal base-pairing that is detected can be, for example, a base-pair mismatch, a base insertion, a base deletion or a pyrimidine dimer. Among the preferred uses of the mutant enzymes is for detection of a single base-pair mismatch. Such mismatches include, but are not limited to, A:A, A:C, A:G, C:C, C:T, G:G, G:T, T:T, C:U, G:U, T:U, U:U, 5-formyluracil (fU):G, 7,8-dihydro-8-oxo-guanine (8-oxoG):C, 8-oxoG:A and any combination thereof. Also preferably, the base insertion or base deletion to be detected is a single base insertion or deletion. For example, the base insertion or base deletion resulting in a single-stranded loop containing about 1-5 bases or a loop containing more than 5 bases can be detected.

Mutant DNA repair enzyme or complexes thereof that can be used in these methods include a mutant of any nucleic acid repair enzyme (or enzyme complex) as long as the mutant retains its ability to specifically bind to the nucleic acid that the wild-type repairs, but lacks substantial catalytic activity. Enzymatic systems capable of recognition and correction of base pairing errors within the DNA helix have been demonstrated in bacteria, fungi and mammalian cells. Enzymes from any such system is contemplated herein. The enzyme can be mutagenized using standard procedures, either directed mutagenesis if the catalytic site is known, or systematic mutagenesis to empirically identify suitable mutations. The resulting enzymes are selected for their ability to bind to abnormally, such as mismatched, paired DNA but to not effect repair or catalytic activity. Exemplary enzymes include, but are not limited to, a mutant mutH, a mutant mutL, a mutant mutM, a mutant mutS, a mutant mutY, a mutant uvrD, a mutant dam, a mutant thymidine DNA glycosylase (TDG), a mutant mismatch-specific DNA glycosylase (MUG), a mutant AlkA, a mutant MLH1, a mutant MSH2, a mutant MSH3, a mutant MSH6, a mutant Exonuclease I, a mutant T4 endonuclease V, a mutant FEN1 (RAD27), a mutant DNA polymerase ä, a mutant DNA polymerase å, a mutant RPA, a mutant PCNA, a mutant RFC, a mutant Exonuclease V, a mutant DNA polymerase III holoenzyme, a mutant DNA helicase, a mutant RecJ exonuclease, a cleavase and combinations thereof (see below for definitions of each enzyme).

Also provided herein are methods for detecting a mutation in a nucleic acid. The methods are performed by hybridizing a strand of a nucleic acid having or suspected of having a mutation with a complementary strand of a wild-type nucleic acid, whereby if a mutation is present, the resulting duplex contains an abnormal base-pairing; contacting the resulting duplex with a mutant nucleic acid repair enzyme or complex thereof; and detecting binding between the nucleic acid duplex and the mutant nucleic acid repair enzyme or complex thereof. The amount of enzyme bound is used to assess the presence or quantity of the mutation. Depending upon the mutant enzyme selected, the identity of the mismatch may be determined as well. The nucleic acid strand to be tested and the complementary wild-type nucleic acid strand, preferably, are DNA strands.

The mutation to be detected encompasses any mutation. Of particular interest are mutations associated with diseases and disorders, or infections, including infection by a pathological agent. In such instances, the methods are used for prognosis or diagnosis of the presence or severity of the disease, disorder or infection.

In other embodiments, the mutation to be detected is associated with a cancer, an immune system disease or disorder, a metabolism disease or disorder, a muscle and bone disease or disorder, a nervous system disease or disorder, a signal disease or disorder or a transporter disease or disorder. Examples of cancers that can be detected by the methods herein include, but are not limited to, breast cancer, Burkitt lymphoma, colon cancer, small cell lung carcinoma, melanoma, multiple endocrine neoplasia (MEN), neurofibromatosis, p53-associated tumor, pancreatic carcinoma, prostate cancer, Ras-associated tumor, retinoblastoma and Von-Hippel Lindau disease (VHL).

Examples of immune system diseases and disorders include, but are not limited to, autoimmune polyglandular syndrome type I (APS 1, also called APECED), inflammatory bowel disease (IBD), DiGeorge syndrome, familial Mediterranean fever (FMF) and severe combined immunodeficiency (SCID). Examples of metabolic disease and disorders, include, acquired disease and inborn errors of metabolism. Such diseases and disorders include, but are not limited to, adrenoleukodystrophy (ALD), atherosclerosis, Gaucher disease, gyrate atrophy of the choroid, diabetes, obesity, paroxysmal nocturnal hemoglobinuria (PNH), phenylketonuria (PKU), Refsum disease and Tangier disease (TD).

Exemplary muscle and bone diseases and disorders include, but are not limited to, Duchenne muscular dystrophy (DMD), Ellis-Van Creveld syndrome (chondroectodermal dysplasia), Marfan syndrome and myotonic dystrophy. Examples of nervous system diseases and disorders include, but are not limited to, Alzheimer disease (AD), amyotrophic lateral sclerosis (ALS), Angelman syndrome (AS), Charcot-Marle-tooth disease (CMT), epilepsy, tremor, fragile X syndrome, Friedreich's ataxia (FRDA), Huntington disease (HD), Niemann-Pick, Parkinson disease, Prader-Willi syndrome (PWS), spinocerebellar atrophy and Williams syndrome. Examples of signal diseases and disorders include, but are not limited to, ataxia telangiectasia (A-T), male pattern baldness, acne, hirsutism, Cockayne syndrome, glaucoma, mammals with abnormal secondary sexual characteristics, tuberous sclerosis, Waardenburg syndrome (WS) and Werner syndrome (WRN).

Exemplary transporter diseases and disorders include, but are not limited to, cystic fibrosis (CF), diastrophic dysplasia (DTD), long-QT syndrome (LQTS), Menkes' syndrome, pendred syndrome, adult polycystic kidney disease (APKD), Wilson's disease and Zellweger syndrome.

Other examples of the diseases and disorders that can be detected by the present methods include, but are not limited to, a disease or disorder associated with an androgen receptor mutation, tetrahydrobiopterin deficiencies, X-Linked agammaglobulinemia, a disease or disorder associated with a factor VII mutation, anemia, a disease or disorder associated with a glucose-6-phosphate mutation, the glycogen storage disease type II (Pompe Disease), hemophilia A, a disease or disorder associated with a hexosaminidase A mutation, a disease or disorder associated with a human type I or type III collagen mutation, a disease or disorder associated with a rhodopsin or RDS mutation, a disease or disorder associated with a L1CAM mutation, a disease or disorder associated with a LDL receptor mutation, a disease or disorder associated with an ornithine transcarbamylase mutation, a disease or disorder associated with a PAX6 mutation and a disease or disorder associated with a von Willebrand factor mutation.

The methods herein can also be used to detect infections and pathogens associated therewith. Such infection include, but are not limited to, infections caused by a virus, a eubacteria, an archaebacteria and a eukaryotic pathogen. The infections can be caused by a mutant strain of a virus, an eubacteria, an archaebacteria or an eukaryotic pathogen.

Exemplary viruses include, but are not limited to, a Delta virus, a dsDNA virus, a retroid virus, a satellite virus, a ssDNA virus, a ssRNA negative-strand virus, ssRNA positive-strand virus (no DNA stage) and a bacteriophage. Eubacteria include, but are not limited to, a green bacteria, a flavobacteria, a spirochetes, a purple bacteria, a gram-positive bacteria, a gram-negative bacteria, a cynobacteria, a deinococci and a thermotogale. Archaebacteria include, but are not limited to, an extreme halophile, a methanogen and an extreme thermophile. Eukaryotic pathogens include, but are not limited to, a fungi such as a yeast, a ciliate, a cellular slime mode, a flagellate and a microsporidia.

In the above methods for detecting mutations, the hybridization between the strand of a nucleic acid having or suspected of having a mutation and the complementary strand of a wild-type nucleic acid can be facilitated by a recombinase. Recombinase, include, but are not limited to, Cre recombinase, RAG-1 V(D)J recombinase, Endonuclease II of coliphage T4 and F1p recombinase.

Also provided herein are methods for detecting polymorphisms, including single nucleotide polymorphisms (SNPs) at a gene locus or loci. The methods include hybridizing a target strand of a nucleic acid molecule that includes the locus to be tested with a complementary reference strand of a nucleic acid that has a known allele of the locus. Allelic identity between the target and the reference strand results in the formation of a nucleic acid duplex without an abnormal base-pairing, and allelic difference between the target and the reference strands results in the formation of a nucleic acid duplex with an abnormal base-pairing. The resulting nucleic acid duplex formed is contacted with a mutant nucleic acid repair enzyme or complex thereof that has binding affinity for the abnormal base-pairing in the duplex but has attenuated catalytic activity. Binding between the nucleic acid duplex and the mutant DNA repair enzyme or complex thereof is detected. The presence of a polymorphism is then assessed. Any polymorphism may be detected by these methods, and include, but are not limited to, a variable nucleotide type polymorphism (“VNTR”), a single nucleotide polymorphism (SNP), preferably a human genome SNP.

In the above methods for detecting polymorphisms, the hybridization between the target strand of a nucleic acid comprising a locus to be tested and the complementary reference strand of a nucleic acid comprising a known allele of the locus can be facilitated by a recombinase. Recombinases include, but are not limited to, Cre recombinase, RAG-1 V(D)J recombinase, Endonuclease II of coliphage T4 or F1p recombinase.

Methods for selecting, purifying or removing a nucleic acid duplex containing one or more abnormal base-pairings in a population of nucleic acid duplexes are also provided. These methods are performed by contacting a population of nucleic acid duplexes having or suspected of including an abnormal base-pairing with a mutant DNA repair enzyme or complex thereof, where the mutant DNA repair enzyme or complex thereof has binding affinity for the abnormal base-pairing in the duplex but has attenuated catalytic activity, whereby the nucleic acid duplex containing one or more abnormal base-pairing binds to the mutant DNA. repair enzyme or complex thereof to form a binding complex. The resulting complex can be removed from the population. The mutant enzyme can be presented and introduced into the population on a solid support, whereby duplexes in the population that contain an abnormal base pairing to which the mutant enzyme binds will bind to the enzyme on the solid support. In a specific embodiment, the population of nucleic acid duplexes contains DNA:DNA, DNA:RNA or RNA:RNA duplexes. The abnormal base-pairing to be removed includes a base-pair mismatch, a base insertion, a base deletion or a pyrimidine dimer. Preferably, the base-pair mismatch to be removed is a single base-pair mismatch.

The population of nucleic acid duplexes is produced by an amplification, such as by a polymerase chain reaction or a reaction using reverse transcription and subsequent DNA amplification of one or more expressed RNA sequences.

Further provided herein are methods for detecting and localizing an abnormal base-pairing in a nucleic acid duplex. These methods are performed by contacting a nucleic acid duplex having or suspected of having an abnormal base-pairing with a mutant DNA repair enzyme or complex thereof, where the mutant DNA repair enzyme or complex thereof has binding affinity for the abnormal base-pairing in the duplex but has attenuated catalytic activity, whereby the nucleic acid duplex containing an abnormal base-pairing binds to the mutant DNA repair enzyme or complex thereof to form a binding complex; subjecting the nucleic acid duplex to hydrolysis with an exonuclease under conditions such that the binding complex blocks hydrolysis; and then determining the location within the nucleic acid duplex protected from the hydrolysis, thereby detecting and localizing the abnormal base-pairing in the nucleic acid duplex. In a specific embodiment, the nucleic acid duplex to be assayed is a DNA:DNA, a DNA:RNA or a RNA:RNA duplex. Preferably, the nucleic acid duplex to be assayed is a DNA:DNA duplex. The abnormal base-pairing to be detected and localized is a base-pair mismatch, a base insertion, a base deletion or a pyrimidine dimer. Preferably, the base-pair mismatch to be detected and localized is a single base-pair mismatch. Exemplary exonucleases, include, but are not limited to, BAL-31 exonuclease, exonuclease III, Mung Bean exonuclease and Lambda exonuclease.

In the above methods for detecting abnormal base-pairings, mutations, and polymorphisms, and the methods for localizing and removing abnormal base-pairings, the mutant DNA repair enzyme or complex thereof can be labelled. Preferably, the mutant DNA repair enzyme or complex thereof used therein is labelled, with a detectable label, such as biotin, a bioluminescence generating reagent, such as a luciferin or luciferase, a fluorescence label or a radiolabel, and the binding between the abnormal base-pairing and the labelled mutant DNA repair enzyme or complex thereof is detected, such as with a streptavidin labeled enzyme, generation of bioluminescence by contacting with luciferin or luciferase, or detection of the fluorescence or bound radioactivity. Labeled enzymes, include but are not limited to, a peroxidase, a urease, an alkaline phosphatase, a luciferase and a glutathione S-transferase. The mutant repair enzyme may also be prepared as a conjugate, such as a chemical conjugate or fusion protein, with a detectable label or tag or enzyme or enzyme substrate.

In the above methods for detecting abnormal base-pairings, mutations, and polymorphisms, and the methods for localizing and removing abnormal base-pairings, the target nucleic acid strand to be assayed, the reference nucleic acid strand, the target nucleic acid duplex to be assayed, the nucleic acid duplex formed via hybridization of the target strand and the reference strand, or the mutant DNA repair enzyme or complex thereof can be immobilized on the surface of a support, either directly or indirectly, such as via a linker. Preferably, the support used is an insoluble support such as a silicon chip. Support geomatrices, include, but are not limited to, beads, pellets, disks, capillaries, hollow fibers, needles, solid fibers, random shapes, thin films, membranes and chips. Also more preferably, the nucleic acid strand, the nucleic acid duplex or the mutant DNA repair enzyme or complex thereof is immobilized in an array or a well format on the surface of a support. Immobilization can be effected via covalent, ionic or other interactions, and can be direct or via a suitable linking moiety, such as heterobifunctional linker.

In the above methods, one sample can be assayed at one time, but preferably, the assays are performed in high-throughput format where a plurality of samples are assayed simultaneously.

In the above methods, the target nucleic acid strand or target nucleic acid duplex can be synthesized or derived from a natural source. In a specific embodiment, the target strand of a nucleic acid or the target nucleic acid duplex is isolated from a natural sample, e.g., a biosample. Preferably, the sample is a body fluid or a biological tissue. More preferably, the body fluid is urine, blood, plasma, serum, saliva, semen, stool, sputum, cerebral spinal fluid, tears, mucus or amniotic fluid. Also more preferably, the biological tissue is connective tissue, epithelium tissue, muscle tissue, nerve tissue, organs, tumors, lymph nodes, arteries and individual cell(s).

Mutant enzymes that substantially retain binding affinity and specificity, but that have reduced catalytic activity are also provided. Compositions containing the mutant enzymes, kits and articles of manufacture containing the mutant enzymes are also provided. In particular a mutant nucleic repair enzyme that retains binding affinity for abnormal base pairs in a nucleic acid duplex, but has reduced catalytic activity compared to wild type, such that the mutant enzyme quantitatively retain a duplex on a solid support, with a Ka of at least about 10⁷, more preferably 10⁸, most preferably 10⁹ M or higher.

The mutant enzymes include a mutant mutL is an E. Coli mutant mutL having a mutation selected from E29K, E32K, A37T, D58N, G60S, G93D, R95C, G96S, G96D, S112L A16T, A16V, P305L, H308Y, G238D, S106F and A271V; a mutant MLH1 that is a human mutant MLH1 having a mutation selected from among of P28L, M35R, S44F, G67R, I68N, I107R, T117R, T117M, R265H, V185G and G224D; a mutant mutS that has a mutation in its catalytic site, dimerization site, mutL interaction site or combinations thereof; a mutM that has a mutation in its catalytic site, mutY interaction site or a combination thereof, including an E. Coli mutant mutM having a K57G or K57R mutation; a mutant mutY that has a mutation in its catalytic site, mutM interaction site or a combination thereof, in an E. Coli mutant mutY having a mutation selected from among E37S, V45N, G116D, D138N and K142A; or is a mutant uvrD that has a mutation in its catalytic site, ATP binding site or a combination thereof, including an E. mutant uvrD having a mutation selected from among K35M, D220NE221Q, E221Q and Q251E; a mutant MSH2 that has a mutation in its catalytic site, ATP binding site, ATPase site or a combination thereof, including an S. cerevisiae mutant MSH2 having a G693D or a G855D mutation and a human mutant MSH2 having a fragment encoding 195 amino acids within the C-terminal domain of hMSH-2 or having a K675R mutation; a mutant MSH6 that has a mutation in its catalytic site, ATP binding site, ATPase site or any combination thereof, including a human mutant MSH6 having a K1140R mutation, a complex of a human mutant MSH2 having a K675R mutation and a human mutant MSH6 having a K1140R mutation; and a mutant T4 endonuclease V that has a E23Q mutation.

Solid supports, such as silicon chips, containing one or a plurality of the same or of different mutant enzymes conjugated, either directly or indirectly, thereto, are also provided.

Kits and articles of manufacture for detecting abnormal base-pairings, mutations, polymorphisms, and for localizing and/or removing abnormal base-pairings are provided herein. The combinations, kits and articles of manufacture typically include one or more of the mutant enzymes, which may be in a composition or provided in an array or in combination with a support with linked nucleic acids.

### BRIEF DESCRIPTION OF DRAWINGS

FIG. 1 illustrates an exemplary SNP-STE candidate screening. Mutant SNP-STE proteins were screened by microplate assay as described in the Example section for mispaired DNA binding proficiencies relative to the “wild-type” or unmutagenized/unmodified enzyme. The data was analyzed and is represented as fold increases over correctly paired DNA. F3=E673K; and F18=H728A.

FIG. 2 shows that further optimization of assay conditions for specific mispair recognition by SNP-STE F18 can provide very high discrimination compared to correctly paired DNA.

### DETAILED DESCRIPTION

Table of Contents

| A. | DEFINITIONS | |
|---|---|---|
| B. | METHODS FOR DETECTING ABNORMAL BASE-PAIRING | |
| | 1. | Mutant DNA repair enzyme or complex thereof |

| | |
|---|---|
| a. | Nucleic acids encoding DNA repair enzymes |
| b. | Selecting and producing mutant DNA repair enzymes |
| c. | Mutant mutL or MLH1 |
| d. | Mutant MutS |
| e. | Mutant MutM |
| f. | Mutant MutY |
| g. | Mutant uvrD |
| h. | Mutant MSH2 |
| i. | Mutant MSH6 |
| j. | Mutant T4 endonuclease V |
| k. | Mutant MSH3 |
| l. | Mutant alkA |
| m. | Mutant Exonuclease I |
| n. | Mutant fen1 |
| o. | Mutant rpa |
| p. | Mutant pcna |
| q. | Mutant Replication factor C |
| r. | Mutant Uracil DNA glycosylase |
| s. | Mutant Thymidine DNA glycosylase |
| t. | Mutant dam |

| | 2. | Detecting the Binding of the Mutant Enzyme |
|---|---|---|
| C. | METHODS FOR DETECTING MUTATIONS IN NUCLEIC | |
| | ACIDS FOR PROGNOSIS AND DIAGNOSIS OF DISEASES, | |
| | DISORDERS AND INFECTIONS | |
| | 1. | Cancer |

| | |
|---|---|
| a. | Breast cancer |
| b. | Burkitt lymphoma |
| c. | Colon cancer |
| d. | Small cell lung carcinoma |
| e. | Melanoma carcinoma |
| f. | Multiple endocrine neoplasia |
| g. | Neurofibromatosis |
| h. | Cancer associated with p53 mutation |
| i. | Pancreatic carcinoma |
| j. | Prostate cancer |
| k. | Cancer associated with Ras oncogene |
| l. | Retinoblastoma |
| m. | Von-Hippel Lindau syndrome |

| | |
|---|---|
| 2. | Immune system diseases and disorders |

| | |
|---|---|
| a. | Autoimmune polyglandular syndrome type I |
| b. | Inflammatory bowel disease |
| c. | DiGeorge syndrome |
| d. | Familial Mediterranean fever |
| e. | Severe combined immunodeficiency |

| | |
|---|---|
| 3. | Metabolism system diseases and disorders |

| | |
|---|---|
| a. | Adrenoleukodystrophy |
| b. | Atherosclerosis |
| c. | Gaucher disease |
| d. | Gyrate atrophy of the choroid |
| e. | Diabetes |
| f. | Obesity |
| g. | Paroxysmal nocturnal hemoglobinuria |
| h. | Phenylketonuria |
| i. | Refsum disease |
| j. | Tangier disease |

| | |
|---|---|
| 4. | Muscle and bone diseases and disorders |

| | |
|---|---|
| a. | Duchenne muscular dystrophy |
| b. | Ellis-Van Creveld syndrome |
| c. | Marfan syndrome |
| d. | Myotonic dystrophy |

| | |
|---|---|
| 5. | Nervous system diseases and disorders |

| | |
|---|---|
| a. | Alzheimer disease |
| b. | Amyotrophic lateral sclerosis |
| c. | Angelman syndrome |
| d. | Charcot-Marle-tooth disease |
| e. | Epilepsy |
| f. | Tremor |
| g. | Fragile X syndrome |
| h. | Friedreich's ataxia |
| i. | Huntington disease |
| j. | Niemann-Pick |
| k. | Parkinson disease |
| l. | Spinocerebellar atrophy |
| m. | Williams syndrome |

| | |
|---|---|
| 6. | Signal diseases and disorders |

| | |
|---|---|
| a. | Ataxia telangiectasia |
| b. | Male pattern baldness, acne or hirsutism |
| c. | Cockayne syndrome |
| d. | Glaucoma |
| e. | Abnormal secondary sexual characteristics |
| f. | Tuberous sclerosis |
| h. | Waardenburg syndrome |
| i. | Werner syndrome |

| | |
|---|---|
| 7. | Transporter diseases and disorders |

| | |
|---|---|
| a. | Cystic fibrosis |
| b. | Diastrophic dysplasia |
| c. | Long-QT syndrome |
| d. | Menkes' syndrome |
| e. | Pendred syndrome |
| f. | Adult polycystic kidney disease |
| g. | Wilson's disease |
| h. | Zellweger syndrome |

| | 8. | Infections |
|---|---|---|
| D. | METHODS FOR DETECTING POLYMORPHISMS | |
| E. | METHODS FOR REMOVING NUCLEIC ACID DUPLEX | |
| | WITH ABNORMAL BASE-PAIRING | |
| F. | METHODS FOR DETECTING AND LOCALIZING ABNORMAL | |
| | BASE-PAIRING IN NUCLEIC ACID DUPLEX | |
| G. | LABELLING OF MUTANT DNA REPAIR ENZYMES | |
| | 1. | Conjugation |

| | |
|---|---|
| a. | Fusion proteins |
| b. | Chemical conjugation |

| | |
|---|---|
| 1) | Heterobifunctional cross-linking reagents |
| 2) | Exemplary Linkers |

| | |
|---|---|
| a) | Acid cleavable, photocleavable and |
| | heat sensitive linkers |
| b) | Other linkers for chemical conjugation |
| c) | Peptide linkers |

| | |
|---|---|
| 2. | Selection of facilitating agents |

| | |
|---|---|
| a. | Protein binding moieties |

| | |
|---|---|
| 1) | Interaction trap/two-hybrid system |
| 2) | Phage-based expression cloning |
| 3) | Detection of protein-protein interactions |

| | |
|---|---|
| b. | Epitope tags |
| c. | IgG binding proteins |

| | |
|---|---|
| 1) | pEZZ 18 Protein A gene fusion vector |
| 2) | pRIT2T Protein A gene fusion vector |
| 3) | The IgG Sepharose 6 fast flow system |

| | |
|---|---|
| d. | â-galactosidase fusion proteins |
| e. | Nucleic acid binding moieties |

| | |
|---|---|
| 1) | DNA binding proteins |
| 2) | RNA binding proteins |
| 3) | Preparation of nucleic acid binding proteins |
| 4) | Assays for identifying nucleic acid binding proteins |

| | |
|---|---|
| a) | Mobility shift DNA-binding assay |
| b) | Basic mobility shift assay procedure |
| c) | Competition mobility shift assay |
| d) | Antibody supershift assay |
| e) | Methylation and uracil interference assay |

| | |
|---|---|
| 1) | Methylation interference assays |
| 2) | Uracil interference assay |
| 3) | DNase I footprint analysis |
| 4) | Screening a ëgt11 expression library with |
| | recognition-site DNA |
| 5) | Rapid separation of protein-bound |
| | DNA from free DNA |

| | |
|---|---|
| f. | Lipid binding moieties |
| g. | Polysaccharide binding moieties |
| h. | Metal binding moieties |
| i. | Other facilitating agents |

| | |
|---|---|
| 1) | Peroxidase |
| 2) | urease |
| 3) | Alkaline phosphatase |
| 4) | Luciferase |
| 5) | Glutathione S-transferase |
| 6) | Defense proteins |
| 7) | Fluorescent moieties |

| H. | IMMOBILIZATION OF MUTANT ENZYMES | |
|---|---|---|
| | AND NUCLEIC ACIDS | |
| | 1. | Immobilization of the mutant enzymes |
| | 2. | Immobilization of nucleic acids |
| I. | HIGH-THROUGHPUT ASSAY FORMAT | |
| | 1. | High-throughput assay instrumentation and capabilities |
| | 2. | Detection technologies |

| | |
|---|---|
| a. | Radiochemical methods |
| b. | Non-isotopic detection methods |

| | |
|---|---|
| 1) | Colorimetry and luminescence |
| 2) | Resonance energy transfer |
| 3) | Time-resolved fluorescence |
| 4) | Cell-based fluorescence assays |
| 5) | Fluorescence polarization |
| 6) | Fluorescence correlation spectroscopy |

| | |
|---|---|
| 3. | Miniaturization |

| | |
|---|---|
| J. | SAMPLE COLLECTION |
| K. | COMBINATIONS, KITS AND ARTICLES OF MANUFACTURE |
| L. | EXAMPLES |

### A. Definitions

Unless defined otherwise, all technical and scientific terms used herein have the same meaning as is commonly understood by one of ordinary skill in the art to which this invention belongs. All patents, applications, published applications and other publications and sequences from GenBank and other data bases referred to herein are incorporated by reference in their entirety.

As used herein, “base-pairing” refers to the specific hydrogen bonding between purines and pyrimidines in double-stranded nucleic acids. In DNA, the pairs are adenine (A) and thymine (T), and guanine (G) and cytosine (C), while in RNA they are adenine (A) and uracil (U), and guanine (G) and cytosine (C). Base-pairing leads to the formation of a nucleic acid double helix from two complementary single strands.

As used herein, “nucleic acid duplex having abnormal base-pairing” refers to a nucleic acid duplex wherein there exists base-pair mismatch, i.e., any base-pairing other than any of the normal A:T(U) and C:G pairs, a single-stranded loop region due to the addition of extra-nucleotide(s) in one strand and/or deletion of nucleotide(s) in the complementary strand, or a combination thereof. Non-limiting examples of base-pair mismatch include A:A, A:C, A:G, C:C, C:T, G:G, G:T, T:T, C:U, G:U, T:U, U:U, 5-formyluracil (fU):G, 7,8-dihydro-8-oxo-guanine (8-oxoG):C, 8-oxoG:A.

As used herein, “enzyme” refers to a protein specialized to catalyze or promote a specific metabolic reaction. Generally, enzymes are catalysts, but for purposes herein, such “enzymes” include those that would be modified during a reaction. Since the enzymes are modified to eliminate or substantially eliminate catalytic activity, they will not be so-modified during a reaction.

As used herein, “DNA repair” refers to a process wherein the sites of mutations in DNA (DNA:DNA duplexes, DNA:RNA and, for purposes herein, also RNA:RNA duplexes) are recognized by a nuclease that excises the damaged or mutated region from the nucleic acid; and then further enzymes or enzymatic activities synthesize a replacement portion of a strand(s) so that the original sequence is preserved.

As used herein, “DNA repair enzyme” refers to an enzyme that corrects errors in nucleic acid structure and sequence, i.e., recognizes, binds and corrects abnormal base-pairing in a nucleic acid duplex. DNA repair enzyme functions to protect genetic information against environmental damage and replication errors. Examples of DNA repair enzyme include mutH, mutL, mutM, mutS, mutY, uvrD, dam, thymidine DNA glycosylase (TDG), mismatch-specific DNA glycosylase (MUG), AlkA, MLH1, MSH2, MSH3, MSH6, Exonuclease I, T4 endonuclease V, FEN1 (RAD27), DNA polymerase ä, DNA polymerase å, RPA, PCNA and RFC. It is intended that DNA repair enzymes encompasses enzymes with conservative amino acid substitutions that do not substantially alter repair activity. Suitable conservative substitutions of amino acids are known to those of skill in this art and may be made generally without altering the biological activity of the resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Bejacmin/Cummings Pub. co., p.224).

Such substitutions are preferably made in accordance with those set forth in TABLE 1 as follows:

TABLE 1

| Original residue | Conservative substitution |
|---|---|
| Ala (A) | Gly; Ser |
| Arg (R) | Lys |
| Asn (N) | Gln; His |
| Cys (C) | Ser |
| Gln (Q) | Asn |
| Glu (E) | Asp |
| Gly (G) | Ala; Pro |
| His (H) | Asn; Gln |
| Ile (I) | Leu; Val |
| Leu (L) | Ile; Val |
| Lys (K) | Arg; Gln; Glu |
| Met (M) | Leu; Tyr; Ile |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu |

Other substitutions are also permissible and may be determined empirically or in accord with known conservative substitutions.

As used herein, the “amino acids,” which occur in the various amino acid sequences appearing herein, are identified according to their well-known, three-letter or one-letter abbreviations. The nucleotides, which occur in the various DNA fragments, are designated with the standard single-letter designations used routinely in the art.

As used herein, “a mutant DNA repair enzyme” (used interchangeably with “abnormal base-pairing trapping enzyme”) refers to a mutant form of an enzyme that can repair errors in duplexes. The mutant, however, has binding affinity for the abnormal base-pairing in a nucleic acid duplex but lacks the catalytic activity whereby the abnormal pairing is excised. The mutant form of the repair enzyme that retains sufficient binding affinity for the abnormal base-pairing to be detected in the process or method, particularly assay, of interest. Typically this is at least about 10%, preferably at least about 50% binding affinity for the abnormal base-pairing, compared to its wildtype counterpart. Preferably, such mutant DNA repair enzyme retains 60%, 70%, 80%, 90%, 100% binding affinity for the abnormal base-pairing compared to its wildtype counterpart, or has a higher binding affinity than its wildtype counterpart. Such mutant DNA repair enzyme is herein referred to as an “abnormal base-pairing trapping enzyme”, i.e., a molecule that specifically binds to a selected abnormal base-pairing, but does not catalyze conversion thereof. The mutant enzyme possess substantially reduced such that the binding of the enzyme to the duplex can be detected. This is typically no more than about 50%, preferably no more than 20%, more preferably no more than about 10%, of the wild-type catalytic activity.

As used herein the term “assessing” is intended to include quantitative and qualitative determination in the sense of obtaining an absolute value for the amount or concentration of the abnormal base-pairing present in the sample, and also of obtaining an index, ratio, percentage, visual or other value indicative of the level of abnormal base-pairing in the sample. Assessment may be direct or indirect and the chemical species actually detected need not of course be the abnormal base-pairing itself but may for example be a derivative thereof or some further substance.

As used herein, “attenuated catalytic activity” refers to a mutant DNA repair enzyme that retains sufficiently reduced catalytic activity to be useful as a “pseudo-antibody”, i.e., a molecule used in place of an antibody in immunoassay formats. The precise reduction in catalytic activity for use in the assays can be empirically determined for each assay. Typically, the enzyme will retain less than about 50% of one of its catalytic activities or less than 50% of its overall catalytic activities compared to its wildtype counterpart. Preferably, a mutant DNA repair enzyme retains less than 40%, 30%, 20%, 10%, 1%, 0. 1%, or 0.01% of one of its catalytic activities or its overall catalytic activities compared to its wildtype counterpart. More preferably, a mutant DNA repair enzyme lacks detectable level of one of its catalytic activities or its overall catalytic activities compared to its wildtype counterpart. In instances in which catalytic activity is retained and/or a further reduction thereof is desired, the contacting step can be effected in the presence of a catalysis inhibitor. Such inhibitors, include, but are not limited to, heavy metals, chelators or other agents that bind to a co-factor required for catalysis, but not for binding, and other such agents.

As used herein, “mutH” refers to a procaryotie latent endonuclease that incises the transiently unmethylated strands of hemimethylated 5′-GATC-3′ sequences. It is intended to encompass mutH with conservative amino acid substitutions that do not substantially alter its activity.

As used herein, “mutS” refers to a procaryotic DNA-mismatch binding protein that can bind to a variety of mispaired bases and small (1-5 bases) single-stranded loops. It is intended to encompass mutS with conservative amino acid substitutions that do not substantially alter its activity.

As used herein, “mutL” refers to a procaryotic protein that couples abnormal base-pairing recognition by mutS to mutH incision at the 5′-GATC-3′ sequences in an ATP-dependent manner. It is intended to encompass mutL with conservative amino acid substitutions that do not substantially alter its activity.

As used herein, “uvrD” refers to a procaryotic DNA helicase II that unwinds DNA in an ATP-dependent manner. It is intended to encompass uvrD with conservative amino acid substitutions that do not substantially alter its activity.

As used herein, “dam” refers to a procaryotic adenine methyltransferases that plays a role in coordinating DNA replication initiation, DNA mismatch repair and the regulation of expression of some genes. It is intended to encompass dam with conservative amino acid substitutions that do not substantially alter its activity.

As used herein, “mutM” refers to an 8-oxoguanine DNA glycosylase that removes 7,8-dihydro-8-oxoguanine (8-oxoG) and formamido pyrimidine (Fapy) lesions from DNA. It is intended to encompass mutM with conservative amino acid substitutions that do not substantially alter its activity.

As used herein, “mutY” refers to an adenine glycosylase that is involved in the repair of 7,8-dihydro-8-oxo-2′-deoxyguanosine (OG):A and G:A mispairs in DNA. It is intended to encompass mutY with conservative amino acid substitutions that do not substantially alter its activity.

As used herein, “TDG” refers to a thymine-DNA glycosylase that corrects G/T mispairs to G/C pairs. It is intended to encompass TDG with conservative amino acid substitutions that do not substantially alter its activity.

As used herein, “MUG” refers to a uracil-DNA glycosylase that corrects G/T and G/U mispairs to G/C pairs. It is intended to encompass MUG with conservative amino acid substitutions that do not substantially alter its activity.

As used herein, “AlkA” refers to a 3-methyladenine DNA glycosylase II that corrects 5-formyluracil (fU)/G mispairs. It is intended to encompass AlkA with conservative amino acid substitutions that do not substantially alter its activity.

As used herein, “MSH2” refers to the common component of the eukaryotic DNA repair complex MSH2-MSH6 (MutSá), which repairs base-base mispairs and insertion/deletion mispairs up to 12 unpaired bases, and the eukaryotic DNA repair complex MSH2-MSH3 (MutSβ), which repairs insertion/deletion mispairs having two or more unpaired bases but does not repair single base insertion/deletion mispairs. As used herein, “MSH3” refers to the unique component of the “MSH2-MSH3” complex and “MSH6” refers to the unique component of the “MSH2-MSH6” complex. It is intended to encompass MSH2, MSH3 and MSH6 with conservative amino acid substitutions that do not substantially alter its respective activity.

As used herein, “MLH1” and “PMS1” (PMS2 in humans) refers to the components of the eukaryotic mutL-related protein complex, MLH1-PMS 1, that interacts with MSH2-containing complexes bound to mispaired bases. It is intended to encompass MLH1 and PSM1 with conservative amino acid substitutions that do not substantially alter its respective activity.

As used herein, “exonuclease I” refers to an eukaryotic 5′→3′ exonuclease that has a preference for degrading double-stranded DNA. Exonuclease I involves in the DNA repair via its interaction with MSH2. It is intended to encompass exonuclease I with conservative amino acid substitutions that do not substantially alter its respective activity.

As used herein, “T4 endonuclease V (EndoV)” refers to a base excision repair enzyme that removes thymine dimers (TD) from damaged DNA. It is intended to encompass T4 endonuclease V with conservative amino acid substitutions that do not substantially alter its respective activity.

As used herein, “FEN1 (rad27)” refers to an evolutionarily conserved component of DNA replication complex. FEN1 processes Okazaki fragments during replication and is involved in base excision repair. FEN1 removes the last primer ribonucleotide on the lagging strand and it cleaves a 5′ flap that may result from strand displacement during replication or during base excision repair. It is intended to encompass FEN1 (rad27) with conservative amino acid substitutions that do not substantially alter its respective activity.

As used herein, “replication protein A (RPA)” refers to a heterotrimeric single-stranded DNA-binding protein that is highly conserved in eukaryotes. RPA plays essential roles in many aspects of nucleic acid metabolism, including DNA replication, nucleotide excision repair, and homologous recombination. It is intended to encompass RPA with conservative amino acid substitutions that do not substantially alter its respective activity.

As used herein, “proliferating cell nuclear antigen A (PCNA)” refers to a DNA sliding clamp for DNA polymerase delta and is an essential component for eukaryotic chromosomal DNA replication. PCNA interacts with multiple partners, involved, for example, in Okazaki fragment joining, DNA repair, DNA methylation and chromatin assembly. PCNA is required for nucleotide excision repair, base excision repair and mismatch repair. DNA polymerases, RFC and PCNA recognize 3′ ends of gaped DNA and fill the gaps by the same mechanism as used for joining of Okazaki fragments. It is intended to encompass PCNA with conservative amino acid substitutions that do not substantially alter its respective activity.

As used herein, “replication factor C (RFC)” refers to a five-subunit protein complex required for coordinate leading and lagging strand DNA synthesis during S phase and DNA repair in eukaryotic cells. RFC functions to load the proliferating cell nuclear antigen (PCNA), a processivity factor for polymerases delta and epsilon, onto primed DNA templates. This process, which is ATP-dependent, is carried out by 1) recognition of the primer terminus by RFC, 2) binding to and disruption of the PCNA trimer, and then 3) topologically linking the PCNA to the DNA. It is intended to encompass RFC with conservative amino acid substitutions that do not substantially alter its respective activity.

As used herein, “DNA polymerase å” refers to a mammalian DNA polymerase that has a tightly associated 3′→5′ exonuclease activity. DNA polymerase a is required at least for the repair synthesis of UV-damaged DNA. It is intended to encompass DNA polymerase å with conservative amino acid substitutions that do not substantially alter its respective activity.

As used herein, “DNA polymerase ä” refers to a DNA polymerase that plays important roles in DNA replication, nucleotide excision repair, base excision repair and VDJ recombination. The function of DNA polymerase ä must be considered in the context of two other factors, PCNA and RFC, two protein complexes that build together the moving platform for DNA polymerase ä. This moving platform provides an important framework for dynamic properties of an accurate DNA polymerase ä, such as its recruitment when its function is needed, the facilitation of DNA polymerase ä binding to the primer terminus, the increase in DNA polymerase ä processivity, the prevention of non-productive binding of the DNA polymerase ä to single-stranded DNA, the release of DNA polymerase ä after DNA synthesis and the bridging of DNA polymerase ä interactions to other replication proteins. It is intended to encompass DNA polymerase ä with conservative amino acid substitutions that do not substantially alter its respective activity.

As used herein, “DNA polymerase III holoenzyme” refers to an enzyme that contains two DNA polymerases embedded in a particle with 9 other subunits. This multisubunit DNA polymerase is the E. Coli chromosomal replicase, and it has several special features that distinguish it as a replicating machine. For example, one of its subunits is a circular protein that slides along DNA while clamping the rest of the machinery to the template. Other subunits act together as a matchmaker to assemble the ring onto DNA. Overall, E. Coli DNA polymerase III holoenzyme is very similar in structure and function to the chromosomal replicases of eukaryotes, from yeast all the way up to humans.

As used herein, “mutation” refers to change(s) in the nucleic acid length and/or sequence in an organism, which may arise in any of a variety of different ways, e.g., frame-shift mutation, non-sense mutation or missense mutation.

As used herein, “disease or disorder” refers to a pathological condition in an organism resulting from, e.g., infection or genetic defect, and characterized by identifiable symptoms.

As used herein, “cancer” refers to a pathological condition that occurs when cell division gets out of control. Usually, the timing of cell division is under strict constraint, involving a network of signals that work together to say when a cell can divide, how often it should happen and how errors can be fixed. Mutations in one or more of the nodes in this network can trigger cancer, be it through exposure to some environmental factor (e.g., tobacco smoke) or because of a genetic predisposition, or both. Usually, several cancer-promoting factors have to add up before a person will develop a malignant growth: with some exceptions, no one risk alone is sufficient. The predominant mechanisms for the cancers are (i) impairment of a DNA repair pathway (ii) the transformation of a normal gene into an oncogene and (iii) the malfunction of a tumor suppressor gene.

As used herein, “an immune system disease or disorder” refers to a pathological condition caused by a defect in the immune system. The immune system is a complex and highly developed system, yet its mission is simple: to seek and kill invaders. If a person is born with a severely defective immune system, death from infection by a virus, bacterium, fungus or parasite will occur. In severe combined immunodeficiency, lack of an enzyme means that toxic waste builds up inside immune system cells, killing them and thus devastating the immune system. A lack of immune system cells is also the basis for DiGeorge syndrome: improper development of the thymus gland means that T cell production is diminished. Most other immune disorders result from either an excessive immune response or an ‘autoimmune attack’. For example, asthma, familial Mediterranean fever and Crohn disease (inflammatory bowel disease) all result from an over-reaction of the immune system, while autoimmune polyglandular syndrome and some facets of diabetes are due to the immune system attacking ‘self’ cells and molecules. A key part of the immune system's role is to differentiate between invaders and the body's own cells—when it fails to make this distinction, a reaction against ‘self’ cells and molecules causes autoimmune disease.

As used herein, “a metabolism disease or disorder” refers to a pathological condition caused by errors in metabolic processes. Metabolism is the means by which the body derives energy and synthesizes the other molecules it needs from the fats, carbohydrates and proteins we eat as food, by enzymatic reactions helped by minerals and vitamins. There is a significant level of tolerance of errors in the system: often, a mutation in one enzyme does not mean that the individual will suffer from a disease. A number of different enzymes may compete to modify the same molecule, and there may be more than one way to achieve the same end result for a variety of metabolic intermediates. Disease will only occur if a critical enzyme is disabled, or if a control mechanism for a metabolic pathway is affected.

As used herein, “a muscle and bone disease or disorder” refers to a pathological condition caused by defects in genes important for the formation and function of muscles, and connective tissues. Connective tissue is used herein as a broad term that includes bones, cartilage and tendons. For example, defects in fibrillin—a connective tissue proteins that is important in making the tissue strong yet flexible—cause Marfan syndrome, while diastrophic dysplasia is caused by a defect in a sulfate transporter found in cartilage. Two diseases that originate through a defect in the muscle cells themselves are Duchenne muscular dystrophy (DMD) and myotonic dystrophy (DM). DM is another ‘dynamic mutation’ disease, similar to Huntington disease, that involves the expansion of a nucleotide repeat, this time in a muscle protein kinase gene. DMD involves a defect in the cytoskeletal protein, dystrophin, which is important for maintaining cell structure.

As used herein, “a nervous system disease or disorder” refers to a pathological condition caused by defects in the nervous system including the central nervous system, i.e., brain, and the peripheral nervous system. The brain and nervous system form an intricate network of electrical signals that are responsible for coordinating muscles, the senses, speech, memories, thought and emotion. Several diseases that directly affect the nervous system have a genetic component: some are due to a mutation in a single gene, others are proving to have a more complex mode of inheritance. As our understanding of the pathogenesis of neurodegenerative disorders deepens, common themes begin to emerge: Alzheimer brain plaques and the inclusion bodies found in Parkinson disease contain at least one common component, while Huntington disease, fragile X syndrome and spinocerebellar atrophy are all ‘dynamic mutation’ diseases in which there is an expansion of a DNA repeat sequence. Apoptosis is emerging as one of the molecular mechanisms invoked in several neurodegenerative diseases, as are other, specific, intracellular signaling events. The biosynthesis of myelin and the regulation of cholesterol traffic are also involved in Charcot-Marie-Tooth and Neimann-Pick disease, respectively.

As used herein, “a signal disease or disorder” refers to a pathological condition caused by defects in the signal transduction process. Signal transduction within and between cells mean that they can communicate important information and act upon it. Hormones released from their site of synthesis carry a message to their target site, as in the case of leptin, which is released from adipose tissue (fat cells) and transported via the blood to the brain. Here, the leptin signals that enough has been eaten. Leptin binds to a receptor on the surface of hypothalamus cells, triggering subsequent intracellular signaling networks. Intracellular signaling defects account for several diseases, including cancers, ataxia telangiectasia and Cockayne syndrome. Faulty DNA repair mechanisms are also invoked in pathogenesis, since control of cell division, DNA synthesis and DNA repair all are inextricably linked. The end-result of many cell signals is to alter the expression of genes (transcription) by acting on DNA-binding proteins. Some diseases are the result of a lack of or a mutation in these proteins, which stop them from binding DNA in the normal way. Since signaling networks impinge on so many aspects of normal function, it is not surprising that so many diseases have at least some basis in a signaling defect.

As used herein, “a transporter disease or disorder” refers to a pathological condition caused by defects in a transporter, channel or pump. Transporters, channels or pumps that reside in cell membranes are key to maintaining the right balance of ions in cells, and are vital for transmitting signals from nerves to tissues. The consequences of defects in ion channels and transporters are diverse, depending on where they are located and what their cargo is. For example, in the heart, defects in potassium channels do not allow proper transmission of electrical impulses, resulting in the arrhythmia seen in long QT syndrome. In the lungs, failure of a sodium and chloride transporter found in epithelial cells leads to the congestion of cystic fibrosis, while one of the most common inherited forms of deafness, Pendred syndrome, looks to be associated with a defect in a sulphate transporter.

As used herein, “virus” refers to obligate intracellular parasites of living but non-cellular nature, that contain DNA or RNA and a protein coat. Viruses range in diameter from about 20 to about 300 nm. Class I viruses (Baltimore classification) have a double-stranded DNA as their genome; Class II viruses have a single-stranded DNA as their genome; Class III viruses have a double-stranded RNA as their genome; Class IV viruses have a positive single-stranded RNA as their genome, the genome itself acting as mRNA; Class V viruses have a negative single-stranded RNA as their genome used as a template for mRNA synthesis; and Class VI viruses have a positive single-stranded RNA genome but with a DNA intermediate not only in replication but also in mRNA synthesis. The majority of viruses are recognized by the diseases they cause in plants, animals and prokaryotes. Viruses of prokaryotes are known as bacteriophages.

As used herein, “bacteria” refers to small prokaryotic organisms (linear dimensions of around 1 im) with non-compartmentalized circular DNA and ribosomes of about 70S. Bacteria protein synthesis differs from that of eukaryotes. Many anti-bacterial antibiotics interfere with bacteria proteins synthesis but do not affect the infected host.

As used herein, “eubacteria” refers to a major subdivision of the bacteria except the archaebacteria. Most Gram-positive bacteria, cyanobacteria, mycoplasmas, enterobacteria, pseudomonas and chloroplasts are eubacteria. The cytoplasmic membrane of eubacteria contains ester-linked lipids; there is peptidoglycan in the cell wall (if present); and no introns have been discovered in eubacteria.

As used herein, “archaebacteria” refers to a major subdivision of the bacteria except the eubacteria. There are 3 main orders of archaebacteria: extreme halophiles, methanogens and sulphur-dependent extreme thermophiles. Archaebacteria differs from eubacteria in ribosomal structure, the possession (in some case) of introns, and other features including membrane composition.

As used herein, “locus” refers to the site in linkage map or on a chromosome where the nucleic acid sequence, e.g., gene, for a particular trait is located. Any one of the alleles of a sequence may be present at this site.

As used herein, “an allele” refers to one of any different forms or variants of a gene found at the same place, or a locus, on a chromosome.

As used herein, “polymorphism” refers to the existence, in a population, of two or more alleles of a nucleic acid sequence, e.g., gene, where the frequency of the rarer alleles is greater than can be explained by recurrent mutation alone (typically greater than 1%).

As used herein, “variable nucleotide type polymorphism (“VNTR”)” refers to polymorphisms arising from spontaneous tandem duplications of di- or trinucleotide repeated motifs of nucleotides.

As used herein, “single nucleotide polymorphism (“SNP”)” refers to polymorphisms arising from the replacement of only a single nucleotide from the initially present gene sequence.

As used herein, “enzymatic amplification” refers to an enzyme-catalyzed reaction by which nucleic acid, e.g., DNA, molecules are amplified. Examples of such reactions include the polymerase chain reaction and reactions utilizing reverse transcription and subsequent DNA amplification of one or more expressed RNA sequences.

As used herein, “exonuclease” refers to an enzyme that cleaves nucleotides one at time from the end of a polynucleotide chain. Exonuclease may be specific for either 5′ or 3′ end of DNA or RNA. If protein is bound to the nucleic acid, exonuclease cleavage stops when the exonuclease encounters the protein.

As used herein, “recombinase” refers to an enzyme that catalyzes the inter-molecular formation of a nucleic acid duplex from single-stranded nucleic acids obtained from different sources, by a renaturation reaction. Such a recombinase is also capable of catalyzing a strand transfer reaction between a single-stranded nucleic acid from one source and double-stranded nucleic acid obtained from a different source.

As used herein, “serum” refers to the fluid portion of the blood obtained after removal of the fibrin clot and blood cells, distinguished from the plasma in circulating blood.

As used herein, “plasma” refers to the fluid, noncellular portion of the blood, distinguished from the serum obtained after coagulation.

As used herein, “substantially pure” means sufficiently homogeneous to appear free of readily detectable impurities as determined by standard methods of analysis, such as thin layer chromatography (TLC), gel electrophoresis and high performance liquid chromatography (HPLC), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound may, however, be a mixture of stereoisomers or isomers. In such instances, further purification might increase the specific activity of the compound.

As used herein, “biological activity” refers to the in vivo activities of a compound or physiological responses that result upon in vivo administration of a compound, composition or other mixture. Biological activity, thus, encompasses therapeutic effects and pharmaceutical activity of such compounds, compositions and mixtures. Biological activities may be observed in vitro systems designed to test or use such activities. Thus, for purposes herein the biological activity of a luciferase is its oxygenase activity whereby, upon oxidation of a substrate, light is produced.

As used herein, a “receptor” refers to a molecule that has an affinity for a given ligand. Receptors may be naturally-occurring or synthetic molecules. Receptors may also be referred to in the art as anti-ligands. As used herein, the receptor and anti-ligand are interchangeable. Receptors can be used in their unaltered state or as aggregates with other species. Receptors may be attached, covalently or noncovalently, or in physical contact with, to a binding member, either directly or indirectly via a specific binding substance or linker. Examples of receptors, include, but are not limited to: antibodies, cell membrane receptors surface receptors and internalizing receptors, monoclonal antibodies and antisera reactive with specific antigenic determinants [such as on viruses, cells, or other materials], drugs, polynucleotides, nucleic acids, peptides, cofactors, lectins, sugars, polysaccharides, cells, cellular membranes, and organelles.

Examples of receptors and applications using such receptors, include but are not restricted to:

a) enzymes: specific transport proteins or enzymes essential to survival of microorganisms, which could serve as targets for antibiotic [ligand] selection;

b) antibodies: identification of a ligand-binding site on the antibody molecule that combines with the epitope of an antigen of interest may be investigated; determination of a sequence that mimics an antigenic epitope may lead to the development of vaccines of which the immunogen is based on one or more of such sequences or lead to the development of related diagnostic agents or compounds useful in therapeutic treatments such as for auto-immune diseases

c) nucleic acids: identification of ligand, such as protein or RNA, binding sites;

d) catalytic polypeptides: polymers, preferably polypeptides, that are capable of promoting a chemical reaction involving the conversion of one or more reactants to one or more products; such polypeptides generally include a binding site specific for at least one reactant or reaction intermediate and an active functionality proximate to the binding site, in which the functionality is capable of chemically modifying the bound reactant [see, e.g., U.S. Pat. No. 5,215,899];

e) hormone receptors: determination of the ligands that bind with high affinity to a receptor is useful in the development of hormone replacement therapies; for example, identification of ligands that bind to such receptors may lead to the development of drugs to control blood pressure; and

f) opiate receptors: determination of ligands that bind to the opiate receptors in the brain is useful in the development of less-addictive replacements for morphine and related drugs.

As used herein, “antibody” includes antibody fragments, such as Fab fragments, which are composed of a light chain and the variable region of a heavy chain.

As used herein, “humanized antibodies” refer to antibodies that are modified to include “human” sequences of amino acids so that administration to a human will not provoke an immune response. Methods for preparation of such antibodies are known. For example, the hybridoma that expresses the monoclonal antibody is altered by recombinant DNA techniques to express an antibody in which the amino acid composition of the non-variable regions is based on human antibodies. Computer programs have been designed to identify such regions.

As used herein, “production by recombinant means” refers to production methods that use recombinant nucleic acid methods that rely on well known methods of molecular biology for expressing proteins encoded by cloned nucleic acids.

As used herein, “substantially identical” to a product means sufficiently similar so that the property of interest is sufficiently unchanged so that the substantially identical product can be used in place of the product.

As used herein, “equivalent,” when referring to two sequences of nucleic acids means that the two sequences in question encode the same sequence of amino acids or equivalent proteins. It also encompasses those that hybridize under conditions of moderate, preferably high stringency, whereby the encoded protein retains desired properties.

As used herein, when “equivalent” is used in referring to two proteins or peptides, it means that the two proteins or peptides have substantially the same amino acid sequence with only conservative amino acid substitutions (see, e.g., Table 1, above) that do not substantially alter the activity or function of the protein or peptide.

When “equivalent” refers to a property, the property does not need to be present to the same extent [e.g., two peptides can exhibit different rates of the same type of enzymatic activity], but the activities are preferably substantially the same. “Complementary,” when referring to two nucleic acid molecules, means that the two sequences of nucleotides are capable of hybridizing, preferably with less than 25%, more preferably with less than 15%, even more preferably with less than 5%, most preferably with no mismatches between opposed nucleotides. Preferably the two molecules will hybridize under conditions of high stringency.

As used herein: “stringency of hybridization” in determining percentage mismatch is as follows:

1) high stringency: 0.1×SSPE, 0.1% SDS, 65?C.;

2) medium stringency: 0.2×SSPE, 0.1% SDS, 50?C. (also referred to as moderate stringency); and

3) low stringency: 1.0×SSPE, 0.1% SDS, 50?C.

It is understood that equivalent stringencies may be achieved using alternative buffers, salts and temperatures.

The term “substantially” identical or homologous or similar varies with the context as understood by those skilled in the relevant art and generally means at least 70%, preferably means at least 80%, more preferably at least 90%, and most preferably at least 95% identity.

As used herein, a “composition” refers to a any mixture of two or more products or compounds. It may be a solution, a suspension, liquid, powder, a paste, aqueous, non-aqueous or any combination thereof.

As used herein, a “combination” refers to any association between two or among more items.

As used herein, “fluid” refers to any composition that can flow. Fluids thus encompass compositions that are in the form of semi-solids, pastes, solutions, aqueous mixtures, gels, lotions, creams and other such compositions.

As used herein, “vector (or plasmid)” refers to discrete elements that are used to introduce heterologous DNA into cells for either expression or replication thereof. Selection and use of such vehicles are well known within the skill of the artisan. An expression vector includes vectors capable of expressing DNAs that are operatively linked with regulatory sequences, such as promoter regions, that are capable of effecting expression of such DNA fragments. Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are well known to those of skill in the art and include those that are replicable in eukaryotic cells and/or prokaryotic cells and those that remain episomal or those which integrate into the host cell genome.

As used herein, “a promoter region or promoter element” refers to a segment of DNA or RNA that controls transcription of the DNA or RNA to which it is operatively linked. The promoter region includes specific sequences that are sufficient for RNA polymerase recognition, binding and transcription initiation. This portion of the promoter region is referred to as the promoter. In addition, the promoter region includes sequences that modulate this recognition, binding and transcription initiation activity of RNA polymerase. These sequences may be cis acting or may be responsive to trans acting factors. Promoters, depending upon the nature of the regulation, may be constitutive or regulated. Exemplary promoters contemplated for use in prokaryotes include the bacteriophage T7 and T3 promoters, and the like.

As used herein, “operatively linked or operationally associated” refers to the functional relationship of DNA with regulatory and effector sequences of nucleotides, such as promoters, enhancers, transcriptional and translational stop sites, and other signal sequences. For example, operative linkage of DNA to a promoter refers to the physical and functional relationship between the DNA and the promoter such that the transcription of such DNA is initiated from the promoter by an RNA polymerase that specifically recognizes, binds to and transcribes the DNA. In order to optimize expression and/or in vitro transcription, it may be necessary to remove, add or alter 5′ untranslated portions of the clones to eliminate extra, potential inappropriate alternative translation initiation (i.e., start) codons or other sequences that may interfere with or reduce expression, either at the level of transcription or translation. Alternatively, consensus ribosome binding sites (see, e.g., Kozak, J. Biol. Chem., 266:19867-19870 (1991)) can be inserted immediately 5′ of the start codon and may enhance expression. The desirability of (or need for) such modification may be empirically determined.

As used herein, “sample” refers to anything which may contain an analyte for which an analyte assay is desired. The sample may be a biological sample, such as a biological fluid or a biological tissue. Examples of biological fluids include urine, blood, plasma, serum, saliva, semen, stool, sputum, cerebral spinal fluid, tears, mucus, amniotic fluid or the like. Biological tissues are aggregates of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s).

As used herein, “replication” refers to a process of DNA-dependent DNA synthesis wherein the DNA molecule is duplicated to give identical copies.

As used herein, “transcription” refers to a process of DNA-dependent RNA synthesis.

As used herein, “recombination” refers to a reaction between homologous sequences of DNA. The critical feature is that the enzymes responsible for recombination can use any pair of homologous sequences as substrates, although some types of sequences may be favored over others. Recombination allows favorable or unfavorable mutations to be separated and tested as individual units in new assortments.

As used herein, “DNA structure maintenance” refers to DNA sequences, through binding to proteins, that maintain the DNA molecule in particular structures such as chromatids, chromatins or chromosomes.

As used herein, “DNA polymerase” refers to an enzyme that synthesizes DNA using a DNA as the template. It is intended to encompass DNA polymerase with conservative amino acid substitutions that do not substantially alter its activity.

As used herein, “DNA-dependent RNA polymerase” or “transcriptase” refers to an enzyme that synthesizes RNA using a DNA as the template. It is intended to encompass DNA-dependent RNA polymerase with conservative amino acid substitutions that do not substantially alter its activity.

As used herein, “DNAase” refers to an enzyme that attacks bonds in DNA. It is intended to encompass DNAase with conservative amino acid substitutions that do not substantially alter its activity.

As used herein, “DNA ligase” refers to an enzyme that catalyses the formation of a phosphodiester bond to link two adjacent bases separated by a nick in one strand of double helix of DNA. It is intended to encompass DNA ligase with conservative amino acid substitutions that do not substantially alter its activity.

As used herein, “DNA topoisomerase” refers to an enzyme that can change the linking number of DNA. It is intended to encompass DNA topoisomerase with conservative amino acid substitutions that do not substantially alter its activity.

As used herein, “DNA transposase” refers to an enzyme that is involved in insertion of a transposon at a new site. It is intended to encompass DNA transposase with conservative amino acid substitutions that do not substantially alter its activity.

As used herein, “Transposon” refers to a DNA sequence that is able to replicate and insert one copy at a new location in the genome.

As used herein, “DNA kinase” refers to an enzyme that phosphorylates DNA. It is intended to encompass DNA kinase with conservative amino acid substitutions that do not substantially alter its activity.

As used herein, “restriction enzyme” refers to an enzyme that recognizes specific short sequences of DNA and cleaves the duplex at the recognition site or other site. It is intended to encompass a restriction enzyme with conservative amino acid substitutions that do not substantially alter its activity.

As used herein, “rRNA” or “ribosomal RNA” refers to the RNA components of the ribosome, a compact ribonucleoprotein particle that assembles amino acids into proteins.

As used herein, “mRNA” or “messenger RNA” refers to the RNA molecule that bears the same sequence of the DNA coding strand and is used as the template in protein synthesis.

As used herein, “tRNA” or “transfer RNA” refers to the RNA molecule that carries amino acids to the ribosome for protein synthesis.

As used herein, “reverse transcription” refers to the RNA-dependent DNA synthesis.

As used herein, “RNA splicing” refers to the removal of introns and joining of exons in RNA so that introns are spliced out and exons are spliced together.

As used herein, “RNA-dependent DNA polymerase” or “reverse transcriptase” refers to an enzyme that synthesizes DNA using a RNA as the template. It is intended to encompass a RNA-dependent DNA polymerase with conservative amino acid substitutions that do not substantially alter its activity.

As used herein, “RNA-dependent RNA polymerase” refers to an enzyme that synthesizes RNA using a RNA as the template. It is intended to encompass a RNA-dependent RNA polymerase with conservative amino acid substitutions that do not substantially alter its activity.

As used herein, “RNA ligase” refers to an enzyme that catalyses the formation of a phosphodiester bond to link two adjacent bases separated by a nick in one strand of RNA. It is intended to encompass a RNA ligase with conservative amino acid substitutions that do not substantially alter its activity.

As used herein, “RNA maturase” refers to an enzyme that catalyses the removal of intron in the RNA splicing. It is intended to encompass a RNA maturase with conservative amino acid substitutions that do not substantially alter its activity.

As used herein, “luminescence” refers to the detectable EM radiation, generally, UV, IR or visible EM radiation that is produced when the excited product of an exergic chemical process reverts to its ground state with the emission of light. Chemiluminescence is luminescence that results from a chemical reaction. Bioluminescence is chemiluminescence that results from a chemical reaction using biological molecules or synthetic versions or analogs thereof as substrates and/or enzymes.

As used herein, “bioluminescence,” which is a type of chemiluminescence, refers to the emission of light by biological molecules, particularly proteins. The essential condition for bioluminescence is molecular oxygen, either bound or free in the presence of an oxygenase, a luciferase, which acts on a substrate, a luciferin. Bioluminescence is generated by an enzyme or other protein (luciferase) that is an oxygenase that acts on a substrate luciferin (a bioluminescence substrate) in the presence of molecular oxygen and transforms the substrate to an excited state, which upon return to a lower energy level releases the energy in the form of light.

As used herein, the substrates and enzymes for producing bioluminescence are generically referred to as luciferin and luciferase, respectively. When reference is made to a particular species thereof, for clarity, each generic term is used with the name of the organism from which it derives, for example, bacterial luciferin or firefly luciferase.

As used herein, “luciferase” refers to oxygenases that catalyze a light emitting reaction. For instance, bacterial luciferases catalyze the oxidation of flavin mononucleotide [FMN] and aliphatic aldehydes, which reaction produces light. Another class of luciferases, found among marine arthropods, catalyzes the oxidation of Cypridina [Vargula] luciferin, and another class of luciferases catalyzes the oxidation of Coleoptera luciferin.

Thus, luciferase refers to an enzyme or photoprotein that catalyzes a bioluminescent reaction [a reaction that produces bioluminescence]. The luciferases, such as firefly and Renilla luciferases, that are enzymes which act catalytically and are unchanged during the bioluminescence generating reaction. The luciferase photoproteins, such as the aequorin photoprotein to which luciferin is non-covalently bound, are changed, such as by release of the luciferin, during bioluminescence generating reaction. The luciferase is a protein that occurs naturally in an organism or a variant or mutant thereof, such as a variant produced by mutagenesis that has one or more properties, such as thermal stability, that differ from the naturally-occurring protein. Luciferases and modified mutant or variant forms thereof are well known. For purposes herein, reference to luciferase refers to either the photoproteins or luciferases.

As used herein, “peroxidase” refers to an enzyme that catalyses a host of reactions in which hydrogen peroxide is a specific oxidizing agent and a wide range of substrates act as electron donors. It is intended to encompass a peroxidase with conservative amino acid substitutions that do not substantially alter its activity. Peroxidases are widely distributed in nature and are produced by a wide variety of plant species. The chief commercially available peroxidase is horseradish peroxidase.

As used herein, “urease” refers to an enzyme that catalyses decomposition of urea to form ammonia and carbon dioxide. It is intended to encompass an urease with conservative amino acid substitutions that do not substantially alter its activity. Urease is widely found in plants, animals and microorganisms.

As used herein, “alkaline phosphatases” refers to a family of functionally related enzymes named after the tissues in which they predominately appear. Alkaline phosphatases carry out hydrolase/transferase reactions on phosphate-containing substrates at a high pH optimum. It is intended to encompass an alkaline phosphatases with conservative amino acid substitutions that do not substantially alter its activity.

As used herein, “glutathione S-transferase” refers to a ubiquitous family of enzymes with dual substrate specificities that perform important biochemical functions of xenobiotic biotransformation and detoxification, drug metabolism, and protection of tissues against peroxidative damage. The basic reaction catalyzed by glutathione S-transferase is the conjugation of an electrophile with reduced glutathione (GSH) and results in either activation or deactivation/detoxification of the chemical. It is intended to encompass a glutathione S-transferase with conservative amino acid substitutions that do not substantially alter its activity.

As used herein, high-throughput screening (HTS) refers to processes that test a large number of samples, such as samples of diverse chemical structures against disease targets to identify “hits” (see, e.g., Broach et al. High throughput screening for drug discovery, Nature, 384:14-16 (1996); Janzen, et al. High throughput screening as a discovery tool in the pharmaceutical industry, Lab Robotics Automation: 8261-265 (1996); Fernandes, P. B., Letter from the society president, J. Biomol. Screening, 2:1 (1997); Burbaum, et al., New technologies for high-throughput screening, Curr. Opin. Chem. Biol., 1:72-78 (1997)]. HTS operations are highly automated and computerized to handle sample preparation, assay procedures and the subsequent processing of large volumes of data.

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochem. 11:1726).

For clarity of disclosure, and not by way of limitation, the detailed description is divided into the subsections that follow.

### B. Methods for Detecting Abnormal Base-Pairing

Provided herein are methods for detecting abnormal base-pairing in a nucleic acid duplex. Detection of abnormal base pairing has numerous applications, such as in diagnostics, mutational analyses and polymorphism identification. The method involves binding a mutant enzyme that specifically binds to mismatched base pairs in a DNA duplex, DNA:RNA duplex, or RNA:RNA duplex, and detecting such binding, which can be quantitative. By virtue of the base specificity of the certain enzymes, the identity of the abnormal base pairing may be determined.

The reactions can be performed in various formats, including solution and solid phase reactions. Solid supports to which nucleic acid or enzyme is bound. In addition, the resulting complexes of enzyme bound to nucleic acid can be captured on solid supports by virtue of interaction of the nucleic acid with other nucleic acids on the supports or the enzyme with moieties on the supports.

The preferred formats herein are those that are amenable to high throughput analyses, such as chip-based reactions in which nucleic acid probes of known sequence are arranged, such as in an array on a support, and reacted with a sample, such as nucleic acid from a body fluid or tissue.

In a particular embodiment, the method is performed by contacting a nucleic acid duplex having or suspected of having an abnormal base-pairing with a mutant DNA repair enzyme or complex thereof, where the mutant DNA repair enzyme or complex thereof has binding affinity for the abnormal base-pairing in the duplex but has attenuated catalytic activity; and then detecting binding between the nucleic acid duplex and the mutant DNA repair enzyme or complex thereof, whereby the presence or quantity of the abnormal base-pairing in the duplex is assessed.

As noted, the nucleic acid duplex to be assayed is a DNA:DNA, a DNA:RNA or a RNA:RNA duplex. Preferably, the nucleic acid duplex to be assayed is a DNA:DNA duplex. The abnormal base-pairing to be detected includes a base-pair mismatch, a base insertion, a base deletion and a pyrimidine dimer. Preferably, the base-pair mismatch to be detected is a single base-pair mismatch. Non-limiting examples of the base-pair mismatch that can be detected include A:A, A:C, A:G, C:C, C:T, G:G, G:T, T:T, C:U, G:U, T:U, U:U, 5-formyluracil (fu):G, 7,8-dihydro-8-oxo-guanine (8-oxoG):C, 8-oxoG:A or a combination thereof. Also preferably, the base insertion or base deletion to be detected is a single base insertion or deletion. For example, the base insertion or base deletion resulting in a single-stranded loop containing about 1-5 bases or a loop containing more than 5 bases can be detected.

1. Mutant DNA Repair Enzyme or Complex Thereof

Any mutant DNA repair enzyme or complex thereof that has binding affinity for the abnormal base-pairing in the duplex but has attenuated catalytic activity can be used in the present methods. Such enzymes may be prepared by mutagenensis of nucleic acids encoding the enzyme and selection of the expressed protein for the requisite binding properties and reduced or absent catalytic activities.

Mutant enzymes having the desired specificity can be prepared using routine mutagenesis methods. Residues to mutate can be identified by systematically mutating residues to different residues, and identifying those that have the desired reduction in catalytic activity and retention of binding activity for a particular abnormal base-pairing. Alternatively or additionally, mutations may be based upon predicted or known 3-D structures of enzymes, including predicted affects of various mutations (see, e.g., Turner et al. (1998) Nature Structural Biol. 5:369-376; Ault-Richié et al. (1994) J. Biol. Chem. 269:31472-31478; Yuan et al. (1996) Biol. Chem. 271:28009-28016; Williams et al. (1998) Biochemistry 37:7096; Steadman et al. (1998) Biochemistry 37:7089-7095; Finer-Moore et al. (1998) J. Mol. Biol. 276:113-129; Strop et al. (1997) Protein Sci. 6:2504-2511; Finer-Moore et al. (1996) Biochemistry 35:5125-5136; Schiffer et al. (1995) Biochemistry 34:16279-16287; Costi et al. (1996) Biochemistry 35:3944-3949; Graves et al. (1992) Biochemistry 31:15-21; Carreras et al. (1992) Biochemistry 31:6038-6044. Such predictions can be made by those of skill in the art of computational chemistry. Hence, for any selected enzyme, the mutations need to inactivate catalytic activity but retain binding activity can be determined empirically.

Mutant enzymes can be selected for example by plating plasmids containing DNA containing mutagenized genes in wells coated with duplexes containing mismatches, expressing the proteins, and looking for binding to the mismatched duplexes, and selecting the nucleic acid that expressed the proteins that bound thereto.

A typical mutant enzyme, is a DNA repair enzyme with a mutation that attenuates the catalytic activity, but that has little or small effects on the binding activity. By selecting the enzymes that bind to duplexes, which are retained on a support, enzymes with the desired specificity and lack of catalytic activity will be selected. Enzymes the retain catalytic activity, will not remain bound.

Exemplary DNA repair enzyme and complexes thereof that can be mutated for use in the methods herein, include, but are not limited to, a mutant mutH, a mutant mutL, a mutant mutM, a mutant mutS, a mutant mutY, a mutant uvrD, a mutant dam, a mutant thymidine DNA glycosylase (TDG), a mutant mismatch-specific DNA glycosylase (MUG), a mutant AlkA, a mutant MLH1, a mutant MSH2, a mutant MSH3, a mutant MSH6, a mutant Exonuclease I, a mutant T4 endonuclease V, a mutant FEN1 (RAD27), a mutant DNA polymerase ä, a mutant DNA polymerase å, a mutant RPA, a mutant PCNA, a mutant RFC, a mutant Exonuclease V, a mutant DNA polymerase III holoenzyme, a mutant DNA helicase, a mutant RecJ exonuclease or a combination thereof.

a. Nucleic Acids Encoding DNA Repair Enzymes

Nucleic acids encoding DNA repair enzymes can be obtained by methods known in the art. Known nucleic acid sequences of DNA repair enzymes can be used in isolating nucleic acids encoding DNA repair enzymes from natural or other sources. Alternatively, complete or partial nucleic acids encoding DNA repair enzymes can be obtained by chemical synthesis according to the known sequences or obtained from commercial or other sources.

Eukaryotic cells and prokaryotic cells can serve as a nucleic acid source for the isolation of nucleic acids encoding DNA repair enzymes. The DNA can be obtained by standard procedures known in the art from cloned DNA (e.g., a DNA “library”), chemical synthesis, cDNA cloning, or by the cloning of genomic DNA, or fragments thereof, purified from the desired cell (see, for example, Sambrook et al., 1989, Molecular Cloning, A Laboratory Manual, 2d Ed., Cold Spring Harbor Laboratory Press, Cold Spring Harbor, N.Y.; Glover, D. M. (ed.), 1985, DNA Cloning: A Practical Approach, MRL Press, Ltd., Oxford, U.K. Vol. I, II.). Clones derived from genomic DNA can contain regulatory and intron DNA regions in addition to coding regions; clones derived from cDNA or RNA contain only exon sequences. Whatever the source, the gene is generally molecularly cloned into a suitable vector for propagation of the gene.

In the molecular cloning of the gene from cDNA, cDNA can be generated from total cellular RNA or mRNA by methods that are known in the art. The gene can also be obtained from genomic DNA, where DNA fragments are generated (e.g., using restriction enzymes or by mechanical shearing), some of which will encode the desired gene. The linear DNA fragments can then be separated according to size by standard techniques, including but not limited to, agarose and polyacrylamide gel electrophoresis and column chromatography.

Once the DNA fragments are generated, identification of the specific DNA fragment containing all or a portion of the DNA repair enzymes gene can be accomplished in a number of ways.

A preferred method for isolating an DNA repair enzyme gene is by the polymerase chain reaction (PCR), which can be used to amplify the desired DNA repair enzyme sequence in a genomic or cDNA library or from genomic DNA or cDNA that has not been incorporated into a library. Oligonucleotide primers which hybridize to the DNA repair enzyme sequences can be used as primers in PCR.

Additionally, a portion of the DNA repair enzyme (of any species) gene or its specific RNA, or a fragment thereof, can be purified (or an oligonucleotide synthesized) and labeled, the generated DNA fragments may be screened by nucleic acid hybridization to the labeled probe (Benton, W. and Davis, R., 1977, Science 196:180; Grunstein, M. And Hogness, D., 1975, Proc. Natl. Acad. Sci. U.S.A. 72:3961). Those DNA fragments with substantial homology to the probe will hybridize. The DNA repair enzyme nucleic acids can be also identified and isolated by expression cloning using, for example, DNA repair activities or anti-DNA repair enzyme antibodies for selection.

Alternatives to obtaining the DNA repair enzyme DNA by cloning or amplification include, but are not limited to, chemically synthesizing the gene sequence itself from the known DNA repair enzyme nucleotide sequence or making cDNA to the mRNA which encodes the DNA repair enzyme. Any suitable method known to those of skill in the art may be employed.

Once a clone has been obtained, its identity can be confirmed by nucleic acid sequencing (by methods known in the art) and comparison to known DNA repair enzyme sequences. DNA sequence analysis can be performed by techniques known in the art, including but not limited to, the method of Maxam and Gilbert (1980, Meth. Enzymol. 65:499-560), the Sanger dideoxy method (Sanger, F., et al., 1977, Proc. Natl. Acad. Sci. U.S.A. 74:5463), the use of T7 DNA polymerase (Tabor and Richardson, U.S. Pat. No. 4,795,699), use of an automated DNA sequenator (e.g., Applied Biosystems, Foster City, Calif.).

Nucleic acids which are hybridizable to a DNA repair enzyme nucleic acid, or to a nucleic acid encoding an DNA repair enzyme derivative can be isolated, by nucleic acid hybridization under conditions of low, high, or medium stringency (Shilo and Weinberg, 1981, Proc. Natl. Acad. Sci. USA 78:6789-6792).

b. Selecting and Producing Mutant DNA Repair Enzymes

Once nucleic acids encoding the DNA repair enzymes are obtained, these nucleic acids can be mutagenized and screened and/or selected for DNA repair enzymes that substantially retain their binding affinity or have enhanced binding affinity for abnormal base-pairing but have attenuated catalytic activity. Insertional, deletional or point mutation(s) can be introduced into nucleic acids encoding the DNA repair enzymes. Techniques for mutagenesis known in the art can be used, including, but not limited to, in vitro site-directed mutagenesis (Hutchinson et al., 1978, J. Biol. Chem 253:6551), use of TAB® linkers (Pharmacia), mutation-containing PCR primers, etc. Mutagenesis can be followed by phenotypic testing of the altered gene product.

Site-directed mutagenesis protocols can take advantage of vectors that provide single stranded as well as double stranded DNA, as needed. Generally, the mutagenesis protocol with such vectors is as follows. A mutagenic primer, i.e., a primer complementary to the sequence to be changed, but including one or a small number of altered, added, or deleted bases, is synthesized. The primer is extended in vitro by a DNA polymerase and, after some additional manipulations, the now double-stranded DNA is transfected into bacterial cells. Next, by a variety of methods, the desired mutated DNA is identified, and the desired protein is purified from clones containing the mutated sequence. For longer sequences, additional cloning steps are often required because long inserts (longer than 2 kilobases) are unstable in those vectors. Protocols are known to one skilled in the art and kits for site-directed mutagenesis are widely available from biotechnology supply companies, for example from Amersham Life Science, Inc. (Arlington Heights, Ill.) and Stratagene Cloning Systems (La Jolla, Calif.).

Information regarding to the structural-function relationship of the DNA repair enzymes can be used in the mutagenesis and selection of DNA repair enzymes that substantially retain their binding affinity or have enhanced binding affinity for the abnormal base-pairing but have attenuated catalytic activity. For example, mutants can be made in the enzyme's binding site for its co-enzyme, co-factor, or in the mutant enzyme's catalytic site, or a combination thereof.

Once a mutant DNA repair enzyme with desired properties, i.e., substantially retaining its binding affinity or having enhanced binding affinity for the abnormal base-pairing but has attenuated catalytic activity, is identified, such mutant DNA repair enzyme can be produced by any methods known in the art including recombinant expression, chemical synthesis or a combination thereof. Preferably, the mutant DNA repair enzyme is obtained by recombinant expression.

For recombinant expression, the mutant DNA repair enzyme gene or portion thereof is inserted into an appropriate cloning vector for expression in a particular host cell. A large number of vector-host systems known in the art may be used. Possible vectors include, but are not limited to, plasmids or modified viruses, but the vector system must be compatible with the host cells used. Such vectors include, but are not limited to, bacteriophages such as lambda derivatives, or plasmids such as pBR322 or pUC plasmid derivatives or the Bluescript vector (Stratagene). The insertion into a cloning vector can, for example, be accomplished by ligating the DNA fragment into a cloning vector which has complementary cohesive termini. If, however, the complementary restriction sites used to fragment the DNA are not present in the cloning vector, the ends of the DNA molecules can be enzymatically modified. Alternatively, a desired site can be produced by ligating sequences of nucleotides (linkers) onto the DNA termini; these ligated linkers can include specific oligonucleotides encoding restriction endonuclease recognition sequences. Recombinant molecules can be introduced into host cells via transformation, transfection, infection, electroporation, etc., so that many copies of the gene sequence are generated.

In an alternative method, the desired gene can be identified and isolated after insertion into a suitable cloning vector in a “shot gun” approach. Enrichment for the desired gene, for example, by size fractionation, can be done before insertion into the cloning vector.

In specific embodiments, transformation of host cells with recombinant DNA molecules that incorporate the isolated mutant DNA repair enzyme gene, cDNA, or synthesized DNA sequence enables generation of multiple copies of the gene. Thus, the gene can be obtained in large quantities by growing transformants, isolating the recombinant DNA molecules from the transformants and, when necessary, retrieving the inserted gene from the isolated recombinant DNA.

The nucleotide sequence coding for a mutant DNA repair enzyme or a functionally active analog or fragment or other derivative thereof, can be inserted into an appropriate expression vector, e.g., a vector which contains the necessary elements for the transcription and translation of the inserted protein-coding sequence. The necessary transcriptional and translational signals can also be supplied by the native mutant DNA repair enzyme gene and/or its flanking regions. A variety of host-vector systems can be utilized to express the protein-coding sequence. These systems include but are not limited to mammalian cell systems infected with virus (e.g., vaccinia virus, adenovirus, etc.); insect cell systems infected with virus (e.g., baculovirus); microorganisms such as yeast containing yeast vectors, or bacteria transformed with bacteriophage, DNA, plasmid DNA, or cosmid DNA. The expression elements of vectors vary in their strengths and specificities. Depending on the host-vector system utilized, suitable transcription and translation elements can be used.

The methods previously described for the insertion of DNA fragments into a vector can be used to construct expression vectors containing a chimeric gene containing appropriate transcriptional/translational control signals and the protein coding sequences. These methods can include in vitro recombinant DNA and synthetic techniques and in vivo recombinants (genetic recombination). Expression of a nucleic acid sequence encoding a mutant DNA repair enzyme or peptide fragment can be regulated by a second nucleic acid sequence so that the mutant DNA repair enzyme or peptide is expressed in a host transformed with the recombinant DNA molecule. For example, expression of a mutant DNA repair enzyme can be controlled by a promoter/enhancer element as is known in the art. Promoters which can be used to control a mutant DNA repair enzyme expression include, but are not limited to, the SV40 early promoter region (Bernoist and Chambon, 1981, Nature 290:304-310), the promoter contained in the 3′ long terminal repeat of Rous sarcoma virus (Yamamoto, et al., 1980, Cell 22:787-797), the herpes thymidine kinase promoter (Wagner et al., 1981, Proc. Natl. Acad. Sci. U.S.A. 78:1441-1445), the regulatory sequences of the metallothioneine gene (Brinster et al., 1982, Nature 296:39-42); prokaryotic expression vectors such as the â-lactamase promoter (Villa-Kamaroff, et al., 1978, Proc. Natl. Acad. Sci. U.S.A. 75:3727-3731), or the tac promoter (DeBoer, et al., 1983, Proc. Natl. Acad. Sci. U.S.A. 80:21-25); see also “Useful proteins from recombinant bacteria” in Scientific American, 1980, 242:74-94; promoter elements from yeast or other fungi such as the Gal 4 promoter, the ADC (alcohol dehydrogenase) promoter, PGK (phosphoglycerol kinase) promoter, alkaline phosphatase promoter, and certain animal transcriptional control regions.

For example, a vector can be used that contains a promoter operably linked to a nucleic acid encoding a mutant DNA repair enzyme, one or more origins of replication, and, optionally, one or more selectable markers (e.g., an antibiotic resistance gene).

In a specific embodiment, an expression construct is made by subcloning a mutant DNA repair enzyme coding sequence into the EcoRI restriction site of each of the three pGEX vectors (Glutathione S-Transferase expression vectors; see, e.g., Smith and Johnson, 1988, Gene 7:31-40). This allows for the expression of a mutant DNA repair enzyme product from the subclone in the correct reading frame.

Expression vectors containing a mutant DNA repair enzyme gene inserts can be identified by three general approaches: (a) nucleic acid hybridization, (b) presence or absence of “marker” gene functions, and (c) expression of inserted sequences. In the first approach, the presence of a mutant DNA repair enzyme gene inserted in an expression vector can be detected by nucleic acid hybridization using probes containing sequences that are homologous to an inserted mutant DNA repair enzyme gene. In the second approach, the recombinant vector/host system can be identified and selected based upon the presence or absence of certain “marker” gene functions (e.g., thymidine kinase activity, resistance to antibiotics, transformation phenotype, occlusion body formation in baculovirus, etc.) caused by the insertion of a mutant DNA repair enzyme gene in the vector. For example, if the mutant DNA repair enzyme gene is inserted within the marker gene sequence of the vector, recombinants containing the mutant DNA repair enzyme insert can be identified by the absence of the marker gene function. In the third approach, recombinant expression vectors can be identified by assaying the mutant DNA repair enzyme product expressed by the recombinant. Such assays can be based, for example, on the physical or functional properties of the mutant DNA repair enzyme in in vitro assay systems, e.g., binding with anti-mutant DNA repair enzyme antibody.

Once a particular recombinant DNA molecule is identified and isolated, several methods known in the art can be used to propagate it. Once a suitable host system and growth conditions are established, recombinant expression vectors can be propagated and prepared in quantity. As previously explained, the expression vectors which can be used include, but are not limited to, the following vectors or their derivatives: human or animal viruses such as vaccinia virus or adenovirus; insect viruses such as baculovirus; yeast vectors; bacteriophage vectors (e.g., lambda), and plasmid and cosmid DNA vectors, to name but a few.

In addition, a host cell strain can be chosen which modulates the expression of the inserted sequences, or modifies and processes the gene product in the specific fashion desired. Expression from certain promoters can be elevated in the presence of certain inducers; thus, expression of the genetically engineered mutant DNA repair enzyme can be controlled. Furthermore, different host cells have characteristic and specific mechanisms for the translational and post-translational processing and modification (e.g., glycosylation, phosphorylation) of proteins. Appropriate cell lines or host systems can be chosen to ensure the desired modification and processing of the foreign protein expressed. For example, expression in a bacterial system can be used to produce an unglycosylated core protein product. Expression in yeast will produce a glycosylated product. Expression in appropriate animal cells can be used to ensure “native” glycosylation of a heterologous protein. Furthermore, different vector/host expression systems can effect processing reactions to different extent.

C. Mutant MutL or MLH1

In a specific embodiment, a mutant mutL or MLH1 is used in the present methods. The nucleic acid molecules containing sequences of nucleotides with the following GenBank accession Nos. can be used in obtaining nucleic acid encoding mutL and in mutagenesis: AF170912 (Caulobacter crescentus), AI518690 (Drosophila melanogaster), AI456947 (Drosophila melanogaster), AI389544 (Drosophila melanogaster), AI387992 (Drosophila melanogaster), AI292490 (Drosophila melanogaster), AF068271 (Drosophila melanogaster), AF068257 (Drosophila melanogaster), U50453 (Thermus aquaticus), U27343 (Bacillus subtilis), U71053 (U71053 (Thermotoga maritima), U71052 (Aquifex pyrophilus), U13696 (Human), U13695 (Human), M29687 (S.typhimurium), M63655 (E. coli) and L19346 (Escherichia coli). The nucleic acid molecules containing sequences of nucleotides with the following GenBank accession Nos. can be used in obtaining nucleic acid encoding MLH1 and in mutagenesis: AI389544 (Drosophila melanogaster), AI387992 (Drosophila melanogaster), AF068257 (Drosophila melanogaster), U80054 (Rattus norvegicus) and U07187 (Saccharomyces cerevisiae).

In a preferred embodiment, mutant mutL or MLH1 used in the present methods has a mutation in its catalytic site, ATP binding site or combination thereof (Ban and Yang, Cell, 95:541-552 (1998)).

In another preferred embodiment, the mutant mutL used in the present methods is an E. Coli mutant mutL having a E29K, E32K, A37T, D58N, G60S, G93D, R95C, G96S, G96D, S112L, A16T, A16V, P305L, H308Y, G238D, S106F or A271V mutation (Aronshtam and Marinus, Nucleic Acids Res., 24(13):2498-504 (1996)).

In still another preferred embodiment, the mutant MLH1 used in the present methods is a human mutant MLH1 having a P28L, M35R, S44F, G67R, I68N, I107R, T117R, T117M, R265H, V185G or G224D mutation (Peltomaki and Vasen, Gastroenterology, 113(4):1146-58 (1997)).

d. Mutant MutS

In another specific embodiment, a mutant mutS is used in the present methods. The nucleic acid molecules containing sequences of nucleotides with the following GenBank accession Nos. can be used in obtaining nucleic acid encoding mutS and in mutagenesis: AF146227 (Mus musculus), AF193018 (Arabidopsis thaliana), AF144608 (Vibrio parahaemolyticus), AF034759 (Homo sapiens), AF104243 (Homo sapiens), AF007553 (Thermus aquaticus caldophilus), AF109905 (Mus musculus), AF070079 (Homo sapiens), AF070071 (Homo sapiens), AH006902 (Homo sapiens), AF048991 (Homo sapiens), AF048986 (Homo sapiens), U33117 (Thermus aquaticus), U16152 (Yersinia enterocolitica), AF000945 (Vibrio cholarae), U698873 (Escherichia coli), AF003252 (Haemophilus influenzae strain b (Eagan), AF003005 (Arabidopsis thaliana), AF002706 (Arabidopsis thaliana), L10319 (Mouse), D63810 (Thermus thermophilus), U27343 (Bacillus subtilis), U71155 (Thermotoga maritima), U71154 (Aquifex pyrophilus), U16303 (Salmonella typhimurium), U21011 (Mus musculus), M84170 (S. cerevisiae), M84169 (S. cerevisiae), M18965 (S. typhimurium) and M63007 (Azotobacter vinelandii).

Preferably, the mutant mutS used in the present methods has a mutation in its catalytic site, dimerization site, mutL interaction site or a combination thereof. Also preferably, the mutant mutS used in the present methods is an E.Coli mutant mutS (see, e.g., Wu et al., J. Biol. Chem., 274(9):5948-52 (1999)).

e. Mutant MutM

In still another specific embodiment, a mutant mutM is used in the present methods. The nucleic acid molecules containing sequences of nucleotides with the following GenBank accession Nos. can be used in obtaining nucleic acid encoding mutM and in mutagenesis: AF148219 (Nostoc PCC8009), AF026468 (Streptococcus mutans), AF093820 (Mastigocladus laminosus), AB010690 (Arabidopsis thaliana), U40620 (Streptococcus mutans), AB008520 (Thermus thermophilus) and AF026691 (Homo sapiens).

Preferably, the mutant mutM used in the present methods has a mutation in its catalytic site, mutY interaction site or combination thereof (Michaels et al., Proc. Natl. Acad. Sci. U.S.A., 89(15):7022-5 (1992)). Also preferably, the mutant mutM used in the present methods is an E.Coli mutant mutM having a K57G or K57R mutation (Sidorkina and Laval, Nucleic Acids Res, 26(23):5351-7 (1998)).

f. Mutant MutY

In yet another specific embodiment, a mutant mutY is used in the present methods. The nucleic acid molecules containing sequences of nucleotides with the following GenBank accession Nos. can be used in obtaining nucleic acid encoding mutY and in mutagenesis: AF121797 (Streptomyces), U63329 (Human), AA409965 (Mus musculus) and AF056199 (Streptomyces).

Preferably, the mutant mutY used in the present methods has a mutation in its catalytic site, mutM interaction site or combination thereof (Michaels et al., Proc. Natl. Acad. Sci. U.S.A., 89(15):7022-5 (1992)). Also preferably, the mutant mutY used in the present methods is an E.Coli mutant mutY having an E37S, V45N, G116D, D138N or K142A mutation (Lu et al., J. Biol. Chem., 271(39):24138-43 (1996); Guan et al., Nat. Struct. Biol., 5(12):1058-64 (1998); and Wright et al., J. Biol. Chem., 274(41):29011-18 (1999)). More preferably, the abnormal base-pairing to be detected is a A:C mismatch and the mutant DNA repair enzyme used in the present methods is a mutant MutY.

g. Mutant UvrD

In yet another specific embodiment, a mutant uvrD is used in the present methods. The nucleic acid molecules containing sequence of nucleotides with the following GenBank accession Nos. can be used in obtaining nucleic acid encoding uvrD and in mutagenesis: L02122 (E. coli), AF028736 (Serratia marcescens), AF010185 (Pseudomonas aeruginosa), D00069 (Escherichia coli), AB001291 (Thermus thermophilus), M38257 (Escherichia coli) and L22432 (Mycoplasma capricolum).

Preferably, the mutant uvrD used in the present methods has a mutation in its catalytic site, ATP binding site or combination thereof. Also preferably, the mutant uvrD used in the present methods is an E.Coli mutant uvrD having a K35M, D220NE221Q, E221Q or Q251E mutation (Brosh and Matson, J. Bacteriol., 177(19):5612-21 (1995); George et al., J. Mol. Biol., 235(2):424-35 (1994); and Brosh and Matson, J. Biol. Chem., 272(1):572-79 (1997)).

h. Mutant MSH2

In yet another specific embodiment, a mutant MSH2 is used in the present methods. The nucleic acid molecules containing sequences of nucleotides with the following GenBank accession Nos. can be used in obtaining nucleic acid encoding MSH2 and in mutagenesis: AF109243 (Arabidopsis thaliana), AF030634 (Neurospora crassa), AF002706 (Arabidopsis thaliana), AF026549 (Arabidopsis thaliana), L47582 (Homo sapiens), L47583 (Homo sapiens), L47581 (Homo sapiens) and M84170 (S. cerevisiae).

Preferably, the mutant MSH2 used in the present methods has a mutation in its catalytic site, ATP binding site, ATPase site or combination thereof. Also preferably, the mutant MSH2 used in the present methods is a S. cerevisiae mutant MSH2 having a G693D or a G855D mutation (Alani et al., Mol. Cell. Biol., 17(5):2436-47 (1997)), or a human mutant MSH2 having a fragment encoding 195 amino acids within the C-terminal domain of hMSH-2 or having a K675R mutation (Whitehouse et al., Biochem. Biophys. Res. Commun., 232(1):10-3 (1997); and laccarino et al., EMBO J., 17(9):2677-86 (1998)).

i. Mutant MSH6

In yet another specific embodiment, a mutant MSH6 is used in the present methods. The nucleic acid molecules containing sequence of nucleotides with the following GenBank accession Nos. can be used in obtaining nucleic acid encoding MSH6 and in mutagenesis: U54777 (Homo sapiens) and AF031087 (Mus musculus).

Preferably, the mutant MSH6 used in the present methods has a mutation in its catalytic site, ATP binding site, ATPase site or combination thereof. Also preferably, the mutant MSH6 used in the present methods is a human mutant MSH6 having a K1140R mutation (laccarino et al., EMBO J., 17(9):2677-86 (1998)). More preferably, the mutant DNA repair complex used in the present methods comprises a human mutant MSH2 having a K675R mutation and a human mutant MSH6 having a K1140R mutation.

i. Mutant T4 Endonuclease V

In yet another specific embodiment, a mutant T4 endonuclease V is used in the present methods. The nucleic acid molecules containing sequences of nucleotides with the following GenBank accession Nos. can be used in obtaining nucleic acid encoding T4 endonuclease V and in mutagenesis: M35392 (Synthetic), U76612 (Coliphage), U48703 (Bacteriophage T4) and M23414 (Synthetic). Preferably, the mutant T4 endonuclease V used in the present methods has a E23Q mutation (Doi et al., Proc. Natl. Acad. Sci. U.S.A., 89(20):9420-4 (1992)).

k. Mutant MSH3

In yet another specific embodiment, a mutant MSH3 is used in the present methods. The nucleic acid molecules containing sequences of nucleoties with the following GenBank accession Nos. can be used in obtaining nucleic acid encoding MSH3 and in mutagenesis: J04810 (Human) and M96250 (Saccharomyces cerevisiae).

l. Mutant alkA

In yet another specific embodiment, a mutant alka is used in the present methods. The nucleic acid molecules containing sequences of nucleotides with the following GenBank accession Nos. can be used in obtaining nucleic acid encoding alkA and in mutagenesis: D14465 (Bacillus subtilis) and K02498 (E. coli).

m. Mutant Exonuclease I

In yet another specific embodiment, a mutant exonuclease I is used in the present methods. The nucleic acid molecules containing sequences of nucleotides with the following GenBank accession Nos. can be used in obtaining nucleic acid encoding exonuclease I and in mutagenesis: AF060479 (Homo sapiens), U86134 (Saccharomyces cerevisiae) and J02641 (E. coli).

n. Mutant fen1

In yet another specific embodiment, a mutant fen1 is used in the present methods. The nucleic acid molecules containing sequences of nucleotides with the following GenBank accession Nos. can be used in obtaining nucleic acid encoding fen1 and in mutagenesis: AF065397 (Xenopus laevis (FEN1)) and AF036327 (Xenopus laevis (FEN1)).

o. Mutant rpa

In yet another specific embodiment, a mutant rpa is used in the present methods. The nucleic acid molecules containing sequences of nucleotides with the following GenBank accession Nos. can be used in obtaining nucleic acid encoding rpa and in mutagenesis: AA955716 (Homo sapiens), AA955320 (Homo sapiens), AA925949 (Homo sapiens), U29383 (Zea mays), U33419 (Orf virus) and L07493 (Homo sapiens).

p. Mutant pcna

In yet another specific embodiment, a mutant pcna is used in the present methods. The nucleic acid molecules containing sequences of nucleotides with the following GenBank accession Nos. can be used in obtaining nucleic acid encoding pcna and in mutagenesis: AB025029 (Nicotiana tabacum), AF038875 (Nicotiana tabacum), AF104412 (Nicotiana tabacum), AA925316 (Rattus norvegicus), AA924358 (Rattus norvegicus), AA923907 (Rattus norvegicus), AA901212 (Rattus norvegicus), AA858643 (Rattus norvegicus), AA441366 (Drosophila melanogaster), AA440162 (Drosophila melanogaster), L42763 (Styela clava), AF085197 (Nicotiana tabacum), AF020427 (Sarcophaga crassipalpis), AB002264 (Bombyx mori), J04718 (Human), M34080 (X.laevis) and M33950 (D.melanogaster).

q. Mutant Replication Factor C

In yet another specific embodiment, a mutant replication factor C is used in the present methods. The nucleic acid molecules containing sequences of nucleotides with the following GenBank accession Nos. can be used in obtaining nucleic acid encoding replication factor C and in mutagenesis: AF139987 (Mus musculus), AA924760 (Homo sapiens), AA901331 (Homo sapiens), AA900852 (Homo sapiens), AA899302 (Homo sapiens), AA819500 (Rattus norvegicus), U60144 (Anas platyrhynchos), U26031 (Saccharomyces cerevisiae), U26030 (Saccharomyces cerevisiae), U26029 (Saccharomyces cerevisiae), U26028 (Saccharomyces cerevisiae), U26027 (Saccharomyces cerevisiae), AF045555 (Homo sapiens), U86620 (Emericella nidulans), U86619 (Emericella nidulans), D28499 (Yeast), U07685 (Drosophila melanogaster), M87338 (Human), M87339 (Human), L07540 (Human), L07541 (Human), L20502 (Saccharomyces cerevisiae), L18755 (Saccharomyces cerevisiae), U12438 (Gallus gallus Leghorn) and L23320 (Human).

r. Mutant Uracil DNA Glycosylase

In yet another specific embodiment, a mutant uracil DNA glycosylase (UDG) is used in the present methods. The nucleic acid molecules containing sequences of nucleotides with the following GenBank accession Nos. can be used in obtaining nucleic acid encoding uracil DNA glycosylase and in mutagenesis: AF174292 (Schizosaccharomyces pombe), AF108378 (Cercopithecine herpesvirus), AF125182 (Homo sapiens), AF125181 (Xenopus laevis), U55041 (Homo sapiens), U55041 (Mus musculus), AF084182 (Guinea pig cytomegalovirus), U31857 (Bovine herpesvirus), AF022391 (Feline herpesvirus), M87499 (Human), J04434 (Bacteriophage PBS2), U13194 (Human herpesvirus 6), L34064 (Gallid herpesvirus 1), U04994 (Gallid herpesvirus 2), L01417 (Rabbit fibroma virus), M25410 (Herpes simplex virus type 2), J04470 (S.cerevisiae), J03725 (E.coli), U02513 (Suid herpesvirus), U02512 (Suid herpesvirus) and L13855 (Pseudorabies virus).

s. Mutant Thymidine DNA Glycosylase

In yet another specific embodiment, a mutant thymidine DNA glycosylase (TDG) is used in the present methods. The nucleic acid molecules containing sequences of nucleotides with the following GenBank accession Nos. can be used in obtaining nucleic acid encoding thymidine DNA glycosylase and in mutagenesis: AF117602 (Ateles paniscus chamek). Preferably, the abnormal base-pairing to be detected is a G:T mismatch and the mutant DNA repair enzyme used in the present methods is a mutant TDG (Hsu et al., Carcinogenesis, 15(8):1657-62 (1994)).

t. Mutant Dam

In yet another specific embodiment, a mutant dam is used in the present methods. The nucleic acid molecules containing sequences of nucleotides with the following GenBank accession Nos. can be used in obtaining nucleic acid encoding dam and in mutagenesis: AF091142 (Neisseria meningitidus strain BF13), AF006263 (Treponema pallidum), U76993 (Salmonella typhimurium) and M22342 (Bacteriphage T2).

2. Detecting the Binding of the Mutant Enzyme

Binding of the mutant enzyme to a duplex can be detected by any method known to those of skill in the art for detection of proteins. The enzyme may be specifically labeled, such as with a fluorescent label, radiolabeled, tagged with a readily tag that can be readily purified, labeled with another enzyme, or antibody. In an exemplary embodiment, biotin is bound to the mutant enzyme, which can then interact with a streptavidin-labeled moiety, such a horse radish peroxidase (HRPO), which upon reaction with an appropriate substrate will form a colored product.

For example, an array of nucleic acid probes, containing for example, from about 20 to about to about 100 nucleotides, are hybridized with single-stranded nucleic acid from a sample. The hybids are contacted with a selected or a plurality of mutant enzymes, which are labeled with biotin. After contacting the biotin reacts with streptavidin which is labeled, such as with HRPO, and the bound mutant enzyme is detected by virtue of the formation of detectable product, such as colored product. If the probes on the array are of known sequence, selected, for example for inclusion of polymorphisms, then upon reaction, the presence or absence of an array of polymorphism in the sample can be rapidly and readily identified.

### C. Methods for Detecting Mutations in Nucleic Acids for Prognosis and Diagnosis of Diseases, Disorders and Infections

Also provided herein are methods for detecting a mutations in a nucleic acid molecule for diagnostic and prognostic applications. These methods involve binding a mutant nucleic acid binding enzyme, such as a mutant repair enzyme to nucleic acids in sample, such as body tissue or fluid sample, and detecting the bound mutant enzyme. These reactions can be performed in solution, or, preferably in solid phase.

In one embodiment, single-stranded nucleic acids, either those known to be wild type or with a mutation indicative of a particular disorder are hybridized with the sample nucleic acid. The resulting duplexes are contacted with a selected mutant enzyme or a plurality thereof that contain different specificities. The resulting complexes, which are indicative a difference in sequence between the strands in the sample from the known strands, are detected. These methods can be performed in solution or preferably in solid phase. In a preferred embodiment, the single-stranded nucleic acids containing known sequences are on the solid support. In others, the enzymes of known specificities can be bound on a solid support. Bound hybrids are indicative of the mutation present.

In a preferred embodiment, the method is performed by hybridizing a strand of a nucleic acid having or suspected of having a mutation with a complementary strand of a wild-type nucleic acid (or with a strand having a known mutation), whereby the mutation results in an abnormal base-pairing in the formed nucleic acid duplex; contacting the nucleic acid duplex with a mutant DNA repair enzyme or complex thereof, where the mutant DNA repair enzyme or complex thereof has binding affinity for the abnormal base-pairing in the duplex but has attenuated catalytic activity; and detecting binding between the nucleic acid duplex and the mutant DNA repair enzyme or complex thereof, whereby the presence or quantity of the mutation is assessed.

Any mutant DNA repair enzymes or complexes thereof that have binding affinity for the abnormal base-pairing in the duplex but have attenuated catalytic activity can be used in the mutation detection. Preferably, the mutant DNA repair enzymes or complexes thereof described in the above Section B can be used. Typically, the nucleic acid strand to be tested and the complementary wild-type nucleic acid strand are DNA strands.

Mutations that can be detected by these methods, include those that are associated with or that are indicative of a disease or disorder or predilection thereto, or infection by a pathological agent. These methods can be used for prognosis or diagnosis of the presence or severity of the disease, disorder or infection.

Any diseases, disorders or infections that are associated with a nucleic acid mutation or for which such mutation serves as a marker or indicator can be diagnosed or the tendency therefor prognosticated using the present methods. Such diseases and disorders include, but are not limited to, cancers, immune system diseases or disorders, metabolism diseases or disorders, muscle and bone diseases or disorders, nervous system diseases or disorders, signal diseases or disorders and transporter diseases or disorders. Infections include, but are not limited to, infections caused by viruses, eubacteria, archaebacteria and eukaryotic pathogens.

Among the diseases or disorders that can be diagnosed or the tendency to develop them, include but are not limited to, a disease or disorder associated with an androgen receptor mutation, tetrahydrobiopterin deficiencies, X-Linked agammaglobulinemia, a disease or disorder associated with a factor VII mutation, anemia, a disease or disorder associated with a glucose-6-phosphate mutation, the glycogen storage disease type II (Pompe Disease), hemophilia A, a disease or disorder associated with a hexosaminidase A mutation, a disease or disorder associated with a human type I or type III collagen mutation, a disease or disorder associated with a rhodopsin or RDS mutation, a disease or disorder associated with a L1CAM mutation, a disease or disorder associated with a LDL receptor mutation, a disease or disorder associated with an ornithine transcarbamylase mutation, a disease or disorder associated with a PAX6 mutation and a disease or disorder associated with a von Willebrand factor mutation.

1. Cancer

Any cancers that are associated with a mutation(s) in a nucleic acid can be predicted or diagnosed using the present methods. For example, breast cancer, Burkitt lymphoma, colon cancer, small cell lung carcinoma, melanoma, multiple endocrine neoplasia (MEN), neurofibromatosis, p53-associated tumor, pancreatic carcinoma, prostate cancer, Ras-associated tumor, retinoblastoma and Von-Hippel Lindau disease (VHL) can be predicted or diagnosed using the present methods.

a. Breast Cancer

Two breast cancer susceptibility genes have been identified: BRCA1 on chromosome 17 and BRCA2 on chromosome 13. When an individual carries a mutation in either BRCA1 or BRCA2, they are at an increased risk of being diagnosed with breast or ovarian cancer at some point in their lives (Albertsen et al., Am. J. Hum. Genet., 54(3):516-25 (1994); and Wooster et al., Nature, 378(6559):789-92 (1995)). Until recently, it was not clear what the function of these genes was, until studies on a related protein in yeast revealed their normal role: they participate in repairing radiation-induced breaks in double-stranded DNA. It is thought that mutations in BRCA1 or BRCA2 might disable this mechanism, leading to more errors in DNA replication and ultimately to cancerous growth.

In a specific embodiment, the breast cancer to be predicted or diagnosed according to the present method is associated with a mutation in BRCA1 or BRCA2.

b. Burkitt Lymphoma

Burkitt lymphoma results from chromosome translocations that involve the Myc gene. A chromosome translocation means that a chromosome is broken, which allows it to associate with parts of other chromosomes (Adams et al., Proc. Natl. Acad. Sci. U.S.A., 80(7):1982-6 (1983); Watt et al., Nature, 303(5919):725-8 (1983); and Cole, Annu. Rev. Genet., 20:361-84 (1986)). The classic chromosome translocation in Burkitt lymphoma involves chromosome 8, the site of the Myc gene. This changes the pattern of Myc's expression, thereby disrupting its usual function in controlling cell growth and proliferation.

In a specific embodiment, the Burkitt lymphoma to be predicted or diagnosed according to the present method is associated with a mutation in Myc.

c. Colon Cancer

Colon cancer is one of the most common inherited cancer syndromes known. Two key genes involved in colon cancer have been found: MSH2, on chromosome 2 and MLH1, on chromosome 3. Normally, the protein products of these genes help to repair mistakes made in DNA replication. If the MSH2 and MLH1 proteins are mutated and therefore don't work properly, the replication mistakes are not repaired, leading to damaged DNA and, in this case, colon cancer (Bronner et al., Nature, 368(6468):258-61 (1994); and Fishel et al, Cell, 75(5):1027-38 (1993)).

In a specific embodiment, the colon cancer to be predicted or diagnosed according to the present method is associated with a mutation in MSH2 or MLH1.

d. Small Cell Lung Carcinoma

Small cell lung carcinoma is distinctive from other kinds of lung cancer (metastases are already present at the time of discovery) and accounts for approximately 110,000 cancer diagnoses annually. A deletion of part of chromosome 3, SCLC1, was first observed in 1982 in small cell lung carcinoma cell lines (Whang-Peng et al., Science, 215(4529):181-2 (1982)).

In a specific embodiment, the small cell lung carcinoma to be predicted or diagnosed according to the present method is associated with a mutation in SCLC 1.

e. Melanoma Carcinoma

In some cases, the risk of developing melanoma runs in families, where a mutation in the CDKN2 gene on chromosome 9 can underlie susceptibility to melanoma (Hussussian et al., Nat. Genet., 8(1):15-21 (1994)). CDKN2 codes for a protein called p16 that is an important regulator of the cell division cycle: it stops the cell from synthesizing DNA before it divides. If p16 is not working properly, the skin cell does not have this brake on the cell division cycle, and so can go on to proliferate unchecked. At some point this proliferation can be seen as a sudden change in skin growth or the appearance of a mole.

In a specific embodiment, the melanoma carcinoma to be predicted or diagnosed according to the present method is associated with a mutation in CDKN2.

f. Multiple Endocrine Neoplasia

Multiple endocrine neoplasia (MEN) is a group of rare diseases caused by genetic defects that lead to hyperplasia (abnormal multiplication or increase in the number of normal cells in normal arrangement in a tissue) and hyperfunction (excessive functioning) of 2 or more components of the endocrine system. Normally, the hormones released by endocrine glands are carefully balanced to met the body's needs. When a person has MEN, specific endocrine glands, such as the parathyroid glands, the pancreas gland and the pituitary gland, tend to become overactive. When these glands go into overdrive, the result can be: excessive calcium in the bloodstream (resulting in kidney stones or kidney damage); fatigue; weakness; muscle or bone pain; constipation; indigestion; and thinning of bones. The MEN1 gene, which has been known for several years to be found on chromosome 11, was more finely mapped in 1997 (Chandrasekharappa et al., Science, 276(5311):404-7 (1997)). In a specific embodiment, the MEN to be diagnosed or predicted according to the present method is associated with a mutation in MEN1.

g. Neurofibromatosis

Neurofibromatosis, type 2 (NF-2), is a rare inherited disorder characterized by the development of benign tumors on auditory nerves (acoustic neuromas). The disease is also characterized by the development of malignant central nervous system tumors as well. The NF2 gene has been mapped to chromosome 22 and is thought to be a ‘tumor-suppressor gene’ (Rouleau et al., Nature, 363(6429):515-21 (1993)). A mutation in NF2 impairs its function, and accounts for the clinical symptoms observed in neurofibromatosis sufferers. NF-2 is an autosomal dominant genetic trait; it affects both genders equally and each child of an affected parent has a 50% chance of inheriting the gene.

In a specific embodiment, the neurofibromatosis to be predicted or diagnosed according to the present method is associated with a mutation in NF2.

h. Cancer Associated with p53 Mutation

The p53 gene is a tumor suppressor gene (Harlowet al., Mol. Cell. Biol., 5(7):1601-10 (1985)). If a person inherits only one functional copy of the p53 gene from their parents, they are predisposed to cancer and usually develop several independent tumors in a variety of tissues in early adulthood. This condition is rare, and is known as Li-Fraumeni syndrome. Mutations in p53 are found in most tumor types, and so contribute to the complex network of molecular events leading to tumor formation. The p53 gene has been mapped to chromosome 17. In the cell, p53 protein binds DNA, which in turn stimulates another gene to produce a protein called p21 that interacts with a cell division-stimulating protein (cdk2). When p21 is complexed with cdk2 the cell cannot pass through to the next stage of cell division. Mutant p53 can no longer bind DNA in an effective way, and as a consequence the p21 protein is not made available to act as the ‘stop signal’ for cell division. Thus cells divide uncontrollably, and form tumors.

In a specific embodiment, the cancer to be predicted or diagnosed according to the present method is associated with a mutation in p53.

i. Pancreatic Carcinoma

About 90% of human pancreatic carcinomas show a loss of part of chromosome 18. In 1996, a possible tumor suppressor gene, DPC4 (Smad4), was discovered from the section that is lost in pancreatic cancer, so may play a role in pancreatic cancer (Hahn et al., Science, 271(5247):350-3 (1996)). There is a whole family of Smad proteins in vertebrates, all involved in signal transduction of transforming growth factor-beta (TGF-beta) related pathways.

In a specific embodiment, the pancreatic carcinoma to be predicted or diagnosed according to the present method is associated with a mutation in DPC4 (Smad4).

j. Prostate Cancer

One of the most promising recent breakthroughs in prostate cancer research is the discovery of a susceptibility locus for prostate cancer on chromosome 1, called HPC1, which may account for about 1 in 500 cases of prostate cancer (Smith et al., Science, 274(5291):1371-4 (1996)).

In a specific embodiment, the prostate cancer to be predicted or diagnosed according to the present method is associated with a mutation in HPC1.

k. Cancer Associated with Ras Oncogene

Ras is an oncogene product that is found on chromosome 11. It is found in normal cells, where it helps to relay signals by acting as a switch (Lowy and Willumsen, Annu. Rev. Biochem., 62:851-91 (1993); Russell et al., Genomics, 35(2):353-60 (1996); and Tong et al., Nature, 337(6202):90-3 (1989)). When receptors on the cell surface are stimulated (by a hormone, for example), Ras is switched on and transduces signals that tell the cell to grow. If the cell-surface receptor is not stimulated, Ras is not activated and so the pathway that results in cell growth is not initiated. In about 30% of human cancers, Ras is mutated so that it is permanently switched on, telling the cell to grow regardless of whether receptors on the cell surface are activated or not.

In a specific embodiment, the cancer to be predicted or diagnosed according to the present method is associated with a mutation in Ras oncogene.

l. Retinoblastoma

Retinoblastoma occurs in early childhood and develops from the immature retina—the part of the eye responsible for detecting light and color. There are hereditary and non-hereditary forms of retinoblastoma. In the hereditary form, multiple tumors are found in both eyes, while in the non-hereditary form only one eye is effected and by only one tumor. In the hereditary form, a gene called Rb is lost from chromosome 13 (Friend et al., Nature, 323(6089):643-6 (1986); and Lee et al., Science, 235(4794):1394-9 (1987)). Rb is found in all cells of the body, where under normal conditions it acts as a brake on the cell division cycle by preventing certain regulatory proteins from triggering DNA replication. If Rb is missing, a cell can replicate itself over and over in an uncontrolled manner, resulting in tumor formation.

In a specific embodiment, the retinoblastoma to be predicted or diagnosed according to the present method is associated with a mutation in Rb gene.

m. Von-Hippel Lindau Syndrome

Von-Hippel Lindau syndrome is an inherited multi-system disorder characterized by abnormal growth of blood vessels. While blood vessels normally grow like trees, in people with VHL little knots of blood capillaries sometimes occur. These knots are called angiomas or hemangioblastomas. Growths may develop in the retina, certain areas of the brain, the spinal cord, the adrenal glands and other parts of the body. The gene for Von-Hippel Lindau disease (VHL) is found on chromosome 3, and is inherited in a dominant fashion (Latif et al., Science, 260(5112):1317-20 (1993)). If one parent has a dominant gene, each child has a 50-50 chance of inheriting that gene. The VHL gene is a tumor suppressor gene.

In a specific embodiment, the Von-Hippel Lindau syndrome to be predicted or diagnosed according to the present method is associated with a mutation in VHL gene.

2. Immune System Disease or Disorder

Any immune system diseases or disorders that are associated with a mutation(s) in a nucleic acid can be predicted or diagnozed using the present methods. For example, autoimmune polyglandular syndrome type I (APS1, also called APECED), inflammatory bowel disease (IBD), DiGeorge syndrome, familial Mediterranean fever (FMF) and severe combined immunodeficiency (SCID) can be predicted or diagnosed using the present methods.

a. Autoimmune Polyglandular Syndrome Type I

Autoimmune polyglandular syndrome type I (APS1, also called APECED) is a rare autosomal recessive disorder that maps to human chromosome 21. At the end of 1997, researchers reported that they isolated a novel gene, which they called AIRE (autoimmune regulator). Database searches revealed that the protein product of this gene is a transcription factor—a protein that plays a role in the regulation of gene expression. The researchers showed that mutations in this gene are responsible for the pathogenesis of APS I (Nagamine et al., Na.t Genet., 17(4):393-8 (1997)).

In a specific embodiment, the autoimmune polyglandular syndrome type I to be predicted or diagnosed according to the present method is associated with a mutation in AIRE gene.

b. Inflammatory Bowel Disease

Inflammatory bowel disease (IBD) is a group of chronic disorders that cause inflammation or ulceration in the small and large intestines. Most often, IBD is classified either as ulcerative colitis or Crohn disease. While ulcerative colitis affects the inner lining of the colon and rectum, Crohn disease extends into the deeper layers of the intestinal wall. It is a chronic condition and may recur at various times over a lifetime. About 20% of cases of Crohn disease appear to run in families. It is a ‘complex trait’, which means that several genes at different locations in the genome may contribute to the disease. A susceptibility locus for the disease was recently mapped to chromosome 16. Candidate genes found in this region include several involved in the inflammatory response, including: CD19, involved in B-lymphocyte function; sialophorin, involved in leukocyte adhesion; the CD11 integrin cluster, involved in microbacteria cell adhesion; and the interleukin-4 receptor, which is interesting, as IL-4-mediated functions are altered in IBDs (Hugot et al., Nature, 379(6568):821-3 (1996)).

In a specific embodiment, the inflammatory bowel disease to be predicted or diagnosed according to the present method is associated with a mutation in CD19, sialophorin, CD11 integrin cluster or interleukin-4 receptor.

c. DiGeorge Syndrome

DiGeorge syndrome is a rare congenital (i.e., present at birth) disease whose symptoms vary greatly between individuals, but commonly include a history of recurrent infection, heart defects and characteristic facial features. DiGeorge syndrome is caused by a large deletion from chromosome 22, produced by an error in recombination at meiosis (the process that creates germ cells and ensures genetic variation in the offspring). This deletion means that several genes from this region are not present in DiGeorge syndrome patients. It appears that the variation in the symptoms of the disease is related to the amount of genetic material lost in the chromosomal deletion (Budarf et al., Nat. Genet., 10(3):269-78 (1995)).

d. Familial Mediterranean Fever

Familial Mediterranean fever (FMF) is an inherited disorder usually characterized by recurrent episodes of fever and peritonitis (inflammation of the abdominal membrane). In 1997, researchers identified the gene for FMF and found several different gene mutations that cause this inherited rheumatic disease. The gene, found on chromosome 16, codes for a protein that is found almost exclusively in granulocytes—white blood cells important in the immune response. The protein is likely to normally assist in keeping inflammation under control by deactivating the immune response—without this ‘brake’, an inappropriate full-blown inflammatory reaction occurs: an attack of FMF (Cell, 90(4):797-807 (1997); and Nat. Genet., 17(1):25-31 (1997)).

In a specific embodiment, the familial Mediterranean fever to be predicted or diagnosed according to the present method is associated with a mutation in FMF gene.

e. Severe Combined Immunodeficiency

Severe combined immunodeficiency (SCID) represents a group of rare, sometimes fatal, congenital disorders characterized by little or no immune response (Valerio et al, EMBO J., 4(2):437-43 (1985); and Noguchi et al., Cell, 73(1):147-57 (1993)). The defining feature of SCID, commonly known as “bubble boy” disease, is a defect in the specialized white blood cells (B- and T-lymphocytes) that defend us from infection by viruses, bacteria and fungi. Without a functional immune system, SCID patients are susceptible to recurrent infections such as pneumonia, meningitis and chicken pox, and can die before the first year of life. All forms of SCID are inherited, with as many as half of SCID cases linked to the X chromosome, passed on by the mother. X-linked SCID results from a mutation in the interleukin 2 receptor gamma (IL2RG) gene which produces the common gamma chain subunit, a component of several IL receptors. Defective IL receptors prevent the proper development of T-lymphocytes that play a key role in identifying invading agents as well as activating and regulating other cells of the immune system. In another form of SCID, there is a lack of the enzyme adenosine deaminase (ADA), coded for by a gene on chromosome 20. This means that the substrates for this enzyme accumulate in cells. Immature lymphoid cells of the immune system are particularly sensitive to the toxic effects of these unused substrates, so fail to reach maturity. As a result, the immune system of the afflicted individual is severely compromised or completely lacking.

In a specific embodiment, the severe combined immunodeficiency to be predicted or diagnosed according to the present method is associated with a mutation in interleukin 2 receptor gamma (IL2RG) or adenosine deaminase (ADA).

3. Metabolism System Diseases and Disorders

Any metabolism diseases or disorders that are associated with a mutation(s) in a nucleic acid can be predicated or diagnosed using the present methods. For example, adrenoleukodystrophy (ALD), atherosclerosis, Gaucher disease, gyrate atrophy of the choroid, diabetes, obesity, paroxysmal nocturnal hemoglobinuria (PNH), phenylketonuria (PKU), Refsum disease and Tangier disease (TD) can be predicted or diagnosed using the present methods.

a. Adrenoleukodystrophy

Adrenoleukodystrophy (ALD) is a rare, inherited metabolic disorder. In this disease the fatty covering (myelin sheath) on nerve fibers in the brain is lost, and the adrenal gland degenerates, leading to progressive neurological disability and death. People with ALD accumulate high levels of saturated, very long chain fatty acids in their brain and adrenal cortex because the fatty acids are not broken down by an enzyme in the normal manner. So, when the ALD gene was discovered in 1993, it was a surprise that the corresponding protein was in fact a member of a family of transporter proteins, not an enzyme (Mosser et al., Nature, 361(6414):726-30 (1993)).

In a specific embodiment, the adrenoleukodystrophy to be predicted or diagnosed according to the present method is associated with a mutation in ALD gene.

b. Atherosclerosis

Atherosclerosis is characterized by a narrowing of the arteries caused by cholesterol-rich plaques of immune-system cells. Key risk factors for atherosclerosis, which can be genetic and/or environmental, include: elevated levels of cholesterol and triglyceride in the blood, high blood pressure and cigarette smoke. A protein called apolipoprotein E, which can exist in several different forms, is coded for by a gene found on chromosome 19. It is important for removing excess cholesterol from the blood, and does so by carrying cholesterol to receptors on the surface of liver cells. Defects in apolipoprotein E sometimes result in its inability to bind to the receptors, which leads to an increase a person's blood cholesterol, and consequently their risk of atherosclerosis (Das et al., J. Biol. Chem., 260(10):6240-7 (1985); and Breslow, Science, 272(5262):685-8 (1996)).

In a specific embodiment, the atherosclerosis to be predicted or diagnosed according to the present method is associated with a mutation in apolipoprotein E.

c. Gaucher Disease

Gaucher disease is an inherited illness caused by a gene mutation (Barneveld et al., Hum. Genet., 64(3):227-31 (1983); and Beutler, Science, 256(5058):794-9 (1992)). Normally, this gene is responsible for an enzyme called glucocerebrosidase that the body needs to break down a particular kind of fat called glucocerebroside. In people with Gaucher disease, the body is not able to properly produce this enzyme and the fat cannot be broken down. It then accumulates, mostly in the liver, spleen and bone marrow. Gaucher disease can result in pain, fatigue, jaundice, bone damage, anemia and even death.

In a specific embodiment, the Gaucher disease to be predicted or diagnosed according to the present method is associated with a mutation in glucocerebrosidase.

d. Gyrate Atrophy of the Choroid

People suffering from gyrate atrophy of the choroid (the thin coating of the eye) and retina face a progressive loss of vision, with total blindness usually occurring between the ages of 40 and 60. The disease is an inborn error of metabolism. The gene whose mutation causes gyrate atrophy is found on chromosome 10, and encodes an enzyme called ornithine ketoacid aminotransferase (OAT) (Akaki et al., J. Biol Chem., 267(18):12950-4 (1992); and O'Donnell et al., Am. J. Hum. Genet., 43(6):922-8 (1988)). Different inherited mutations in OAT cause differences in the severity of symptoms of the disease. OAT converts the amino acid ornithine from the urea cycle ultimately into glutamate. In gyrate atrophy, where OAT function is affected, there is an increase in plasma levels of ornithine.

In a specific embodiment, the gyrate atrophy of the choroid to be predicted or diagnosed according to the present method is associated with a mutation in ornithine ketoacid aminotransferase (OAT).

e. Diabetes

Diabetes is a chronic metabolic disorder that adversely affects the body's ability to manufacture and use insulin, a hormone necessary for the conversion of food into energy. The disease greatly increases the risk of blindness, heart disease, kidney failure, neurological disease and other conditions for the approximately 16 million Americans who are affected by it. Type I, or juvenile onset diabetes, is the more severe form of the illness. Type I diabetes is what is known as a ‘complex trait’, which means that mutations in several genes likely contribute to the disease (Nuffield et al., Nature, 371(6493):130-6 (1994)). For example, it is now known that the insulin-dependent diabetes mellitus (IDDM1) locus on chromosome 6 may harbor at least one susceptibility gene for Type I diabetes. In Type I diabetes, the body's immune system mounts an immunological assault on its own insulin and the pancreatic cells that manufacture it. About 10 loci in the human genome have now been found that seem to confer susceptibility to Type I diabetes. Among these are (1) a gene at the locus IDDM2 on chromosome 11 and (2) the gene for glucokinase (GCK), an enzyme that is key to glucose metabolism which helps modulate insulin secretion, on chromosome 7.

In a specific embodiment, the diabetes of the choroid to be predicted or diagnosed according to the present method is associated with a mutation in insulin-dependent diabetes mellitus (IDDM1) locus, a gene at the locus IDDM2, or glucokinase (GCK).

f. Obesity

Obesity is an excess of body fat that frequently results in a significant impairment of health. Evidence suggests that obesity has more than one cause: genetic, environmental, psychological and other factors may all play a part. The hormone leptin, produced by adipocytes (fat cells), was discovered about three years ago in mice (Zhang et al., Nature, 372(6505):425-32 (1994)). Subsequently the human Ob gene was mapped to chromosome 7. Leptin is thought to act as a lipostat: as the amount of fat stored in adipocytes rises, leptin is released into the blood and signals to the brain that the body has enough to eat. Most overweight people have high levels of leptin in their bloodstream, indicating that other molecules also effect feelings of salty and contribute to the regulation of body weight.

In a specific embodiment, the obesity to be predicted or diagnosed according to the present method is associated with a mutation in leptin or human Ob gene.

g. Paroxysmal Nocturnal Hemoglobinuria

The paroxysmal nocturnal hemoglobinuria (PNH) is characterized by a decreased number of red blood cells (anemia), and the presence of blood in the urine (hemoglobinuria) and plasma (hemoglobinemia), which is evident after sleeping. PNH is associated with a high risk of major thrombotic events, most commonly thrombosis of large intra-abdominal veins. Most patients who die of their disease die of thrombosis. PNH blood cells are deficient in an enzyme known as PIG-A, which is required for the biosynthesis of cellular anchors (Bessler et al., EMBO J., 13(1):110-7 (1994); and Miyata et al., Science, 259(5099):1318-20 (1993)). Proteins that are partly on the outside of cells are often attached to the cell membrane by a glycosylphosphatidylinositol (GPI) anchor, and PIG-A is required for the synthesis of a key anchor component. If PIG-A is defective, surface proteins that protect the cell from destructive components in the blood (complement) are not anchored and therefore absent, so the blood cells are broken down. The PIG-A gene is found on the X chromosome. Although not an inherited disease, PNH is a genetic disorder, known as an acquired genetic disorder. The affected blood cell clone passes the altered PIG-A to all its descendants--red cells, leukocytes (including lymphocytes), and platelets. The proportion of abnormal red blood cells in the blood determines the severity of the disease.

In a specific embodiment, the paroxysmal nocturnal hemoglobinuria to be predicted or diagnosed according to the present method is associated with a mutation in PIG-A.

h. Phenylketonuria

Phenylketonuria (PKU) is an inherited error of metabolism caused by a deficiency in the enzyme phenylalanine hydroxylase (DiLella et al., Nature, 327(6120):333-6 (1987); and Kwok et al., Biochemistry, 24(3):556-61 (1985)). Loss of this enzyme results in mental retardation, organ damage, unusual posture and can, in cases of maternal PKU, severely compromise pregnancy. Classical PKU is an autosomal recessive disorder, caused by mutations in both alleles of the gene for phenylalanine hydroxylase (PAH), found on chromosome 12. In the body, phenylalanine hydroxylase converts the amino acid phenylalanine to tyrosine, another amino acid. Mutations in both copies of the gene for PAH means that the enzyme is inactive or is less efficient, and the concentration of phenylalanine in the body can build up to toxic levels. In some cases, mutations in PAH will result in a phenotypically mild form of PKU called hyperphenylalanemia. Both diseases are the result of a variety of mutations in the PAH locus; in those cases where a patient is heterozygous for two mutations of PAH (i.e., each copy of the gene has a different mutation), the milder mutation will predominate.

In a specific embodiment, the phenylketonuria to be predicted or diagnosed according to the present method is associated with a mutation in phenylalanine hydroxylase.

i. Refsum Disease

Refsum disease is a rare disorder of lipid metabolism that is inherited as a recessive trait. Symptoms may include a degenerative nerve disease (peripheral neuropathy), failure of muscle coordination (ataxia), retinitis pigmentosa (a progressive vision disorder), and bone and skin changes. Refsum disease is characterized by an accumulation of phytanic acid in the plasma and tissues. is a derivative of phytol, a component of chlorophyll. In 1997 the gene for Refsum disease was identified and mapped to chromosome 10 (Jansen et al., Nat. Genet., 17(2):190-3 (1997); and Mihalik et al., Nat. Genet., 17(2):185-9 (1997)). The protein product of the gene, PAHX, is an enzyme that is required for the metabolism of phytanic acid. Refsum disease patients have impaired PAHX—phytanic acid hydrolase.

In a specific embodiment, the Refsum disease to be predicted or diagnosed according to the present method is associated with a mutation in PAHX.

j. Tangier Disease

Tangier disease (TD) is a genetic disorder of cholesterol transport named for the secluded island of Tangier, located off the coast of Virginia. TD was first identified in a five-year-old inhabitant of the island who had characteristic orange tonsils, very low levels of high density lipoprotein (HDL) or ‘good cholesterol’, and an enlarged liver and spleen. TD is caused by mutations in the ABC1 (ATP-binding cassette) gene on chromosome 9q31 (Rust et al., Nat. Genet., 22(4):352-5 (1999); Bodzioch et al., Nat. Genet., 22(4):347-51 (1999); Brooks-Wilson et al., Nat. Genet., 22(4):336-45 (1999); and Rust et al., Nat. Genet., 20(1):96-8 (1998)). ABC1 codes for a protein that helps rid cells of excess cholesterol. This cholesterol is then picked up by HDL particles in the blood and carried to the liver, which processes the cholesterol to be reused in cells throughout the body. Individuals with TD are unable to eliminate cholesterol from cells, leading to its buildup in the tonsils and other organs.

In a specific embodiment, the Tangier disease to be predicted or diagnosed according to the present method is associated with a mutation in ABC1 (ATP-binding cassette) gene on chromosome 9q31.

4. Muscle and Bone Diseases and Disorders

Any muscle and bone diseases or disorders that are associated with a mutation(s) in a nucleic acid can be predicted or diagnosed using the present methods. For example, Duchenne muscular dystrophy (DMD), ELLIS-VAN CREVELD syndrome (chondroectodermal dysplasia), Marfan syndrome and myotonic dystrophy can be predicted or diagnosed using the present methods.

a. Duchenne Muscular Dystrophy

Duchenne muscular dystrophy (DMD) is one of a group of muscular dystrophies characterized by the enlargement of muscles. The gene for DMD, found on the X chromosome, encodes a large protein—dystrophin (Koenig et al., Cell, 53(2):219-26 (1988)). Dystrophin is required inside muscle cells for structural support: it is thought to strengthen muscle cells by anchoring elements of the internal cytoskeleton to the surface membrane. Without it, the cell membrane becomes permeable, so that extracellular components enter the cell, increasing the internal pressure until the muscle cell ‘explodes’ and dies. The subsequent immune response can add to the damage.

In a specific embodiment, the Duchenne muscular dystrophy to be predicted or diagnosed according to the present method is associated with a mutation in dystrophin.

b. Ellis-Van Creveld Syndrome

Ellis-Van Creveld syndrome, also known as ‘chondroectodermal dysplasia’, is a rare genetic disorder characterized by short-limb dwarfism, polydactyly (additional fingers or toes), malformation of the bones of the wrist, dystrophy of the fingernails, partial hare-lip, cardiac malformation and often prenatal eruption of the teeth. The gene causing Ellis-van Creveld syndrome, EVC, has been mapped to the short arm of chromosome 4 (Polymeropoulos et al., Genomics, 35(1):1-5 (1996)). A pattern of inheritance can be observed that has indicated the disease is autosomal-recessive (i.e., a mutated gene form both parents is required before the effects of the disease to become apparent).

In a specific embodiment, the Ellis-Van Creveld syndrome to be predicted or diagnosed according to the present method is associated with a mutation in EVC gene.

c. Marfan Syndrome

Marfan syndrome is a connective tissue disorder, so affects many structures, including the skeleton, lungs, eyes, heart and blood vessels. The disease is characterized by unusually long limbs. Marfan syndrome is an autosomal dominant disorder that has been linked to the FBN1 gene on chromosome 15 (Dietz et al., Nature, 352(6333):337-9 (1991); and Kainulainen et al., N. Engl. J. Med., 323(14):935-9 (1990)). FBN1 encodes a protein called fibrillin, which is essential for the formation of elastic fibers found in connective tissue. Without the structural support provided by fibrillin, many tissues are weakened, which can have severe consequences, for example, ruptures in the walls of major arteries.

In a specific embodiment, the Marfan syndrome to be predicted or diagnosed according to the present method is associated with a mutation in FBN1.

d. Myotonic Dystrophy

Myotonic dystrophy is an inherited disorder in which the muscles contract but have decreasing power to relax. With this condition, the muscles also become weak and waste away. Myotonic dystrophy can cause mental deficiency, hair loss and cataracts. Onset of this rare disorder commonly occurs during young adulthood. It can occur at any age and is extremely variable in degree of severity. The myotonic dystrophy gene, found on chromosome 19, codes for a protein kinase that is found in skeletal muscle, where it likely plays a regulatory role (Aslanidis et al, Nature, 355(6360):548-51 (1992)). An unusual feature of this illness is that its symptoms usually become more severe with each successive generation. This is because mistakes in the faithful copying of the gene from one generation to the next result in the amplification of a ‘AGC triplet repeat’, similar to that found in Huntington disease. Unaffected individuals have between 5 and 27 copies of AGC, myotonic dystrophy patients who are minimally affected have at least 50 repeats, while more severely affected patients have an expansion of up to several kilobase pairs.

In a specific embodiment, the myotonic dystrophy to be predicted or diagnosed according to the present method is associated with a mutation in myotonic dystrophy gene.

5. Nervous System Diseases and Disorders

Any nervous system diseases and disorders that are associated with a mutation(s) in a nucleic acid can be predicted or diagnosed using the present methods. For example, Alzheimer disease (AD), amyotrophic lateral sclerosis (ALS), Angelman syndrome (AS), Charcot-Marle-tooth disease (CMT), epilepsy, tremor, fragile X syndrome, Friedreich's ataxia (FRDA), Huntington disease (HD), Niemann-Pick, Parkinson disease, Prader-Willi syndrome (PWS), spinocerebellar atrophy and Williams syndrome can be predicted or diagnosed using the present methods.

a. Alzheimer's Disease

Alzheimer' Disease (AD) is the fourth leading cause of death in adults. The incidence of the disease rises steeply with age. Some of the most frequently observed symptoms of the disease include a progressive inability to remember facts and events and, later, to recognize friends and family. Certain types of AD run in families: currently, mutations in four genes, situated on chromosomes 1, 14, 19 and 21, are believed to play a role in the disease. The best-characterized of these are PS1 (or AD3) on chromosome 14 and PS2 (or AD4) on chromosome 1 (Levy-Lahad et al., Science, 269(5226):973-7 (1995); and Sherrington et al., Nature, 375(6534):754-60 (1995)). The formation of lesions made of fragmented brain cells surrounded by amyloid-family proteins are characteristic of the disease. These lesions and their associated proteins are closely related to similar structures found in Down's Syndrome. Tangles of filaments largely made up of a protein associated with the cytoskeleton have also been observed in samples taken from Alzheimer brain tissue.

In a specific embodiment, the Alzheimer disease to be predicted or diagnosed according to the present method is associated with a mutation in the AD1, AD2, AD3 or AD4 gene.

b. Amyotrophic Lateral Sclerosis

Amyotrophic lateral sclerosis (ALS) is a neurological disorder characterized by progressive degeneration of motor neuron cells in the spinal cord and brain, which ultimately results in paralysis and death. The SOD1 gene was identified as being associated with many cases of familial ALS (Rosen et al., Nature, 362(6415):59-62 (1993)). The enzyme coded for by SOD1 carries out a very important function in cells: it removes dangerous superoxide radicals by converting them into non-harmful substances. Defects in the action of this enzyme mean that the superoxide radicals attack cells from the inside, causing their death. Several different mutations in this enzyme all result in ALS, making the exact molecular cause of the disease difficult to ascertain.

In a specific embodiment, the amyotrophic lateral sclerosis to be predicted or diagnosed according to the present method is associated with a mutation in SOD 1.

c. Angelman Syndrome

Angelman syndrome (AS) is an uncommon neurogenetic disorder characterized by mental retardation, abnormal gait, speech impairment, seizures, and an inappropriate happy demeanor which includes frequent laughing, smiling, and excitability. The genetic basis of AS is very complex, but the majority of cases are due to a deletion of segment 15q11-q13 on the maternally derived chromosome 15. When this same region is missing from the paternally derived chromosome, an entirely different disorder, Prader-Willi syndrome, results. This phenomenon—when the expression of genetic material depends on whether it has been inherited from the mother or the father—is termed genomic imprinting. The ubiquitin ligase gene (UBE3A) is found in the AS chromosomal region (Jiang et al., Am. J. Hum. Genet., 65(1):1-6 (1999); Albrecht et al, Nat. Genet., 17(1):75-8 (1997); and Kishino et al., Nat. Genet., 15(1):70-3 (1997)). It codes for an enzyme that is a key part of a cellular protein degradation system. AS is thought to occur when mutations in UBE3A disrupt protein break down during brain development.

In a specific embodiment, the Angelman syndrome to be predicted or diagnosed according to the present method is associated with a mutation in ubiquitin ligase gene (UBE3A).

d. Charcot-Marle-Tooth Disease

Charcot-Marle-tooth disease (CMT) disease is characterized by a slowly progressive degeneration of the muscles in the foot, lower leg, hand and forearm, and a mild loss of sensation in the limbs, fingers and toes. CMT is a genetically heterogeneous disorder, in which mutations in different genes can produce the same clinical symptoms (Lagemann, ROFO Fortschr Geb Rontgenstr Nuklearmed, 124(1):69-75 (1976); and Hayasaka et al., Genomics, 17(3):755-8 (1993)). In CMT, there are not only different genes but different patterns of inheritance. One of the most common forms of CMT is Type 1A. The gene for Type 1A CMT maps to chromosome 17 and is thought to code for a protein (PMP22) involved in coating peripheral nerves with myelin, a fatty sheath that is important for their conductance. Other types of CMT include Type 1B, autosomal-recessive and X-linked. The same proteins involved in the Type 1A and Type 1B CMT are also involved in a disease called Dejerine-Sottas syndrome (DSS), in which similar clinical symptoms are presented, but they are more severe.

In a specific embodiment, the Charcot-Marle-tooth disease to be predicted or diagnosed according to the present method is associated with a mutation in type 1A or type 1B CMT gene.

e. Epilepsy

Epilepsy is characterized by recurring seizures resulting from abnormal cell firing in the brain. There are many forms of epilepsy—most are rare. To date, twelve forms of epilepsy have been demonstrated to possess some genetic basis. For example, LaFora Disease (progressive myoclonic, type 2) is a particularly aggressive epilepsy inherited in an autosomal recessive fashion (Minassian et al., Nat. Genet., 20(2):171-4 (1998)). LaFora Disease is thought to result from a mutation in the EPM2A gene, which is located on chromosome 6. This gene is thought to produce laforin, a protein similar to a group of protein-tyrosine phosphatases that help maintain a balance of sugars in the blood stream. Too much laforin may destroy brain cells, which may then lead to the development of LaFora Disease.

In a specific embodiment, the epilepsy to be predicted or diagnosed according to the present method is associated with a mutation in EPM2A.

f. Tremor

Tremor, or uncontrollable shaking, is a common symptom of neurological disorders such as Parkinson's disease, head trauma and stroke. Many people with tremor have what is called idiopathic or essential tremor. In these cases, the tremor itself is the only symptom of the disorder. While essential tremor may involve other parts of the body, the hands and head are most often affected. In more than half of cases, essential tremor is inherited as an autosomal dominant trait, which means that children of an affected individual will have a 50 percent chance of also developing the disorder. In 1997, the ETM1 gene (also called FET1) was mapped to chromosome 3 in a study of Icelandic families, while another gene, called ETM2, was mapped to chromosome 2 in a large American family of Czech descent (Gulcher et al., Nat. Genet., 17(1):84-7 (1997)). That two genes for essential tremor have been found on two different chromosomes demonstrates that mutations in a variety of genes may lead to essential tremor.

In a specific embodiment, the tremor to be predicted or diagnosed according to the present method is associated with a mutation in ETM1 or ETM2.

g. Fragile X Syndrome

Fragile X syndrome is the most common inherited form of mental retardation currently known. Fragile X syndrome is a defect in the X chromosome and its effects are seen more frequently, and with greater severity, in males than females. In normal individuals, the FMR1 gene is transmitted stably from parent to child. In Fragile X individuals, there is a mutation in one end of the gene (the 5′ untranslated region), that involves amplification of a CGG repeat (Siomi et al., Cell, 74(2):291-8 (1993)). Patients with fragile X syndrome have 200 or more copies of the CGG motif. The huge expansion of this repeat means that the FMR1 gene is not expressed, so no FMR1 protein is made. Although the exact function of FMR1 protein in the cell is unclear, it is known that it binds RNA.

In a specific embodiment, the fragile X syndrome to be predicted or diagnosed according to the present method is associated with a mutation in FMR1 gene.

h. Friedreich's Ataxia

Friedreich's ataxia (FRDA) is a rare inherited disease characterized by the progressive loss of voluntary muscular coordination (ataxia) and heart enlargement. FRDA is an autosomal recessive disease caused by a mutation of a gene called frataxin, which is located on chromosome 9 (Campuzano et al., Science, 271(5254):1423-7 (1996); and Babcock et al., Science, 276(5319):1709-12 (1997)). This mutation means that there are many extra copies of a DNA segment, the trinucleotide GAA. A normal individual has 8 to 30 copies of this trinucleotide, while FRDA patients have as many as 1000. The larger the number of GAA copies, the earlier the onset of the disease and the quicker the decline of the patient.

In a specific embodiment, the Friedreich's ataxia to be predicted or diagnosed according to the present method is associated with a mutation in frataxin.

Huntington Disease

Huntington disease (HD) is an inherited, degenerative neurological disease that leads to dementia. The HD gene, whose mutation results in Huntington disease, was mapped to chromosome 4 in 1983 and cloned in 1993 (Cell, 72(6):971-83 (1993)). The mutation is a characteristic expansion of a nucleotide triplet repeat in the DNA that codes for the protein huntingtin. The number of repeated triplets—CAG (cytosine, adenine, guanine)—increases with the age of the patient. Since people who have those repeats always suffer from Huntington disease, it suggests that the mutation causes a gain-of-function, in which the mRNA or protein takes on a new property or is expressed inappropriately.

In a specific embodiment, the Huntington disease to be predicted or diagnosed according to the present method is associated with a mutation in the HD gene.

j. Niemann-Pick's Disease

In 1914, German Pediatrician Albert Niemann described a young child with brain and nervous system impairment. Later, in the 1920's, Luddwick Pick studied tissues after the death of such children and provided evidence of a new disorder, distinct from those storage disorders previously described. Today, there are three separate diseases that carry the name Niemann-Pick: Type A is the acute infantile form, Type B is a less common, chronic, non-neurological form, while Type C is a biochemically and genetically distinct form of the disease. Recently, the major locus responsible for Niemann-Pick type C (NP-C) was cloned from chromosome 18, and found to be similar to proteins that play a role in cholesterol homeostasis (Carstea, Science, 277(5323):228-31 (1997); and Loftus, Science, 277(5323):232-5 (1997)). Usually, cellular cholesterol is imported into lysosomes—‘bags of enzymes’ in the cell—for processing, after which it is released. Cells taken from NP-C patients have been shown to be defective in releasing cholesterol from lysosomes. This leads to an excessive build-up of cholesterol inside lysosomes, causing processing errors. NPC1 was found to have known sterol-sensing regions similar to those in other proteins, which suggests it plays a role in regulating cholesterol traffic.

In a specific embodiment, the Niemann-Pick to be predicted or diagnosed according to the present method is associated with a mutation in NPC1.

k. Parkinson Disease

Parkinson disease is a neurodegenerative disease that manifests as a tremor, muscular stiffness and difficulty with balance and walking. A classic pathological feature of the disease is the presence of an inclusion body, called the Lewy body, in many regions of the brain. A candidate gene for some cases of Parkinson disease was mapped to chromosome 4 (Polymeropoulos et al., Science, 276(5321):2045-7 (1997)). Mutations in this gene have now been linked to several Parkinson disease families. The product of this gene, a protein called alpha-synuclein, is a familiar culprit: a fragment of it is a known constituent of Alzheimer disease plaques.

In a specific embodiment, the Parkinson disease to be predicted or diagnosed according to the present method is associated with a mutation in á-synuclein.

l. Spinocerebellar Atrophy

Persons with spinocerebellar atrophy, of which there are several types, experience a degeneration of the spinal cord and the cerebellum, the small fissured mass at the base of the brain, behind the brain stem. The cerebellum is concerned with coordination of movements, so atrophy or “wasting away” of this critical control center results in a loss of muscle coordination. Atrophy in the spine can bring spasticity. The basic defect in all types of spinocerebellar atrophy is a an expansion of a CAG triplet repeat. In this way, it is similar to fragile-X syndrome, Huntington disease and myotonic dystrophy, all of which exhibit a triplet repeat expansion of a gene. In the case of spinocerebellar atrophy I, the gene is SCA1, found on chromosome 6 (Banfi et al, Nat. Genet., 7(4):513-20 (1994)). The protein product of the gene—called ataxin-1—varies in size, depending on the size of the CAG triplet repeat.

In a specific embodiment, the Prader-Willi syndrome to be predicted or diagnosed according to the present method is associated with a mutation in the small ribonucleoprotein N (SNRPN).

m. Williams Syndrome

Williams syndrome is a rare congenital disorder characterized by physical and development problems. Common features include characteristic “elfin-like” facial features, heart and blood vessel problems, irritability during infancy, dental and kidney abnormalities, hyperacusis (sensitive hearing) and musculoskeletal problems. In Williams syndrome individuals, the gene for elastin and an enzyme called LIM kinase are deleted (Frangiskakis et al., Cell, 86(1):59-69 (1996); and Lenhoff et al., Sci. Am., 277(6):68-73 (1997)). Both genes map to the same small area on chromosome 7. In normal cells, elastin is a key component of connective tissue, conferring its elastic properties. Mutation or deletion of elastin lead to the vascular disease observed in Williams syndrome. On the other hand, LIM kinase is strongly expressed in the brain, and deletion of LIM kinase is thought to account for the impaired visuospatial constructive cognition in Williams syndrome. Williams syndrome is a contiguous disease, meaning that the deletion of this section of chromosome 7 may involve several more genes. Further study will be required to round up all the genes deleted in this disease.

In a specific embodiment, the Williams syndrome to be predicted or diagnosed according to the present method is associated with a mutation in elastin and LIM kinase.

6. Signal Disease or Disorder

Any signal diseases or disorders that are associated with a mutation(s) in a nucleic acid can be predicted or diagnozed using the present methods. For example, ataxia telangiectasia (A-T), male pattern baldness, acne, hirsutism, Cockayne syndrome, glaucoma, mammals with abnormal secondary sexual characteristics, tuberous sclerosis, Waardenburg syndrome (WS) and Werner syndrome (WRN) can be predicted or diagnozed using the present methods.

a. Ataxia Telangiectasia

The first signs of ataxia telangiectasia (A-T) usually appear in the second year of life as a lack of balance and slurred speech. It is a progressive, degenerative disease characterized by cerebellar degeneration, immunodeficiency, radiosensitivity (sensitivity to radiant energy, such as x-ray) and a predisposition to cancer. The gene responsible for A-T was mapped to chromosome 11. The subsequent identification of the gene proved difficult: it was seven more years until the human ATM gene was cloned (Savitsky, Science, 268(5218):1749-53 (1995); and Barlow Cell, 86(1):159-71 (1996)). The diverse symptoms seen in A-T reflect the main role of ATM, which is to induce several cellular responses to DNA damage. When the ATM gene is mutated, these signaling networks are impaired and so the cell does not respond correctly to minimize the damage.

In a specific embodiment, the ataxia telangiectasia to be predicted or diagnosed according to the present method is associated with a mutation in ATM.

b. Male Pattern Baldness, Acne or Hirsutism

Five-á reductase is an enzyme that was first discovered in the male prostate. Here, it catalyzes the conversion of testosterone to dihydrotestosterone, which in turn binds to the androgen receptor and initiates development of the external genitalia and prostate. The gene for 5-alpha reductase has been mapped to chromosome 5 (Andersson and Russell, Proc. Natl. Acad. Sci., 87(10):3640-4 (1990); and Jenkins Genomics, 11(4):1102-12 (1991)). More recently, 5-alpha reductase was found in human scalp and elsewhere in the skin, where it carries out the same reaction as in the prostate. It is thought that disturbances in 5-alpha reductase activity in skin cells might contribute to male pattern baldness, acne or hirsutism.

In a specific embodiment, the male pattern baldness, acne or hirsutism to be predicted or diagnosed according to the present method is associated with a mutation in 5-á reductase.

c. Cockayne Syndrome

Cockayne syndrome is a rare inherited disorder in which people are sensitive to sunlight, have short stature and have the appearance of premature aging. In the classical form of Cockayne syndrome (Type I), the symptoms are progressive and typically become apparent after the age of one year. An early onset or congenital form of Cockayne syndrome (Type II) is apparent at birth. Interestingly, unlike other DNA repair diseases, Cockayne syndrome is not linked to cancer. After exposure to UV radiation (found in sunlight), people with Cockayne syndrome can no longer perform a certain type of DNA repair, known as ‘transcription-coupled repair’. This type of DNA repair occurs ‘on the fly’, right as the DNA that codes for proteins is being replicated. Two genes defective in Cockayne syndrome, CSA and CSB, have been identified so far. The CSA gene is found on chromosome 5. Both genes code for proteins that interacts with components of the transcriptional machinery and with DNA repair proteins (van Gool, EMBO J., 16(14):4155-62 (1997)).

In a specific embodiment, the Cockayne syndrome to be predicted or diagnosed according to the present method is associated with a mutation in CSA or CSB.

d. Glaucoma

Glaucoma is a term used for a group of diseases that can lead to damage to the eye's optic nerve and result in blindness. The most common form of the disease is open-angle glaucoma, which affects about three million Americans, half of whom don't know they have it. Glaucoma has no symptoms at first but over the years can steal its victims' sight, with side vision being effected first. It is estimated that nearly 100,000 individuals in the US suffer from glaucoma due to a mutation in the GLC1A gene, found on chromosome 1 (Stone, Science, 275(5300):668-70 (1997)). There has been some speculation as to the role of the gene product in the eye. As it is found in the structures of the eye involved in pressure regulation, it may cause increased pressure in the eye by obstructing the aqueous outflow.

In a specific embodiment, the glaucoma to be predicted or diagnosed according to the present method is associated with a mutation in GLC1A.

e. Abnormal Secondary Sexual Characteristics

Usually, a woman has two X chromosomes (XX) and a man one X and one Y (XY). Male and female characteristics sometimes can be found in one individual, and it is possible to have XY women and XX men. Analysis of such individuals has revealed some of the molecules involved in sex determination, including one called SRY, which is important for testis formation. SRY (which stands for sex-determining region Y gene) is found on the Y chromosome (Berta, Nature, 348(6300):448-50 (1990); and Goodfellow and Lovell-Badge, Annu. Rev. Genet., 27:71-92 (1993)). In the cell, it binds to DNA and in doing so distorts it dramatically out of shape. This alters the properties of the DNA and likely alters the expression of a number of genes, leading to testis formation. Therefore XX men who lack a Y chromosome also lack SRY and frequently do not develop secondary sexual characteristics in the usual way.

In a specific embodiment, the abnormal secondary sexual characteristics to be predicted or diagnosed according to the present method is associated with a mutation in sex-determining region Y gene (SRY).

f. Tuberous Sclerosis

Tuberous sclerosis is an hereditary disorder characterized by benign, tumor-like nodules of the brain and/or retinas, skin lesions, seizures and/or mental retardation. Patients may experience a few or all of the symptoms with varying degrees of severity. Two loci for tuberous sclerosis have been found: TSC1 on chromosome 9, and TSC2 on chromosome 16 (Cell, 75(7):1305-15 (1993)). It took four years to pin down a specific gene from the TSC1 region of chromosome 9: in 1997, a promising candidate was found. Called hamartin by the discoverers, it is similar to a yeast protein of unknown function, and appears to act as a tumor suppressor: without TSC1, growth of cells proceeds in an unregulated fashion, resulting in tumor formation (van Slegtenhorst, Science, 277(5327):805-8 (1997)). TSC2 codes for a protein called tuberin, which, through database searches, was found to have a region of homology to a protein found in pathways that regulate the cell (GAP3, a GTPase-activation protein).

In a specific embodiment, the tuberous sclerosis to be predicted or diagnosed according to the present method is associated with a mutation in TSC1 or TSC2.

g. Waardenburg Syndrome

The main characteristics of Waardenburg syndrome (WS) include: a wide bridge of the nose; pigmentary disturbances such as two different colored eyes, white forelock and eyelashes and premature graying of the hair; and some degree of cochlear deafness. The several types of WS are inherited in dominant fashion, so researchers typically see families with several generations who have inherited one or more of the features. Type I of the disorder is characterized by displacement of the fold of the eyelid, while Type II does not include this feature, but instead has a higher frequency of deafness. The discovery of the human gene that causes Type I WS came about after scientists speculated that the gene that causes ‘splotch mice’ (mice with a splotchy coat coloring) might be the same gene that causes WS in humans. They located the human gene to chromosome 2 and found it was the same as mouse Pax3 (Tassabehji et al., Nature, 355(6361):635-6 (1992)).

In a specific embodiment, the Waardenburg syndrome to be predicted or diagnosed according to the present method is associated with a mutation in human homolog of mouse Pax3.

h. Werner Syndrome

Werner syndrome is a premature aging disease that begins in adolescence or early adulthood and results in the appearance of old age by 30-40 years of age. Its physical characteristics may include short stature (common from childhood on) and other features usually developing during adulthood: wrinkled skin, baldness, cataracts, muscular atrophy and a tendency to diabetes mellitus, among others. The disorder is inherited and transmitted as an autosomal recessive trait. Cells from WS patients have a shorter lifespan in culture than do normal cells. The gene for Werner disease (WRN) was mapped to chromosome 8 and cloned: by comparing its sequence to existing sequences in GenBank, it is a predicted helicase belonging to the RecQ family (Gray et al., Nat. Genet., 17(1):100-3 (1997); and Sinclair et al., Science, 277(5330):1313-6 (1997)).

In a specific embodiment, the Werner syndrome to be predicted or diagnosed according to the present method is associated with a mutation in WRN gene.

7. Transporter Diseases and Disorders

Any transporter diseases and disorders that are associated with a mutation(s) in a nucleic acid can be predicted or diagnosed using the present methods. For example, cystic fibrosis (CF), diastrophic dysplasia (DTD), long-QT syndrome (LQTS), Menkes' syndrome, pendred syndrome, adult polycystic kidney disease (APKD), Wilson's disease and Zellweger syndrome can be predicted or diagnosed using the present methods.

a. Cystic Fibrosis

Cystic fibrosis (CF) is the most common fatal genetic disease in the US today. It causes the body to produce a thick, sticky mucus that clogs the lungs, leading to infection, and blocks the pancreas, stopping digestive enzymes from reaching the intestines where they are required to digest food. CF is caused by a defective gene, which codes for a sodium and chloride (salt) transporter found on the surface of the epithelial cells that line the lungs and other organs (Riordan et al., Science, 245(4922):1066-73 (1989)). Several hundred mutations have been found in this gene, all of which result in defective transport of sodium and chloride by epithelial cells. The severity of the disease symptoms of CF is directly related to the characteristic effects of the particular mutation(s) that have been inherited by the sufferer.

In a specific embodiment, the cystic fibrosis to be predicted or diagnosed according to the present method is associated with a mutation in the CF gene.

b. Diastrophic Dysplasia

Diastrophic dysplasia (DTD) is a rare growth disorder in which patients are usually short, have club feet and have malformed hands and joints. Although found in all populations, it is particularly prevalent in Finland. The gene whose mutation results in DTD maps to chromosome 5 and encodes a novel sulfate transporter (Hastbacka et al., Genomics, 11(4):968-73 (1991); and Hastbacka et al, Cell, 78(6):1073-87 (1994)). This ties in with the observation of unusual concentrations of sulfate in various tissues of DTD patients. Sulfate is important for skeletal joints because cartilage—the shock-absorber of joints—requires sulfur during its manufacture. Adding sulfur increases the negative charge within cartilage, which contributes to its shock-absorbing properties.

In a specific embodiment, the diastrophic dysplasia to be predicted or diagnosed according to the present method is associated with a mutation in the DTD gene.

c. Long-QT Syndrome

Long-QT syndrome (LQTS) results from structural abnormalities in the potassium channels of the heart, which predispose affected persons to an accelerated heart rhythm (arrhythmia). This can lead to sudden loss of consciousness and may cause sudden cardiac death in teenagers and young adults who are faced with stressors ranging from exercise to loud sounds. LQTS is usually inherited as an autosomal dominant trait (Wang et al., Nat. Genet., 12(1):17-23 (1996); and Barhanin et al., Nature, 384(6604):78-80 (1996)). In the case of LQT1, which has been mapped to chromosome 11, mutations lead to serious structural defects in the person's cardiac potassium channels that do not allow proper transmission of the electrical impulses throughout the heart. There also appear to be other genes, tentatively located on chromosomes 3, 6 and 11 whose mutated products may contribute to, or cause, LQT syndrome.

In a specific embodiment, the long-QT syndrome to be predicted or diagnosed according to the present method is associated with a mutation in LQT1.

d. Menkes' Syndrome

Menkes' syndrome is an inborn error of metabolism that markedly decreases the cells' ability to absorb copper. The disorder causes severe cerebral degeneration and arterial changes, resulting in death in infancy. The disease can often be diagnosed by looking at a victim's hair, which appears to be whitish and kinked when viewed under a microscope. Menkes' disease is transmitted as an X-linked recessive trait. Sufferers can not transport copper, which is needed by enzymes involved in making bone, nerve and other structures (Chelly et al., Nat. Genet., 3(1):14-9 (1993)). A number of other diseases, including type IX Ehlers-Danlos syndrome, may be the result of allelic mutations (i.e., mutations in the same gene, but having slightly different symptoms) and it is hoped that research into these diseases may prove useful in fighting Menkes' disease.

In a specific embodiment, the Menkes' syndrome to be predicted or diagnosed according to the present method is associated with a mutation in the copper transporter.

e. Pendred Syndrome

Pendred syndrome is an inherited disorder that accounts for as much as 10% of hereditary deafness. Patients usually also suffer from thyroid goiter. In December of 1997, scientists at NIH's National Human Genome Research Institute used the physical map of human chromosome 7 to help identify an altered gene thought to cause pendred syndrome (Everett et al., Nat. Genet., 17(4):411-22 (1997)). The normal gene makes a protein, called pendrin, that is found at significant levels only in the thyroid and is closely related to a number of sulfate transporters. When the gene for this protein is mutated, the person carrying it will exhibit the symptoms of Pendred syndrome.

In a specific embodiment, the pendred syndrome to be predicted or diagnosed according to the present method is associated with a mutation in pendrin.

f. Adult Polycystic Kidney Disease

Adult polycystic kidney disease (APKD) is characterized by large cysts in one or both kidneys and a gradual loss of normal kidney tissue. The role of the kidneys in the body is to filter the blood, excreting the end-products of metabolism in the form of urine and regulating the concentrations of hydrogen, sodium, potassium, phosphate and other ions in the extracellular fluid. Patients with APKD can die from renal failure, or from the consequences of hypertension (high arterial blood pressure). In 1994 the European Polycystic Kidney Disease Consortium isolated a gene from chromosome 16 that was disrupted in a family with APCD (Cell, 77(6):881-94 (1994) (Published errata appear in Cell 1994 Aug. 26;78(4):following 724 and 1995 Jun. 30;81(7):following 1170); and Cell, 81(2):289-98 (1995)). The protein encoded by the PKD1 gene is an integral membrane protein involved in cell-cell interactions and cell-matrix interactions. The role of PKD1 in the normal cell may be linked to microtubule-mediated functions, such as the placement of Na(+), K(+)-ATPase ion pumps in the membrane. Programmed cell death, or apoptosis, may also be invoked in APKD.

In a specific embodiment, the adult polycystic kidney disease to be predicted or diagnosed according to the present method is associated with a mutation in PKD1.

g. Wilson's Disease

Wilson's disease is a rare autosomal recessive disorder of copper transport, resulting in copper accumulation and toxicity to the liver and brain. Liver disease is the most common symptom in children; neurological disease is most common in young adults. The cornea of the eye can also be affected: the ‘Kayser-Fleischer ring’ is a deep copper-colored ring at the periphery of the cornea, and is thought to represent copper deposits. The gene for Wilson's disease (ATP7B) was mapped to chromosome 13. The sequence of the gene was found to be similar to sections of the gene defective in Menkes disease, another disease caused by defects in copper transport. The similar sequences code for copper-binding regions, which are part of a transmembrane pump called a P-type ATPase that is very similar to the Menkes disease protein (Bull et al., Nat. Genet., 5(4):327-37 (1993) (Published erratum appears in Nat Genet 1994 Feb.;6(2):214).

In a specific embodiment, the Wilson's disease to be predicted or diagnosed according to the present method is associated with a mutation in ATP7B.

h. Zellweger Syndrome

Zellweger syndrome is a rare hereditary disorder affecting infants, and usually results in death. Unusual problems in prenatal development, an enlarged liver, high levels of iron and copper in the blood, and vision disturbances are among the major manifestations of Zellweger syndrome. The PXR1 gene has been mapped to chromosome 12; mutations in this gene cause Zellweger syndrome. The PXR1 gene product is a receptor found on the surface of peroxisomes—microbodies found in animal cells, especially liver, kidney and brain cells (Dodt et al., Nat. Genet., 9(2):115-25 (1995); and Marynen et al., Genomics, 30(2):366-8 (1995)). The PXR1 receptor is vital for the import of these enzymes into the peroxisomes: without it functioning properly, the peroxisomes can not use the enzymes to carry out their important functions, such as cellular lipid metabolism and metabolic oxidations.

In a specific embodiment, the Zellweger syndrome to be predicted or diagnosed according to the present method is associated with a mutation in PXR1.

8. Infections

Any infections by pathological agents can be predicted or diagnozed using the present methods. For example, infections by viruses, eubacteria, archaebacteria and eukaryotic pathogens can be predicted or diagnosed using the present methods.

In a specific embodiment, the viral infection to be predicted or diagnosed according to the present method is caused by a Delta virus, a dsDNA virus, a retroid virus, a satellite virus, a ssDNA virus, a ssRNA negative-strand virus, ssRNA positive-strand virus (no DNA stage) or a bacteriophage.

In another specific embodiment, the eubacteria infection to be predicted or diagnosed according to the present method is caused by a green bacteria, a flavobacteria, a spirochetes, a purple bacteria, a gram-positive bacteria, a gram-negative bacteria, a cynobacteria, a deinococci or a thermotogale.

In still another specific embodiment, the archaebacteria infection to be predicted or diagnosed according to the present method is caused by an extreme halophile, a methanogen or an extreme thermophile.

In yet another specific embodiment, the infection to be predicted or diagnosed according to the present method is caused by an eukaryotic pathogen such as a fungi, a ciliate, a cellular slime mode, a flagellate or a microsporidia.

### D. Methods for Detecting Polymorphisms

Provided herein is a method for detecting polymorphism in a locus, which method comprises: a) hybridizing a target strand of a nucleic acid comprising a locus to be tested with a complementary reference strand of a nucleic acid comprising a known allele of the locus, whereby the allelic-identity between the target and the reference strands results in the formation of a nucleic acid duplex without an abnormal base-pairing and the allelic difference between the target and the reference strands results in the formation of a nucleic acid duplex with an abnormal base-pairing; b) contacting the nucleic acid duplex formed in step a) with a mutant DNA repair enzyme or complex thereof, wherein the mutant DNA repair enzyme or complex thereof has binding affinity for the abnormal base-pairing in the duplex but has attenuated catalytic activity; and c) detecting binding between the nucleic acid duplex and the mutant DNA repair enzyme or complex thereof, whereby the polymorphism in the locus is assessed.

In a specific embodiment, the polymorphism to be detected is a variable nucleotide type polymorphism (“VNTR”).

In another specific embodiment, the polymorphism to be detected is a single nucleotide polymorphism (SNP). Preferably, a polymorphism in a genome, e.g., a viral, bacterial, eukaryotic, mammalian or human genome, is detected by the present methods. More preferably, the human genome SNPs listed in the following Table 2 can be detected by the present methods (see e.g., http://www.ncbi.nlm.gov/SNP).

TABLE 2
Examples of human genome polymorphisms

| | | dbSNP | |
|---|---|---|---|
| CHROMOSOME | FINE MAP LOCATION | ASSAY ID | HANDLE\|LOCAL SNP ID |
| 1 | 0.00 cR from top of Chr1 linka | 1946 | WIAF\|WIAF-3885 |
| 1 | 0.00 cR from top of Chr1 linka | 2870 | WIAF\|WIAF-768 |
| 1 | 0.60 cR from top of Chr1 linka | 1196 | WIAF\|WIAF-2083 |
| 1 | 6.20 cR from top of Chr1 linka | 1861 | WIAF\|WIAF-3800 |
| 1 | 7.8 cR from top of Chr1 linkag | 2383 | WIAF\|WIAF-2674 |
| 1 | 12.1 cR from top of Chr1 linka | 3083 | WIAF\|WIAF-984 |
| 1 | 16.40 cR from top of Chr1 link | 1921 | WIAF\|WIAF-3860 |
| 1 | 21.2 cR from top of Chr1 linka | 3061 | WIAF\|WIAF-962 |
| 1 | 23.3 cR from top of Chr1 linka | 2762 | WIAF\|WIAF-501 |
| 1 | 27.10 cR from top of Chr1 link | 1421 | WIAF\|WIAF-3349 |
| 1 | 33.30 cR from top of Chr1 link | 2934 | WIAF\|WIAF-833 |
| 1 | 34.50 cR from top of Chr1 link | 3318 | WIAF\|WIAF-1771 |
| 1 | 50.0 cR from top of Chr1 linka | 2566 | WIAF\|WIAF-195 |
| 1 | 50.40 cR from top of Chr1 link | 1954 | WIAF\|WIAF-3893 |
| 1 | 51.20 cR from top of Chr1 link | 3248 | WIAF\|WIAF-1663 |
| 1 | 54.9 cR from top of Chr1 linka | 3124 | WIAF\|WIAF-1025 |
| 1 | 55.5 cR from top of Chr1 linka | 2576 | WIAF\|WIAF-206 |
| 1 | 55.80 cR from top of Chr1 link | 1130 | WIAF\|WIAF-1577 |
| 1 | 55.80 cR from top of Chr1 link | 1131 | WIAF\|WIAF-1578 |
| 1 | 55.80 cR from top of Chr1 link | 2951 | WIAF\|WIAF-850 |
| 1 | 55.90 cR from top of Chr1 link | 670 | WIAF\|WIAF-1348 |
| 1 | 57.00 cR from top of Chr1 link | 3255 | WIAF\|WIAF-1677 |
| 1 | 59.80 cR from top of Chr1 link | 2526 | WIAF\|WIAF-135 |
| 1 | 60 cM | 4319 | UWGC\|138 |
| 1 | 60.70 cR from top of Chr1 link | 1498 | WIAF\|WIAF-3437 |
| 1 | 62.8 cR from top of Chr1 linka | 2079 | WIAF\|WIAF-28 |
| 1 | 68.5 cR from top of Chr1 linka | 3138 | WIAF\|WIAF-1039 |
| 1 | 69.00 cR from top of Chr1 link | 3043 | WIAF\|WIAF-944 |
| 1 | 71.30 cR from top of Chr1 link | 3188 | WIAF\|WIAF-1504 |
| 1 | 75.30 cR from top of Chr1 link | 3479 | WIAF\|WIAF-1934 |
| 1 | 75.90 cR from top of Chr1 link | 1886 | WIAF\|WIAF-3825 |
| 1 | 77.20 cR from top of Chr1 link | 1275 | WIAF\|WIAF-2162 |
| 1 | 77.90 cR from top of Chr1 link | 677 | WIAF\|WIAF-1443 |
| 1 | 78.30 cR from top of Chr1 link | 2876 | WIAF\|WIAF-774 |
| 1 | 78.60 cR from top of Chr1 link | 1179 | WIAF\|WIAF-1708 |
| 1 | 84.30 cR from top of Chr1 link | 1756 | WIAF\|WIAF-3695 |
| 1 | 91.5 cR from top of Chr1 linka | 743 | WIAF\|WIAF-1191 |
| 1 | 92.60 cR from top of Chr1 link | 1388 | WIAF\|WIAF-3293 |
| 1 | 97.8 cR from top of Chr1 linka | 2273 | WIAF\|WIAF-734 |
| 1 | 103.20 cR from top of Chr1 lin | 1622 | WIAF\|WIAF-3561 |
| 1 | 103.20 cR from top of Chr1 lin | 1626 | WIAF\|WIAF-3565 |
| 1 | 106.90 cR from top of Chr1 lin | 1577 | WIAF\|WIAF-3516 |
| 1 | 113.3 cR from top of Chr1 link | 2554 | WIAF\|WIAF-178 |
| 1 | 117.4 cR from top of Chr1 link | 975 | WIAF\|WIAF-1388 |
| 1 | 118.70 cR from top of Chr1 lin | 2527 | WIAF\|WIAF-136 |
| 1 | 118.70 cR from top of Chr1 lin | 1952 | WIAF\|WIAF-3891 |
| 1 | 119.10 cR from top of Chr1 lin | 2032 | WIAF\|WIAF-1590 |
| 1 | 120.30 cR from top of Chr1 lin | 3229 | WIAF\|WIAF-1630 |
| 1 | 129.30 cR from top of Chr1 lin | 1873 | WIAF\|WIAF-3812 |
| 1 | 129.30 cR from top of Chr1 lin | 1876 | WIAF\|WIAF-3815 |
| 1 | 129.30 cR from top of Chr1 lin | 1877 | WIAF\|WIAF-3816 |
| 1 | 129.40 cR from top of Chr1 lin | 1157 | WIAF\|WIAF-1642 |
| 1 | 141.60 cR from top of Chr1 lin | 1110 | WIAF\|WIAF-1543 |
| 1 | 142.9 cR from top of Chr1 link | 2123 | WIAF\|WIAF-298 |
| 1 | 142.9 cR from top of Chr1 link | 2124 | WIAF\|WIAF-299 |
| 1 | 146.90 cR from top of Chr1 lin | 1859 | WIAF\|WIAF-3798 |
| 1 | 147.90 cR from top of Chr1 lin | 3552 | WIAF\|WIAF-2007 |
| 1 | 147.90 cR from top of Chr1 lin | 1693 | WIAF\|WIAF-3632 |
| 1 | 148.10 cR from top of Chr1 lin | 3053 | WIAF\|WIAF-954 |
| 1 | 148.30 cR from top of Chr1 lin | 1186 | WIAF\|WIAF-2073 |
| 1 | 154.00 cR from top of Chr1 lin | 1263 | WIAF\|WIAF-2150 |
| 1 | 156.10 cR from top of Chr1 lin | 1266 | WIAF\|WIAF-2153 |
| 1 | 156.10 cR from top of Chr1 lin | 1267 | WIAF\|WIAF-2154 |
| 1 | 160.30 cR from top of Chr1 lin | 1945 | WIAF\|WIAF-3884 |
| 1 | 160.50 cR from top of Chr1 lin | 1369 | WIAF\|WIAF-3272 |
| 1 | 161.9 cR from top of Chr1 link | 1077 | WIAF\|WIAF-2040 |
| 1 | 162.40 cR from top of Chr1 lin | 1140 | WIAF\|WIAF-1603 |
| 1 | 162.90 cR from top of Chr1 lin | 3038 | WIAF\|WIAF-939 |
| 1 | 164.10 cR from top of Chr1 lin | 3574 | WIAF\|WIAF-2029 |
| 1 | 164.10 cR from top of Chr1 lin | 3575 | WIAF\|WIAF-2030 |
| 1 | 164.10 cR from top of Chr1 lin | 1357 | WIAF\|WIAF-3260 |
| 1 | 164.60 cR from top of Chr1 lin | 1566 | WIAF\|WIAF-3505 |
| 1 | 166.90 cR from top of Chr1 lin | 3466 | WIAF\|WIAF-1921 |
| 1 | 168.60 cR from top of Chr1 lin | 1295 | WIAF\|WIAF-2182 |
| 1 | 168.60 cR from top of Chr1 lin | 1296 | WIAF\|WIAF-2183 |
| 1 | 169.40 cR from top of Chr1 lin | 1930 | WIAF\|WIAF-3869 |
| 1 | 170.30 cR from top of Chr1 lin | 1641 | WIAF\|WIAF-3580 |
| 1 | 170.30 cR from top of Chr1 lin | 1644 | WIAF\|WIAF-3583 |
| 1 | 171.5 cR from top of Chr1 link | 2853 | WIAF\|WIAF-740 |
| 1 | 174.50 cR from top of Chr1 lin | 1751 | WIAF\|WIAF-3690 |
| 1 | 182.20 cR from top of Chr1 lin | 1731 | WIAF\|WIAF-3670 |
| 1 | 182.30 cR from top of Chr1 lin | 2034 | WIAF\|WIAF-1595 |
| 1 | 182.80 cR from top of Chr1 lin | 3437 | WIAF\|WIAF-1892 |
| 1 | 183.30 cR from top of Chr1 lin | 1982 | WIAF\|WIAF-3921 |
| 1 | 183.8 cR from top of Chr1 link | 3593 | WIAF\|WIAF-2069 |
| 1 | 187.20 cR from top of Chr1 lin | 2450 | WIAF\|WIAF-38 |
| 1 | 188.30 cR from top of Chr1 lin | 2868 | WIAF\|WIAF-766 |
| 1 | 191.30 cR from top of Chr1 lin | 1521 | WIAF\|WIAF-3460 |
| 1 | 192.40 cR from top of Chr1 lin | 1458 | WIAF\|WIAF-3391 |
| 1 | 192.50 cR from top of Chr1 lin | 1445 | WIAF\|WIAF-3375 |
| 1 | 198.30 cR from top of Chr1 lin | 1360 | WIAF\|WIAF-3263 |
| 1 | 198.7 cR from top of Chr1 link | 2224 | WIAF\|WIAF-653 |
| 1 | 199.30 cR from top of Chr1 lin | 3393 | WIAF\|WIAF-1848 |
| 1 | 200.80 cR from top of Chr1 lin | 1224 | WIAF\|WIAF-2111 |
| 1 | 201.00 cR from top of Chr1 lin | 1245 | WIAF\|WIAF-2132 |
| 1 | 204.40 cR from top of Chr1 lin | 1235 | WIAF\|WIAF-2122 |
| 1 | 209.90 cR from top of Chr1 lin | 2911 | WIAF\|WIAF-809 |
| 1 | 213.0 cR from top of Chr1 link | 983 | WIAF\|WIAF-1409 |
| 1 | 216.50 cR from top of Chr1 lin | 1477 | WIAF\|WIAF-3415 |
| 1 | 217.60 cR from top of Chr1 lin | 1995 | WIAF\|WIAF-3934 |
| 1 | 218.0 cR from top of Chr1 link | 2947 | WIAF\|WIAF-846 |
| 1 | 221.70 cR from top of Chr1 lin | 1191 | WIAF\|WIAF-2078 |
| 1 | 224.60 cR from top of Chr1 lin | 2006 | WIAF\|WIAF-1470 |
| 1 | 224.70 cR from top of Chr1 lin | 1823 | WIAF\|WIAF-3762 |
| 1 | 228.50 cR from top of Chr1 lin | 1585 | WIAF\|WIAF-3524 |
| 1 | 228.50 cR from top of Chr1 lin | 1590 | WIAF\|WIAF-3529 |
| 1 | 231.2 cR from top of Chr1 link | 3142 | WIAF\|WIAF-1043 |
| 1 | 231.2 cR from top of Chr1 link | 3544 | WIAF\|WIAF-1999 |
| 1 | 232.00 cR from top of Chr1 lin | 3326 | WIAF\|WIAF-1779 |
| 1 | 232.40 cR from top of Chr1 lin | 3518 | WIAF\|WIAF-1973 |
| 1 | 235.30 cR from top of Chr1 lin | 1262 | WIAF\|WIAF-2149 |
| 1 | 236.3 cR from top of Chr1 link | 2877 | WIAF\|WIAF-775 |
| 1 | 246.20 cR from top of Chr1 lin | 1491 | WIAF\|WIAF-3430 |
| 1 | 247.30 cR from top of Chr1 lin | 1747 | WIAF\|WIAF-3686 |
| 1 | 247.4 cR from top of Chr1 link | 2654 | WIAF\|WIAF-328 |
| 1 | 247.4 cR from top of Chr1 link | 2655 | WIAF\|WIAF-329 |
| 1 | 248.00 cR from top of Chr1 lin | 1211 | WIAF\|WIAF-2098 |
| 1 | 249.00 cR from top of Chr1 lin | 1508 | WIAF\|WIAF-3447 |
| 1 | 249.80 cR from top of Chr1 lin | 3112 | WIAF\|WIAF-1013 |
| 1 | 249.80 cR from top of Chr1 lin | 3113 | WIAF\|WIAF-1014 |
| 1 | 250.10 cR from top of Chr1 lin | 704 | WIAF\|WIAF-1344 |
| 1 | 250.10 cR from top of Chr1 lin | 1113 | WIAF\|WIAF-1548 |
| 1 | 251.00 cR from top of Chr1 lin | 3559 | WIAF\|WIAF-2014 |
| 1 | 253.2 cR from top of Chr1 link | 3399 | WIAF\|WIAF-1854 |
| 1 | 254.7 cR from top of Chr1 link | 2643 | WIAF\|WIAF-312 |
| 1 | 254.7 cR from top of Chr1 link | 2966 | WIAF\|WIAF-866 |
| 1 | 256.10 cR from top of Chr1 lin | 1102 | WIAF\|WIAF-1521 |
| 1 | 258.70 cR from top of Chr1 lin | 1185 | WIAF\|WIAF-2072 |
| 1 | 263.8 cR from top of Chr1 link | 3295 | WIAF\|WIAF-1748 |
| 1 | 273.20 cR from top of Chr1 lin | 1236 | WIAF\|WIAF-2123 |
| 1 | 281.00 cR from top of Chr1 lin | 3224 | WIAF\|WIAF-1616 |
| 1 | 282.70 cR from top of Chr1 lin | 3348 | WIAF\|WIAF-1801 |
| 1 | 284.3 cR from top of Chr1 link | 3388 | WIAF\|WIAF-1842 |
| 1 | 286.6 cR from top of Chr1 link | 2075 | WIAF\|WIAF-11 |
| 1 | 292.70 cR from top of Chr1 lin | 1630 | WIAF\|WIAF-3569 |
| 1 | 369.7 cR from top of Chr1 link | 2941 | WIAF\|WIAF-840 |
| 1 | 454.8 cR from top of Chr1 link | 2910 | WIAF\|WIAF-808 |
| 1 | 458.7 cR from top of Chr1 link | 2462 | WIAF\|WIAF-53 |
| 1 | 477.3 cR from top of Chr1 link | 3922 | WIAF\|WIAF-4010 |
| 1 | 557.1 cR from top of Chr1 link | 2381 | WIAF\|WIAF-2667 |
| 1 | 573.5 cR from top of Chr1 link | 2741 | WIAF\|WIAF-455 |
| 1 | 629.9 cR from top of Chr1 link | 3592 | WIAF\|WIAF-2068 |
| 1 | 639.0 cR from top of Chr1 link | 772 | WIAF\|WIAF-1403 |
| 1 | 646.6 cR from top of Chr1 link | 1078 | WIAF\|WIAF-2044 |
| 1 | 674.3 cR from top of Chr1 link | 3856 | WIAF\|WIAF-2644 |
| 1 | 675.4 cR from top of Chr1 link | 2482 | WIAF\|WIAF-79 |
| 1 | 676.5 cR from top of Chr1 link | 2555 | WIAF\|WIAF-179 |
| 1 | 676.5 cR from top of Chr1 link | 3501 | WIAF\|WIAF-1956 |
| 1 | 680.0 cR from top of Chr1 link | 4585 | HU-CHINA\|1-1328 |
| 1 | 80.0 cR from top of Chr1 link | 4558 | HU-CHINA\|1-1328-2 |
| 1 | 680.0 cR from top of Chr1 link | 4559 | HU-CHINA\|1-1328-3 |
| 1 | 680.0 cR from top of Chr1 link | 759 | WIAF\|WIAF-1328 |
| 1 | 684.2 cR from top of Chr1 link | 3067 | WIAF\|WIAF-968 |
| 1 | 684.2 cR from top of Chr1 link | 3068 | WIAF\|WIAF-969 |
| 1 | 692.5 cR from top of Chr1 link | 2715 | WIAF\|WIAF-413 |
| 1 | 695.0 cR from top of Chr1 link | 2959 | WIAF\|WIAF-858 |
| 1 | 702.0 cR from top of Chr1 link | 2623 | WIAF\|WIAF-282 |
| 1 | 732.4 cR from top of Chr1 link | 2223 | WIAF\|WIAF-652 |
| 1 | 749.9 cR from top of Chr1 link | 2250 | WIAF\|WIAF-696 |
| 1 | 759.2 cR from top of Chr1 link | 2586 | WIAF\|WIAF-221 |
| 1 | 769.0 cR from top of Chr1 link | 2810 | WIAF\|WIAF-590 |
| 1 | 769.1 cR from top of Chr1 link | 769 | WIAF\|WIAF-1389 |
| 1 | 770.3 cR from top of Chr1 link | 3448 | WIAF\|WIAF-1903 |
| 1 | 781.7 cR from top of Chr1 link | 3004 | WIAF\|WIAF-904 |
| 1 | 783.2 cR from top of Chr1 link | 2086 | WIAF\|WIAF-95 |
| 1 | 817.5 cR from top of Chr1 link | 976 | WIAF\|WIAF-1390 |
| 1 | 819.6 cR from top of Chr1 link | 3395 | WIAF\|WIAF-1850 |
| 1 | 820.1 cR from top of Chr1 link | 895 | WIAF\|WIAF-1143 |
| 1 | 820.1 cR from top of Chr1 link | 1006 | WIAF\|WIAF-4029 |
| 1 | 823.3 cR from top of Chr1 link | 2088 | WIAF\|WIAF-102 |
| 1 | 823.3 cR from top of Chr1 link | 2089 | WIAF\|WIAF-103 |
| 1 | 838.6 cR from top of Chr1 link | 2232 | WIAF\|WIAF-665 |
| 1 | 873.2 cR from top of Chr1 link | 2618 | WIAF\|WIAF-269 |
| 1 | 873.2 cR from top of Chr1 link | 2619 | WIAF\|WIAF-270 |
| 1 | 875.1 cR from top of Chr1 link | 3850 | WIAF\|WIAF-2636 |
| 1 | 883.1 cR from top of Chr1 link | 2540 | WIAF\|WIAF-154 |
| 1 | 884.8 cR from top of Chr1 link | 2867 | WIAF\|WIAF-765 |
| 1 | 889.8 cR from top of Chr1 link | 3051 | WIAF\|WIAF-952 |
| 1 | 890.2 cR from top of Chr1 link | 3116 | WIAF\|WIAF-1017 |
| 1 | 890.3 cR from top of Chr1 link | 3841 | WIAF\|WIAF-2617 |
| 1 | 910.7 cR from top of Chr1 link | 2983 | WIAF\|WIAF-883 |
| 1 | 917.7 cR from top of Chr1 link | 3042 | WIAF\|WIAF-943 |
| 1 | 943.9 cR from top of Chr1 link | 2525 | WIAF\|WIAF-134 |
| 1 | 947.6 cR from top of Chr1 link | 2885 | WIAF\|WIAF-783 |
| 1 | 951.7 cR from top of Chr1 link | 2935 | WIAF\|WIAF-834 |
| 1 | 959.3 cR from top of Chr1 link | 3283 | WIAF\|WIAF-1736 |
| 1 | 959.3 cR from top of Chr1 link | 2424 | WIAF\|WIAF-4 |
| 1 | 961.2 cR from top of Chr1 link | 2570 | WIAF\|WIAF-200 |
| 1 | 961.3 cR from top of Chr1 link | 2782 | WIAF\|WIAF-531 |
| 1 | 961.3 cR from top of Chr1 link | 2479 | WIAF\|WIAF-75 |
| 1 | 962.8 cR from top of Chr1 link | 2637 | WIAF\|WIAF-297 |
| 1 | 969.0 cR from top of Chr1 link | 3114 | WIAF\|WIAF-1015 |
| 1 | 980.4 cR from top of Chr1 link | 2976 | WIAF\|WIAF-876 |
| 1 | 980.4 cR from top of Chr1 link | 2977 | WIAF\|WIAF-877 |
| 1 | 996.9 cR from top of Chr1 link | 2897 | WIAF\|WIAF-795 |
| 1 | 998.5 cR from top of Chr1 link | 2541 | WIAF\|WIAF-155 |
| 1 | | 4221 | MARSHFIELD\|MID-13 |
| 1 | | 4222 | MARSHFIELD\|MID-14 |
| 1 | | 3996 | SHGC/AFFYMETRIX\|SNP-SHGC-10870 |
| 1 | | 4004 | SHGC/AFFYMETRIX\|SNP-SHGC-12999 |
| 1 | | 4155 | SHGC/AFFYMETRIX\|SNP-SHGC-14385 |
| 1 | | 4082 | SHGC/AFFYMETRIX\|SNP-SHGC-16847 |
| 1 | | 4098 | SHGC/AFFYMETRIX\|SNP-SHGC-18912 |
| 1 | | 4037 | SHGC/AFFYMETRIX\|SNP-SHGC-8109 |
| 1 | | 4041 | SHGC/AFFYMETRIX\|SNP-SHGC-8491 |
| 1 | | 4043 | SHGC/AFFYMETRIX\|SNP-SHGC-8995 |
| 1 | | 4049 | SHGC/AFFYMETRIX\|SNP-SHGC-9374 |
| 1 | | 3117 | WIAF\|WIAF-1018 |
| 1 | | 3203 | WIAF\|WIAF-1546 |
| 1 | | 3204 | WIAF\|WIAF-1547 |
| 1 | | 3222 | WIAF\|WIAF-1610 |
| 1 | | 3315 | WIAF\|WIAF-1768 |
| 1 | | 3432 | WIAF\|WIAF-1887 |
| 1 | | 3515 | WIAF\|WIAF-1970 |
| 1 | | 3578 | WIAF\|WIAF-2033 |
| 1 | | 1519 | WIAF\|WIAF-3458 |
| 1 | | 3887 | WIAF\|WIAF-3948 |
| 1 | | 3914 | WIAF\|WIAF-3998 |
| 1 | | 3915 | WIAF\|WIAF-4000 |
| 1 | | 2955 | WIAF\|WIAF-854 |
| 1 | | 2969 | WIAF\|WIAF-869 |
| 2 | 0.00 cR from top of Chr2 linka | 2010 | WIAF\|WIAF-1492 |
| 2 | 6 cM | 4326 | UWGC\|145 |
| 2 | 6.00 cR from top of Chr2 linka | 706 | WIAF\|WIAF-1363 |
| 2 | 6.00 cR from top of Chr2 linka | 1446 | WIAF\|WIAF-3376 |
| 2 | 9.40 cR from top of Chr2 linka | 2676 | WIAF\|WIAF-358 |
| 2 | 12.10 cR from top of Chr2 link | 3383 | WIAF\|WIAF-1836 |
| 2 | 12.10 cR from top of Chr2 link | 3384 | WIAF\|WIAF-1837 |
| 2 | 24.50 cR from top of Chr2 link | 1276 | WIAF\|WIAF-2163 |
| 2 | 32.90 cR from top of Chr2 link | 1334 | WIAF\|WIAF-2224 |
| 2 | 36.60 cR from top of Chr2 link | 1201 | WIAF\|WIAF-2088 |
| 2 | 40.20 cR from top of Chr2 link | 1203 | WIAF\|WIAF-2090 |
| 2 | 41.5 cR from top of Chr2 linka | 2517 | WIAF\|WIAF-125 |
| 2 | 44.40 cR from top of Chr2 link | 698 | WIAF\|WIAF-1268 |
| 2 | 44.6 cR from top of Chr2 linka | 2750 | WIAF\|WIAF-469 |
| 2 | 46.00 cR from top of Chr2 link | 1228 | WIAF\|WIAF-2115 |
| 2 | 46.1 cR from top of Chr2 linka | 3385 | WIAF\|WIAF-1839 |
| 2 | 47.90 cR from top of Chr2 link | 3236 | WIAF\|WIAF-1645 |
| 2 | 47.90 cR from top of Chr2 link | 3237 | WIAF\|WIAF-1646 |
| 2 | 50.30 cR from top of Chr2 link | 1420 | WIAF\|WIAF-3348 |
| 2 | 50.70 cR from top of Chr2 link | 1129 | WIAF\|WIAF-1573 |
| 2 | 51.10 cR from top of Chr2 link | 2925 | WIAF\|WIAF-824 |
| 2 | 51.40 cR from top of Chr2 link | 3223 | WIAF\|WIAF-1612 |
| 2 | 51.40 cR from top of Chr2 link | 1311 | WIAF\|WIAF-2200 |
| 2 | 54.7 cR from top of Chr2 linka | 3033 | WIAF\|WIAF-933 |
| 2 | 55.00 cR from top of Chr2 link | 1975 | WIAF\|WIAF-3914 |
| 2 | 64.90 cR from top of Chr2 link | 3345 | WIAF\|WIAF-1798 |
| 2 | 64.90 cR from top of Chr2 link | 1529 | WIAF\|WIAF-3468 |
| 2 | 66.80 cR from top of Chr2 link | 2014 | WIAF\|WIAF-1508 |
| 2 | 69.00 cR from top of Chr2 link | 1177 | WIAF\|WIAF-1705 |
| 2 | 70.30 cR from top of Chr2 link | 1920 | WIAF\|WIAF-3859 |
| 2 | 70.30 cR from top of Chr2 link | 1922 | WIAF\|WIAF-3861 |
| 2 | 70.60 cR from top of Chr2 link | 2023 | WIAF\|WIAF-1562 |
| 2 | 71.70 cR from top of Chr2 link | 1347 | WIAF\|WIAF-3250 |
| 2 | 76.60 cR from top of Chr2 link | 1104 | WIAF\|WIAF-1528 |
| 2 | 79.70 cR from top of Chr2 link | 1257 | WIAF\|WIAF-2144 |
| 2 | 82.20 cR from top of Chr2 link | 1694 | WIAF\|WIAF-3633 |
| 2 | 84.8 cR from top of Chr2 linka | 2850 | WIAF\|WIAF-714 |
| 2 | 87.10 cR from top of Chr2 link | 1599 | WIAF\|WIAF-3538 |
| 2 | 89.70 cR from top of Chr2 link | 1280 | WIAF\|WIAF-2167 |
| 2 | 89.70 cR from top of Chr2 link | 1594 | WIAF\|WIAF-3533 |
| 2 | 90.10 cR from top of Chr2 link | 692 | WIAF\|WIAF-1226 |
| 2 | 91.60 cR from top of Chr2 link | 1412 | WIAF\|WIAF-3333 |
| 2 | 92.2 cR from top of Chr2 linka | 3103 | WIAF\|WIAF-1004 |
| 2 | 92.20 cR from top of Chr2 link | 1423 | WIAF\|WIAF-3351 |
| 2 | 93.80 cR from top of Chr2 link | 1243 | WIAF\|WIAF-2130 |
| 2 | 96.00 cR from top of Chr2 link | 1162 | WIAF\|WIAF-1665 |
| 2 | 106.10 cR from top of Chr2 lin | 3324 | WIAF\|WIAF-1777 |
| 2 | 106.10 cR from top of Chr2 lin | 1955 | WIAF\|WIAF-3894 |
| 2 | 110.00 cR from top of Chr2 lin | 1684 | WIAF\|WIAF-3623 |
| 2 | 112.40 cR from top of Chr2 lin | 1611 | WIAF\|WIAF-3550 |
| 2 | 112.40 cR from top of Chr2 lin | 1613 | WIAF\|WIAF-3552 |
| 2 | 115.30 cR from top of Chr2 lin | 1286 | WIAF\|WIAF-2173 |
| 2 | 115.30 cR from top of Chr2 lin | 1287 | WIAF\|WIAF-2174 |
| 2 | 117.60 cR from top of Chr2 lin | 3509 | WIAF\|WIAF-1964 |
| 2 | 117.60 cR from top of Chr2 lin | 3510 | WIAF\|WIAF-1965 |
| 2 | 118.60 cR from top of Chr2 lin | 1327 | WIAF\|WIAF-2217 |
| 2 | 118.80 cR from top of Chr2 lin | 3458 | WIAF\|WIAF-1913 |
| 2 | 118.80 cR from top of Chr2 lin | 3459 | WIAF\|WIAF-1914 |
| 2 | 118.80 cR from top of Chr2 lin | 3460 | WIAF\|WIAF-1915 |
| 2 | 119.20 cR from top of Chr2 lin | 2017 | WIAF\|WIAF-1518 |
| 2 | 119.30 cR from top of Chr2 lin | 1653 | WIAF\|WIAF-3592 |
| 2 | 122.40 cR from top of Chr2 lin | 702 | WIAF\|WIAF-1311 |
| 2 | 123.10 cR from top of Chr2 lin | 1503 | WIAF\|WIAF-3442 |
| 2 | 123.10 cR from top of Chr2 lin | 1504 | WIAF\|WIAF-3443 |
| 2 | 123.4 cR from top of Chr2 link | 2638 | WIAF\|WIAF-304 |
| 2 | 124.50 cR from top of Chr2 lin | 3014 | WIAF\|WIAF-914 |
| 2 | 134.30 cR from top of Chr2 lin | 1091 | WIAF\|WIAF-1467 |
| 2 | 134.30 cR from top of Chr2 lin | 1915 | WIAF\|WIAF-3854 |
| 2 | 135.80 cR from top of Chr2 lin | 1724 | WIAF\|WIAF-3663 |
| 2 | 149.50 cR from top of Chr2 lin | 1617 | WIAF\|WIAF-3556 |
| 2 | 152.6 cR from top of Chr2 link | 2284 | WIAF\|WIAF-757 |
| 2 | 158.40 cR from top of Chr2 lin | 3208 | WIAF\|WIAF-1559 |
| 2 | 158.40 cR from top of Chr2 lin | 3209 | WIAF\|WIAF-1560 |
| 2 | 159.40 cR from top of Chr2 lin | 1824 | WIAF\|WIAF-3763 |
| 2 | 162.90 cR from top of Chr2 lin | 1699 | WIAF\|WIAF-3638 |
| 2 | 164.60 cR from top of Chr2 lin | 1947 | WIAF\|WIAF-3886 |
| 2 | 166.4 cR from top of Chr2 link | 3054 | WIAF\|WIAF-955 |
| 2 | 166.50 cR from top of Chr2 lin | 3173 | WIAF\|WIAF-1487 |
| 2 | 169.10 cR from top of Chr2 lin | 1455 | WIAF\|WIAF-3388 |
| 2 | 180.30 cR from top of Chr2 lin | 1368 | WIAF\|WIAF-3271 |
| 2 | 188.20 cR from top of Chr2 lin | 1728 | WIAF\|WIAF-3667 |
| 2 | 188.40 cR from top of Chr2 lin | 3431 | WIAF\|WIAF-1886 |
| 2 | 188.60 cR from top of Chr2 lin | 1206 | WIAF\|WIAF-2093 |
| 2 | 188.70 cR from top of Chr2 lin | 1356 | WIAF\|WIAF-3259 |
| 2 | 190.80 cR from top of Chr2 lin | 1677 | WIAF\|WIAF-3616 |
| 2 | 191.20 cR from top of Chr2 lin | 2025 | WIAF\|WIAF-1570 |
| 2 | 191.20 cR from top of Chr2 lin | 1164 | WIAF\|WIAF-1675 |
| 2 | 191.40 cR from top of Chr2 lin | 1509 | WIAF\|WIAF-3448 |
| 2 | 191.50 cR from top of Chr2 lin | 2636 | WIAF\|WIAF-296 |
| 2 | 192.9 cR from top of Chr2 link | 2454 | WIAF\|WIAF-45 |
| 2 | 192.9 cR from top of Chr2 link | 2455 | WIAF\|WIAF-46 |
| 2 | 195.10 cR from top of Chr2 lin | 1193 | WIAF\|WIAF-2080 |
| 2 | 200.30 cR from top of Chr2 lin | 1248 | WIAF\|WIAF-2135 |
| 2 | 200.40 cR from top of Chr2 lin | 1619 | WIAF\|WIAF-3558 |
| 2 | 201.5 cR from top of Chr2 link | 2968 | WIAF\|WIAF-868 |
| 2 | 202.7 cR from top of Chr2 link | 2503 | WIAF\|WIAF-107 |
| 2 | 208.30 cR from top of Chr2 lin | 1676 | WIAF\|WIAF-3615 |
| 2 | 208.30 cR from top of Chr2 lin | 1678 | WIAF\|WIAF-3617 |
| 2 | 213.00 cR from top of Chr2 lin | 3813 | WIAF\|WIAF-2565 |
| 2 | 214.50 cR from top of Chr2 lin | 3487 | WIAF\|WIAF-1942 |
| 2 | 219.30 cR from top of Chr2 lin | 1288 | WIAF\|WIAF-2175 |
| 2 | 219.30 cR from top of Chr2 lin | 1289 | WIAF\|WIAF-2176 |
| 2 | 220.10 cR from top of Chr2 lin | 1736 | WIAF\|WIAF-3675 |
| 2 | 221.1 cR from top of Chr2 link | 909 | WIAF\|WIAF-1184 |
| 2 | 221.1 cR from top of Chr2 link | 1046 | WIAF\|WIAF-4141 |
| 2 | 221.50 cR from top of Chr2 lin | 3310 | WIAF\|WIAF-1763 |
| 2 | 222.6 cR from top of Chr2 link | 3321 | WIAF\|WIAF-1774 |
| 2 | 223.40 cR from top of Chr2 lin | 3512 | WIAF\|WIAF-1967 |
| 2 | 229.80 cR from top of Chr2 lin | 1510 | WIAF\|WIAF-3449 |
| 2 | 229.80 cR from top of Chr2 lin | 1511 | WIAF\|WIAF-3450 |
| 2 | 234.50 cR from top of Chr2 lin | 1523 | WIAF\|WIAF-3462 |
| 2 | 236.10 cR from top of Chr2 lin | 2020 | WIAF\|WIAF-1526 |
| 2 | 236.10 cR from top of Chr2 lin | 1844 | WIAF\|WIAF-3783 |
| 2 | 236.10 cR from top of Chr2 lin | 1846 | WIAF\|WIAF-3785 |
| 2 | 240.20 cR from top of Chr2 lin | 1384 | WIAF\|WIAF-3289 |
| 2 | 242.40 cR from top of Chr2 lin | 1663 | WIAF\|WIAF-3602 |
| 2 | 246.10 cR from top of Chr2 lin | 1303 | WIAF\|WIAF-2192 |
| 2 | 247.10 cR from top of Chr2 lin | 713 | WIAF\|WIAF-1451 |
| 2 | 247.20 cR from top of Chr2 lin | 1502 | WIAF\|WIAF-3441 |
| 2 | 253.00 cR from top of Chr2 lin | 1309 | WIAF\|WIAF-2198 |
| 2 | 269.50 cR from top of Chr2 lin | 1750 | WIAF\|WIAF-3689 |
| 2 | 272.50 cR from top of Chr2 lin | 1534 | WIAF\|WIAF-3473 |
| 2 | 272.50 cR from top of Chr2 lin | 1702 | WIAF\|WIAF-3641 |
| 2 | 272.60 cR from top of Chr2 lin | 2875 | WIAF\|WIAF-773 |
| 2 | 278.8 cR from top of Chr2 link | 3825 | WIAF\|WIAF-2590 |
| 2 | 285.6 cR from top of Chr2 link | 3539 | WIAF\|WIAF-1994 |
| 2 | 285.7 cR from top of Chr2 link | 3849 | WIAF\|WIAF-2635 |
| 2 | 287.2 cR from top of Chr2 link | 3587 | WIAF\|WIAF-2052 |
| 2 | 287.2 cR from top of Chr2 link | 1071 | WIAF\|WIAF-4203 |
| 2 | 290.4 cR from top of Chr2 link | 2697 | WIAF\|WIAF-383 |
| 2 | 293.7 cR from top of Chr2 link | 2154 | WIAF\|WIAF-486 |
| 2 | 293.7 cR from top of Chr2 link | 2155 | WIAF\|WIAF-487 |
| 2 | 300.1 cR from top of Chr2 link | 2923 | WIAF\|WIAF-822 |
| 2 | 318.2 cR from top of Chr2 link | 966 | WIAF\|WIAF-1371 |
| 2 | 325.6 cR from top of Chr2 link | 4081 | SHGC/AFFYMETRIX\|SNP-SHGC-16802 |
| 2 | 341.6 cR from top of Chr2 link | 2281 | WIAF\|WIAF-751 |
| 2 | 375.3 cR from top of Chr2 link | 2863 | WIAF\|WIAF-760 |
| 2 | 742.6 cR from top of Chr2 link | 2213 | WIAF\|WIAF-635 |
| 2 | 750.0 cR from top of Chr2 link | 2639 | WIAF\|WIAF-305 |
| 2 | 750.1 cR from top of Chr2 link | 2954 | WIAF\|WIAF-853 |
| 2 | 758.7 cR from top of Chr2 link | 893 | WIAF\|WIAF-1140 |
| 2 | 758.7 cR from top of Chr2 link | 1059 | WIAF\|WIAF-4175 |
| 2 | 780.9 cR from top of Chr2 link | 2253 | WIAF\|WIAF-701 |
| 2 | 783.9 cR from top of Chr2 link | 2673 | WIAF\|WIAF-353 |
| 2 | 796.0 cR from top of Chr2 link | 2493 | WIAF\|WIAF-91 |
| 2 | 824.7 cR from top of Chr2 link | 2472 | WIAF\|WIAF-66 |
| 2 | 838.5 cR from top of Chr2 link | 2194 | WIAF\|WIAF-594 |
| 2 | 854.9 cR from top of Chr2 link | 2209 | WIAF\|WIAF-629 |
| 2 | 881.0 cR from top of Chr2 link | 2878 | WIAF\|WIAF-776 |
| 2 | 900.2 cR from top of Chr2 link | 3858 | WIAF\|WIAF-2647 |
| 2 | 902.1 cR from top of Chr2 link | 2661 | WIAF\|WIAF-337 |
| 2 | 910.6 cR from top of Chr2 link | 2961 | WIAF\|WIAF-860 |
| 2 | 910.6 cR from top of Chr2 link | 2962 | WIAF\|WIAF-861 |
| 2 | 910.6 cR from top of Chr2 link | 2963 | WIAF\|WIAF-862 |
| 2 | 915.7 cR from top of Chr2 link | 2500 | WIAF\|WIAF-99 |
| 2 | 920.4 cR from top of Chr2 link | 726 | WIAF\|WIAF-1066 |
| 2 | 931.1 cR from top of Chr2 link | 2104 | WIAF\|WIAF-177 |
| 2 | 952.3 cR from top of Chr2 link | 2516 | WIAF\|WIAF-124 |
| 2 | 956.7 cR from top of Chr2 link | 2629 | WIAF\|WIAF-289 |
| 2 | 961.8 cR from top of Chr2 link | 3109 | WIAF\|WIAF-1010 |
| 2 | 981.1 cR from top of Chr2 link | 2470 | WIAF\|WIAF-64 |
| 2 | 986.9 cR from top of Chr2 link | 2125 | WIAF\|WIAF-300 |
| 2 | 986.9 cR from top of Chr2 link | 2126 | WIAF\|WIAF-301 |
| 2 | 1009.4 cR from top of Chr2 lin | 2978 | WIAF\|WIAF-878 |
| 2 | 1026.1 cR from top of Chr2 lin | 3871 | WIAF\|WIAF-2670 |
| 2 | 1026.1 cR from top of Chr2 lin | 3872 | WIAF\|WIAF-2671 |
| 2 | 1074.0 cR from top of Chr2 lin | 2738 | WIAF\|WIAF-450 |
| 2 | 1089.0 cR from top of Chr2 lin | 2052 | WIAF\|WIAF-1700 |
| 2 | 1092.0 cR from top of Chr2 lin | 3474 | WIAF\|WIAF-1929 |
| 2 | 1092.0 cR from top of Chr2 lin | 3475 | WIAF\|WIAF-1930 |
| 2 | 1104.9 cR from top of Chr2 lin | 3309 | WIAF\|WIAF-1762 |
| 2 | | 4223 | MARSHFIELD\|MID-15 |
| 2 | | 4224 | MARSHFIELD\|MID-16 |
| 2 | | 3962 | SHGC/AFFYMETRIX\|SNP-SHGC-11130 |
| 2 | | 4069 | SHGC/AFFYMETRIX\|SNP-SHGC-13615 |
| 2 | | 3967 | SHGC/AFFYMETRIX\|SNP-SHGC-13867 |
| 2 | | 3968 | SHGC/AFFYMETRIX\|SNP-SHGC-13934 |
| 2 | | 4164 | SHGC/AFFYMETRIX\|SNP-SHGC-15247 |
| 2 | | 4074 | SHGC/AFFYMETRIX\|SNP-SHGC-15661 |
| 2 | | 4087 | SHGC/AFFYMETRIX\|SNP-SHGC-17089 |
| 2 | | 4016 | SHGC/AFFYMETRIX\|SNP-SHGC-3987 |
| 2 | | 4040 | SHGC/AFFYMETRIX\|SNP-SHGC-8478 |
| 2 | | 4044 | SHGC/AFFYMETRIX\|SNP-SHGC-9017 |
| 2 | | 4048 | SHGC/AFFYMETRIX\|SNP-SHGC-9366 |
| 2 | | 3122 | WIAF\|WIAF-1023 |
| 2 | | 3130 | WIAF\|WIAF-1031 |
| 2 | | 3159 | WIAF\|WIAF-1458 |
| 2 | | 1218 | WIAF\|WIAF-2105 |
| 2 | | 1231 | WIAF\|WIAF-2118 |
| 2 | | 1253 | WIAF\|WIAF-2140 |
| 2 | | 1254 | WIAF\|WIAF-2141 |
| 2 | | 3672 | WIAF\|WIAF-2400 |
| 2 | | 3683 | WIAF\|WIAF-2411 |
| 2 | | 3705 | WIAF\|WIAF-2433 |
| 2 | | 3781 | WIAF\|WIAF-2509 |
| 2 | | 3782 | WIAF\|WIAF-2510 |
| 2 | | 2447 | WIAF\|WIAF-35 |
| 2 | | 2448 | WIAF\|WIAF-36 |
| 2 | | 2449 | WIAF\|WIAF-37 |
| 2 | | 2480 | WIAF\|WIAF-76 |
| 2 | | 3080 | WIAF\|WIAF-981 |
| 2 | | 3097 | WIAF\|WIAF-998 |
| 2 | | 3098 | WIAF\|WIAF-999 |
| 3 | 12.90 cR from top of Chr3 link | 1522 | WIAF\|WIAF-3461 |
| 3 | 12.90 cR from top of Chr3 link | 1524 | WIAF\|WIAF-3463 |
| 3 | 14.5 cR from top of Chr3 linka | 2098 | WIAF\|WIAF-144 |
| 3 | 18.4 cR from top of Chr3 linka | 3339 | WIAF\|WIAF-1792 |
| 3 | 18.4 cR from top of Chr3 linka | 3340 | WIAF\|WIAF-1793 |
| 3 | 19.3 cR from top of Chr3 linka | 2244 | WIAF\|WIAF-685 |
| 3 | 33.50 cR from top of Chr3 link | 3811 | WIAF\|WIAF-2563 |
| 3 | 33.50 cR from top of Chr3 link | 1926 | WIAF\|WIAF-3865 |
| 3 | 36.50 cR from top of Chr3 link | 2886 | WIAF\|WIAF-784 |
| 3 | 36.90 cR from top of Chr3 link | 1893 | WIAF\|WIAF-3832 |
| 3 | 37.90 cR from top of Chr3 link | 1142 | WIAF\|WIAF-1605 |
| 3 | 43.20 cR from top of Chr3 link | 1494 | WIAF\|WIAF-3433 |
| 3 | 44.1 cR from top of Chr3 linka | 2939 | WIAF\|WIAF-838 |
| 3 | 45.30 cR from top of Chr3 link | 3491 | WIAF\|WIAF-1946 |
| 3 | 46.90 cR from top of Chr3 link | 3312 | WIAF\|WIAF-1765 |
| 3 | 49.00 cR from top of Chr3 link | 1449 | WIAF\|WIAF-3380 |
| 3 | 49.00 cR from top of Chr3 link | 1450 | WIAF\|WIAF-3382 |
| 3 | 51.7 cR from top of Chr3 linka | 2191 | WIAF\|WIAF-587 |
| 3 | 54.9 cR from top of Chr3 linka | 2456 | WIAF\|WIAF-47 |
| 3 | 55.0 cR from top of Chr3 linka | 3863 | WIAF\|WIAF-2656 |
| 3 | 55.40 cR from top of Chr3 link | 3471 | WIAF\|WIAF-1926 |
| 3 | 55.60 cR from top of Chr3 link | 3336 | WIAF\|WIAF-1789 |
| 3 | 56.8 cR from top of Chr3 linka | 2508 | WIAF\|WIAF-114 |
| 3 | 56.8 cR from top of Chr3 linka | 2509 | WIAF\|WIAF-115 |
| 3 | 57.80 cR from top of Chr3 link | 2037 | WIAF\|WIAF-1617 |
| 3 | 57.80 cR from top of Chr3 link | 1825 | WIAF\|WIAF-3764 |
| 3 | 57.80 cR from top of Chr3 link | 2707 | WIAF\|WIAF-398 |
| 3 | 58.00 cR from top of Chr3 link | 2984 | WIAF\|WIAF-884 |
| 3 | 66.40 cR from top of Chr3 link | 1308 | WIAF\|WIAF-2197 |
| 3 | 66.80 cR from top of Chr3 link | 3225 | WIAF\|WIAF-1624 |
| 3 | 66.80 cR from top of Chr3 link | 3483 | WIAF\|WIAF-1938 |
| 3 | 67.20 cR from top of Chr3 link | 683 | WIAF\|WIAF-1074 |
| 3 | 67.50 cR from top of Chr3 link | 3245 | WIAF\|WIAF-1655 |
| 3 | 67.50 cR from top of Chr3 link | 1602 | WIAF\|WIAF-3541 |
| 3 | 72.1 cR from top of Chr3 linka | 3308 | WIAF\|WIAF-1761 |
| 3 | 72.30 cR from top of Chr3 link | 3193 | WIAF\|WIAF-1522 |
| 3 | 72.30 cR from top of Chr3 link | 3194 | WIAF\|WIAF-1523 |
| 3 | 72.40 cR from top of Chr3 link | 826 | WIAF\|WIAF-1489 |
| 3 | 72.60 cR from top of Chr3 link | 3410 | WIAF\|WIAF-1865 |
| 3 | 72.8 cR from top of Chr3 linka | 2622 | WIAF\|WIAF-281 |
| 3 | 73.3 cR from top of Chr3 linka | 3868 | WIAF\|WIAF-2663 |
| 3 | 74.00 cR from top of Chr3 link | 1595 | WIAF\|WIAF-3534 |
| 3 | 77.40 cR from top of Chr3 link | 1690 | WIAF\|WIAF-3629 |
| 3 | 80 cM | 4314 | UWGC\|133 |
| 3 | 80.80 cR from top of Chr3 link | 3378 | WIAF\|WIAF-1831 |
| 3 | 92.80 cR from top of Chr3 link | 1452 | WIAF\|WIAF-3385 |
| 3 | 96.60 cR from top of Chr3 link | 1770 | WIAF\|WIAF-3709 |
| 3 | 111.00 cR from top of Chr3 lin | 3341 | WIAF\|WIAF-1794 |
| 3 | 111.10 cR from top of Chr3 lin | 3189 | WIAF\|WIAF-1512 |
| 3 | 111.10 cR from top of Chr3 lin | 3190 | WIAF\|WIAF-1513 |
| 3 | 111.40 cR from top of Chr3 lin | 1956 | WIAF\|WIAF-3895 |
| 3 | 122.30 cR from top of Chr3 lin | 1549 | WIAF\|WIAF-3488 |
| 3 | 124.00 cR from top of Chr3 lin | 1182 | WIAF\|WIAF-1714 |
| 3 | 126.3 cR from top of Chr3 link | 2854 | WIAF\|WIAF-741 |
| 3 | 126.8 cR from top of Chr3 link | 3123 | WIAF\|WIAF-1024 |
| 3 | 126.90 cR from top of Chr3 lin | 1454 | WIAF\|WIAF-3387 |
| 3 | 129.30 cR from top of Chr3 lin | 1395 | WIAF\|WIAF-3300 |
| 3 | 131.30 cR from top of Chr3 lin | 1923 | WIAF\|WIAF-3862 |
| 3 | 134.60 cR from top of Chr3 lin | 1259 | WIAF\|WIAF-2146 |
| 3 | 134.9 cR from top of Chr3 link | 917 | WIAF\|WIAF-1207 |
| 3 | 134.9 cR from top of Chr3 link | 918 | WIAF\|WIAF-1208 |
| 3 | 134.9 cR from top of Chr3 link | 919 | WIAF\|WIAF-1209 |
| 3 | 136.00 cR from top of Chr3 lin | 1931 | WIAF\|WIAF-3870 |
| 3 | 138.00 cR from top of Chr3 lin | 3228 | WIAF\|WIAF-1629 |
| 3 | 138.30 cR from top of Chr3 lin | 1963 | WIAF\|WIAF-3902 |
| 3 | 138.40 cR from top of Chr3 lin | 1725 | WIAF\|WIAF-3664 |
| 3 | 140.70 cR from top of Chr3 lin | 1092 | WIAF\|WIAF-1473 |
| 3 | 141.0 cR from top of Chr3 link | 3147 | WIAF\|WIAF-1048 |
| 3 | 141.20 cR from top of Chr3 lin | 1970 | WIAF\|WIAF-3909 |
| 3 | 142.20 cR from top of Chr3 lin | 1229 | WIAF\|WIAF-2116 |
| 3 | 142.40 cR from top of Chr3 lin | 1187 | WIAF\|WIAF-2074 |
| 3 | 143.80 cR from top of Chr3 lin | 3263 | WIAF\|WIAF-1702 |
| 3 | 143.80 cR from top of Chr3 lin | 3264 | WIAF\|WIAF-1703 |
| 3 | 143.90 cR from top of Chr3 lin | 1195 | WIAF\|WIAF-2082 |
| 3 | 144.20 cR from top of Chr3 lin | 1158 | WIAF\|WIAF-1656 |
| 3 | 144.70 cR from top of Chr3 lin | 1722 | WIAF\|WIAF-3661 |
| 3 | 147.8 cR from top of Chr3 link | 2572 | WIAF\|WIAF-202 |
| 3 | 151.20 cR from top of Chr3 lin | 1152 | WIAF\|WIAF-1636 |
| 3 | 151.20 cR from top of Chr3 lin | 1153 | WIAF\|WIAF-1637 |
| 3 | 153.80 cR from top of Chr3 lin | 1890 | WIAF\|WIAF-3829 |
| 3 | 156.30 cR from top of Chr3 lin | 1716 | WIAF\|WIAF-3655 |
| 3 | 156.60 cR from top of Chr3 lin | 1734 | WIAF\|WIAF-3673 |
| 3 | 164.20 cR from top of Chr3 lin | 3296 | WIAF\|WIAF-1749 |
| 3 | 164.20 cR from top of Chr3 lin | 1629 | WIAF\|WIAF-3568 |
| 3 | 166.0 cR from top of Chr3 link | 2898 | WIAF\|WIAF-796 |
| 3 | 170.20 cR from top of Chr3 lin | 1852 | WIAF\|WIAF-3791 |
| 3 | 171.7 cR from top of Chr3 link | 2917 | WIAF\|WIAF-816 |
| 3 | 173.30 cR from top of Chr3 lin | 1351 | WIAF\|WIAF-3254 |
| 3 | 173.50 cR from top of Chr3 lin | 1407 | WIAF\|WIAF-3327 |
| 3 | 186.60 cR from top of Chr3 lin | 1819 | WIAF\|WIAF-3758 |
| 3 | 186.7 cR from top of Chr3 link | 2571 | WIAF\|WIAF-201 |
| 3 | 187.60 cR from top of Chr3 lin | 1305 | WIAF\|WIAF-2194 |
| 3 | 187.9 cR from top of Chr3 link | 2114 | WIAF\|WIAF-236 |
| 3 | 189.80 cR from top of Chr3 lin | 1463 | WIAF\|WIAF-3398 |
| 3 | 195.7 cR from top of Chr3 link | 3847 | WIAF\|WIAF-2626 |
| 3 | 228.30 cR from top of Chr3 lin | 1887 | WIAF\|WIAF-3826 |
| 3 | 228.30 cR from top of Chr3 lin | 1888 | WIAF\|WIAF-3827 |
| 3 | 228.4 cR from top of Chr3 link | 3472 | WIAF\|WIAF-1927 |
| 3 | 228.4 cR from top of Chr3 link | 3473 | WIAF\|WIAF-1928 |
| 3 | 228.60 cR from top of Chr3 lin | 1161 | WIAF\|WIAF-1664 |
| 3 | 233.00 cR from top of Chr3 lin | 1383 | WIAF\|WIAF-3288 |
| 3 | 233.00 cR from top of Chr3 lin | 1470 | WIAF\|WIAF-3405 |
| 3 | 233.00 cR from top of Chr3 lin | 1471 | WIAF\|WIAF-3406 |
| 3 | 233.00 cR from top of Chr3 lin | 1587 | WIAF\|WIAF-3526 |
| 3 | 233.00 cR from top of Chr3 lin | 1627 | WIAF\|WIAF-3566 |
| 3 | 233.8 cR from top of Chr3 link | 3446 | WIAF\|WIAF-1901 |
| 3 | 239.5 cR from top of Chr3 link | 1032 | WIAF\|WIAF-4092 |
| 3 | 240.4 cR from top of Chr3 link | 2234 | WIAF\|WIAF-669 |
| 3 | 240.4 cR from top of Chr3 link | 2235 | WIAF\|WIAF-670 |
| 3 | 269.6 cR from top of Chr3 link | 868 | WIAF\|WIAF-1081 |
| 3 | 269.6 cR from top of Chr3 link | 1003 | WIAF\|WIAF-4026 |
| 3 | 463.3 cR from top of Chr3 link | 2342 | WIAF\|WIAF-2572 |
| 3 | 477.2 cR from top of Chr3 link | 4560 | HU-CHINA\|1-1176-2 |
| 3 | 477.2 cR from top of Chr3 link | 4587 | HU-CHINA\|3-1176 |
| 3 | 477.2 cR from top of Chr3 link | 741 | WIAF\|WIAF-1176 |
| 3 | 533.1 cR from top of Chr3 link | 3839 | WIAF\|WIAF-2612 |
| 3 | 534.4 cR from top of Chr3 link | 2855 | WIAF\|WIAF-745 |
| 3 | 546.0 cR from top of Chr3 link | 3085 | WIAF\|WIAF-986 |
| 3 | 552.8 cR from top of Chr3 link | 1753 | WIAF\|WIAF-3692 |
| 3 | 569.6 cR from top of Chr3 link | 3354 | WIAF\|WIAF-1807 |
| 3 | 569.6 cR from top of Chr3 link | 2440 | WIAF\|WIAF-25 |
| 3 | 604.7 cR from top of Chr3 link | 2358 | WIAF\|WIAF-2606 |
| 3 | 611.1 cR from top of Chr3 link | 2905 | WIAF\|WIAF-803 |
| 3 | 616.0 cR from top of Chr3 link | 788 | WIAF\|WIAF-2056 |
| 3 | 640.6 cR from top of Chr3 link | 2185 | WIAF\|WIAF-568 |
| 3 | 640.6 cR from top of Chr3 link | 2186 | WIAF\|WIAF-569 |
| 3 | 672.1 cR from top of Chr3 link | 2830 | WIAF\|WIAF-650 |
| 3 | 672.1 cR from top of Chr3 link | 2831 | WIAF\|WIAF-651 |
| 3 | 680.5 cR from top of Chr3 link | 2379 | WIAF\|WIAF-2664 |
| 3 | 680.5 cR from top of Chr3 link | 2380 | WIAF\|WIAF-2665 |
| 3 | 690.2 cR from top of Chr3 link | 2656 | WIAF\|WIAF-330 |
| 3 | 718.0 cR from top of Chr3 link | 2207 | WIAF\|WIAF-625 |
| 3 | 718.0 cR from top of Chr3 link | 2208 | WIAF\|WIAF-626 |
| 3 | 718.5 cR from top of Chr3 link | 2217 | WIAF\|WIAF-639 |
| 3 | 775.9 cR from top of Chr3 link | 2461 | WIAF\|WIAF-52 |
| 3 | 791.4 cR from top of Chr3 link | 2574 | WIAF\|WIAF-204 |
| 3 | 792.2 cR from top of Chr3 link | 1256 | WIAF\|WIAF-2143 |
| 3 | 793.4 cR from top of Chr3 link | 2948 | WIAF\|WIAF-847 |
| 3 | 793.7 cR from top of Chr3 link | 2779 | WIAF\|WIAF-523 |
| 3 | 796.7 cR from top of Chr3 link | 2788 | WIAF\|WIAF-542 |
| 3 | 802.4 cR from top of Chr3 link | 2173 | WIAF\|WIAF-543 |
| 3 | 808.9 cR from top of Chr3 link | 2246 | WIAF\|WIAF-690 |
| 3 | 838.9 cR from top of Chr3 link | 2604 | WIAF\|WIAF-249 |
| 3 | 838.9 cR from top of Chr3 link | 2605 | WIAF\|WIAF-250 |
| 3 | 842.9 cR from top of Chr3 link | 2703 | WIAF\|WIAF-392 |
| 3 | 848.1 cR from top of Chr3 link | 2630 | WIAF\|WIAF-290 |
| 3 | 848.1 cR from top of Chr3 link | 2631 | WIAF\|WIAF-291 |
| 3 | 848.1 cR from top of Chr3 link | 2632 | WIAF\|WIAF-292 |
| 3 | 868.2 cR from top of Chr3 link | 3814 | WIAF\|WIAF-2568 |
| 3 | 868.6 cR from top of Chr3 link | 3366 | WIAF\|WIAF-1819 |
| 3 | 879.8 cR from top of Chr3 link | 224 | KWOK\|D3S2344-1 |
| 3 | 879.8 cR from top of Chr3 link | 225 | KWOK\|D3S2344-2 |
| 3 | 879.8 cR from top of Chr3 link | 766 | WIAF\|WIAF-1365 |
| 3 | 896.5 cR from top of Chr3 link | 3333 | WIAF\|WIAF-1786 |
| 3 | 897.8 cR from top of Chr3 link | 3451 | WIAF\|WIAF-1906 |
| 3 | 903.2 cR from top of Chr3 link | 3360 | WIAF\|WIAF-1813 |
| 3 | 907.0 cR from top of Chr3 link | 2513 | WIAF\|WIAF-119 |
| 3 | 907.0 cR from top of Chr3 link | 2514 | WIAF\|WIAF-120 |
| 3 | 917.9 cR from top of Chr3 link | 3078 | WIAF\|WIAF-979 |
| 3 | 918.0 cR from top of Chr3 link | 2543 | WIAF\|WIAF-162 |
| 3 | 921.8 cR from top of Chr3 link | 3106 | WIAF\|WIAF-1007 |
| 3 | | 4225 | MARSHFIELD\|MID-17 |
| 3 | | 3998 | SHGC/AFFYMETRIX\|SNP-SHGC-11665 |
| 3 | | 3999 | SHGC/AFFYMETRIX\|SNP-SHGC-1204 |
| 3 | | 4138 | SHGC/AFFYMETRIX\|SNP-SHGC-13087 |
| 3 | | 4067 | SHGC/AFFYMETRIX\|SNP-SHGC-13482 |
| 3 | | 4147 | SHGC/AFFYMETRIX\|SNP-SHGC-14087 |
| 3 | | 4151 | SHGC/AFFYMETRIX\|SNP-SHGC-14182 |
| 3 | | 4156 | SHGC/AFFYMETRIX\|SNP-SHGC-14457 |
| 3 | | 4162 | SHGC/AFFYMETRIX\|SNP-SHGC-14769 |
| 3 | | 3970 | SHGC/AFFYMETRIX\|SNP-SHGC-16777 |
| 3 | | 4089 | SHGC/AFFYMETRIX\|SNP-SHGC-17103 |
| 3 | | 4097 | SHGC/AFFYMETRIX\|SNP-SHGC-18889 |
| 3 | | 4106 | SHGC/AFFYMETRIX\|SNP-SHGC-32258 |
| 3 | | 4012 | SHGC/AFFYMETRIX\|SNP-SHGC-3249 |
| 3 | | 3974 | SHGC/AFFYMETRIX\|SNP-SHGC-33980 |
| 3 | | 4107 | SHGC/AFFYMETRIX\|SNP-SHGC-35481 |
| 3 | | 4035 | SHGC/AFFYMETRIX\|SNP-SHGC-7204 |
| 3 | | 3144 | WIAF\|WIAF-1045 |
| 3 | | 3146 | WIAF\|WIAF-1047 |
| 3 | | 3530 | WIAF\|WIAF-1985 |
| 3 | | 3740 | WIAF\|WIAF-2468 |
| 3 | | 2061 | WIAF\|WIAF-2547 |
| 3 | | 1500 | WIAF\|WIAF-3439 |
| 3 | | 1673 | WIAF\|WIAF-3612 |
| 3 | | 3925 | WIAF\|WIAF-4013 |
| 3 | | 2996 | WIAF\|WIAF-896 |
| 3 | | 3006 | WIAF\|WIAF-906 |
| 3 | | 3017 | WIAF\|WIAF-917 |
| 3 | | 3048 | WIAF\|WIAF-949 |
| 4 | 3.70 cR from top of Chr4 linka | 1204 | WIAF\|WIAF-2091 |
| 4 | 3.70 cR from top of Chr4 linka | 1919 | WIAF\|WIAF-3858 |
| 4 | 4.4 cR from top of Chr4 linkag | 3866 | WIAF\|WIAF-2660 |
| 4 | 4.70 cR from top of Chr4 linka | 3215 | WIAF\|WIAF-1591 |
| 4 | 4.70 cR from top of Chr4 linka | 3216 | WIAF\|WIAF-1592 |
| 4 | 4.70 cR from top of Chr4 linka | 3217 | WIAF\|WIAF-1593 |
| 4 | 4.70 cR from top of Chr4 linka | 1210 | WIAF\|WIAF-2097 |
| 4 | 5.30 cR from top of Chr4 linka | 1120 | WIAF\|WIAF-1555 |
| 4 | 8.60 cR from top of Chr4 linka | 3233 | WIAF\|WIAF-1639 |
| 4 | 15.00 cR from top of Chr4 link | 1332 | WIAF\|WIAF-2222 |
| 4 | 16.1 cR from top of Chr4 linka | 3809 | WIAF\|WIAF-2561 |
| 4 | 18.70 cR from top of Chr4 link | 1307 | WIAF\|WIAF-2196 |
| 4 | 19.8 cR from top of Chr4 linka | 3503 | WIAF\|WIAF-1958 |
| 4 | 22.00 cR from top of Chr4 link | 3250 | WIAF\|WIAF-1668 |
| 4 | 26.80 cR from top of Chr4 link | 1811 | WIAF\|WIAF-3750 |
| 4 | 26.80 cR from top of Chr4 link | 1814 | WIAF\|WIAF-3753 |
| 4 | 27.7 cR from top of Chr4 linka | 2070 | WIAF\|WIAF-2557 |
| 4 | 28.20 cR from top of Chr4 link | 3161 | WIAF\|WIAF-1464 |
| 4 | 28.90 cR from top of Chr4 link | 3555 | WIAF\|WIAF-2010 |
| 4 | 29.80 cR from top of Chr4 link | 3507 | WIAF\|WIAF-1962 |
| 4 | 29.80 cR from top of Chr4 link | 3508 | WIAF\|WIAF-1963 |
| 4 | 35.40 cR from top of Chr4 link | 3163 | WIAF\|WIAF-1466 |
| 4 | 36.90 cR from top of Chr4 link | 1520 | WIAF\|WIAF-3459 |
| 4 | 39.3 cR from top of Chr4 linka | 2076 | WIAF\|WIAF-14 |
| 4 | 43.50 cR from top of Chr4 link | 707 | WIAF\|WIAF-1395 |
| 4 | 45.9 cR from top of Chr4 linka | 3003 | WIAF\|WIAF-903 |
| 4 | 51.00 cR from top of Chr4 link | 3470 | WIAF\|WIAF-1925 |
| 4 | 51.90 cR from top of Chr4 link | 2021 | WIAF\|WIAF-1527 |
| 4 | 55.2 cR from top of Chr4 linka | 231 | KWOK\|D4S2341 |
| 4 | 55.2 cR from top of Chr4 linka | 2092 | WIAF\|WIAF-109 |
| 4 | 55.2 cR from top of Chr4 linka | 780 | WIAF\|WIAF-1433 |
| 4 | 55.2 cR from top of Chr4 linka | 2340 | WIAF\|WIAF-2566 |
| 4 | 55.2 cR from top of Chr4 linka | 806 | WIAF\|WIAF-4142 |
| 4 | 58.40 cR from top of Chr4 link | 1475 | WIAF\|WIAF-3412 |
| 4 | 61.8 cR from top of Chr4 linka | 2210 | WIAF\|WIAF-630 |
| 4 | 65.2 cR from top of Chr4 linka | 2129 | WIAF\|WIAF-316 |
| 4 | 74.30 cR from top of Chr4 link | 1461 | WIAF\|WIAF-3395 |
| 4 | 78.50 cR from top of Chr4 link | 3131 | WIAF\|WIAF-1032 |
| 4 | 80.70 cR from top of Chr4 link | 672 | WIAF\|WIAF-1370 |
| 4 | 91.50 cR from top of Chr4 link | 1145 | WIAF\|WIAF-1611 |
| 4 | 91.70 cR from top of Chr4 link | 1433 | WIAF\|WIAF-3361 |
| 4 | 96.60 cR from top of Chr4 link | 1097 | WIAF\|WIAF-1485 |
| 4 | 98.5 cR from top of Chr4 linka | 3520 | WIAF\|WIAF-1975 |
| 4 | 98.5 cR from top of Chr4 linka | 3521 | WIAF\|WIAF-1976 |
| 4 | 98.80 cR from top of Chr4 link | 1226 | WIAF\|WIAF-2113 |
| 4 | 98.80 cR from top of Chr4 link | 1227 | WIAF\|WIAF-2114 |
| 4 | 106.40 cR from top of Chr4 lin | 1826 | WIAF\|WIAF-3765 |
| 4 | 108.70 cR from top of Chr4 lin | 703 | WIAF\|WIAF-1333 |
| 4 | 112.10 cR from top of Chr4 lin | 1252 | WIAF\|WIAF-2139 |
| 4 | 120.20 cR from top of Chr4 lin | 1304 | WIAF\|WIAF-2193 |
| 4 | 121.00 cR from top of Chr4 lin | 2019 | WIAF\|WIAF-1524 |
| 4 | 121.40 cR from top of Chr4 lin | 3165 | WIAF\|WIAF-1469 |
| 4 | 121.50 cR from top of Chr4 lin | 1348 | WIAF\|WIAF-3251 |
| 4 | 121.50 cR from top of Chr4 lin | 1871 | WIAF\|WIAF-3810 |
| 4 | 121.50 cR from top of Chr4 lin | 1872 | WIAF\|WIAF-3811 |
| 4 | 122.60 cR from top of Chr4 lin | 1575 | WIAF\|WIAF-3514 |
| 4 | 122.60 cR from top of Chr4 lin | 1576 | WIAF\|WIAF-3515 |
| 4 | 124.40 cR from top of Chr4 lin | 1354 | WIAF\|WIAF-3257 |
| 4 | 125.80 cR from top of Chr4 lin | 1879 | WIAF\|WIAF-3818 |
| 4 | 125.80 cR from top of Chr4 lin | 1882 | WIAF\|WIAF-3821 |
| 4 | 126.10 cR from top of Chr4 lin | 1953 | WIAF\|WIAF-3892 |
| 4 | 128.3 cR from top of Chr4 link | 2467 | WIAF\|WIAF-60 |
| 4 | 139.80 cR from top of Chr4 lin | 1906 | WIAF\|WIAF-3845 |
| 4 | 140.70 cR from top of Chr4 lin | 1966 | WIAF\|WIAF-3905 |
| 4 | 141.70 cR from top of Chr4 lin | 1466 | WIAF\|WIAF-3401 |
| 4 | 144.1 cR from top of Chr4 link | 3129 | WIAF\|WIAF-1030 |
| 4 | 145.50 cR from top of Chr4 lin | 1093 | WIAF\|WIAF-1475 |
| 4 | 146.00 cR from top of Chr4 lin | 2902 | WIAF\|WIAF-800 |
| 4 | 148.40 cR from top of Chr4 lin | 1135 | WIAF\|WIAF-1589 |
| 4 | 148.60 cR from top of Chr4 lin | 2464 | WIAF\|WIAF-57 |
| 4 | 149.40 cR from top of Chr4 lin | 1362 | WIAF\|WIAF-3265 |
| 4 | 149.40 cR from top of Chr4 lin | 1364 | WIAF\|WIAF-3267 |
| 4 | 153 Cm | 4306 | UWGC\|125 |
| 4 | 153.60 cR from top of Chr4 lin | 3535 | WIAF\|WIAF-1990 |
| 4 | 174.10 cR from top of Chr4 lin | 1479 | WIAF\|WIAF-3418 |
| 4 | 182.20 cR from top of Chr4 lin | 1821 | WIAF\|WIAF-3760 |
| 4 | 193.30 cR from top of Chr4 lin | 1353 | WIAF\|WIAF-3256 |
| 4 | 193.8 cR from top of Chr4 link | 1984 | WIAF\|WIAF-3923 |
| 4 | 197.2 cR from top of Chr4 link | 2913 | WIAF\|WIAF-812 |
| 4 | 199.00 cR from top of Chr4 lin | 3219 | WIAF\|WIAF-1597 |
| 4 | 199.10 cR from top of Chr4 lin | 1618 | WIAF\|WIAF-3557 |
| 4 | 199.70 cR from top of Chr4 lin | 1865 | WIAF\|WIAF-3804 |
| 4 | 243.0 cR from top of Chr4 link | 2739 | WIAF\|WIAF-452 |
| 4 | 401.1 cR from top of Chr4 link | 2152 | WIAF\|WIAF-482 |
| 4 | 401.1 cR from top of Chr4 link | 2153 | WIAF\|WIAF-483 |
| 4 | 412.2 cR from top of Chr4 link | 3031 | WIAF\|WIAF-931 |
| 4 | 415.9 cR from top of Chr4 link | 4564 | HU-CHINA\|4-197 |
| 4 | 415.9 cR from top of Chr4 link | 2568 | WIAF\|WIAF-197 |
| 4 | 419.4 cR from top of Chr4 link | 880 | WIAF\|WIAF-1116 |
| 4 | 426.7 cR from top of Chr4 link | 982 | WIAF\|WIAF-1408 |
| 4 | 474.6 cR from top of Chr4 link | 3019 | WIAF\|WIAF-919 |
| 4 | 474.6 cR from top of Chr4 link | 3020 | WIAF\|WIAF-920 |
| 4 | 483.0 cR from top of Chr4 link | 2712 | WIAF\|WIAF-407 |
| 4 | 497.5 cR from top of Chr4 link | 3981 | SHGC/AFFYMETRIX\|SNP-SHGC-51763 |
| 4 | 499.2 cR from top of Chr4 link | 2780 | WIAF\|WIAF-524 |
| 4 | 508.3 cR from top of Chr4 link | 2815 | WIAF\|WIAF-610 |
| 4 | 508.3 cR from top of Chr4 link | 2816 | WIAF\|WIAF-611 |
| 4 | 522.1 cR from top of Chr4 link | 2756 | WIAF\|WIAF-484 |
| 4 | 523.9 cR from top of Chr4 link | 2688 | WIAF\|WIAF-373 |
| 4 | 526.7 cR from top of Chr4 link | 2914 | WIAF\|WIAF-813 |
| 4 | 533.1 cR from top of Chr4 link | 4181 | SHGC/AFFYMETRIX\|SNP-SHGC-50672 |
| 4 | 533.1 cR from top of Chr4 link | 785 | WIAF\|WIAF-2048 |
| 4 | 538.1 cR from top of Chr4 link | 1312 | WIAF\|WIAF-2201 |
| 4 | 543.1 cR from top of Chr4 link | 729 | WIAF\|WIAF-1078 |
| 4 | 563.3 cR from top of Chr4 link | 2179 | WIAF\|WIAF-561 |
| 4 | 572.4 cR from top of Chr4 link | 3300 | WIAF\|WIAF-1753 |
| 4 | 572.9 cR from top of Chr4 link | 2344 | WIAF\|WIAF-2575 |
| 4 | 602.7 cR from top of Chr4 link | 2995 | WIAF\|WIAF-895 |
| 4 | 626.5 cR from top of Chr4 link | 2094 | WIAF\|WIAF-121 |
| 4 | 626.5 cR from top of Chr4 link | 2095 | WIAF\|WIAF-122 |
| 4 | 631.4 cR from top of Chr4 link | 2364 | WIAF\|WIAF-2621 |
| 4 | 631.4 cR from top of Chr4 link | 2365 | WIAF\|WIAF-2623 |
| 4 | 642.1 cR from top of Chr4 link | 2074 | WIAF\|WIAF-8 |
| 4 | 644.6 cR from top of Chr4 link | 3823 | WIAF\|WIAF-2587 |
| 4 | 644.6 cR from top of Chr4 link | 3826 | WIAF\|WIAF-2591 |
| 4 | 4p | 3986 | SHGC/AFFYMETRIX\|SNPA-SHGC4-1659 |
| 4 | 4p | 3987 | SHGC/AFFYMETRIX\|SNPA-SHGC-51324 |
| 4 | 4p | 3991 | SHGC/AFFYMETRIX\|SNPB-SHGC4-1659 |
| 4 | 4p | 3992 | SHGC/AFFYMETRIX\|SNPB-SHGC-51324 |
| 4 | 4p | 3994 | SHGC/AFFYMETRIX\|SNPC-SHGC-51324 |
| 4 | 4p | 4019 | SHGC/AFFYMETRIX\|SNP-SHGC4-1525 |
| 4 | 4p | 4200 | SHGC/AFFYMETRIX\|SNP-SHGC-51310 |
| 4 | 4p | 4201 | SHGC/AFFYMETRIX\|SNP-SHGC-51312 |
| 4 | 4p | 4204 | SHGC/AFFYMETRIX\|SNP-SHGC-51346 |
| 4 | | 4252 | MARSHFIELD\|MID-7 |
| 4 | | 4119 | SHGC/AFFYMETRIX\|SNPA-SHGC-14934 |
| 4 | | 4121 | SHGC/AFFYMETRIX\|SNPA-SHGC-24080 |
| 4 | | 4055 | SHGC/AFFYMETRIX\|SNPA-SHGC-50187 |
| 4 | | 4056 | SHGC/AFFYMETRIX\|SNPA-SHGC-50252 |
| 4 | | 4122 | SHGC/AFFYMETRIX\|SNPA-SHGC-50922 |
| 4 | | 4057 | SHGC/AFFYMETRIX\|SNPA-SHGC-50928 |
| 4 | | 4123 | SHGC/AFFYMETRIX\|SNPA-SHGC-51072 |
| 4 | | 4124 | SHGC/AFFYMETRIX\|SNPA-SHGC-51160 |
| 4 | | 4125 | SHGC/AFFYMETRIX\|SNPA-SHGC-51438 |
| 4 | | 4126 | SHGC/AFFYMETRIX\|SNPA-SHGC-51690 |
| 4 | | 4129 | SHGC/AFFYMETRIX\|SNPB-SHGC-14934 |
| 4 | | 4131 | SHGC/AFFYMETRIX\|SNPB-SHGC-24080 |
| 4 | | 4061 | SHGC/AFFYMETRIX\|SNPB-SHGC-50187 |
| 4 | | 4062 | SHGC/AFFYMETRIX\|SNPB-SHGC-50252 |
| 4 | | 4132 | SHGC/AFFYMETRIX\|SNPB-SHGC-50922 |
| 4 | | 4063 | SHGC/AFFYMETRIX\|SNPB-SHGC-50928 |
| 4 | | 4133 | SHGC/AFFYMETRIX\|SNPB-SHGC-51072 |
| 4 | | 4134 | SHGC/AFFYMETRIX\|SNPB-SHGC-51160 |
| 4 | | 4135 | SHGC/AFFYMETRIX\|SNPB-SHGC-51438 |
| 4 | | 4136 | SHGC/AFFYMETRIX\|SNPB-SHGC-51690 |
| 4 | | 3958 | SHGC/AFFYMETRIX\|SNP-SHGC-10699 |
| 4 | | 4005 | SHGC/AFFYMETRIX\|SNP-SHGC-13008 |
| 4 | | 4150 | SHGC/AFFYMETRIX\|SNP-SHGC-14139 |
| 4 | | 4077 | SHGC/AFFYMETRIX\|SNP-SHGC-16028 |
| 4 | | 4091 | SHGC/AFFYMETRIX\|SNP-SHGC-17200 |
| 4 | | 4167 | SHGC/AFFYMETRIX\|SNP-SHGC-23754 |
| 4 | | 4169 | SHGC/AFFYMETRIX\|SNP-SHGC-24086 |
| 4 | | 4170 | SHGC/AFFYMETRIX\|SNP-SHGC-24090 |
| 4 | | 4171 | SHGC/AFFYMETRIX\|SNP-SHGC-25057 |
| 4 | | 4172 | SHGC/AFFYMETRIX\|SNP-SHGC-25080 |
| 4 | | 4173 | SHGC/AFFYMETRIX\|SNP-SHGC-25091 |
| 4 | | 4174 | SHGC/AFFYMETRIX\|SNP-SHGC-25112 |
| 4 | | 4175 | SHGC/AFFYMETRIX\|SNP-SHGC-25184 |
| 4 | | 4017 | SHGC/AFFYMETRIX\|SNP-SHGC4-1137 |
| 4 | | 4018 | SHGC/AFFYMETRIX\|SNP-SHGC4-1459 |
| 4 | | 4020 | SHGC/AFFYMETRIX\|SNP-SHGC4-1597 |
| 4 | | 4021 | SHGC/AFFYMETRIX\|SNP-SHGC4-1678 |
| 4 | | 4023 | SHGC/AFFYMETRIX\|SNP-SHGC4-851 |
| 4 | | 3978 | SHGC/AFFYMETRIX\|SNP-SHGC-50175 |
| 4 | | 3979 | SHGC/AFFYMETRIX\|SNP-SHGC-50177 |
| 4 | | 4109 | SHGC/AFFYMETRIX\|SNP-SHGC-50262 |
| 4 | | 3980 | SHGC/AFFYMETRIX\|SNP-SHGC-50274 |
| 4 | | 4110 | SHGC/AFFYMETRIX\|SNP-SHGC-50293 |
| 4 | | 4176 | SHGC/AFFYMETRIX\|SNP-SHGC-50311 |
| 4 | | 4177 | SHGC/AFFYMETRIX\|SNP-SHGC-50320 |
| 4 | | 4111 | SHGC/AFFYMETRIX\|SNP-SHGC-50369 |
| 4 | | 4024 | SHGC/AFFYMETRIX\|SNP-SHGC-50475 |
| 4 | | 4179 | SHGC/AFFYMETRIX\|SNP-SHGC-50477 |
| 4 | | 4180 | SHGC/AFFYMETRIX\|SNP-SHGC-50629 |
| 4 | | 4182 | SHGC/AFFYMETRIX\|SNP-SHGC-50730 |
| 4 | | 4113 | SHGC/AFFYMETRIX\|SNP-SHGC-50803 |
| 4 | | 4114 | SHGC/AFFYMETRIX\|SNP-SHGC-50804 |
| 4 | | 4115 | SHGC/AFFYMETRIX\|SNP-SHGC-50810 |
| 4 | | 4183 | SHGC/AFFYMETRIX\|SNP-SHGC-50857 |
| 4 | | 4184 | SHGC/AFFYMETRIX\|SNP-SHGC-50859 |
| 4 | | 4185 | SHGC/AFFYMETRIX\|SNP-SHGC-50880 |
| 4 | | 4186 | SHGC/AFFYMETRIX\|SNP-SHGC-50921 |
| 4 | | 4187 | SHGC/AFFYMETRIX\|SNP-SHGC-50993 |
| 4 | | 4025 | SHGC/AFFYMETRIX\|SNP-SHGC-51011 |
| 4 | | 4188 | SHGC/AFFYMETRIX\|SNP-SHGC-51034 |
| 4 | | 4189 | SHGC/AFFYMETRIX\|SNP-SHGC-51046 |
| 4 | | 4190 | SHGC/AFFYMETRIX\|SNP-SHGC-51122 |
| 4 | | 4191 | SHGC/AFFYMETRIX\|SNP-SHGC-51140 |
| 4 | | 4192 | SHGC/AFFYMETRIX\|SNP-SHGC-51173 |
| 4 | | 4193 | SHGC/AFFYMETRIX\|SNP-SHGC-51187 |
| 4 | | 4194 | SHGC/AFFYMETRIX\|SNP-SHGC-51200 |
| 4 | | 4026 | SHGC/AFFYMETRIX\|SNP-SHGC-51209 |
| 4 | | 4195 | SHGC/AFFYMETRIX\|SNP-SHGC-51227 |
| 4 | | 4196 | SHGC/AFFYMETRIX\|SNP-SHGC-51237 |
| 4 | | 4197 | SHGC/AFFYMETRIX\|SNP-SHGC-51240 |
| 4 | | 4198 | SHGC/AFFYMETRIX\|SNP-SHGC-51242 |
| 4 | | 4199 | SHGC/AFFYMETRIX\|SNP-SHGC-51249 |
| 4 | | 4202 | SHGC/AFFYMETRIX\|SNP-SHGC-51323 |
| 4 | | 4203 | SHGC/AFFYMETRIX\|SNP-SHGC-51340 |
| 4 | | 4205 | SHGC/AFFYMETRIX\|SNP-SHGC-51387 |
| 4 | | 4027 | SHGC/AFFYMETRIX\|SNP-SHGC-51411 |
| 4 | | 4028 | SHGC/AFFYMETRIX\|SNP-SHGC-51435 |
| 4 | | 4029 | SHGC/AFFYMETRIX\|SNP-SHGC-51467 |
| 4 | | 4206 | SHGC/AFFYMETRIX\|SNP-SHGC-51477 |
| 4 | | 4207 | SHGC/AFFYMETRIX\|SNP-SHGC-51520 |
| 4 | | 4208 | SHGC/AFFYMETRIX\|SNP-SHGC-51554 |
| 4 | | 4209 | SHGC/AFFYMETRIX\|SNP-SHGC-51579 |
| 4 | | 4116 | SHGC/AFFYMETRIX\|SNP-SHGC-51662 |
| 4 | | 4210 | SHGC/AFFYMETRIX\|SNP-SHGC-51721 |
| 4 | | 3983 | SHGC/AFFYMETRIX\|SNP-SHGC-9709 |
| 4 | | 2528 | WIAF\|WIAF-138 |
| 4 | | 3531 | WIAF\|WIAF-1986 |
| 4 | | 3686 | WIAF\|WIAF-2414 |
| 4 | | 3688 | WIAF\|WIAF-2416 |
| 4 | | 2950 | WIAF\|WIAF-849 |
| 5 | 0.00 cR from top of Chr5 linka | 3349 | WIAF\|WIAF-1802 |
| 5 | 5.2 cR from top of Chr5 linkag | 2285 | WIAF\|WIAF-1331 |
| 5 | 16.30 cR from top of Chr5 link | 3330 | WIAF\|WIAF-1783 |
| 5 | 16.30 cR from top of Chr5 link | 3331 | WIAF\|WIAF-1784 |
| 5 | 18.60 cR from top of Chr5 link | 1359 | WIAF\|WIAF-3262 |
| 5 | 19.50 cR from top of Chr5 link | 1410 | WIAF\|WIAF-3331 |
| 5 | 19.70 cR from top of Chr5 link | 2013 | WIAF\|WIAF-1507 |
| 5 | 36.8 cR from top of Chr5 linka | 2953 | WIAF\|WIAF-852 |
| 5 | 39.10 cR from top of Chr5 link | 1810 | WIAF\|WIAF-3749 |
| 5 | 39.10 cR from top of Chr5 link | 1813 | WIAF\|WIAF-3752 |
| 5 | 44.5 cR from top of Chr5 linka | 3076 | WIAF\|WIAF-977 |
| 5 | 45.40 cR from top of Chr5 link | 1621 | WIAF\|WIAF-3560 |
| 5 | 51.60 cR from top of Chr5 link | 1105 | WIAF\|WIAF-1532 |
| 5 | 51.60 cR from top of Chr5 link | 1415 | WIAF\|WIAF-3342 |
| 5 | 57.30 cR from top of Chr5 link | 1464 | WIAF\|WIAF-3399 |
| 5 | 62.80 cR from top of Chr5 link | 1636 | WIAF\|WIAF-3575 |
| 5 | 65.00 cR from top of Chr5 link | 3148 | WIAF\|WIAF-1049 |
| 5 | 69.40 cR from top of Chr5 link | 1986 | WIAF\|WIAF-3925 |
| 5 | 69.40 cR from top of Chr5 link | 1987 | WIAF\|WIAF-3926 |
| 5 | 79.40 cR from top of Chr5 link | 3414 | WIAF\|WIAF-1869 |
| 5 | 80.20 cR from top of Chr5 link | 1512 | WIAF\|WIAF-3451 |
| 5 | 80.30 cR from top of Chr5 link | 1665 | WIAF\|WIAF-3604 |
| 5 | 82.30 cR from top of Chr5 link | 3010 | WIAF\|WIAF-910 |
| 5 | 82.80 cR from top of Chr5 link | 3249 | WIAF\|WIAF-1667 |
| 5 | 82.80 cR from top of Chr5 link | 1514 | WIAF\|WIAF-3453 |
| 5 | 84.10 cR from top of Chr5 link | 1591 | WIAF\|WIAF-3530 |
| 5 | 84.10 cR from top of Chr5 link | 1605 | WIAF\|WIAF-3544 |
| 5 | 86.10 cR from top of Chr5 link | 3180 | WIAF\|WIAF-1496 |
| 5 | 87.20 cR from top of Chr5 link | 1525 | WIAF\|WIAF-3464 |
| 5 | 92.30 cR from top of Chr5 link | 1608 | WIAF\|WIAF-3547 |
| 5 | 93.80 cR from top of Chr5 link | 1614 | WIAF\|WIAF-3553 |
| 5 | 97.90 cR from top of Chr5 link | 1515 | WIAF\|WIAF-3454 |
| 5 | 97.90 cR from top of Chr5 link | 1517 | WIAF\|WIAF-3456 |
| 5 | 103.0 cR from top of Chr5 link | 2113 | WIAF\|WIAF-235 |
| 5 | 104.50 cR from top of Chr5 lin | 2033 | WIAF\|WIAF-1594 |
| 5 | 104.50 cR from top of Chr5 lin | 3367 | WIAF\|WIAF-1820 |
| 5 | 104.50 cR from top of Chr5 lin | 3368 | WIAF\|WIAF-1821 |
| 5 | 104.50 cR from top of Chr5 lin | 3369 | WIAF\|WIAF-1822 |
| 5 | 109.00 cR from top of Chr5 lin | 3107 | WIAF\|WIAF-1008 |
| 5 | 117.3 cR from top of Chr5 link | 2229 | WIAF\|WIAF-662 |
| 5 | 117.3 cR from top of Chr5 link | 2230 | WIAF\|WIAF-663 |
| 5 | 121.60 cR from top of Chr5 lin | 997 | WIAF\|WIAF-2053 |
| 5 | 122.30 cR from top of Chr5 lin | 1221 | WIAF\|WIAF-2108 |
| 5 | 122.30 cR from top of Chr5 lin | 1222 | WIAF\|WIAF-2109 |
| 5 | 122.60 cR from top of Chr5 lin | 1939 | WIAF\|WIAF-3878 |
| 5 | 124.3 cR from top of Chr5 link | 3145 | WIAF\|WIAF-1046 |
| 5 | 131.7 cR from top of Chr5 link | 3835 | WIAF\|WIAF-2608 |
| 5 | 131.7 cR from top of Chr5 link | 3836 | WIAF\|WIAF-2609 |
| 5 | 132.90 cR from top of Chr5 lin | 1832 | WIAF\|WIAF-3771 |
| 5 | 140.90 cR from top of Chr5 lin | 1215 | WIAF\|WIAF-2102 |
| 5 | 141.00 cR from top of Chr5 lin | 2577 | WIAF\|WIAF-209 |
| 5 | 141.40 cR from top of Chr5 lin | 1467 | WIAF\|WIAF-3402 |
| 5 | 142.30 cR from top of Chr5 lin | 1620 | WIAF\|WIAF-3559 |
| 5 | 144.10 cR from top of Chr5 lin | 3118 | WIAF\|WIAF-1019 |
| 5 | 153.50 cR from top of Chr5 lin | 3282 | WIAF\|WIAF-1735 |
| 5 | 156.10 cR from top of Chr5 lin | 1902 | WIAF\|WIAF-3841 |
| 5 | 156.40 cR from top of Chr5 lin | 1849 | WIAF\|WIAF-3788 |
| 5 | 163.00 cR from top of Chr5 lin | 1272 | WIAF\|WIAF-2159 |
| 5 | 163.00 cR from top of Chr5 lin | 1273 | WIAF\|WIAF-2160 |
| 5 | 163.00 cR from top of Chr5 lin | 1274 | WIAF\|WIAF-2161 |
| 5 | 169.80 cR from top of Chr5 lin | 1788 | WIAF\|WIAF-3727 |
| 5 | 169.80 cR from top of Chr5 lin | 1790 | WIAF\|WIAF-3729 |
| 5 | 182.0 cR from top of Chr5 link | 2478 | WIAF\|WIAF-74 |
| 5 | 186.40 cR from top of Chr5 lin | 691 | WIAF\|WIAF-1220 |
| 5 | 187.9 cR from top of Chr5 link | 3343 | WIAF\|WIAF-1796 |
| 5 | 194.00 cR from top of Chr5 lin | 1918 | WIAF\|WIAF-3857 |
| 5 | 195.80 cR from top of Chr5 lin | 1323 | WIAF\|WIAF-2213 |
| 5 | 261.7 cR from top of Chr5 link | 3269 | WIAF\|WIAF-1721 |
| 5 | 266.6 cR from top of Chr5 link | 2575 | WIAF\|WIAF-205 |
| 5 | 282.2 cR from top of Chr5 link | 2805 | WIAF\|WIAF-575 |
| 5 | 310.9 cR from top of Chr5 link | 2860 | WIAF\|WIAF-755 |
| 5 | 334.8 cR from top of Chr5 link | 2666 | WIAF\|WIAF-346 |
| 5 | 334.8 cR from top of Chr5 link | 2667 | WIAF\|WIAF-347 |
| 5 | 334.8 cR from top of Chr5 link | 2668 | WIAF\|WIAF-348 |
| 5 | 351.7 cR from top of Chr5 link | 2792 | WIAF\|WIAF-548 |
| 5 | 357.7 cR from top of Chr5 link | 2890 | WIAF\|WIAF-788 |
| 5 | 368.6 cR from top of Chr5 link | 3598 | WIAF\|WIAF-2267 |
| 5 | 368.6 cR from top of Chr5 link | 2701 | WIAF\|WIAF-389 |
| 5 | 372.6 cR from top of Chr5 link | 3351 | WIAF\|WIAF-1804 |
| 5 | 378.7 cR from top of Chr5 link | 2627 | WIAF\|WIAF-287 |
| 5 | 401.5 cR from top of Chr5 link | 3523 | WIAF\|WIAF-1978 |
| 5 | 406.7 cR from top of Chr5 link | 3568 | WIAF\|WIAF-2023 |
| 5 | 425.1 cR from top of Chr5 link | 2106 | WIAF\|WIAF-186 |
| 5 | 425.1 cR from top of Chr5 link | 2107 | WIAF\|WIAF-187 |
| 5 | 431.5 cR from top of Chr5 link | 2894 | WIAF\|WIAF-792 |
| 5 | 431.5 cR from top of Chr5 link | 2895 | WIAF\|WIAF-793 |
| 5 | 437.0 cR from top of Chr5 link | 2118 | WIAF\|WIAF-276 |
| 5 | 441.2 cR from top of Chr5 link | 3492 | WIAF\|WIAF-1947 |
| 5 | 500.0 cR from top of Chr5 link | 3374 | WIAF\|WIAF-1827 |
| 5 | 510.2 cR from top of Chr5 link | 1200 | WIAF\|WIAF-2087 |
| 5 | 532.5 cR from top of Chr5 link | 4578 | HU-CHINA\|5-787 |
| 5 | 532.5 cR from top of Chr5 link | 4550 | HU-CHINA\|5-787-2 |
| 5 | 532.5 cR from top of Chr5 link | 2889 | WIAF\|WIAF-787 |
| 5 | 532.7 cR from top of Chr5 link | 1121 | WIAF\|WIAF-1561 |
| 5 | 534.1 cR from top of Chr5 link | 2442 | WIAF\|WIAF-27 |
| 5 | 537.3 cR from top of Chr5 link | 3455 | WIAF\|WIAF-1910 |
| 5 | 537.4 cR from top of Chr5 link | 2677 | WIAF\|WIAF-359 |
| 5 | 569.8 cR from top of Chr5 link | 2211 | WIAF\|WIAF-631 |
| 5 | | 4253 | MARSHFIELD\|MID-8 |
| 5 | | 4052 | SHGC/AFFYMETRIX\|SNPA-SHGC-16519 |
| 5 | | 4058 | SHGC/AFFYMETRIX\|SNPB-SHGC-16519 |
| 5 | | 3961 | SHGC/AFFYMETRIX\|SNP-SHGC-10972 |
| 5 | | 4142 | SHGC/AFFYMETRIX\|SNP-SHGC-13353 |
| 5 | | 4160 | SHGC/AFFYMETRIX\|SNP-SHGC-14742 |
| 5 | | 4080 | SHGC/AFFYMETRIX\|SNP-SHGC-16780 |
| 5 | | 4050 | SHGC/AFFYMETRIX\|SNP-SHGC-9420 |
| 5 | | 1101 | WIAF\|WIAF-1520 |
| 5 | | 1492 | WIAF\|WIAF-3431 |
| 5 | | 3881 | WIAF\|WIAF-3942 |
| 6 | 0.0 cR from top of Chr6 linkag | 3133 | WIAF\|WIAF-1034 |
| 6 | 1.40 cR from top of Chr6 linka | 2028 | WIAF\|WIAF-1583 |
| 6 | 1.40 cR from top of Chr6 linka | 1497 | WIAF\|WIAF-3436 |
| 6 | 1.40 cR from top of Chr6 linka | 1674 | WIAF\|WIAF-3613 |
| 6 | 1.40 cR from top of Chr6 linka | 1782 | WIAF\|WIAF-3721 |
| 6 | 1.40 cR from top of Chr6 linka | 1827 | WIAF\|WIAF-3766 |
| 6 | 1.6 cR from top of Chr6 linkag | 2958 | WIAF\|WIAF-857 |
| 6 | 6.40 cR from top of Chr6 linka | 1209 | WIAF\|WIAF-2096 |
| 6 | 9.80 cR from top of Chr6 linka | 1657 | WIAF\|WIAF-3596 |
| 6 | 9.80 cR from top of Chr6 linka | 1658 | WIAF\|WIAF-3597 |
| 6 | 9.80 cR from top of Chr6 linka | 1659 | WIAF\|WIAF-3598 |
| 6 | 17.70 cR from top of Chr6 link | 3119 | WIAF\|WIAF-1020 |
| 6 | 17.80 cR from top of Chr6 link | 1373 | WIAF\|WIAF-3277 |
| 6 | 17.80 cR from top of Chr6 link | 1933 | WIAF\|WIAF-3872 |
| 6 | 17.80 cR from top of Chr6 link | 1936 | WIAF\|WIAF-3875 |
| 6 | 20.50 cR from top of Chr6 link | 1124 | WIAF\|WIAF-1567 |
| 6 | 21.30 cR from top of Chr6 link | 1551 | WIAF\|WIAF-3490 |
| 6 | 24.50 cR from top of Chr6 link | 3066 | WIAF\|WIAF-967 |
| 6 | 31.7 cR from top of Chr6 linka | 2366 | WIAF\|WIAF-2627 |
| 6 | 34.20 cR from top of Chr6 link | 1109 | WIAF\|WIAF-1541 |
| 6 | 34.90 cR from top of Chr6 link | 3511 | WIAF\|WIAF-1966 |
| 6 | 38.00 cR from top of Chr6 link | 1733 | WIAF\|WIAF-3672 |
| 6 | 41.5 cR from top of Chr6 linka | 2522 | WIAF\|WIAF-131 |
| 6 | 41.5 cR from top of Chr6 linka | 2523 | WIAF\|WIAF-132 |
| 6 | 43.50 cR from top of Chr6 link | 3504 | WIAF\|WIAF-1959 |
| 6 | 44.20 cR from top of Chr6 link | 3211 | WIAF\|WIAF-1574 |
| 6 | 46.00 cR from top of Chr6 link | 2003 | WIAF\|WIAF-1460 |
| 6 | 46.00 cR from top of Chr6 link | 2004 | WIAF\|WIAF-1461 |
| 6 | 46.00 cR from top of Chr6 link | 2005 | WIAF\|WIAF-1462 |
| 6 | 46.60 cR from top of Chr6 link | 1116 | WIAF\|WIAF-1551 |
| 6 | 46.60 cR from top of Chr6 link | 1117 | WIAF\|WIAF-1552 |
| 6 | 46.60 cR from top of Chr6 link | 1118 | WIAF\|WIAF-1553 |
| 6 | 46.60 cR from top of Chr6 link | 1119 | WIAF\|WIAF-1554 |
| 6 | 46.60 cR from top of Chr6 link | 1546 | WIAF\|WIAF-3485 |
| 6 | 46.60 cR from top of Chr6 link | 1548 | WIAF\|WIAF-3487 |
| 6 | 46.60 cR from top of Chr6 link | 1866 | WIAF\|WIAF-3805 |
| 6 | 46.60 cR from top of Chr6 link | 1992 | WIAF\|WIAF-3931 |
| 6 | 46.70 cR from top of Chr6 link | 3270 | WIAF\|WIAF-1722 |
| 6 | 46.80 cR from top of Chr6 link | 1729 | WIAF\|WIAF-3668 |
| 6 | 46.80 cR from top of Chr6 link | 1732 | WIAF\|WIAF-3671 |
| 6 | 46.80 cR from top of Chr6 link | 1735 | WIAF\|WIAF-3674 |
| 6 | 46.90 cR from top of Chr6 link | 678 | WIAF\|WIAF-1453 |
| 6 | 47.00 cR from top of Chr6 link | 3260 | WIAF\|WIAF-1696 |
| 6 | 47.00 cR from top of Chr6 link | 3480 | WIAF\|WIAF-1935 |
| 6 | 47.00 cR from top of Chr6 link | 1385 | WIAF\|WIAF-3290 |
| 6 | 47.00 cR from top of Chr6 link | 1601 | WIAF\|WIAF-3540 |
| 6 | 47.00 cR from top of Chr6 link | 1905 | WIAF\|WIAF-3844 |
| 6 | 47.00 cR from top of Chr6 link | 1907 | WIAF\|WIAF-3846 |
| 6 | 47.00 cR from top of Chr6 link | 2997 | WIAF\|WIAF-897 |
| 6 | 47.00 cR from top of Chr6 link | 2998 | WIAF\|WIAF-898 |
| 6 | 47.10 cR from top of Chr6 link | 1769 | WIAF\|WIAF-3708 |
| 6 | 47.20 cR from top of Chr6 link | 3108 | WIAF\|WIAF-1009 |
| 6 | 47.30 cR from top of Chr6 link | 1754 | WIAF\|WIAF-3693 |
| 6 | 47.30 cR from top of Chr6 link | 1755 | WIAF\|WIAF-3694 |
| 6 | 47.30 cR from top of Chr6 link | 1757 | WIAF\|WIAF-3696 |
| 6 | 47.70 cR from top of Chr6 link | 1219 | WIAF\|WIAF-2106 |
| 6 | 47.80 cR from top of Chr6 link | 1216 | WIAF\|WIAF-2103 |
| 6 | 47.90 cR from top of Chr6 link | 1472 | WIAF\|WIAF-3409 |
| 6 | 47.90 cR from top of Chr6 link | 1474 | WIAF\|WIAF-3411 |
| 6 | 50 cM | 4317 | UWGC\|136 |
| 6 | 51.1 cR from top of Chr6 linka | 3049 | WIAF\|WIAF-950 |
| 6 | 52.80 cR from top of Chr6 link | 2030 | WIAF\|WIAF-1586 |
| 6 | 52.80 cR from top of Chr6 link | 2031 | WIAF\|WIAF-1587 |
| 6 | 54.6 cR from top of Chr6 linka | 3484 | WIAF\|WIAF-1939 |
| 6 | 59.5 cR from top of Chr6 linka | 2990 | WIAF\|WIAF-890 |
| 6 | 65.50 cR from top of Chr6 link | 1188 | WIAF\|WIAF-2075 |
| 6 | 66.60 cR from top of Chr6 link | 3220 | WIAF\|WIAF-1608 |
| 6 | 66.60 cR from top of Chr6 link | 3221 | WIAF\|WIAF-1609 |
| 6 | 69.90 cR from top of Chr6 link | 3096 | WIAF\|WIAF-997 |
| 6 | 71.3 cR from top of Chr6 linka | 2981 | WIAF\|WIAF-881 |
| 6 | 79.90 cR from top of Chr6 link | 1322 | WIAF\|WIAF-2212 |
| 6 | 85.50 cR from top of Chr6 link | 1850 | WIAF\|WIAF-3789 |
| 6 | 86.40 cR from top of Chr6 link | 1761 | WIAF\|WIAF-3700 |
| 6 | 89.5 cR from top of Chr6 linka | 2510 | WIAF\|WIAF-116 |
| 6 | 89.60 cR from top of Chr6 link | 3022 | WIAF\|WIAF-922 |
| 6 | 90.50 cR from top of Chr6 link | 1528 | WIAF\|WIAF-3467 |
| 6 | 90.50 cR from top of Chr6 link | 1532 | WIAF\|WIAF-3471 |
| 6 | 93.60 cR from top of Chr6 link | 3172 | WIAF\|WIAF-1486 |
| 6 | 95.50 cR from top of Chr6 link | 3251 | WIAF\|WIAF-1669 |
| 6 | 95.50 cR from top of Chr6 link | 3252 | WIAF\|WIAF-1670 |
| 6 | 95.50 cR from top of Chr6 link | 1818 | WIAF\|WIAF-3757 |
| 6 | 96.00 cR from top of Chr6 link | 1807 | WIAF\|WIAF-3746 |
| 6 | 99.8 cR from top of Chr6 linka | 2090 | WIAF\|WIAF-105 |
| 6 | 99.8 cR from top of Chr6 linka | 2091 | WIAF\|WIAF-106 |
| 6 | 102.40 cR from top of Chr6 lin | 3244 | WIAF\|WIAF-1654 |
| 6 | 105.40 cR from top of Chr6 lin | 1783 | WIAF\|WIAF-3722 |
| 6 | 106.40 cR from top of Chr6 lin | 3167 | WIAF\|WIAF-1476 |
| 6 | 106.90 cR from top of Chr6 lin | 1417 | WIAF\|WIAF-3344 |
| 6 | 116.20 cR from top of Chr6 lin | 1809 | WIAF\|WIAF-3748 |
| 6 | 116.60 cR from top of Chr6 lin | 3420 | WIAF\|WIAF-1875 |
| 6 | 116.60 cR from top of Chr6 lin | 3421 | WIAF\|WIAF-1876 |
| 6 | 116.60 cR from top of Chr6 lin | 3422 | WIAF\|WIAF-1877 |
| 6 | 116.60 cR from top of Chr6 lin | 3423 | WIAF\|WIAF-1878 |
| 6 | 116.60 cR from top of Chr6 lin | 3424 | WIAF\|WIAF-1879 |
| 6 | 116.60 cR from top of Chr6 lin | 3425 | WIAF\|WIAF-1880 |
| 6 | 116.60 cR from top of Chr6 lin | 3426 | WIAF\|WIAF-1881 |
| 6 | 116.60 cR from top of Chr6 lin | 1329 | WIAF\|WIAF-2219 |
| 6 | 116.90 cR from top of Chr6 lin | 1239 | WIAF\|WIAF-2126 |
| 6 | 117.00 cR from top of Chr6 lin | 1582 | WIAF\|WIAF-3521 |
| 6 | 124.20 cR from top of Chr6 lin | 1284 | WIAF\|WIAF-2171 |
| 6 | 125.4 cR from top of Chr6 link | 3441 | WIAF\|WIAF-1896 |
| 6 | 125.4 cR from top of Chr6 link | 3828 | WIAF\|WIAF-2597 |
| 6 | 125.4 cR from top of Chr6 link | 2716 | WIAF\|WIAF-415 |
| 6 | 125.40 cR from top of Chr6 lin | 1592 | WIAF\|WIAF-3531 |
| 6 | 125.80 cR from top of Chr6 lin | 3254 | WIAF\|WIAF-1674 |
| 6 | 131.20 cR from top of Chr6 lin | 1535 | WIAF\|WIAF-3474 |
| 6 | 137.90 cR from top of Chr6 lin | 1706 | WIAF\|WIAF-3645 |
| 6 | 144.50 cR from top of Chr6 lin | 1141 | WIAF\|WIAF-1604 |
| 6 | 144.50 cR from top of Chr6 lin | 1860 | WIAF\|WIAF-3799 |
| 6 | 144.90 cR from top of Chr6 lin | 3418 | WIAF\|WIAF-1873 |
| 6 | 145.60 cR from top of Chr6 lin | 3579 | WIAF\|WIAF-2034 |
| 6 | 147.30 cR from top of Chr6 lin | 1771 | WIAF\|WIAF-3710 |
| 6 | 150.40 cR from top of Chr6 lin | 1150 | WIAF\|WIAF-1623 |
| 6 | 150.40 cR from top of Chr6 lin | 1176 | WIAF\|WIAF-1701 |
| 6 | 154.90 cR from top of Chr6 lin | 1563 | WIAF\|WIAF-3502 |
| 6 | 155.70 cR from top of Chr6 lin | 1737 | WIAF\|WIAF-3676 |
| 6 | 155.70 cR from top of Chr6 lin | 1738 | WIAF\|WIAF-3677 |
| 6 | 159.00 cR from top of Chr6 lin | 1867 | WIAF\|WIAF-3806 |
| 6 | 165.70 cR from top of Chr6 lin | 3499 | WIAF\|WIAF-1954 |
| 6 | 165.70 cR from top of Chr6 lin | 3500 | WIAF\|WIAF-1955 |
| 6 | 166.40 cR from top of Chr6 lin | 1465 | WIAF\|WIAF-3400 |
| 6 | 166.80 cR from top of Chr6 lin | 1513 | WIAF\|WIAF-3452 |
| 6 | 175.90 cR from top of Chr6 lin | 3015 | WIAF\|WIAF-915 |
| 6 | 177.0 cR from top of Chr6 link | 2798 | WIAF\|WIAF-556 |
| 6 | 177.0 cR from top of Chr6 link | 2799 | WIAF\|WIAF-557 |
| 6 | 177.0 cR from top of Chr6 link | 2800 | WIAF\|WIAF-558 |
| 6 | 178.40 cR from top of Chr6 lin | 1896 | WIAF\|WIAF-3835 |
| 6 | 180.3 cR from top of Chr6 link | 2611 | WIAF\|WIAF-257 |
| 6 | 180.3 cR from top of Chr6 link | 2612 | WIAF\|WIAF-258 |
| 6 | 180.4 cR from top of Chr6 link | 951 | WIAF\|WIAF-1335 |
| 6 | 180.4 cR from top of Chr6 link | 952 | WIAF\|WIAF-1336 |
| 6 | 180.4 cR from top of Chr6 link | 953 | WIAF\|WIAF-1337 |
| 6 | 180.4 cR from top of Chr6 link | 954 | WIAF\|WIAF-1338 |
| 6 | 180.4 cR from top of Chr6 link | 955 | WIAF\|WIAF-1339 |
| 6 | 180.4 cR from top of Chr6 link | 956 | WIAF\|WIAF-1340 |
| 6 | 180.4 cR from top of Chr6 link | 957 | WIAF\|WIAF-1341 |
| 6 | 180.4 cR from top of Chr6 link | 958 | WIAF\|WIAF-1342 |
| 6 | 180.4 cR from top of Chr6 link | 1038 | WIAF\|WIAF-4120 |
| 6 | 184.80 cR from top of Chr6 lin | 1426 | WIAF\|WIAF-3354 |
| 6 | 187.1 cR from top of Chr6 link | 3565 | WIAF\|WIAF-2020 |
| 6 | 187.1 cR from top of Chr6 link | 3566 | WIAF\|WIAF-2021 |
| 6 | 187.7 cR from top of Chr6 link | 1993 | WIAF\|WIAF-3932 |
| 6 | 187.7 cR from top of Chr6 link | 1996 | WIAF\|WIAF-3935 |
| 6 | 187.8 cR from top of Chr6 link | 2529 | WIAF\|WIAF-139 |
| 6 | 188.2 cR from top of Chr6 link | 1660 | WIAF\|WIAF-3599 |
| 6 | 188.40 cR from top of Chr6 lin | 1427 | WIAF\|WIAF-3355 |
| 6 | 189.00 cR from top of Chr6 lin | 1299 | WIAF\|WIAF-2186 |
| 6 | 190.30 cR from top of Chr6 lin | 1440 | WIAF\|WIAF-3369 |
| 6 | 190.30 cR from top of Chr6 lin | 1442 | WIAF\|WIAF-3371 |
| 6 | 190.30 cR from top of Chr6 lin | 1443 | WIAF\|WIAF-3373 |
| 6 | 201.1 cR from top of Chr6 link | 1290 | WIAF\|WIAF-2177 |
| 6 | 201.10 cR from top of Chr6 lin | 1763 | WIAF\|WIAF-3702 |
| 6 | 201.10 cR from top of Chr6 lin | 1765 | WIAF\|WIAF-3704 |
| 6 | 203.9 cR from top of Chr6 link | 2505 | WIAF\|WIAF-110 |
| 6 | 212.4 cR from top of Chr6 link | 3105 | WIAF\|WIAF-1006 |
| 6 | 212.6 cR from top of Chr6 link | 3837 | WIAF\|WIAF-2610 |
| 6 | 217.6 cR from top of Chr6 link | 3016 | WIAF\|WIAF-916 |
| 6 | 218.7 cR from top of Chr6 link | 3293 | WIAF\|WIAF-1746 |
| 6 | 249.6 cR from top of Chr6 link | 857 | WIAF\|WIAF-1060 |
| 6 | 249.6 cR from top of Chr6 link | 1063 | WIAF\|WIAF-4188 |
| 6 | 256.2 cR from top of Chr6 link | 1845 | WIAF\|WIAF-3784 |
| 6 | 275.6 cR from top of Chr6 link | 2499 | WIAF\|WIAF-98 |
| 6 | 276.6 cR from top of Chr6 link | 3327 | WIAF\|WIAF-1780 |
| 6 | 331.1 cR from top of Chr6 link | 2907 | WIAF\|WIAF-805 |
| 6 | 487.0 cR from top of Chr6 link | 2809 | WIAF\|WIAF-589 |
| 6 | 576.9 cR from top of Chr6 link | 2296 | WIAF\|WIAF-2281 |
| 6 | 625.0 cR from top of Chr6 link | 2084 | WIAF\|WIAF-78 |
| 6 | 706.1 cR from top of Chr6 link | 2945 | WIAF\|WIAF-844 |
| 6 | 706.1 cR from top of Chr6 link | 2946 | WIAF\|WIAF-845 |
| 6 | 711.4 cR from top of Chr6 link | 2808 | WIAF\|WIAF-584 |
| 6 | 729.2 cR from top of Chr6 link | 4588 | HU-CHINA\|6-985 |
| 6 | 729.2 cR from top of Chr6 link | 3084 | WIAF\|WIAF-985 |
| 6 | 734.7 cR from top of Chr6 link | 2274 | WIAF\|WIAF-736 |
| 6 | 734.7 cR from top of Chr6 link | 2275 | WIAF\|WIAF-737 |
| 6 | 736.4 cR from top of Chr6 link | 1784 | WIAF\|WIAF-3723 |
| 6 | 739.6 cR from top of Chr6 link | 2158 | WIAF\|WIAF-492 |
| 6 | 799.2 cR from top of Chr6 link | 2073 | WIAF\|WIAF-3 |
| 6 | 812.0 cR from top of Chr6 link | 2747 | WIAF\|WIAF-466 |
| 6 | 822.5 cR from top of Chr6 link | 2193 | WIAF\|WIAF-592 |
| 6 | 837.8 cR from top of Chr6 link | 3081 | WIAF\|WIAF-982 |
| 6 | 846.2 cR from top of Chr6 link | 2386 | WIAF\|WIAF-2680 |
| 6 | 856.6 cR from top of Chr6 link | 2498 | WIAF\|WIAF-97 |
| 6 | 858.4 cR from top of Chr6 link | 2080 | WIAF\|WIAF-30 |
| 6 | 860.5 cR from top of Chr6 link | 2865 | WIAF\|WIAF-762 |
| 6 | | 4218 | MARSHFIELD\|MID-10 |
| 6 | | 4219 | MARSHFIELD\|MID-11 |
| 6 | | 4254 | MARSHFIELD\|MID-9 |
| 6 | | 4117 | SHGC/AFFYMETRIX\|SNPA-SHGC-13699 |
| 6 | | 3988 | SHGC/AFFYMETRIX\|SNPA-SHGC-6809 |
| 6 | | 4127 | SHGC/AFFYMETRIX\|SNPB-SHGC-13699 |
| 6 | | 3993 | SHGC/AFFYMETRIX\|SNPB-SHGC-6809 |
| 6 | | 3960 | SHGC/AFFYMETRIX\|SNP-SHGC-10969 |
| 6 | | 4002 | SHGC/AFFYMETRIX\|SNP-SHGC-12214 |
| 6 | | 4149 | SHGC/AFFYMETRIX\|SNP-SHGC-14111 |
| 6 | | 4152 | SHGC/AFFYMETRIX\|SNP-SHGC-14233 |
| 6 | | 4158 | SHGC/AFFYMETRIX\|SNP-SHGC-14719 |
| 6 | | 3975 | SHGC/AFFYMETRIX\|SNP-SHGC-34704 |
| 6 | | 3977 | SHGC/AFFYMETRIX\|SNP-SHGC-44682 |
| 6 | | 4042 | SHGC/AFFYMETRIX\|SNP-SHGC-8858 |
| 6 | | 3149 | WIAF\|WIAF-1050 |
| 6 | | 1107 | WIAF\|WIAF-1539 |
| 6 | | 3257 | WIAF\|WIAF-1685 |
| 6 | | 3877 | WIAF\|WIAF-2678 |
| 6 | | 1505 | WIAF\|WIAF-3444 |
| 6 | | 1545 | WIAF\|WIAF-3484 |
| 6 | | 1616 | WIAF\|WIAF-3555 |
| 6 | | 1781 | WIAF\|WIAF-3720 |
| 6 | | 1787 | WIAF\|WIAF-3726 |
| 6 | | 1789 | WIAF\|WIAF-3728 |
| 6 | | 1791 | WIAF\|WIAF-3730 |
| 6 | | 1793 | WIAF\|WIAF-3732 |
| 6 | | 1795 | WIAF\|WIAF-3734 |
| 6 | | 1801 | WIAF\|WIAF-3740 |
| 6 | | 1851 | WIAF\|WIAF-3790 |
| 6 | | 1904 | WIAF\|WIAF-3843 |
| 6 | | 1932 | WIAF\|WIAF-3871 |
| 6 | | 1935 | WIAF\|WIAF-3874 |
| 6 | | 1938 | WIAF\|WIAF-3877 |
| 6 | | 2972 | WIAF\|WIAF-872 |
| 6 | | 2973 | WIAF\|WIAF-873 |
| 6 | | 3063 | WIAF\|WIAF-964 |
| 6 | | 3086 | WIAF\|WIAF-987 |
| 7 | 2.20 cR from top of Chr7 linka | 1804 | WIAF\|WIAF-3743 |
| 7 | 5.20 cR from top of Chr7 linka | 1300 | WIAF\|WIAF-2189 |
| 7 | 18.10 cR from top of Chr7 link | 1759 | WIAF\|WIAF-3698 |
| 7 | 19.00 cR from top of Chr7 link | 1457 | WIAF\|WIAF-3390 |
| 7 | 22.00 cR from top of Chr7 link | 1913 | WIAF\|WIAF-3852 |
| 7 | 26.4 cR from top of Chr7 linka | 2926 | WIAF\|WIAF-825 |
| 7 | 29.10 cR from top of Chr7 link | 1717 | WIAF\|WIAF-3656 |
| 7 | 29.10 cR from top of Chr7 link | 1796 | WIAF\|WIAF-3735 |
| 7 | 29.10 cR from top of Chr7 link | 1808 | WIAF\|WIAF-3747 |
| 7 | 34.8 cR from top of Chr7 linka | 3832 | WIAF\|WIAF-2601 |
| 7 | 37.3 cR from top of Chr7 linka | 4579 | HU-CHINA\|7-349 |
| 7 | 37.3 cR from top of Chr7 linka | 4551 | HU-CHINA\|7-349-2 |
| 7 | 37.3 cR from top of Chr7 linka | 2669 | WIAF\|WIAF-349 |
| 7 | 37.3 cR from top of Chr7 linka | 2670 | WIAF\|WIAF-350 |
| 7 | 39.90 cR from top of Chr7 link | 1493 | WIAF\|WIAF-3432 |
| 7 | 50.00 cR from top of Chr7 link | 1526 | WIAF\|WIAF-3465 |
| 7 | 58.9 cR from top of Chr7 linka | 774 | WIAF\|WIAF-1405 |
| 7 | 64.6 cR from top of Chr7 linka | 3807 | WIAF\|WIAF-2559 |
| 7 | 70.70 cR from top of Chr7 link | 2928 | WIAF\|WIAF-827 |
| 7 | 70.70 cR from top of Chr7 link | 2929 | WIAF\|WIAF-828 |
| 7 | 71.50 cR from top of Chr7 link | 1220 | WIAF\|WIAF-2107 |
| 7 | 77.10 cR from top of Chr7 link | 1291 | WIAF\|WIAF-2178 |
| 7 | 77.10 cR from top of Chr7 link | 1292 | WIAF\|WIAF-2179 |
| 7 | 83.60 cR from top of Chr7 link | 1401 | WIAF\|WIAF-3321 |
| 7 | 89.0 cR from top of Chr7 linka | 2616 | WIAF\|WIAF-263 |
| 7 | 90.20 cR from top of Chr7 link | 2042 | WIAF\|WIAF-1631 |
| 7 | 93.2 cR from top of Chr7 linka | 2771 | WIAF\|WIAF-514 |
| 7 | 93.90 cR from top of Chr7 link | 1949 | WIAF\|WIAF-3888 |
| 7 | 98.00 cR from top of Chr7 link | 1768 | WIAF\|WIAF-3707 |
| 7 | 102.30 cR from top of Chr7 lin | 1664 | WIAF\|WIAF-3603 |
| 7 | 105.20 cR from top of Chr7 lin | 668 | WIAF\|WIAF-1240 |
| 7 | 105.20 cR from top of Chr7 lin | 669 | WIAF\|WIAF-1332 |
| 7 | 106 cM | 4324 | UWGC\|143 |
| 7 | 109.50 cR from top of Chr7 lin | 4573 | HU-CHINA\|7-1100 |
| 7 | 109.50 cR from top of Chr7 lin | 667 | WIAF\|WIAF-1100 |
| 7 | 109.90 cR from top of Chr7 lin | 4572 | HU-CHINA\|7-1495 |
| 7 | 109.90 cR from top of Chr7 lin | 3179 | WIAF\|WIAF-1495 |
| 7 | 110.9 cR from top of Chr7 link | 3375 | WIAF\|WIAF-1828 |
| 7 | 110.9 cR from top of Chr7 link | 3376 | WIAF\|WIAF-1829 |
| 7 | 111.60 cR from top of Chr7 lin | 3168 | WIAF\|WIAF-1477 |
| 7 | 111.60 cR from top of Chr7 lin | 3169 | WIAF\|WIAF-1478 |
| 7 | 112.00 cR from top of Chr7 lin | 3563 | WIAF\|WIAF-2018 |
| 7 | 112.00 cR from top of Chr7 lin | 1815 | WIAF\|WIAF-3754 |
| 7 | 112.00 cR from top of Chr7 lin | 1816 | WIAF\|WIAF-3755 |
| 7 | 112.30 cR from top of Chr7 lin | 4574 | HU-CHINA\|7-1510 |
| 7 | 112.30 cR from top of Chr7 lin | 1098 | WIAF\|WIAF-1510 |
| 7 | 112.90 cR from top of Chr7 lin | 1199 | WIAF\|WIAF-2086 |
| 7 | 113.40 cR from top of Chr7 lin | 1927 | WIAF\|WIAF-3866 |
| 7 | 117.20 cR from top of Chr7 lin | 4593 | HU-CHINA\|7-1680 |
| 7 | 117.20 cR from top of Chr7 lin | 4594 | HU-CHINA\|7-1680-2 |
| 7 | 117.20 cR from top of Chr7 lin | 4595 | HU-CHINA\|7-1680-3 |
| 7 | 117.20 cR from top of Chr7 lin | 1167 | WIAF\|WIAF-1679 |
| 7 | 117.20 cR from top of Chr7 lin | 1168 | WIAF\|WIAF-1680 |
| 7 | 117.20 cR from top of Chr7 lin | 1169 | WIAF\|WIAF-1681 |
| 7 | 119.80 cR from top of Chr7 lin | 1180 | WIAF\|WIAF-1710 |
| 7 | 122.6 cR from top of Chr7 link | 2546 | WIAF\|WIAF-167 |
| 7 | 125.50 cR from top of Chr7 lin | 1381 | WIAF\|WIAF-3285 |
| 7 | 126.60 cR from top of Chr7 lin | 1980 | WIAF\|WIAF-3919 |
| 7 | 126.60 cR from top of Chr7 lin | 1981 | WIAF\|WIAF-3920 |
| 7 | 129.10 cR from top of Chr7 lin | 1387 | WIAF\|WIAF-3292 |
| 7 | 129.90 cR from top of Chr7 lin | 1711 | WIAF\|WIAF-3650 |
| 7 | 135.3 cR from top of Chr7 link | 3002 | WIAF\|WIAF-902 |
| 7 | 136.50 cR from top of Chr7 lin | 3861 | WIAF\|WIAF-2651 |
| 7 | 139.70 cR from top of Chr7 lin | 1839 | WIAF\|WIAF-3778 |
| 7 | 147.7 cR from top of Chr7 link | 3357 | WIAF\|WIAF-1810 |
| 7 | 150.1 cR from top of Chr7 link | 3571 | WIAF\|WIAF-2026 |
| 7 | 150.1 cR from top of Chr7 link | 3572 | WIAF\|WIAF-2027 |
| 7 | 155.00 cR from top of Chr7 lin | 711 | WIAF\|WIAF-1447 |
| 7 | 155.00 cR from top of Chr7 lin | 712 | WIAF\|WIAF-1448 |
| 7 | 165.60 cR from top of Chr7 lin | 1777 | WIAF\|WIAF-3716 |
| 7 | 165.60 cR from top of Chr7 lin | 1778 | WIAF\|WIAF-3717 |
| 7 | 169.00 cR from top of Chr7 lin | 1136 | WIAF\|WIAF-1599 |
| 7 | 172.90 cR from top of Chr7 lin | 3227 | WIAF\|WIAF-1627 |
| 7 | 176.60 cR from top of Chr7 lin | 1743 | WIAF\|WIAF-3682 |
| 7 | 182.40 cR from top of Chr7 lin | 2039 | WIAF\|WIAF-1620 |
| 7 | 182.40 cR from top of Chr7 lin | 1298 | WIAF\|WIAF-2185 |
| 7 | 183.2 cR from top of Chr7 link | 3505 | WIAF\|WIAF-1960 |
| 7 | 184.00 cR from top of Chr7 lin | 1727 | WIAF\|WIAF-3666 |
| 7 | 184.00 cR from top of Chr7 lin | 2901 | WIAF\|WIAF-799 |
| 7 | 187.2 cR from top of Chr7 link | 2214 | WIAF\|WIAF-636 |
| 7 | 390.2 cR from top of Chr7 link | 2603 | WIAF\|WIAF-247 |
| 7 | 399.5 cR from top of Chr7 link | 2215 | WIAF\|WIAF-637 |
| 7 | 446.9 cR from top of Chr7 link | 3370 | WIAF\|WIAF-1823 |
| 7 | 453.2 cR from top of Chr7 link | 3060 | WIAF\|WIAF-961 |
| 7 | 455.7 cR from top of Chr7 link | 2908 | WIAF\|WIAF-806 |
| 7 | 467.5 cR from top of Chr7 link | 2769 | WIAF\|WIAF-509 |
| 7 | 467.6 cR from top of Chr7 link | 2828 | WIAF\|WIAF-644 |
| 7 | 476.3 cR from top of Chr7 link | 4580 | HU-CHINA\|7-1773 |
| 7 | 476.3 cR from top of Chr7 link | 4552 | HU-CHINA\|7-1773-2 |
| 7 | 476.3 cR from top of Chr7 link | 2502 | WIAF\|WIAF-104 |
| 7 | 476.3 cR from top of Chr7 link | 3319 | WIAF\|WIAF-1772 |
| 7 | 476.3 cR from top of Chr7 link | 3320 | WIAF\|WIAF-1773 |
| 7 | 479.2 cR from top of Chr7 link | 2187 | WIAF\|WIAF-570 |
| 7 | 491.0 cR from top of Chr7 link | 4565 | HU-CHINA\|7-1781 |
| 7 | 491.0 cR from top of Chr7 link | 3328 | WIAF\|WIAF-1781 |
| 7 | 493.0 cR from top of Chr7 link | 2354 | WIAF\|WIAF-2594 |
| 7 | 493.0 cR from top of Chr7 link | 2355 | WIAF\|WIAF-2595 |
| 7 | 495.4 cR from top of Chr7 link | 867 | WIAF\|WIAF-1080 |
| 7 | 495.4 cR from top of Chr7 link | 1265 | WIAF\|WIAF-2152 |
| 7 | 495.4 cR from top of Chr7 link | 1058 | WIAF\|WIAF-4174 |
| 7 | 497.2 cR from top of Chr7 link | 2548 | WIAF\|WIAF-169 |
| 7 | 502.4 cR from top of Chr7 link | 2909 | WIAF\|WIAF-807 |
| 7 | 514.6 cR from top of Chr7 link | 2487 | WIAF\|WIAF-85 |
| 7 | 522.1 cR from top of Chr7 link | 3329 | WIAF\|WIAF-1782 |
| 7 | 530.0 cR from top of Chr7 link | 2866 | WIAF\|WIAF-764 |
| 7 | 568.6 cR from top of Chr7 link | 2678 | WIAF\|WIAF-360 |
| 7 | 568.6 cR from top of Chr7 link | 853 | WIAF\|WIAF-361 |
| 7 | 598.6 cR from top of Chr7 link | 3338 | WIAF\|WIAF-1791 |
| 7 | 602.0 cR from top of Chr7 link | 2534 | WIAF\|WIAF-147 |
| 7 | 602.0 cR from top of Chr7 link | 2535 | WIAF\|WIAF-148 |
| 7 | 603.1 cR from top of Chr7 link | 3284 | WIAF\|WIAF-1737 |
| 7 | 603.1 cR from top of Chr7 link | 3285 | WIAF\|WIAF-1738 |
| 7 | 621.5 cR from top of Chr7 link | 730 | WIAF\|WIAF-1087 |
| 7 | 646.3 cR from top of Chr7 link | 3154 | WIAF\|WIAF-1055 |
| 7 | 663.1 cR from top of Chr7 link | 2444 | WIAF\|WIAF-32 |
| 7 | 663.1 cR from top of Chr7 link | 2445 | WIAF\|WIAF-33 |
| 7 | 668.3 cR from top of Chr7 link | 3332 | WIAF\|WIAF-1785 |
| 7 | 669.9 cR from top of Chr7 link | 3536 | WIAF\|WIAF-1991 |
| 7 | 670.2 cR from top of Chr7 link | 2751 | WIAF\|WIAF-473 |
| 7 | 670.6 cR from top of Chr7 link | 3522 | WIAF\|WIAF-1977 |
| 7 | | 333 | EXAMPLE\|CTFR-tttdel |
| 7 | | 3954 | MARSHFIELD\|MID-1 |
| 7 | | 3955 | MARSHFIELD\|MID-2 |
| 7 | | 3956 | MARSHFIELD\|MID-3 |
| 7 | | 4247 | MARSHFIELD\|MID-4 |
| 7 | | 4250 | MARSHFIELD\|MID-5 |
| 7 | | 4251 | MARSHFIELD\|MID-6 |
| 7 | | 4144 | SHGC/AFFYMETRIX\|SNP-SHGC-13664 |
| 7 | | 4084 | SHGC/AFFYMETRIX\|SNP-SHGC-16934 |
| 7 | | 4090 | SHGC/AFFYMETRIX\|SNP-SHGC-17167 |
| 7 | | 4100 | SHGC/AFFYMETRIX\|SNP-SHGC-19036 |
| 7 | | 3973 | SHGC/AFFYMETRIX\|SNP-SHGC-32515 |
| 7 | | 3195 | WIAF\|WIAF-1530 |
| 7 | | 1132 | WIAF\|WIAF-1579 |
| 7 | | 2559 | WIAF\|WIAF-183 |
| 7 | | 1264 | WIAF\|WIAF-2151 |
| 7 | | 1688 | WIAF\|WIAF-3627 |
| 7 | | 2425 | WIAF\|WIAF-5 |
| 7 | | 2840 | WIAF\|WIAF-678 |
| 7 | | 3082 | WIAF\|WIAF-983 |
| 8 | 0.1 cR from top of Chr8 linkag | 2267 | WIAF\|WIAF-724 |
| 8 | 0.1 cR from top of Chr8 linkag | 2268 | WIAF\|WIAF-725 |
| 8 | 0.70 cR from top of Chr8 linka | 1785 | WIAF\|WIAF-3724 |
| 8 | 6.50 cR from top of Chr8 linka | 3442 | WIAF\|WIAF-1897 |
| 8 | 6.50 cR from top of Chr8 linka | 3443 | WIAF\|WIAF-1898 |
| 8 | 8.20 cR from top of Chr8 linka | 1895 | WIAF\|WIAF-3834 |
| 8 | 11.1 cR from top of Chr8 linka | 3840 | WIAF\|WIAF-2614 |
| 8 | 13.40 cR from top of Chr8 link | 1281 | WIAF\|WIAF-2168 |
| 8 | 13.40 cR from top of Chr8 link | 1282 | WIAF\|WIAF-2169 |
| 8 | 15.50 cR from top of Chr8 link | 1418 | WIAF\|WIAF-3345 |
| 8 | 15.50 cR from top of Chr8 link | 1419 | WIAF\|WIAF-3346 |
| 8 | 20.40 cR from top of Chr8 link | 3537 | WIAF\|WIAF-1992 |
| 8 | 20.40 cR from top of Chr8 link | 3538 | WIAF\|WIAF-1993 |
| 8 | 22.7 cR from top of Chr8 linka | 2624 | WIAF\|WIAF-283 |
| 8 | 30.70 cR from top of Chr8 link | 2053 | WIAF\|WIAF-1709 |
| 8 | 31.9 cR from top of Chr8 linka | 3476 | WIAF\|WIAF-1931 |
| 8 | 33.2 cR from top of Chr8 linka | 2613 | WIAF\|WIAF-259 |
| 8 | 33.2 cR from top of Chr8 linka | 2614 | WIAF\|WIAF-260 |
| 8 | 37.0 cR from top of Chr8 linka | 2476 | WIAF\|WIAF-72 |
| 8 | 39.90 cR from top of Chr8 link | 1233 | WIAF\|WIAF-2120 |
| 8 | 40.90 cR from top of Chr8 link | 3218 | WIAF\|WIAF-1596 |
| 8 | 42.70 cR from top of Chr8 link | 1100 | WIAF\|WIAF-1517 |
| 8 | 43.70 cR from top of Chr8 link | 1149 | WIAF\|WIAF-1622 |
| 8 | 43.90 cR from top of Chr8 link | 1894 | WIAF\|WIAF-3833 |
| 8 | 44.40 cR from top of Chr8 link | 1481 | WIAF\|WIAF-3420 |
| 8 | 47.90 cR from top of Chr8 link | 1857 | WIAF\|WIAF-3796 |
| 8 | 55.4 cR from top of Chr8 linka | 2457 | WIAF\|WIAF-48 |
| 8 | 55.4 cR from top of Chr8 linka | 2458 | WIAF\|WIAF-49 |
| 8 | 60.1 cR from top of Chr8 linka | 1040 | WIAF\|WIAF-4128 |
| 8 | 62.60 cR from top of Chr8 link | 1174 | WIAF\|WIAF-1693 |
| 8 | 62.70 cR from top of Chr8 link | 3275 | WIAF\|WIAF-1728 |
| 8 | 62.80 cR from top of Chr8 link | 1870 | WIAF\|WIAF-3809 |
| 8 | 63.30 cR from top of Chr8 link | 1682 | WIAF\|WIAF-3621 |
| 8 | 68.70 cR from top of Chr8 link | 1843 | WIAF\|WIAF-3782 |
| 8 | 80.90 cR from top of Chr8 link | 2024 | WIAF\|WIAF-1565 |
| 8 | 81.50 cR from top of Chr8 link | 1133 | WIAF\|WIAF-1580 |
| 8 | 81.50 cR from top of Chr8 link | 3302 | WIAF\|WIAF-1755 |
| 8 | 88.30 cR from top of Chr8 link | 1197 | WIAF\|WIAF-2084 |
| 8 | 95.3 cR from top of Chr8 linka | 2674 | WIAF\|WIAF-356 |
| 8 | 95.3 cR from top of Chr8 linka | 2675 | WIAF\|WIAF-357 |
| 8 | 96.20 cR from top of Chr8 link | 1971 | WIAF\|WIAF-3910 |
| 8 | 96.3 cR from top of Chr8 linka | 3092 | WIAF\|WIAF-993 |
| 8 | 98.0 cR from top of Chr8 linka | 2920 | WIAF\|WIAF-819 |
| 8 | 101.00 cR from top of Chr8 lin | 708 | WIAF\|WIAF-1406 |
| 8 | 101.3 cR from top of Chr8 link | 2967 | WIAF\|WIAF-867 |
| 8 | 103.7 cR from top of Chr8 link | 2933 | WIAF\|WIAF-832 |
| 8 | 105.80 cR from top of Chr8 lin | 1205 | WIAF\|WIAF-2092 |
| 8 | 108.90 cR from top of Chr8 lin | 1739 | WIAF\|WIAF-3678 |
| 8 | 109.00 cR from top of Chr8 lin | 1163 | WIAF\|WIAF-1666 |
| 8 | 109.80 cR from top of Chr8 lin | 1713 | WIAF\|WIAF-3652 |
| 8 | 113.70 cR from top of Chr8 lin | 2029 | WIAF\|WIAF-1584 |
| 8 | 113.70 cR from top of Chr8 lin | 1336 | WIAF\|WIAF-2226 |
| 8 | 115.8 cR from top of Chr8 link | 2262 | WIAF\|WIAF-716 |
| 8 | 115.8 cR from top of Chr8 link | 2263 | WIAF\|WIAF-717 |
| 8 | 118.30 cR from top of Chr8 lin | 1350 | WIAF\|WIAF-3253 |
| 8 | 118.30 cR from top of Chr8 lin | 1352 | WIAF\|WIAF-3255 |
| 8 | 118.8 cR from top of Chr8 link | 2722 | WIAF\|WIAF-426 |
| 8 | 118.8 cR from top of Chr8 link | 2723 | WIAF\|WIAF-427 |
| 8 | 119.00 cR from top of Chr8 lin | 1447 | WIAF\|WIAF-3377 |
| 8 | 119.00 cR from top of Chr8 lin | 1448 | WIAF\|WIAF-3378 |
| 8 | 121.4 cR from top of Chr8 link | 3629 | WIAF\|WIAF-2357 |
| 8 | 124.1 cR from top of Chr8 link | 2686 | WIAF\|WIAF-369 |
| 8 | 124.1 cR from top of Chr8 link | 2687 | WIAF\|WIAF-370 |
| 8 | 126.30 cR from top of Chr8 lin | 1863 | WIAF\|WIAF-3802 |
| 8 | 126.30 cR from top of Chr8 lin | 1864 | WIAF\|WIAF-3803 |
| 8 | 126.50 cR from top of Chr8 lin | 1213 | WIAF\|WIAF-2100 |
| 8 | 126.50 cR from top of Chr8 lin | 1234 | WIAF\|WIAF-2121 |
| 8 | 126.60 cR from top of Chr8 lin | 1326 | WIAF\|WIAF-2216 |
| 8 | 132.00 cR from top of Chr8 lin | 1672 | WIAF\|WIAF-3611 |
| 8 | 133.90 cR from top of Chr8 lin | 1742 | WIAF\|WIAF-3681 |
| 8 | 147.10 cR from top of Chr8 lin | 3192 | WIAF\|WIAF-1516 |
| 8 | 164.20 cR from top of Chr8 lin | 3128 | WIAF\|WIAF-1029 |
| 8 | 164.70 cR from top of Chr8 lin | 3546 | WIAF\|WIAF-2001 |
| 8 | 166.40 cR from top of Chr8 lin | 3240 | WIAF\|WIAF-1650 |
| 8 | 166.40 cR from top of Chr8 lin | 3241 | WIAF\|WIAF-1651 |
| 8 | 166.40 cR from top of Chr8 lin | 1847 | WIAF\|WIAF-3786 |
| 8 | 166.40 cR from top of Chr8 lin | 3009 | WIAF\|WIAF-909 |
| 8 | 233.1 cR from top of Chr8 link | 3639 | WIAF\|WIAF-2367 |
| 8 | 410.4 cR from top of Chr8 link | 3028 | WIAF\|WIAF-928 |
| 8 | 416.4 cR from top of Chr8 link | 2474 | WIAF\|WIAF-69 |
| 8 | 416.4 cR from top of Chr8 link | 2475 | WIAF\|WIAF-70 |
| 8 | 435.6 cR from top of Chr8 link | 2133 | WIAF\|WIAF-354 |
| 8 | 435.6 cR from top of Chr8 link | 2134 | WIAF\|WIAF-355 |
| 8 | 441.8 cR from top of Chr8 link | 3430 | WIAF\|WIAF-1885 |
| 8 | 466.7 cR from top of Chr8 link | 3391 | WIAF\|WIAF-1846 |
| 8 | 514.9 cR from top of Chr8 link | 2497 | WIAF\|WIAF-96 |
| 8 | 541.5 cR from top of Chr8 link | 3392 | WIAF\|WIAF-1847 |
| 8 | 579.6 cR from top of Chr8 link | 3873 | WIAF\|WIAF-2672 |
| 8 | 588.3 cR from top of Chr8 link | 3413 | WIAF\|WIAF-1868 |
| 8 | 591.7 cR from top of Chr8 link | 3526 | WIAF\|WIAF-1981 |
| 8 | 592.1 cR from top of Chr8 link | 2157 | WIAF\|WIAF-490 |
| 8 | 592.1 cR from top of Chr8 link | 829 | WIAF\|WIAF-491 |
| 8 | 592.4 cR from top of Chr8 link | 762 | WIAF\|WIAF-1351 |
| 8 | 625.1 cR from top of Chr8 link | 2459 | WIAF\|WIAF-50 |
| 8 | 625.4 cR from top of Chr8 link | 2664 | WIAF\|WIAF-341 |
| 8 | 628.6 cR from top of Chr8 link | 2202 | WIAF\|WIAF-615 |
| 8 | 653.0 cR from top of Chr8 link | 2864 | WIAF\|WIAF-761 |
| 8 | 656.3 cR from top of Chr8 link | 934 | WIAF\|WIAF-1291 |
| 8 | 659.2 cR from top of Chr8 link | 3088 | WIAF\|WIAF-989 |
| 8 | 670.0 cR from top of Chr8 link | 2807 | WIAF\|WIAF-583 |
| 8 | 681.6 cR from top of Chr8 link | 2839 | WIAF\|WIAF-677 |
| 8 | 689.6 cR from top of Chr8 link | 775 | WIAF\|WIAF-1410 |
| 8 | 708.8 cR from top of Chr8 link | 3126 | WIAF\|WIAF-1027 |
| 8 | 717.8 cR from top of Chr8 link | 2504 | WIAF\|WIAF-108 |
| 8 | 731.6 cR from top of Chr8 link | 2239 | WIAF\|WIAF-676 |
| 8 | 791.3 cR from top of Chr8 link | 742 | WIAF\|WIAF-1186 |
| 8 | | 4220 | MARSHFIELD\|MID-12 |
| 8 | | 4000 | SHGC/AFFYMETRIX\|SNP-SHGC-12093 |
| 8 | | 4140 | SHGC/AFFYMETRIX\|SNP-SHGC-13126 |
| 8 | | 4065 | SHGC/AFFYMETRIX\|SNP-SHGC-13448 |
| 8 | | 3984 | SHGC/AFFYMETRIX\|SNP-SHGC-9711 |
| 8 | | 3143 | WIAF\|WIAF-1044 |
| 8 | | 3171 | WIAF\|WIAF-1482 |
| 8 | | 3292 | WIAF\|WIAF-1745 |
| 8 | | 3347 | WIAF\|WIAF-1800 |
| 8 | | 3447 | WIAF\|WIAF-1902 |
| 8 | | 3700 | WIAF\|WIAF-2428 |
| 8 | | 3779 | WIAF\|WIAF-2507 |
| 8 | | 1634 | WIAF\|WIAF-3573 |
| 8 | | 3890 | WIAF\|WIAF-3951 |
| 8 | | 3908 | WIAF\|WIAF-3985 |
| 8 | | 2742 | WIAF\|WIAF-457 |
| 8 | | 2743 | WIAF\|WIAF-458 |
| 8 | | 2488 | WIAF\|WIAF-86 |
| 8 | | 2965 | WIAF\|WIAF-865 |
| 8 | | 3095 | WIAF\|WIAF-996 |
| 9 | 0.00 cR from top of Chr9 linka | 1358 | WIAF\|WIAF-3261 |
| 9 | 0.00 cR from top of Chr9 linka | 1393 | WIAF\|WIAF-3298 |
| 9 | 7.40 cR from top of Chr9 linka | 1942 | WIAF\|WIAF-3881 |
| 9 | 12.10 cR from top of Chr9 link | 1400 | WIAF\|WIAF-3320 |
| 9 | 13.00 cR from top of Chr9 link | 1155 | WIAF\|WIAF-1640 |
| 9 | 13.00 cR from top of Chr9 link | 1156 | WIAF\|WIAF-1641 |
| 9 | 15.60 cR from top of Chr9 link | 2027 | WIAF\|WIAF-1582 |
| 9 | 15.60 cR from top of Chr9 link | 1661 | WIAF\|WIAF-3600 |
| 9 | 19.60 cR from top of Chr9 link | 1279 | WIAF\|WIAF-2166 |
| 9 | 25.00 cR from top of Chr9 link | 688 | WIAF\|WIAF-1194 |
| 9 | 28.90 cR from top of Chr9 link | 1749 | WIAF\|WIAF-3688 |
| 9 | 29.5 cR from top of Chr9 linka | 2105 | WIAF\|WIAF-185 |
| 9 | 30.0 cR from top of Chr9 linka | 2777 | WIAF\|WIAF-521 |
| 9 | 30.0 cR from top of Chr9 linka | 3056 | WIAF\|WIAF-957 |
| 9 | 44.00 cR from top of Chr9 link | 3464 | WIAF\|WIAF-1919 |
| 9 | 55.6 cR from top of Chr9 linka | 3298 | WIAF\|WIAF-1751 |
| 9 | 55.6 cR from top of Chr9 linka | 3299 | WIAF\|WIAF-1752 |
| 9 | 57.40 cR from top of Chr9 link | 1283 | WIAF\|WIAF-2170 |
| 9 | 57.50 cR from top of Chr9 link | 1403 | WIAF\|WIAF-3323 |
| 9 | 57.80 cR from top of Chr9 link | 2556 | WIAF\|WIAF-180 |
| 9 | 62.0 cR from top of Chr9 linka | 4575 | HU-CHINA\|9-870 |
| 9 | 62.0 cR from top of Chr9 linka | 2970 | WIAF\|WIAF-870 |
| 9 | 62.70 cR from top of Chr9 link | 3246 | WIAF\|WIAF-1657 |
| 9 | 62.70 cR from top of Chr9 link | 3247 | WIAF\|WIAF-1658 |
| 9 | 64.0 cR from top of Chr9 linka | 2272 | WIAF\|WIAF-731 |
| 9 | 64.10 cR from top of Chr9 link | 2043 | WIAF\|WIAF-1634 |
| 9 | 65.20 cR from top of Chr9 link | 1148 | WIAF\|WIAF-1619 |
| 9 | 67.40 cR from top of Chr9 link | 1318 | WIAF\|WIAF-2208 |
| 9 | 67.40 cR from top of Chr9 link | 1319 | WIAF\|WIAF-2209 |
| 9 | 68.80 cR from top of Chr9 link | 1874 | WIAF\|WIAF-3813 |
| 9 | 68.9 cR from top of Chr9 linka | 932 | WIAF\|WIAF-1280 |
| 9 | 69.70 cR from top of Chr9 link | 3433 | WIAF\|WIAF-1888 |
| 9 | 74.9 cR from top of Chr9 linka | 3444 | WIAF\|WIAF-1899 |
| 9 | 74.9 cR from top of Chr9 linka | 3445 | WIAF\|WIAF-1900 |
| 9 | 82.30 cR from top of Chr9 link | 1246 | WIAF\|WIAF-2133 |
| 9 | 83.90 cR from top of Chr9 link | 1278 | WIAF\|WIAF-2165 |
| 9 | 84.50 cR from top of Chr9 link | 3482 | WIAF\|WIAF-1937 |
| 9 | 84.60 cR from top of Chr9 link | 1579 | WIAF\|WIAF-3518 |
| 9 | 84.60 cR from top of Chr9 link | 1581 | WIAF\|WIAF-3520 |
| 9 | 86.80 cR from top of Chr9 link | 1192 | WIAF\|WIAF-2079 |
| 9 | 95.90 cR from top of Chr9 link | 1892 | WIAF\|WIAF-3831 |
| 9 | 100.60 cR from top of Chr9 lin | 3311 | WIAF\|WIAF-1764 |
| 9 | 100.60 cR from top of Chr9 lin | 1405 | WIAF\|WIAF-3325 |
| 9 | 105.20 cR from top of Chr9 lin | 1615 | WIAF\|WIAF-3554 |
| 9 | 105.80 cR from top of Chr9 lin | 1237 | WIAF\|WIAF-2124 |
| 9 | 105.80 cR from top of Chr9 lin | 1238 | WIAF\|WIAF-2125 |
| 9 | 106.20 cR from top of Chr9 lin | 686 | WIAF\|WIAF-1177 |
| 9 | 106.20 cR from top of Chr9 lin | 687 | WIAF\|WIAF-1178 |
| 9 | 109.3 cR from top of Chr9 link | 2237 | WIAF\|WIAF-674 |
| 9 | 109.3 cR from top of Chr9 link | 2238 | WIAF\|WIAF-675 |
| 9 | 118.20 cR from top of Chr9 lin | 2881 | WIAF\|WIAF-779 |
| 9 | 122.20 cR from top of Chr9 lin | 1536 | WIAF\|WIAF-3475 |
| 9 | 122.20 cR from top of Chr9 lin | 1538 | WIAF\|WIAF-3477 |
| 9 | 123.20 cR from top of Chr9 lin | 1430 | WIAF\|WIAF-3358 |
| 9 | 124.00 cR from top of Chr9 lin | 1268 | WIAF\|WIAF-2155 |
| 9 | 124.00 cR from top of Chr9 lin | 1269 | WIAF\|WIAF-2156 |
| 9 | 129.0 cR from top of Chr9 link | 3830 | WIAF\|WIAF-2599 |
| 9 | 132.60 cR from top of Chr9 lin | 1999 | WIAF\|WIAF-3938 |
| 9 | 136.30 cR from top of Chr9 lin | 1154 | WIAF\|WIAF-1638 |
| 9 | 137.00 cR from top of Chr9 lin | 1533 | WIAF\|WIAF-3472 |
| 9 | 137.70 cR from top of Chr9 lin | 1301 | WIAF\|WIAF-2190 |
| 9 | 137.70 cR from top of Chr9 lin | 1302 | WIAF\|WIAF-2191 |
| 9 | 138.0 cR from top of Chr9 link | 2222 | WIAF\|WIAF-649 |
| 9 | 142.10 cR from top of Chr9 lin | 1640 | WIAF\|WIAF-3579 |
| 9 | 142.70 cR from top of Chr9 lin | 1331 | WIAF\|WIAF-2221 |
| 9 | 142.70 cR from top of Chr9 lin | 1929 | WIAF\|WIAF-3868 |
| 9 | 143.50 cR from top of Chr9 lin | 1207 | WIAF\|WIAF-2094 |
| 9 | 143.50 cR from top of Chr9 lin | 1208 | WIAF\|WIAF-2095 |
| 9 | 143.90 cR from top of Chr9 lin | 2558 | WIAF\|WIAF-182 |
| 9 | 144.60 cR from top of Chr9 lin | 1797 | WIAF\|WIAF-3736 |
| 9 | 148.7 cR from top of Chr9 link | 891 | WIAF\|WIAF-1136 |
| 9 | 148.7 cR from top of Chr9 link | 1042 | WIAF\|WIAF-4131 |
| 9 | 164.70 cR from top of Chr9 lin | 1994 | WIAF\|WIAF-3933 |
| 9 | 166.50 cR from top of Chr9 lin | 1829 | WIAF\|WIAF-3768 |
| 9 | 210.3 cR from top of Chr9 link | 3074 | WIAF\|WIAF-975 |
| 9 | 264.4 cR from top of Chr9 link | 786 | WIAF\|WIAF-2049 |
| 9 | 293.7 cR from top of Chr9 link | 2245 | WIAF\|WIAF-689 |
| 9 | 326.9 cR from top of Chr9 link | 2621 | WIAF\|WIAF-277 |
| 9 | 328.5 cR from top of Chr9 link | 2642 | WIAF\|WIAF-309 |
| 9 | 328.5 cR from top of Chr9 link | 2725 | WIAF\|WIAF-430 |
| 9 | 336.4 cR from top of Chr9 link | 2439 | WIAF\|WIAF-24 |
| 9 | 342.6 cR from top of Chr9 link | 2341 | WIAF\|WIAF-2567 |
| 9 | 345.4 cR from top of Chr9 link | 3059 | WIAF\|WIAF-960 |
| 9 | 367.4 cR from top of Chr9 link | 2899 | WIAF\|WIAF-797 |
| 9 | 374.1 cR from top of Chr9 link | 2453 | WIAF\|WIAF-41 |
| 9 | 389.8 cR from top of Chr9 link | 2845 | WIAF\|WIAF-691 |
| 9 | 409.6 cR from top of Chr9 link | 2145 | WIAF\|WIAF-433 |
| 9 | 437.9 cR from top of Chr9 link | 2806 | WIAF\|WIAF-578 |
| 9 | 447.0 cR from top of Chr9 link | 3050 | WIAF\|WIAF-951 |
| 9 | 449.9 cR from top of Chr9 link | 3005 | WIAF\|WIAF-905 |
| 9 | 450.0 cR from top of Chr9 link | 2601 | WIAF\|WIAF-245 |
| 9 | 480.5 cR from top of Chr9 link | 2446 | WIAF\|WIAF-34 |
| 9 | 483.4 cR from top of Chr9 link | 2565 | WIAF\|WIAF-194 |
| 9 | 493.6 cR from top of Chr9 link | 2547 | WIAF\|WIAF-168 |
| 9 | 511.3 cR from top of Chr9 link | 3468 | WIAF\|WIAF-1923 |
| 9 | 512.4 cR from top of Chr9 link | 2521 | WIAF\|WIAF-130 |
| 9 | 515.6 cR from top of Chr9 link | 2524 | WIAF\|WIAF-133 |
| 9 | 516.3 cR from top of Chr9 link | 2102 | WIAF\|WIAF-166 |
| 9 | 518.9 cR from top of Chr9 link | 3816 | WIAF\|WIAF-2570 |
| 9 | 523.2 cR from top of Chr9 link | 2489 | WIAF\|WIAF-87 |
| 9 | 526.5 cR from top of Chr9 link | 998 | WIAF\|WIAF-2346 |
| 9 | 526.5 cR from top of Chr9 link | 999 | WIAF\|WIAF-2349 |
| 9 | 526.5 cR from top of Chr9 link | 1000 | WIAF\|WIAF-2353 |
| 9 | 526.5 cR from top of Chr9 link | 1001 | WIAF\|WIAF-2355 |
| 9 | | 4226 | MARSHFIELD\|MID-18 |
| 9 | | 4227 | MARSHFIELD\|MID-19 |
| 9 | | 3995 | SHGC/AFFYMETRIX\|SNP-SHGC-10262 |
| 9 | | 4007 | SHGC/AFFYMETRIX\|SNP-SHGC-1334 |
| 9 | | 4071 | SHGC/AFFYMETRIX\|SNP-SHGC-14625 |
| 9 | | 4075 | SHGC/AFFYMETRIX\|SNP-SHGC-15679 |
| 9 | | 4079 | SHGC/AFFYMETRIX\|SNP-SHGC-16528 |
| 9 | | 4014 | SHGC/AFFYMETRIX\|SNP-SHGC-3934 |
| 9 | | 3150 | WIAF\|WIAF-1051 |
| 9 | | 3210 | WIAF\|WIAF-1563 |
| 9 | | 3580 | WIAF\|WIAF-2035 |
| 9 | | 3581 | WIAF\|WIAF-2036 |
| 9 | | 1588 | WIAF\|WIAF-3527 |
| 9 | | 1862 | WIAF\|WIAF-3801 |
| 9 | | 2465 | WIAF\|WIAF-58 |
| 9 | | 2466 | WIAF\|WIAF-59 |
| 9 | | 2477 | WIAF\|WIAF-73 |
| 9 | | 3087 | WIAF\|WIAF-988 |
| 10 | −6 cM | 4315 | UWGC\|134 |
| 10 | 6.10 cR from top of Chr10 link | 1310 | WIAF\|WIAF-2199 |
| 10 | 17.30 cR from top of Chr10 lin | 1598 | WIAF\|WIAF-3537 |
| 10 | 17.30 cR from top of Chr10 lin | 1600 | WIAF\|WIAF-3539 |
| 10 | 19.70 cR from top of Chr10 lin | 1271 | WIAF\|WIAF-2158 |
| 10 | 22.20 cR from top of Chr10 lin | 2538 | WIAF\|WIAF-152 |
| 10 | 28.50 cR from top of Chr10 lin | 3181 | WIAF\|WIAF-1497 |
| 10 | 28.50 cR from top of Chr10 lin | 3182 | WIAF\|WIAF-1498 |
| 10 | 29.00 cR from top of Chr10 lin | 4576 | HU-CHINA\|10-1729 |
| 10 | 29.00 cR from top of Chr10 lin | 3276 | WIAF\|WIAF-1729 |
| 10 | 31.40 cR from top of Chr10 lin | 2016 | WIAF\|WIAF-1511 |
| 10 | 31.80 cR from top of Chr10 lin | 1603 | WIAF\|WIAF-3542 |
| 10 | 32.00 cR from top of Chr10 lin | 3265 | WIAF\|WIAF-1704 |
| 10 | 36.30 cR from top of Chr10 lin | 2680 | WIAF\|WIAF-363 |
| 10 | 41.10 cR from top of Chr10 lin | 3268 | WIAF\|WIAF-1713 |
| 10 | 43.3 cR from top of Chr10 link | 3289 | WIAF\|WIAF-1742 |
| 10 | 43.80 cR from top of Chr10 lin | 1675 | WIAF\|WIAF-3614 |
| 10 | 44.90 cR from top of Chr10 lin | 1820 | WIAF\|WIAF-3759 |
| 10 | 45.10 cR from top of Chr10 lin | 1969 | WIAF\|WIAF-3908 |
| 10 | 45.50 cR from top of Chr10 lin | 1183 | WIAF\|WIAF-1715 |
| 10 | 45.50 cR from top of Chr10 lin | 1184 | WIAF\|WIAF-1716 |
| 10 | 52.00 cR from top of Chr10 lin | 1363 | WIAF\|WIAF-3266 |
| 10 | 61.60 cR from top of Chr10 lin | 1178 | WIAF\|WIAF-1707 |
| 10 | 67.90 cR from top of Chr10 lin | 1858 | WIAF\|WIAF-3797 |
| 10 | 79.40 cR from top of Chr10 lin | 1962 | WIAF\|WIAF-3901 |
| 10 | 80.20 cR from top of Chr10 lin | 3230 | WIAF\|WIAF-1632 |
| 10 | 83.30 cR from top of Chr10 lin | 1960 | WIAF\|WIAF-3899 |
| 10 | 85.60 cR from top of Chr10 lin | 1772 | WIAF\|WIAF-3711 |
| 10 | 89.40 cR from top of Chr10 lin | 3517 | WIAF\|WIAF-1972 |
| 10 | 96.30 cR from top of Chr10 lin | 1198 | WIAF\|WIAF-2085 |
| 10 | 96.90 cR from top of Chr10 lin | 3213 | WIAF\|WIAF-1585 |
| 10 | 96.90 cR from top of Chr10 lin | 1542 | WIAF\|WIAF-3481 |
| 10 | 96.90 cR from top of Chr10 lin | 1651 | WIAF\|WIAF-3590 |
| 10 | 97.40 cR from top of Chr10 lin | 1698 | WIAF\|WIAF-3637 |
| 10 | 97.60 cR from top of Chr10 lin | 1554 | WIAF\|WIAF-3493 |
| 10 | 97.60 cR from top of Chr10 lin | 1556 | WIAF\|WIAF-3495 |
| 10 | 105.3 cR from top of Chr10 lin | 2428 | WIAF\|WIAF-9 |
| 10 | 106.70 cR from top of Chr10 li | 3187 | WIAF\|WIAF-1503 |
| 10 | 107.90 cR from top of Chr10 li | 1597 | WIAF\|WIAF-3536 |
| 10 | 110.30 cR from top of Chr10 li | 1499 | WIAF\|WIAF-3438 |
| 10 | 110.50 cR from top of Chr10 li | 3561 | WIAF\|WIAF-2016 |
| 10 | 112.50 cR from top of Chr10 li | 1390 | WIAF\|WIAF-3295 |
| 10 | 112.70 cR from top of Chr10 li | 1413 | WIAF\|WIAF-3334 |
| 10 | 112.70 cR from top of Chr10 li | 1414 | WIAF\|WIAF-3335 |
| 10 | 113.50 cR from top of Chr10 li | 1752 | WIAF\|WIAF-3691 |
| 10 | 122.50 cR from top of Chr10 li | 1462 | WIAF\|WIAF-3396 |
| 10 | 123 cM | 4321 | UWGC\|140 |
| 10 | 123.00 cR from top of Chr10 li | 1703 | WIAF\|WIAF-3642 |
| 10 | 130.50 cR from top of Chr10 li | 1762 | WIAF\|WIAF-3701 |
| 10 | 130.50 cR from top of Chr10 li | 1764 | WIAF\|WIAF-3703 |
| 10 | 133.6 cR from top of Chr10 li | 3035 | WIAF\|WIAF-935 |
| 10 | 134.80 cR from top of Chr10 li | 2045 | WIAF\|WIAF-1661 |
| 10 | 134.80 cR from top of Chr10 li | 1389 | WIAF\|WIAF-3294 |
| 10 | 138.90 cR from top of Chr10 li | 1165 | WIAF\|WIAF-1676 |
| 10 | 146.60 cR from top of Chr10 li | 1394 | WIAF\|WIAF-3299 |
| 10 | 146.80 cR from top of Chr10 li | 1822 | WIAF\|WIAF-3761 |
| 10 | 150.50 cR from top of Chr10 li | 3412 | WIAF\|WIAF-1867 |
| 10 | 155.30 cR from top of Chr10 li | 3337 | WIAF\|WIAF-1790 |
| 10 | 180.3 cR from top of Chr10 lin | 3065 | WIAF\|WIAF-966 |
| 10 | 185.3 cR from top of Chr10 lin | 2938 | WIAF\|WIAF-837 |
| 10 | 293.4 cR from top of Chr10 lin | 798 | WIAF\|WIAF-4055 |
| 10 | 306.5 cR from top of Chr10 lin | 2552 | WIAF\|WIAF-175 |
| 10 | 343.7 cR from top of Chr10 lin | 939 | WIAF\|WIAF-1297 |
| 10 | 356.8 cR from top of Chr10 lin | 2199 | WIAF\|WIAF-609 |
| 10 | 359.1 cR from top of Chr10 lin | 3529 | WIAF\|WIAF-1984 |
| 10 | 366.6 cR from top of Chr10 lin | 3141 | WIAF\|WIAF-1042 |
| 10 | 382.1 cR from top of Chr10 lin | 2127 | WIAF\|WIAF-303 |
| 10 | 384.4 cR from top of Chr10 lin | 2247 | WIAF\|WIAF-692 |
| 10 | 389.5 cR from top of Chr10 lin | 2536 | WIAF\|WIAF-149 |
| 10 | 425.7 cR from top of Chr10 lin | 2912 | WIAF\|WIAF-811 |
| 10 | 431.3 cR from top of Chr10 lin | 2641 | WIAF\|WIAF-308 |
| 10 | 433.0 cR from top of Chr10 lin | 2081 | WIAF\|WIAF-31 |
| 10 | 437.2 cR from top of Chr10 lin | 2128 | WIAF\|WIAF-310 |
| 10 | 440.2 cR from top of Chr10 lin | 3346 | WIAF\|WIAF-1799 |
| 10 | 442.3 cR from top of Chr10 lin | 2277 | WIAF\|WIAF-744 |
| 10 | 467.6 cR from top of Chr10 lin | 3434 | WIAF\|WIAF-1889 |
| 10 | 467.6 cR from top of Chr10 lin | 3435 | WIAF\|WIAF-1890 |
| 10 | 496.3 cR from top of Chr10 lin | 2991 | WIAF\|WIAF-891 |
| 10 | 505.8 cR from top of Chr10 lin | 2351 | WIAF\|WIAF-2588 |
| 10 | 505.8 cR from top of Chr10 lin | 2353 | WIAF\|WIAF-2593 |
| 10 | 515.2 cR from top of Chr10 lin | 2882 | WIAF\|WIAF-780 |
| 10 | 515.7 cR from top of Chr10 lin | 3334 | WIAF\|WIAF-1787 |
| 10 | 537.8 cR from top of Chr10 lin | 3416 | WIAF\|WIAF-1871 |
| 10 | 542.2 cR from top of Chr10 lin | 3037 | WIAF\|WIAF-937 |
| 10 | 551.7 cR from top of Chr10 lin | 3102 | WIAF\|WIAF-1003 |
| 10 | 557.3 cR from top of Chr10 lin | 3506 | WIAF\|WIAF-1961 |
| 10 | 558.3 cR from top of Chr10 lin | 3155 | WIAF\|WIAF-1056 |
| 10 | 567.5 cR from top of Chr10 lin | 3853 | WIAF\|WIAF-2640 |
| 10 | 598.4 cR from top of Chr10 lin | 2620 | WIAF\|WIAF-271 |
| 10 | 620.5 cR from top of Chr10 lin | 3558 | WIAF\|WIAF-2013 |
| 10 | 623.8 cR from top of Chr10 lin | 2879 | WIAF\|WIAF-777 |
| 10 | 646.1 cR from top of Chr10 lin | 894 | WIAF\|WIAF-1142 |
| 10 | | 4153 | SHGC/AFFYMETRIX\|SNP-SHGC-14257 |
| 10 | | 4159 | SHGC/AFFYMETRIX\|SNP-SHGC-14726 |
| 10 | | 4076 | SHGC/AFFYMETRIX\|SNP-SHGC-15732 |
| 10 | | 4166 | SHGC/AFFYMETRIX\|SNP-SHGC-23692 |
| 10 | | 4103 | SHGC/AFFYMETRIX\|SNP-SHGC-30908 |
| 10 | | 4104 | SHGC/AFFYMETRIX\|SNP-SHGC-31374 |
| 10 | | 3976 | SHGC/AFFYMETRIX\|SNP-SHGC-35401 |
| 10 | | 3724 | WIAF\|WIAF-2452 |
| 10 | | 1530 | WIAF\|WIAF-3469 |
| 10 | | 1691 | WIAF\|WIAF-3630 |
| 10 | | 3882 | WIAF\|WIAF-3943 |
| 10 | | 2483 | WIAF\|WIAF-80 |
| 10 | | 3001 | WIAF\|WIAF-901 |
| 10 | | 3079 | WIAF\|WIAF-980 |
| 11 | 3.10 cR from top of Chr11 link | 1544 | WIAF\|WIAF-3483 |
| 11 | 3.10 cR from top of Chr11 link | 1683 | WIAF\|WIAF-3622 |
| 11 | 3.90 cR from top of Chr11 link | 685 | WIAF\|WIAF-1160 |
| 11 | 3.90 cR from top of Chr11 link | 694 | WIAF\|WIAF-1245 |
| 11 | 4.60 cR from top of Chr11 link | 1123 | WIAF\|WIAF-1566 |
| 11 | 15.50 cR from top of Chr11 lin | 1900 | WIAF\|WIAF-3839 |
| 11 | 15.60 cR from top of Chr11 lin | 665 | WIAF\|WIAF-1061 |
| 11 | 15.60 cR from top of Chr11 lin | 1710 | WIAF\|WIAF-3649 |
| 11 | 16.80 cR from top of Chr11 lin | 1392 | WIAF\|WIAF-3297 |
| 11 | 18.50 cR from top of Chr11 lin | 1568 | WIAF\|WIAF-3507 |
| 11 | 18.50 cR from top of Chr11 lin | 1571 | WIAF\|WIAF-3510 |
| 11 | 23.50 cR from top of Chr11 lin | 699 | WIAF\|WIAF-1271 |
| 11 | 23.60 cR from top of Chr11 lin | 2987 | WIAF\|WIAF-887 |
| 11 | 23.70 cR from top of Chr11 lin | 1580 | WIAF\|WIAF-3519 |
| 11 | 24.50 cR from top of Chr11 lin | 1355 | WIAF\|WIAF-3258 |
| 11 | 26.50 cR from top of Chr11 lin | 1553 | WIAF\|WIAF-3492 |
| 11 | 30.7 cR from top of Chr11 link | 2615 | WIAF\|WIAF-262 |
| 11 | 34.9 cR from top of Chr11 link | 784 | WIAF\|WIAF-2043 |
| 11 | 38.00 cR from top of Chr11 lin | 3494 | WIAF\|WIAF-1949 |
| 11 | 38.3 cR from top of Chr11 link | 2921 | WIAF\|WIAF-820 |
| 11 | 39.80 cR from top of Chr11 lin | 1531 | WIAF\|WIAF-3470 |
| 11 | 43.7 cR from top of Chr11 link | 3316 | WIAF\|WIAF-1769 |
| 11 | 45.7 cR from top of Chr11 link | 3400 | WIAF\|WIAF-1855 |
| 11 | 52.1 cR from top of Chr11 link | 2192 | WIAF\|WIAF-591 |
| 11 | 52.2 cR from top of Chr11 link | 2176 | WIAF\|WIAF-551 |
| 11 | 58.7 cR from top of Chr11 link | 3502 | WIAF\|WIAF-1957 |
| 11 | 62.20 cR from top of Chr11 lin | 1958 | WIAF\|WIAF-3897 |
| 11 | 62.30 cR from top of Chr11 lin | 1371 | WIAF\|WIAF-3274 |
| 11 | 62.50 cR from top of Chr11 lin | 1560 | WIAF\|WIAF-3499 |
| 11 | 62.50 cR from top of Chr11 lin | 1917 | WIAF\|WIAF-3856 |
| 11 | 63.20 cR from top of Chr11 lin | 3198 | WIAF\|WIAF-1535 |
| 11 | 63.20 cR from top of Chr11 lin | 3199 | WIAF\|WIAF-1536 |
| 11 | 67.00 cR from top of Chr11 lin | 1277 | WIAF\|WIAF-2164 |
| 11 | 68.0 cR from top of Chr11 link | 2233 | WIAF\|WIAF-668 |
| 11 | 68.30 cR from top of Chr11 lin | 2056 | WIAF\|WIAF-1719 |
| 11 | 68.30 cR from top of Chr11 lin | 1330 | WIAF\|WIAF-2220 |
| 11 | 74.30 cR from top of Chr11 lin | 3158 | WIAF\|WIAF-1059 |
| 11 | 76.50 cR from top of Chr11 lin | 1361 | WIAF\|WIAF-3264 |
| 11 | 76.50 cR from top of Chr11 lin | 1720 | WIAF\|WIAF-3659 |
| 11 | 76.60 cR from top of Chr11 lin | 1306 | WIAF\|WIAF-2195 |
| 11 | 77.10 cR from top of Chr11 lin | 1901 | WIAF\|WIAF-3840 |
| 11 | 77.50 cR from top of Chr11 lin | 2022 | WIAF\|WIAF-1529 |
| 11 | 80.10 cR from top of Chr11 lin | 1645 | WIAF\|WIAF-3584 |
| 11 | 81.90 cR from top of Chr11 lin | 1202 | WIAF\|WIAF-2089 |
| 11 | 82.90 cR from top of Chr11 lin | 2001 | WIAF\|WIAF-3940 |
| 11 | 83.20 cR from top of Chr11 lin | 3091 | WIAF\|WIAF-992 |
| 11 | 84.20 cR from top of Chr11 lin | 3170 | WIAF\|WIAF-1480 |
| 11 | 86.90 cR from top of Chr11 lin | 3162 | WIAF\|WIAF-1465 |
| 11 | 89.8 cR from top of Chr11 link | 996 | WIAF\|WIAF-2045 |
| 11 | 89.8 cR from top of Chr11 link | 3585 | WIAF\|WIAF-2046 |
| 11 | 93.30 cR from top of Chr11 lin | 1217 | WIAF\|WIAF-2104 |
| 11 | 94.10 cR from top of Chr11 lin | 3497 | WIAF\|WIAF-1952 |
| 11 | 97.90 cR from top of Chr11 lin | 1122 | WIAF\|WIAF-1564 |
| 11 | 98.40 cR from top of Chr11 lin | 1095 | WIAF\|WIAF-1483 |
| 11 | 98.40 cR from top of Chr11 lin | 1096 | WIAF\|WIAF-1484 |
| 11 | 102.60 cR from top of Chr11 li | 3495 | WIAF\|WIAF-1950 |
| 11 | 106.6 cR from top of Chr11 lin | 864 | WIAF\|WIAF-1075 |
| 11 | 106.80 cR from top of Chr11 li | 1868 | WIAF\|WIAF-3807 |
| 11 | 106.80 cR from top of Chr11 li | 1869 | WIAF\|WIAF-3808 |
| 11 | 107.90 cR from top of Chr11 li | 3202 | WIAF\|WIAF-1542 |
| 11 | 108.00 cR from top of Chr11 li | 2049 | WIAF\|WIAF-1688 |
| 11 | 108.10 cR from top of Chr11 li | 1478 | WIAF\|WIAF-3417 |
| 11 | 112.50 cR from top of Chr11 li | 1103 | WIAF\|WIAF-1525 |
| 11 | 113 cM | 4311 | UWGC\|130 |
| 11 | 116.20 cR from top of Chr11 li | 1909 | WIAF\|WIAF-3848 |
| 11 | 117.40 cR from top of Chr11 li | 675 | WIAF\|WIAF-1440 |
| 11 | 118.40 cR from top of Chr11 li | 1714 | WIAF\|WIAF-3653 |
| 11 | 118.40 cR from top of Chr11 li | 1715 | WIAF\|WIAF-3654 |
| 11 | 120.00 cR from top of Chr11 li | 1830 | WIAF\|WIAF-3769 |
| 11 | 120.00 cR from top of Chr11 li | 1831 | WIAF\|WIAF-3770 |
| 11 | 120.10 cR from top of Chr11 li | 1943 | WIAF\|WIAF-3882 |
| 11 | 120.70 cR from top of Chr11 li | 3258 | WIAF\|WIAF-1694 |
| 11 | 126.00 cR from top of Chr11 li | 1854 | WIAF\|WIAF-3793 |
| 11 | 126.60 cR from top of Chr11 li | 1386 | WIAF\|WIAF-3291 |
| 11 | 131.70 cR from top of Chr11 li | 1899 | WIAF\|WIAF-3838 |
| 11 | 145.60 cR from top of Chr11 li | 1806 | WIAF\|WIAF-3745 |
| 11 | 149.80 cR from top of Chr11 li | 3267 | WIAF\|WIAF-1712 |
| 11 | 150.6 cR from top of Chr11 lin | 1089 | WIAF\|WIAF-3183 |
| 11 | 166.3 cR from top of Chr11 lin | 2942 | WIAF\|WIAF-841 |
| 11 | 171.5 cR from top of Chr11 lin | 3829 | WIAF\|WIAF-2598 |
| 11 | 305.1 cR from top of Chr11 lin | 3831 | WIAF\|WIAF-2600 |
| 11 | 314.5 cR from top of Chr11 lin | 2892 | WIAF\|WIAF-790 |
| 11 | 323.0 cR from top of Chr11 lin | 2606 | WIAF\|WIAF-252 |
| 11 | 323.0 cR from top of Chr11 lin | 2607 | WIAF\|WIAF-253 |
| 11 | 344.4 cR from top of Chr11 lin | 3407 | WIAF\|WIAF-1862 |
| 11 | 359.1 cR from top of Chr11 lin | 2530 | WIAF\|WIAF-141 |
| 11 | 359.1 cR from top of Chr11 lin | 2531 | WIAF\|WIAF-142 |
| 11 | 359.1 cR from top of Chr11 lin | 2532 | WIAF\|WIAF-143 |
| 11 | 376.1 cR from top of Chr11 lin | 3411 | WIAF\|WIAF-1866 |
| 11 | 377.9 cR from top of Chr11 lin | 2533 | WIAF\|WIAF-146 |
| 11 | 379.5 cR from top of Chr11 lin | 2484 | WIAF\|WIAF-81 |
| 11 | 387.7 cR from top of Chr11 lin | 2592 | WIAF\|WIAF-228 |
| 11 | 387.7 cR from top of Chr11 lin | 2593 | WIAF\|WIAF-229 |
| 11 | 389.8 cR from top of Chr11 lin | 2857 | WIAF\|WIAF-750 |
| 11 | 392.6 cR from top of Chr11 lin | 2943 | WIAF\|WIAF-842 |
| 11 | 403.4 cR from top of Chr11 lin | 3815 | WIAF\|WIAF-2569 |
| 11 | 419.0 cR from top of Chr11 lin | 2590 | WIAF\|WIAF-226 |
| 11 | 419.0 cR from top of Chr11 lin | 2591 | WIAF\|WIAF-227 |
| 11 | 421.1 cR from top of Chr11 lin | 3371 | WIAF\|WIAF-1824 |
| 11 | 428.6 cR from top of Chr11 lin | 3069 | WIAF\|WIAF-970 |
| 11 | 432.6 cR from top of Chr11 lin | 2103 | WIAF\|WIAF-174 |
| 11 | 458.3 cR from top of Chr11 lin | 3478 | WIAF\|WIAF-1933 |
| 11 | 466.7 cR from top of Chr11 lin | 3608 | WIAF\|WIAF-2322 |
| 11 | 488.1 cR from top of Chr11 lin | 3134 | WIAF\|WIAF-1035 |
| 11 | 506.0 cR from top of Chr11 lin | 1086 | WIAF\|WIAF-2071 |
| 11 | 522.5 cR from top of Chr11 lin | 2485 | WIAF\|WIAF-82 |
| 11 | 573.0 cR from top of Chr11 lin | 2736 | WIAF\|WIAF-447 |
| 11 | 604.0 cR from top of Chr11 lin | 2727 | WIAF\|WIAF-432 |
| 11 | 604.0 cR from top of Chr11 lin | 3070 | WIAF\|WIAF-971 |
| 11 | 624.2 cR from top of Chr11 lin | 3379 | WIAF\|WIAF-1832 |
| 11 | | 4228 | MARSHFIELD\|MID-21 |
| 11 | | 3959 | SHGC/AFFYMETRIX\|SNP-SHGC-10796 |
| 11 | | 3965 | SHGC/AFFYMETRIX\|SNP-SHGC-11902 |
| 11 | | 4064 | SHGC/AFFYMETRIX\|SNP-SHGC-13369 |
| 11 | | 4073 | SHGC/AFFYMETRIX\|SNP-SHGC-15155 |
| 11 | | 4093 | SHGC/AFFYMETRIX\|SNP-SHGC-17309 |
| 11 | | 4013 | SHGC/AFFYMETRIX\|SNP-SHGC-3925 |
| 11 | | 4046 | SHGC/AFFYMETRIX\|SNP-SHGC-9225 |
| 11 | | 2429 | WIAF\|WIAF-10 |
| 11 | | 1090 | WIAF\|WIAF-1463 |
| 11 | | 1134 | WIAF\|WIAF-1581 |
| 11 | | 3214 | WIAF\|WIAF-1588 |
| 11 | | 3259 | WIAF\|WIAF-1695 |
| 11 | | 3313 | WIAF\|WIAF-1766 |
| 11 | | 3314 | WIAF\|WIAF-1767 |
| 11 | | 3543 | WIAF\|WIAF-1998 |
| 11 | | 1638 | WIAF\|WIAF-3577 |
| 11 | | 1666 | WIAF\|WIAF-3605 |
| 11 | | 3090 | WIAF\|WIAF-991 |
| 12 | 0.70 cR from top of Chr12 link | 1662 | WIAF\|WIAF-3601 |
| 12 | 10.70 cR from top of Chr12 lin | 3553 | WIAF\|WIAF-2008 |
| 12 | 13.80 cR from top of Chr12 lin | 3353 | WIAF\|WIAF-1806 |
| 12 | 15.10 cR from top of Chr12 lin | 1573 | WIAF\|WIAF-3512 |
| 12 | 19.60 cR from top of Chr12 lin | 3352 | WIAF\|WIAF-1805 |
| 12 | 20.40 cR from top of Chr12 lin | 674 | WIAF\|WIAF-1430 |
| 12 | 28.30 cR from top of Chr12 lin | 1967 | WIAF\|WIAF-3906 |
| 12 | 28.40 cR from top of Chr12 lin | 2862 | WIAF\|WIAF-759 |
| 12 | 29.30 cR from top of Chr12 lin | 1625 | WIAF\|WIAF-3564 |
| 12 | 31.80 cR from top of Chr12 lin | 1848 | WIAF\|WIAF-3787 |
| 12 | 32 cM | 4322 | UWGC\|141 |
| 12 | 32.80 cR from top of Chr12 lin | 2979 | WIAF\|WIAF-879 |
| 12 | 40.50 cR from top of Chr12 lin | 1391 | WIAF\|WIAF-3296 |
| 12 | 52.40 cR from top of Chr12 lin | 1976 | WIAF\|WIAF-3915 |
| 12 | 53.70 cR from top of Chr12 lin | 3477 | WIAF\|WIAF-1932 |
| 12 | 53.90 cR from top of Chr12 lin | 3569 | WIAF\|WIAF-2024 |
| 12 | 54.00 cR from top of Chr12 lin | 1320 | WIAF\|WIAF-2210 |
| 12 | 54.40 cR from top of Chr12 lin | 3461 | WIAF\|WIAF-1916 |
| 12 | 54.40 cR from top of Chr12 lin | 3462 | WIAF\|WIAF-1917 |
| 12 | 57.40 cR from top of Chr12 lin | 3262 | WIAF\|WIAF-1698 |
| 12 | 57.40 cR from top of Chr12 lin | 3532 | WIAF\|WIAF-1987 |
| 12 | 57.40 cR from top of Chr12 lin | 3533 | WIAF\|WIAF-1988 |
| 12 | 62.80 cR from top of Chr12 lin | 2051 | WIAF\|WIAF-1692 |
| 12 | 62.90 cR from top of Chr12 lin | 1692 | WIAF\|WIAF-3631 |
| 12 | 65.70 cR from top of Chr12 lin | 1800 | WIAF\|WIAF-3739 |
| 12 | 66.90 cR from top of Chr12 lin | 913 | WIAF\|WIAF-1192 |
| 12 | 67.00 cR from top of Chr12 lin | 1632 | WIAF\|WIAF-3571 |
| 12 | 67.20 cR from top of Chr12 lin | 2046 | WIAF\|WIAF-1662 |
| 12 | 69.60 cR from top of Chr12 lin | 3597 | WIAF\|WIAF-2202 |
| 12 | 69.90 cR from top of Chr12 lin | 2561 | WIAF\|WIAF-188 |
| 12 | 70.60 cR from top of Chr12 lin | 3089 | WIAF\|WIAF-990 |
| 12 | 71.10 cR from top of Chr12 lin | 3525 | WIAF\|WIAF-1980 |
| 12 | 72.30 cR from top of Chr12 lin | 2495 | WIAF\|WIAF-93 |
| 12 | 74.50 cR from top of Chr12 lin | 2050 | WIAF\|WIAF-1691 |
| 12 | 75.40 cR from top of Chr12 lin | 1853 | WIAF\|WIAF-3792 |
| 12 | 75.80 cR from top of Chr12 lin | 1313 | WIAF\|WIAF-2203 |
| 12 | 75.80 cR from top of Chr12 lin | 1314 | WIAF\|WIAF-2204 |
| 12 | 75.80 cR from top of Chr12 lin | 1315 | WIAF\|WIAF-2205 |
| 12 | 75.80 cR from top of Chr12 lin | 1730 | WIAF\|WIAF-3669 |
| 12 | 76.50 cR from top of Chr12 lin | 1799 | WIAF\|WIAF-3738 |
| 12 | 78.60 cR from top of Chr12 lin | 3234 | WIAF\|WIAF-1643 |
| 12 | 78.60 cR from top of Chr12 lin | 3235 | WIAF\|WIAF-1644 |
| 12 | 79.10 cR from top of Chr12 lin | 1705 | WIAF\|WIAF-3644 |
| 12 | 80.10 cR from top of Chr12 lin | 1409 | WIAF\|WIAF-3330 |
| 12 | 83.4 cR from top of Chr12 link | 3335 | WIAF\|WIAF-1788 |
| 12 | 97.00 cR from top of Chr12 lin | 3377 | WIAF\|WIAF-1830 |
| 12 | 100.9 cR from top of Chr12 lin | 2507 | WIAF\|WIAF-113 |
| 12 | 101.90 cR from top of Chr12 li | 1610 | WIAF\|WIAF-3549 |
| 12 | 103.9 cR from top of Chr12 lin | 3034 | WIAF\|WIAF-934 |
| 12 | 108.70 cR from top of Chr12 li | 1397 | WIAF\|WIAF-3302 |
| 12 | 111.0 cR from top of Chr12 lin | 2689 | WIAF\|WIAF-374 |
| 12 | 116.60 cR from top of Chr12 li | 1972 | WIAF\|WIAF-3911 |
| 12 | 117.50 cR from top of Chr12 li | 1991 | WIAF\|WIAF-3930 |
| 12 | 119.00 cR from top of Chr12 li | 676 | WIAF\|WIAF-1442 |
| 12 | 119.00 cR from top of Chr12 li | 1639 | WIAF\|WIAF-3578 |
| 12 | 119.00 cR from top of Chr12 li | 1642 | WIAF\|WIAF-3581 |
| 12 | 123.90 cR from top of Chr12 li | 3554 | WIAF\|WIAF-2009 |
| 12 | 134.40 cR from top of Chr12 li | 3576 | WIAF\|WIAF-2031 |
| 12 | 136.30 cR from top of Chr12 li | 2452 | WIAF\|WIAF-40 |
| 12 | 146.30 cR from top of Chr12 li | 1685 | WIAF\|WIAF-3624 |
| 12 | 146.30 cR from top of Chr12 li | 1687 | WIAF\|WIAF-3626 |
| 12 | 152.2 cR from top of Chr12 lin | 3317 | WIAF\|WIAF-1770 |
| 12 | 152.4 cR from top of Chr12 lin | 959 | WIAF\|WIAF-1343 |
| 12 | 152.4 cR from top of Chr12 lin | 1008 | WIAF\|WIAF-4037 |
| 12 | 165.90 cR from top of Chr12 li | 2957 | WIAF\|WIAF-856 |
| 12 | 169.10 cR from top of Chr12 li | 1562 | WIAF\|WIAF-3501 |
| 12 | 169.10 cR from top of Chr12 li | 1567 | WIAF\|WIAF-3506 |
| 12 | 182.2 cR from top of Chr12 lin | 2506 | WIAF\|WIAF-111 |
| 12 | 239.3 cR from top of Chr12 lin | 2385 | WIAF\|WIAF-2679 |
| 12 | 295.9 cR from top of Chr12 lin | 3044 | WIAF\|WIAF-945 |
| 12 | 317.2 cR from top of Chr12 lin | 2597 | WIAF\|WIAF-237 |
| 12 | 317.2 cR from top of Chr12 lin | 2598 | WIAF\|WIAF-238 |
| 12 | 323.2 cR from top of Chr12 lin | 2549 | WIAF\|WIAF-170 |
| 12 | 327.8 cR from top of Chr12 lin | 2744 | WIAF\|WIAF-463 |
| 12 | 364.1 cR from top of Chr12 lin | 3055 | WIAF\|WIAF-956 |
| 12 | 368.3 cR from top of Chr12 lin | 2270 | WIAF\|WIAF-728 |
| 12 | 378.5 cR from top of Chr12 lin | 2377 | WIAF\|WIAF-2658 |
| 12 | 378.5 cR from top of Chr12 lin | 2378 | WIAF\|WIAF-2661 |
| 12 | 378.7 cR from top of Chr12 lin | 2919 | WIAF\|WIAF-818 |
| 12 | 390.2 cR from top of Chr12 lin | 2927 | WIAF\|WIAF-826 |
| 12 | 396.1 cR from top of Chr12 lin | 3140 | WIAF\|WIAF-1041 |
| 12 | 396.2 cR from top of Chr12 lin | 2883 | WIAF\|WIAF-781 |
| 12 | 396.2 cR from top of Chr12 lin | 2884 | WIAF\|WIAF-782 |
| 12 | 419.5 cR from top of Chr12 lin | 3151 | WIAF\|WIAF-1052 |
| 12 | 419.5 cR from top of Chr12 lin | 3152 | WIAF\|WIAF-1053 |
| 12 | 439.4 cR from top of Chr12 lin | 2583 | WIAF\|WIAF-216 |
| 12 | 476.9 cR from top of Chr12 lin | 731 | WIAF\|WIAF-1088 |
| 12 | 478.9 cR from top of Chr12 lin | 2117 | WIAF\|WIAF-251 |
| 12 | 483.3 cR from top of Chr12 lin | 2137 | WIAF\|WIAF-377 |
| 12 | 526.5 cR from top of Chr12 lin | 3463 | WIAF\|WIAF-1918 |
| 12 | 557.3 cR from top of Chr12 lin | 2794 | WIAF\|WIAF-552 |
| 12 | 580.4 cR from top of Chr12 lin | 3157 | WIAF\|WIAF-1058 |
| 12 | 600.8 cR from top of Chr12 lin | 2737 | WIAF\|WIAF-449 |
| 12 | 603.7 cR from top of Chr12 lin | 2681 | WIAF\|WIAF-364 |
| 12 | 614.7 cR from top of Chr12 lin | 745 | WIAF\|WIAF-1214 |
| 12 | 617.8 cR from top of Chr12 lin | 2690 | WIAF\|WIAF-375 |
| 12 | 621.0 cR from top of Chr12 lin | 1261 | WIAF\|WIAF-2148 |
| 12 | 627.9 cR from top of Chr12 lin | 2814 | WIAF\|WIAF-607 |
| 12 | 628.7 cR from top of Chr12 lin | 2964 | WIAF\|WIAF-864 |
| 12 | 644.5 cR from top of Chr12 lin | 2180 | WIAF\|WIAF-562 |
| 12 | | 4118 | SHGC/AFFYMETRIX\| |
| | | | SNPA-SHGC-13972 |
| 12 | | 4128 | SHGC/AFFYMETRIX\| |
| | | | SNPB-SHGC-13972 |
| 12 | | 4003 | SHGC/AFFYMETRIX\|SNP-SHGC-12981 |
| 12 | | 4066 | SHGC/AFFYMETRIX\|SNP-SHGC-13464 |
| 12 | | 4070 | SHGC/AFFYMETRIX\|SNP-SHGC-13943 |
| 12 | | 4163 | SHGC/AFFYMETRIX\|SNP-SHGC-14942 |
| 12 | | 4078 | SHGC/AFFYMETRIX\|SNP-SHGC-16483 |
| 12 | | 4108 | SHGC/AFFYMETRIX\|SNP-SHGC-35771 |
| 12 | | 4036 | SHGC/AFFYMETRIX\|SNP-SHGC-7632 |
| 12 | | 4051 | SHGC/AFFYMETRIX\|SNP-SHGC-9454 |
| 12 | | 3498 | WIAF\|WIAF-1953 |
| 12 | | 3772 | WIAF\|WIAF-2500 |
| 12 | | 2988 | WIAF\|WIAF-888 |
| 13 | 18.60 cR from top of Chr13 lin | 1655 | WIAF\|WIAF-3594 |
| 13 | 23.30 cR from top of Chr13 lin | 1855 | WIAF\|WIAF-3794 |
| 13 | 24.00 cR from top of Chr13 lin | 3527 | WIAF\|WIAF-1982 |
| 13 | 24.00 cR from top of Chr13 lin | 3528 | WIAF\|WIAF-1983 |
| 13 | 27.70 cR from top of Chr13 lin | 2044 | WIAF\|WIAF-1647 |
| 13 | 41.20 cR from top of Chr13 lin | 1251 | WIAF\|WIAF-2138 |
| 13 | 42.30 cR from top of Chr13 lin | 1438 | WIAF\|WIAF-3367 |
| 13 | 44.10 cR from top of Chr13 lin | 3342 | WIAF\|WIAF-1795 |
| 13 | 46.60 cR from top of Chr13 lin | 3185 | WIAF\|WIAF-1501 |
| 13 | 46.60 cR from top of Chr13 lin | 3186 | WIAF\|WIAF-1502 |
| 13 | 46.7 cR from top of Chr13 link | 923 | WIAF\|WIAF-1227 |
| 13 | 47.50 cR from top of Chr13 lin | 2551 | WIAF\|WIAF-173 |
| 13 | 47.60 cR from top of Chr13 lin | 3403 | WIAF\|WIAF-1858 |
| 13 | 47.90 cR from top of Chr13 lin | 1837 | WIAF\|WIAF-3776 |
| 13 | 58.00 cR from top of Chr13 lin | 1451 | WIAF\|WIAF-3384 |
| 13 | 58.50 cR from top of Chr13 lin | 1444 | WIAF\|WIAF-3374 |
| 13 | 59.40 cR from top of Chr13 lin | 1604 | WIAF\|WIAF-3543 |
| 13 | 59.80 cR from top of Chr13 lin | 1422 | WIAF\|WIAF-3350 |
| 13 | 59.80 cR from top of Chr13 lin | 1424 | WIAF\|WIAF-3352 |
| 13 | 59.80 cR from top of Chr13 lin | 1425 | WIAF\|WIAF-3353 |
| 13 | 62.00 cR from top of Chr13 lin | 1897 | WIAF\|WIAF-3836 |
| 13 | 66.20 cR from top of Chr13 lin | 2542 | WIAF\|WIAF-159 |
| 13 | 69.80 cR from top of Chr13 lin | 3196 | WIAF\|WIAF-1531 |
| 13 | 72.00 cR from top of Chr13 lin | 1317 | WIAF\|WIAF-2207 |
| 13 | 72.50 cR from top of Chr13 lin | 1453 | WIAF\|WIAF-3386 |
| 13 | 76.1 cR from top of Chr13 link | 3864 | WIAF\|WIAF-2657 |
| 13 | 76.1 cR from top of Chr13 link | 3865 | WIAF\|WIAF-2659 |
| 13 | 77.10 cR from top of Chr13 lin | 1365 | WIAF\|WIAF-3268 |
| 13 | 78.30 cR from top of Chr13 lin | 1817 | WIAF\|WIAF-3756 |
| 13 | 79.2 cR from top of Chr13 link | 930 | WIAF\|WIAF-1270 |
| 13 | 83.4 cR from top of Chr13 link | 2610 | WIAF\|WIAF-256 |
| 13 | 87.1 cR from top of Chr13 link | 2047 | WIAF\|WIAF-1686 |
| 13 | 87.1 cR from top of Chr13 link | 2048 | WIAF\|WIAF-1687 |
| 13 | 89.10 cR from top of Chr13 lin | 2930 | WIAF\|WIAF-829 |
| 13 | 92.80 cR from top of Chr13 lin | 1612 | WIAF\|WIAF-3551 |
| 13 | 117.50 cR from top of Chr13 li | 1411 | WIAF\|WIAF-3332 |
| 13 | 122.3 cR from top of Chr13 lin | 3139 | WIAF\|WIAF-1040 |
| 13 | 125.1 cR from top of Chr13 lin | 781 | WIAF\|WIAF-1455 |
| 13 | 125.1 cR from top of Chr13 lin | 782 | WIAF\|WIAF-1456 |
| 13 | 125.1 cR from top of Chr13 lin | 783 | WIAF\|WIAF-1457 |
| 13 | 134.3 cR from top of Chr13 lin | 3156 | WIAF\|WIAF-1057 |
| 13 | 143.1 cR from top of Chr13 lin | 2471 | WIAF\|WIAF-65 |
| 13 | 144.1 cR from top of Chr13 in | 2099 | WIAF\|WIAF-156 |
| 13 | 144.1 cR from top of Chr13 lin | 2100 | WIAF\|WIAF-157 |
| 13 | 145.4 cR from top of Chr13 lin | 3011 | WIAF\|WIAF-911 |
| 13 | 145.4 cR from top of Chr13 lin | 3012 | WIAF\|WIAF-912 |
| 13 | 145.4 cR from top of Chr13 lin | 3064 | WIAF\|WIAF-965 |
| 13 | 149.7 cR from top of Chr13 lin | 3481 | WIAF\|WIAF-1936 |
| 13 | 152.4 cR from top of Chr13 lin | 3137 | WIAF\|WIAF-1038 |
| 13 | 192.1 cR from top of Chr13 lin | 2786 | WIAF\|WIAF-540 |
| 13 | 192.1 cR from top of Chr13 lin | 2787 | WIAF\|WIAF-541 |
| 13 | 195.4 cR from top of Chr13 lin | 789 | WIAF\|WIAF-2062 |
| 13 | 275.9 cR from top of Chr13 lin | 2197 | WIAF\|WIAF-600 |
| 13 | 288.1 cR from top of Chr13 lin | 2159 | WIAF\|WIAF-497 |
| 13 | 295.6 cR from top of Chr13 lin | 3842 | WIAF\|WIAF-2619 |
| 13 | 296.6 cR from top of Chr13 lin | 4562 | HU-CHINA\|13-401-1 |
| 13 | | 4229 | MARSHFIELD\|MID-22 |
| 13 | | 4143 | SHGC/AFFYMETRIX\|SNP-SHGC-13649 |
| 13 | | 4146 | SHGC/AFFYMETRIX\|SNP-SHGC-13999 |
| 13 | | 4083 | SHGC/AFFYMETRIX\|SNP-SHGC-16887 |
| 13 | | 4096 | SHGC/AFFYMETRIX\|SNP-SHGC-18881 |
| 13 | | 4008 | SHGC/AFFYMETRIX\|SNP-SHGC-2426 |
| 13 | | 4102 | SHGC/AFFYMETRIX\|SNP-SHGC-30142 |
| 13 | | 4022 | SHGC/AFFYMETRIX\|SNP-SHGC-4718 |
| 13 | | 4034 | SHGC/AFFYMETRIX\|SNP-SHGC-6784 |
| 13 | | 4039 | SHGC/AFFYMETRIX\|SNP-SHGC-8465 |
| 13 | | 3389 | WIAF\|WIAF-1843 |
| 13 | | 2982 | WIAF\|WIAF-882 |
| 13 | | 2491 | WIAF\|WIAF-89 |
| 13 | | 2492 | WIAF\|WIAF-90 |
| 13 | | 3072 | WIAF\|WIAF-973 |
| 14 | 3.30 cR from top of Chr14 link | 3304 | WIAF\|WIAF-1757 |
| 14 | 3.30 cR from top of Chr14 link | 3305 | WIAF\|WIAF-1758 |
| 14 | 3.30 cR from top of Chr14 link | 3306 | WIAF\|WIAF-1759 |
| 14 | 3.30 cR from top of Chr14 link | 3307 | WIAF\|WIAF-1760 |
| 14 | 5.90 cR from top of Chr14 link | 2785 | WIAF\|WIAF-536 |
| 14 | 13.5 cR from top of Chr14 link | 2796 | WIAF\|WIAF-554 |
| 14 | 17.00 cR from top of Chr14 lin | 1948 | WIAF\|WIAF-3887 |
| 14 | 20.6 cR from top of Chr14 link | 4567 | HU-CHINA\|14-729 |
| 14 | 20.6 cR from top of Chr14 link | 2271 | WIAF\|WIAF-729 |
| 14 | 22.4 cR from top of Chr14 link | 3845 | WIAF\|WIAF-2624 |
| 14 | 27.7 cR from top of Chr14 link | 2696 | WIAF\|WIAF-382 |
| 14 | 27.7 cR from top of Chr14 link | 2906 | WIAF\|WIAF-804 |
| 14 | 32.10 cR from top of Chr14 lin | 2915 | WIAF\|WIAF-814 |
| 14 | 36.50 cR from top of Chr14 lin | 1428 | WIAF\|WIAF-3356 |
| 14 | 36.50 cR from top of Chr14 lin | 1429 | WIAF\|WIAF-3357 |
| 14 | 37.00 cR from top of Chr14 lin | 1709 | WIAF\|WIAF-3648 |
| 14 | 37.10 cR from top of Chr14 lin | 701 | WIAF\|WIAF-1296 |
| 14 | 42.40 cR from top of Chr14 lin | 1260 | WIAF\|WIAF-2147 |
| 14 | 46.10 cR from top of Chr14 lin | 1689 | WIAF\|WIAF-3628 |
| 14 | 53.80 cR from top of Chr14 lin | 1834 | WIAF\|WIAF-3773 |
| 14 | 54.60 cR from top of Chr14 lin | 1379 | WIAF\|WIAF-3283 |
| 14 | 55.20 cR from top of Chr14 lin | 1701 | WIAF\|WIAF-3640 |
| 14 | 59.70 cR from top of Chr14 lin | 1961 | WIAF\|WIAF-3900 |
| 14 | 62.00 cR from top of Chr14 lin | 1838 | WIAF\|WIAF-3777 |
| 14 | 63.60 cR from top of Chr14 lin | 3281 | WIAF\|WIAF-1734 |
| 14 | 66.50 cR from top of Chr14 lin | 3206 | WIAF\|WIAF-1557 |
| 14 | 66.50 cR from top of Chr14 lin | 3207 | WIAF\|WIAF-1558 |
| 14 | 66.80 cR from top of Chr14 lin | 1402 | WIAF\|WIAF-3322 |
| 14 | 66.80 cR from top of Chr14 lin | 1406 | WIAF\|WIAF-3326 |
| 14 | 66.80 cR from top of Chr14 lin | 1408 | WIAF\|WIAF-3329 |
| 14 | 69.0 cR from top of Chr14 link | 1080 | WIAF\|WIAF-2051 |
| 14 | 71.50 cR from top of Chr14 lin | 1561 | WIAF\|WIAF-3500 |
| 14 | 75 cM | 4309 | UWGC\|128 |
| 14 | 86.30 cR from top of Chr14 lin | 3390 | WIAF\|WIAF-1845 |
| 14 | 95.5 cR from top of Chr14 link | 2900 | WIAF\|WIAF-798 |
| 14 | 99.60 cR from top of Chr14 lin | 1669 | WIAF\|WIAF-3608 |
| 14 | 101.00 cR from top of Chr14 li | 1137 | WIAF\|WIAF-1600 |
| 14 | 101.00 cR from top of Chr14 li | 1138 | WIAF\|WIAF-1601 |
| 14 | 101.00 cR from top of Chr14 li | 1139 | WIAF\|WIAF-1602 |
| 14 | 109.00 cR from top of Chr14 li | 1539 | WIAF\|WIAF-3478 |
| 14 | 109.00 cR from top of Chr14 li | 1541 | WIAF\|WIAF-3480 |
| 14 | 121.60 cR from top of Chr14 li | 1398 | WIAF\|WIAF-3303 |
| 14 | 124.20 cR from top of Chr14 li | 1432 | WIAF\|WIAF-3360 |
| 14 | 124.20 cR from top of Chr14 li | 1631 | WIAF\|WIAF-3570 |
| 14 | 124.20 cR from top of Chr14 li | 1803 | WIAF\|WIAF-3742 |
| 14 | 124.20 cR from top of Chr14 li | 1805 | WIAF\|WIAF-3744 |
| 14 | 124.20 cR from top of Chr14 li | 1908 | WIAF\|WIAF-3847 |
| 14 | 125.8 cR from top of Chr14 lin | 3545 | WIAF\|WIAF-2000 |
| 14 | 126.2 cR from top of Chr14 lin | 744 | WIAF\|WIAF-1211 |
| 14 | 141.7 cR from top of Chr14 lin | 2249 | WIAF\|WIAF-694 |
| 14 | 167.2 cR from top of Chr14 lin | 751 | WIAF\|WIAF-1263 |
| 14 | 168.5 cR from top of Chr14 lin | 863 | WIAF\|WIAF-1073 |
| 14 | 168.5 cR from top of Chr14 lin | 1011 | WIAF\|WIAF-4046 |
| 14 | 174.5 cR from top of Chr14 lin | 3301 | WIAF\|WIAF-1754 |
| 14 | 179.1 cR from top of Chr14 lin | 3542 | WIAF\|WIAF-1997 |
| 14 | 197.4 cR from top of Chr14 lin | 2640 | WIAF\|WIAF-307 |
| 14 | 197.6 cR from top of Chr14 lin | 3465 | WIAF\|WIAF-1920 |
| 14 | 228.7 cR from top of Chr14 lin | 2859 | WIAF\|WIAF-754 |
| 14 | 248.8 cR from top of Chr14 lin | 2709 | WIAF\|WIAF-402 |
| 14 | 252.9 cR from top of Chr14 lin | 3135 | WIAF\|WIAF-1036 |
| 14 | 252.9 cR from top of Chr14 lin | 3136 | WIAF\|WIAF-1037 |
| 14 | 253.1 cR from top of Chr14 lin | 2924 | WIAF\|WIAF-823 |
| 14 | 253.4 cR from top of Chr14 lin | 3876 | WIAF\|WIAF-2677 |
| 14 | 255.0 cR from top of Chr14 lin | 3361 | WIAF\|WIAF-1814 |
| 14 | 255.1 cR from top of Chr14 lin | 3325 | WIAF\|WIAF-1778 |
| 14 | 255.3 cR from top of Chr14 lin | 2602 | WIAF\|WIAF-246 |
| 14 | 263.3 cR from top of Chr14 lin | 2599 | WIAF\|WIAF-239 |
| 14 | 278.2 cR from top of Chr14 lin | 3132 | WIAF\|WIAF-1033 |
| 14 | 298.2 cR from top of Chr14 lin | 2240 | WIAF\|WIAF-680 |
| 14 | 308.8 cR from top of Chr14 lin | 961 | WIAF\|WIAF-1352 |
| 14 | 324.3 cR from top of Chr14 lin | 2936 | WIAF\|WIAF-835 |
| 14 | 324.3 cR from top of Chr14 lin | 2937 | WIAF\|WIAF-836 |
| 14 | 335.6 cR from top of Chr14 lin | 4582 | HU-CHINA\|14-2041 |
| 14 | 335.6 cR from top of Chr14 lin | 4555 | HU-CH1NA\|14-2041-2 |
| 14 | 335.6 cR from top of Chr14 lin | 3583 | WIAF\|WIAF-2041 |
| 14 | 352.4 cR from top of Chr14 lin | 3362 | WIAF\|WIAF-1815 |
| 14 | 355.5 cR from top of Chr14 lin | 2653 | WIAF\|WIAF-327 |
| 14 | 355.6 cR from top of Chr14 lin | 3440 | WIAF\|WIAF-1895 |
| 14 | 359.0 cR from top of Chr14 lin | 3294 | WIAF\|WIAF-1747 |
| 14 | 363.7 cR from top of Chr14 lin | 2647 | WIAF\|WIAF-318 |
| 14 | | 3997 | SHGC/AFFYMETRIX\|SNP-SHGC-1127 |
| 14 | | 4137 | SHGC/AFFYMETRIX\|SNP-SHGC-13065 |
| 14 | | 4157 | SHGC/AFFYMETRIX\|SNP-SHGC-14530 |
| 14 | | 4088 | SHGC/AFFYMETRIX\|SNP-SHGC-17097 |
| 14 | | 4101 | SHGC/AFFYMETRIX\|SNP-SHGC-19244 |
| 14 | | 4168 | SHGC/AFFYMETRIX\|SNP-SHGC-23875 |
| 14 | | 4032 | SHGC/AFFYMETRIX\|SNP-SHGC-6098 |
| 14 | | 4045 | SHGC/AFFYMETRIX\|SNP-SHGC-9043 |
| 14 | | 3125 | WIAF\|WIAF-1026 |
| 14 | | 666 | WIAF\|WIAF-1072 |
| 14 | | 3595 | WIAF\|WIAF-2187 |
| 14 | | 3596 | WIAF\|WIAF-2188 |
| 14 | | 1328 | WIAF\|WIAF-2218 |
| 14 | | 1589 | WIAF\|WIAF-3528 |
| 14 | | 2728 | WIAF\|WIAF-434 |
| 14 | | 2729 | WIAF\|WIAF-435 |
| 14 | | 2974 | WIAF\|WIAF-874 |
| 15 | 0.00 cR from top of Chr15 link | 3577 | WIAF\|WIAF-2032 |
| 15 | 4.70 cR from top of Chr15 link | 1924 | WIAF\|WIAF-3863 |
| 15 | 5.40 cR from top of Chr15 link | 1712 | WIAF\|WIAF-3651 |
| 15 | 9.60 cR from top of Chr15 link | 2880 | WIAF\|WIAF-778 |
| 15 | 11.00 cR from top of Chr15 lin | 696 | WIAF\|WIAF-1254 |
| 15 | 13.7 cR from top of Chr15 link | 2931 | WIAF\|WIAF-830 |
| 15 | 13.7 cR from top of Chr15 link | 2932 | WIAF\|WIAF-831 |
| 15 | 17.70 cR from top of Chr15 lin | 710 | WIAF\|WIAF-1439 |
| 15 | 21.70 cR from top of Chr15 lin | 3547 | WIAF\|WIAF-2002 |
| 15 | 22.90 cR from top of Chr15 lin | 3153 | WIAF\|WIAF-1054 |
| 15 | 25.50 cR from top of Chr15 lin | 2904 | WIAF\|WIAF-802 |
| 15 | 28.9 cR from top of Chr15 link | 2058 | WIAF\|WIAF-2543 |
| 15 | 28.9 cR from top of Chr15 link | 3032 | WIAF\|WIAF-932 |
| 15 | 37.30 cR from top of Chr15 lin | 1968 | WIAF\|WIAF-3907 |
| 15 | 38.20 cR from top of Chr15 lin | 2473 | WIAF\|WIAF-68 |
| 15 | 42.0 cR from top of Chr15 link | 2625 | WIAF\|WIAF-284 |
| 15 | 42.70 cR from top of Chr15 lin | 2469 | WIAF\|WIAF-62 |
| 15 | 46 cM | 4304 | UWGC\|123 |
| 15 | 46.20 cR from top of Chr15 lin | 3231 | WIAF\|WIAF-1633 |
| 15 | 46.30 cR from top of Chr15 lin | 3513 | WIAF\|WIAF-1968 |
| 15 | 46.30 cR from top of Chr15 lin | 1670 | WIAF\|WIAF-3609 |
| 15 | 46.30 cR from top of Chr15 lin | 2426 | WIAF\|WIAF-6 |
| 15 | 46.40 cR from top of Chr15 lin | 1944 | WIAF\|WIAF-3883 |
| 15 | 46.8 cR from top of Chr15 link | 2956 | WIAF\|WIAF-855 |
| 15 | 46.90 cR from top of Chr15 lin | 1382 | WIAF\|WIAF-3287 |
| 15 | 47 cM | 4305 | UWGC\|124 |
| 15 | 48.10 cR from top of Chr15 lin | 1951 | WIAF\|WIAF-3890 |
| 15 | 48.20 cR from top of Chr15 lin | 3232 | WIAF\|WIAF-1635 |
| 15 | 49.70 cR from top of Chr15 lin | 695 | WIAF\|WIAF-1248 |
| 15 | 49.9 cR from top of Chr15 link | 2893 | WIAF\|WIAF-791 |
| 15 | 53 cM | 4318 | UWGC\|137 |
| 15 | 53.70 cR from top of Chr15 lin | 3176 | WIAF\|WIAF-1491 |
| 15 | 60.90 cR from top of Chr15 lin | 1695 | WIAF\|WIAF-3634 |
| 15 | 65.30 cR from top of Chr15 lin | 1434 | WIAF\|WIAF-3362 |
| 15 | 65.30 cR from top of Chr15 lin | 1436 | WIAF\|WIAF-3364 |
| 15 | 65.8 cR from top of Chr15 link | 3093 | WIAF\|WIAF-994 |
| 15 | 65.8 cR from top of Chr15 link | 3094 | WIAF\|WIAF-995 |
| 15 | 70.7 cR from top of Chr15 link | 3573 | WIAF\|WIAF-2028 |
| 15 | 71.30 cR from top of Chr15 lin | 3489 | WIAF\|WIAF-1944 |
| 15 | 71.30 cR from top of Chr15 lin | 3490 | WIAF\|WIAF-1945 |
| 15 | 71.80 cR from top of Chr15 lin | 3557 | WIAF\|WIAF-2012 |
| 15 | 72.20 cR from top of Chr15 lin | 1578 | WIAF\|WIAF-3517 |
| 15 | 72.70 cR from top of Chr15 lin | 1680 | WIAF\|WIAF-3619 |
| 15 | 73.70 cR from top of Chr15 lin | 3175 | WIAF\|WIAF-1490 |
| 15 | 74.90 cR from top of Chr15 lin | 1974 | WIAF\|WIAF-3913 |
| 15 | 75.50 cR from top of Chr15 lin | 3452 | WIAF\|WIAF-1907 |
| 15 | 75.70 cR from top of Chr15 lin | 3160 | WIAF\|WIAF-1459 |
| 15 | 76.40 cR from top of Chr15 lin | 1937 | WIAF\|WIAF-3876 |
| 15 | 76.6 cR from top of Chr15 link | 3719 | WIAF\|WIAF-2447 |
| 15 | 77.30 cR from top of Chr15 lin | 1835 | WIAF\|WIAF-3774 |
| 15 | 77.40 cR from top of Chr15 lin | 2015 | WIAF\|WIAF-1509 |
| 15 | 78.60 cR from top of Chr15 lin | 1744 | WIAF\|WIAF-3683 |
| 15 | 85.4 cR from top of Chr15 link | 2101 | WIAF\|WIAF-163 |
| 15 | 94.40 cR from top of Chr15 lin | 1190 | WIAF\|WIAF-2077 |
| 15 | 97.60 cR from top of Chr15 lin | 1760 | WIAF\|WIAF-3699 |
| 15 | 102.60 cR from top of Chr15 li | 1648 | WIAF\|WIAF-3587 |
| 15 | 104.40 cR from top of Chr15 li | 1910 | WIAF\|WIAF-3849 |
| 15 | 105.4 cR from top of Chr15 lin | 2646 | WIAF\|WIAF-317 |
| 15 | 105.5 cR from top of Chr15 lin | 2711 | WIAF\|WIAF-406 |
| 15 | 108.70 cR from top of Chr15 li | 1911 | WIAF\|WIAF-3850 |
| 15 | 108.70 cR from top of Chr15 li | 1914 | WIAF\|WIAF-3853 |
| 15 | 121.4 cR from top of Chr15 lin | 3496 | WIAF\|WIAF-1951 |
| 15 | 121.7 cR from top of Chr15 lin | 3406 | WIAF\|WIAF-1861 |
| 15 | 133.5 cR from top of Chr15 lin | 3812 | WIAF\|WIAF-2564 |
| 15 | 139.7 cR from top of Chr15 lin | 2174 | WIAF\|WIAF-547 |
| 15 | 142.1 cR from top of Chr15 lin | 2097 | WIAF\|WIAF-140 |
| 15 | 144.2 cR from top of Chr15 lin | 2660 | WIAF\|WIAF-336 |
| 15 | 152.5 cR from top of Chr15 lin | 2490 | WIAF\|WIAF-88 |
| 15 | 159.6 cR from top of Chr15 lin | 2345 | WIAF\|WIAF-2576 |
| 15 | 180.8 cR from top of Chr15 lin | 2679 | WIAF\|WIAF-362 |
| 15 | 194.2 cR from top of Chr15 lin | 2585 | WIAF\|WIAF-219 |
| 15 | 195.6 cR from top of Chr15 lin | 3099 | WIAF\|WIAF-1000 |
| 15 | 202.7 cR from top of Chr15 lin | 2721 | WIAF\|WIAF-425 |
| 15 | 207.0 cR from top of Chr15 lin | 2539 | WIAF\|WIAF-153 |
| 15 | 228.4 cR from top of Chr15 lin | 3110 | WIAF\|WIAF-1011 |
| 15 | 228.4 cR from top of Chr15 lin | 3111 | WIAF\|WIAF-1012 |
| 15 | 228.6 cR from top of Chr15 lin | 2949 | WIAF\|WIAF-848 |
| 15 | 247.4 cR from top of Chr15 lin | 1053 | WIAF\|WIAF-4162 |
| 15 | 286.2 cR from top of Chr15 lin | 2369 | WIAF\|WIAF-2630 |
| 15 | 306.5 cR from top of Chr15 lin | 856 | WIAF\|WIAF-453 |
| 15 | 306.5 cR from top of Chr15 lin | 2740 | WIAF\|WIAF-454 |
| 15 | 308.4 cR from top of Chr15 lin | 3969 | SHGC/AFFYMETRIX\|SNP-SHGC-14665 |
| 15 | 332.8 cR from top of Chr15 lin | 2115 | WIAF\|WIAF-243 |
| 15 | 332.8 cR from top of Chr15 lin | 2116 | WIAF\|WIAF-244 |
| 15 | 344.1 cR from top of Chr15 lin | 2584 | WIAF\|WIAF-218 |
| 15 | 355.1 cR from top of Chr15 lin | 2733 | WIAF\|WIAF-443 |
| 15 | 355.1 cR from top of Chr15 lin | 2734 | WIAF\|WIAF-444 |
| 15 | 363.7 cR from top of Chr15 lin | 3457 | WIAF\|WIAF-1912 |
| 15 | 388.6 cR from top of Chr15 lin | 2557 | WIAF\|WIAF-181 |
| 15 | 396.8 cR from top of Chr15 lin | 1054 | WIAF\|WIAF-4163 |
| 15 | 396.8 cR from top of Chr15 lin | 2980 | WIAF\|WIAF-880 |
| 15 | | 4120 | SHGC/AFFYMETRIX\| |
| | | | SNPA-SHGC-15063 |
| 15 | | 4130 | SHGC/AFFYMETRIX\| |
| | | | SNPB-SHGC-15063 |
| 15 | | 4139 | SHGC/AFFYMETRIX\|SNP-SHGC-13105 |
| 15 | | 4148 | SHGC/AFFYMETRIX\|SNP-SHGC-14096 |
| 15 | | 4154 | SHGC/AFFYMETRIX\|SNP-SHGC-14356 |
| 15 | | 3971 | SHGC/AFFYMETRIX\|SNP-SHGC-17150 |
| 15 | | 4047 | SHGC/AFFYMETRIX\|SNP-SHGC-9310 |
| 15 | | 3386 | WIAF\|WIAF-1840 |
| 15 | | 3684 | WIAF\|WIAF-2412 |
| 15 | | 3761 | WIAF\|WIAF-2489 |
| 15 | | 1774 | WIAF\|WIAF-3713 |
| 15 | | 1928 | WIAF\|WIAF-3867 |
| 15 | | 3906 | WIAF\|WIAF-3980 |
| 15 | | 3920 | WIAF\|WIAF-4007 |
| 15 | | 3025 | WIAF\|WIAF-925 |
| 15 | | 3026 | WIAF\|WIAF-926 |
| 16 | 5.10 cR from top of Chr16 link | 1151 | WIAF\|WIAF-1628 |
| 16 | 5.60 cR from top of Chr16 link | 3191 | WIAF\|WIAF-1514 |
| 16 | 5.80 cR from top of Chr16 link | 1773 | WIAF\|WIAF-3712 |
| 16 | 9.00 cR from top of Chr16 link | 693 | WIAF\|WIAF-1244 |
| 16 | 10.40 cR from top of Chr16 lin | 3024 | WIAF\|WIAF-924 |
| 16 | 14.60 cR from top of Chr16 lin | 1404 | WIAF\|WIAF-3324 |
| 16 | 15.50 cR from top of Chr16 lin | 1889 | WIAF\|WIAF-3828 |
| 16 | 20.60 cR from top of Chr16 lin | 1223 | WIAF\|WIAF-2110 |
| 16 | 21.4 cR from top of Chr16 link | 2710 | WIAF\|WIAF-403 |
| 16 | 22.1 cR from top of Chr16 link | 2766 | WIAF\|WIAF-506 |
| 16 | 25.2 cR from top of Chr16 link | 2481 | WIAF\|WIAF-77 |
| 16 | 25.5 cR from top of Chr16 link | 2891 | WIAF\|WIAF-789 |
| 16 | 30.60 cR from top of Chr16 lin | 3550 | WIAF\|WIAF-2005 |
| 16 | 37.6 cR from top of Chr16 link | 2760 | WIAF\|WIAF-495 |
| 16 | 41.60 cR from top of Chr16 lin | 2036 | WIAF\|WIAF-1613 |
| 16 | 42.70 cR from top of Chr16 lin | 1485 | WIAF\|WIAF-3424 |
| 16 | 42.70 cR from top of Chr16 lin | 1489 | WIAF\|WIAF-3428 |
| 16 | 43.30 cR from top of Chr16 lin | 1649 | WIAF\|WIAF-3588 |
| 16 | 44.0 cR from top of Chr16 link | 3021 | WIAF\|WIAF-921 |
| 16 | 69.70 cR from top of Chr16 lin | 4566 | HU-CHINA\|16-1697 |
| 16 | 69.70 cR from top of Chr16 lin | 3261 | WIAF\|WIAF-1697 |
| 16 | 70.3 cR from top of Chr16 link | 2770 | WIAF\|WIAF-510 |
| 16 | 72.60 cR from top of Chr16 lin | 1249 | WIAF\|WIAF-2136 |
| 16 | 74.20 cR from top of Chr16 lin | 690 | WIAF\|WIAF-1210 |
| 16 | 75.80 cR from top of Chr16 lin | 1965 | WIAF\|WIAF-3904 |
| 16 | 86.50 cR from top of Chr16 lin | 1775 | WIAF\|WIAF-3714 |
| 16 | 87.50 cR from top of Chr16 lin | 1704 | WIAF\|WIAF-3643 |
| 16 | 91.50 cR from top of Chr16 lin | 1495 | WIAF\|WIAF-3434 |
| 16 | 91.50 cR from top of Chr16 lin | 1496 | WIAF\|WIAF-3435 |
| 16 | 92.40 cR from top of Chr16 lin | 3359 | WIAF\|WIAF-1812 |
| 16 | 97.90 cR from top of Chr16 lin | 3166 | WIAF\|WIAF-1474 |
| 16 | 98.0 cR from top of Chr16 link | 2759 | WIAF\|WIAF-494 |
| 16 | 98.10 cR from top of Chr16 lin | 3454 | WIAF\|WIAF-1909 |
| 16 | 98.20 cR from top of Chr16 lin | 1671 | WIAF\|WIAF-3610 |
| 16 | 103.10 cR from top of Chr16 li | 1555 | WIAF\|WIAF-3494 |
| 16 | 103.10 cR from top of Chr16 li | 1654 | WIAF\|WIAF-3593 |
| 16 | 107.60 cR from top of Chr16 li | 3121 | WIAF\|WIAF-1022 |
| 16 | 109.20 cR from top of Chr16 li | 1435 | WIAF\|WIAF-3363 |
| 16 | 109.20 cR from top of Chr16 li | 1437 | WIAF\|WIAF-3366 |
| 16 | 109.20 cR from top of Chr16 li | 1439 | WIAF\|WIAF-3368 |
| 16 | 109.40 cR from top of Chr16 li | 2251 | WIAF\|WIAF-697 |
| 16 | 112.3 cR from top of Chr16 lin | 2375 | WIAF\|WIAF-2652 |
| 16 | 113.8 cR from top of Chr16 lin | 3057 | WIAF\|WIAF-958 |
| 16 | 113.9 cR from top of Chr16 lin | 3046 | WIAF\|WIAF-947 |
| 16 | 113.9 cR from top of Chr16 lin | 3047 | WIAF\|WIAF-948 |
| 16 | 119.10 cR from top of Chr16 li | 1912 | WIAF\|WIAF-3851 |
| 16 | 119.10 cR from top of Chr16 li | 1916 | WIAF\|WIAF-3855 |
| 16 | 122.1 cR from top of Chr16 lin | 2791 | WIAF\|WIAF-546 |
| 16 | 123.30 cR from top of Chr16 li | 2054 | WIAF\|WIAF-1717 |
| 16 | 123.30 cR from top of Chr16 li | 2055 | WIAF\|WIAF-1718 |
| 16 | 130.80 cR from top of Chr16 li | 1255 | WIAF\|WIAF-2142 |
| 16 | 130.80 cR from top of Chr16 li | 1776 | WIAF\|WIAF-3715 |
| 16 | 131.4 cR from top of Chr16 lin | 2989 | WIAF\|WIAF-889 |
| 16 | 140.1 cR from top of Chr16 lin | 2122 | WIAF\|WIAF-285 |
| 16 | 227.3 cR from top of Chr16 lin | 3409 | WIAF\|WIAF-1864 |
| 16 | 235.6 cR from top of Chr16 lin | 3516 | WIAF\|WIAF-1971 |
| 16 | 242.9 cR from top of Chr16 lin | 2241 | WIAF\|WIAF-681 |
| 16 | 242.9 cR from top of Chr16 lin | 2242 | WIAF\|WIAF-682 |
| 16 | 305.1 cR from top of Chr16 lin | 1270 | WIAF\|WIAF-2157 |
| 16 | 312.9 cR from top of Chr16 lin | 3058 | WIAF\|WIAF-959 |
| 16 | 320.4 cR from top of Chr16 lin | 2468 | WIAF\|WIAF-61 |
| 16 | 327.3 cR from top of Chr16 lin | 3556 | WIAF\|WIAF-2011 |
| 16 | 330.5 cR from top of Chr16 lin | 3860 | WIAF\|WIAF-2650 |
| 16 | 333.4 cR from top of Chr16 lin | 2515 | WIAF\|WIAF-123 |
| 16 | 333.6 cR from top of Chr16 lin | 2560 | WIAF\|WIAF-184 |
| 16 | 338.2 cR from top of Chr16 lin | 2519 | WIAF\|WIAF-127 |
| 16 | 348.6 cR from top of Chr16 lin | 892 | WIAF\|WIAF-1139 |
| 16 | 351.6 cR from top of Chr16 lin | 2146 | WIAF\|WIAF-437 |
| 16 | 351.6 cR from top of Chr16 lin | 2147 | WIAF\|WIAF-438 |
| 16 | 351.6 cR from top of Chr16 lin | 2182 | WIAF\|WIAF-564 |
| 16 | | 4230 | MARSHFIELD\|MID-23 |
| 16 | | 3966 | SHGC/AFFYMETRIX\|SNP-SHGC-12011 |
| 16 | | 4038 | SHGC/AFFYMETRIX\|SNP-SHGC-8152 |
| 16 | | 1146 | WIAF\|WIAF-1614 |
| 16 | | 3671 | WIAF\|WIAF-2399 |
| 16 | | 2066 | WIAF\|WIAF-2553 |
| 16 | | 3810 | WIAF\|WIAF-2562 |
| 16 | | 1482 | WIAF\|WIAF-3421 |
| 16 | | 1486 | WIAF\|WIAF-3425 |
| 16 | | 1527 | WIAF\|WIAF-3466 |
| 16 | | 1565 | WIAF\|WIAF-3504 |
| 16 | | 2960 | WIAF\|WIAF-859 |
| 16 | | 2992 | WIAF\|WIAF-892 |
| 17 | 0.60 cR from top of Chr17 link | 2435 | WIAF\|WIAF-18 |
| 17 | 0.60 cR from top of Chr17 link | 3467 | WIAF\|WIAF-1922 |
| 17 | 0.60 cR from top of Chr17 link | 1399 | WIAF\|WIAF-3305 |
| 17 | 1.40 cR from top of Chr17 link | 1726 | WIAF\|WIAF-3665 |
| 17 | 2.10 cR from top of Chr17 link | 1108 | WIAF\|WIAF-1540 |
| 17 | 4.50 cR from top of Chr17 link | 3115 | WIAF\|WIAF-1016 |
| 17 | 5.90 cR from top of Chr17 link | 3549 | WIAF\|WIAF-2004 |
| 17 | 7.60 cR from top of Chr17 link | 1741 | WIAF\|WIAF-3680 |
| 17 | 7.60 cR from top of Chr17 link | 2451 | WIAF\|WIAF-39 |
| 17 | 15.00 cR from top of Chr17 lin | 1240 | WIAF\|WIAF-2127 |
| 17 | 16.30 cR from top of Chr17 lin | 1586 | WIAF\|WIAF-3525 |
| 17 | 16.50 cR from top of Chr17 lin | 1175 | WIAF\|WIAF-1699 |
| 17 | 16.80 cR from top of Chr17 lin | 2035 | WIAF\|WIAF-1598 |
| 17 | 16.80 cR from top of Chr17 lin | 1721 | WIAF\|WIAF-3660 |
| 17 | 16.80 cR from top of Chr17 lin | 2460 | WIAF\|WIAF-51 |
| 17 | 19 cM | 4313 | UWGC\|132 |
| 17 | 29.30 cR from top of Chr17 lin | 1812 | WIAF\|WIAF-3751 |
| 17 | 33.5 cR from top of Chr17 link | 2922 | WIAF\|WIAF-821 |
| 17 | 36.40 cR from top of Chr17 lin | 2553 | WIAF\|WIAF-176 |
| 17 | 45.20 cR from top of Chr17 lin | 1473 | WIAF\|WIAF-3410 |
| 17 | 45.40 cR from top of Chr17 lin | 1950 | WIAF\|WIAF-3889 |
| 17 | 45.5 cR from top of Chr17 link | 3018 | WIAF\|WIAF-918 |
| 17 | 45.5 cR from top of Chr17 link | 3041 | WIAF\|WIAF-942 |
| 17 | 51.8 cR from top of Chr17 link | 3062 | WIAF\|WIAF-963 |
| 17 | 51.90 cR from top of Chr17 lin | 709 | WIAF\|WIAF-1419 |
| 17 | 53.10 cR from top of Chr17 lin | 2018 | WIAF\|WIAF-1519 |
| 17 | 57.30 cR from top of Chr17 lin | 3541 | WIAF\|WIAF-1996 |
| 17 | 59.9 cR from top of Chr17 link | 2846 | WIAF\|WIAF-699 |
| 17 | 60.10 cR from top of Chr17 lin | 1258 | WIAF\|WIAF-2145 |
| 17 | 60.60 cR from top of Chr17 lin | 1441 | WIAF\|WIAF-3370 |
| 17 | 61.10 cR from top of Chr17 lin | 1696 | WIAF\|WIAF-3635 |
| 17 | 62.8 cR from top of Chr17 link | 2564 | WIAF\|WIAF-193 |
| 17 | 62.80 cR from top of Chr17 lin | 3387 | WIAF\|WIAF-1841 |
| 17 | 63.10 cR from top of Chr17 lin | 3183 | WIAF\|WIAF-1499 |
| 17 | 63.40 cR from top of Chr17 lin | 684 | WIAF\|WIAF-1108 |
| 17 | 64.80 cR from top of Chr17 lin | 1997 | WIAF\|WIAF-3936 |
| 17 | 65.00 cR from top of Chr17 lin | 1840 | WIAF\|WIAF-3779 |
| 17 | 65.00 cR from top of Chr17 lin | 1841 | WIAF\|WIAF-3780 |
| 17 | 66.10 cR from top of Chr17 lin | 3453 | WIAF\|WIAF-1908 |
| 17 | 67.00 cR from top of Chr17 lin | 1637 | WIAF\|WIAF-3576 |
| 17 | 68.10 cR from top of Chr17 lin | 3242 | WIAF\|WIAF-1652 |
| 17 | 73.00 cR from top of Chr17 lin | 3350 | WIAF\|WIAF-1803 |
| 17 | 83.90 cR from top of Chr17 lin | 3397 | WIAF\|WIAF-1852 |
| 17 | 84.10 cR from top of Chr17 lin | 1094 | WIAF\|WIAF-1479 |
| 17 | 84.90 cR from top of Chr17 lin | 700 | WIAF\|WIAF-1276 |
| 17 | 84.90 cR from top of Chr17 lin | 673 | WIAF\|WIAF-1376 |
| 17 | 86.30 cR from top of Chr17 lin | 671 | WIAF\|WIAF-1361 |
| 17 | 86.70 cR from top of Chr17 lin | 1416 | WIAF\|WIAF-3343 |
| 17 | 87.60 cR from top of Chr17 lin | 1898 | WIAF\|WIAF-3837 |
| 17 | 94.1 cR from top of Chr17 link | 2278 | WIAF\|WIAF-746 |
| 17 | 94.1 cR from top of Chr17 link | 2279 | WIAF\|WIAF-747 |
| 17 | 94.1 cR from top of Chr17 link | 2280 | WIAF\|WIAF-748 |
| 17 | 103.5 cR from top of Chr17 lin | 2087 | WIAF\|WIAF-101 |
| 17 | 250.6 cR from top of Chr17 lin | 3008 | WIAF\|WIAF-908 |
| 17 | 304.7 cR from top of Chr17 lin | 2975 | WIAF\|WIAF-875 |
| 17 | 307.9 cR from top of Chr17 lin | 3288 | WIAF\|WIAF-1741 |
| 17 | 311.1 cR from top of Chr17 lin | 2844 | WIAF\|WIAF-688 |
| 17 | 317.4 cR from top of Chr17 lin | 2858 | WIAF\|WIAF-752 |
| 17 | 329.4 cR from top of Chr17 lin | 2861 | WIAF\|WIAF-758 |
| 17 | 338.1 cR from top of Chr17 lin | 2869 | WIAF\|WIAF-767 |
| 17 | 338.6 cR from top of Chr17 lin | 2567 | WIAF\|WIAF-196 |
| 17 | 355.3 cR from top of Chr17 lin | 2767 | WIAF\|WIAF-507 |
| 17 | 355.5 cR from top of Chr17 lin | 3000 | WIAF\|WIAF-900 |
| 17 | 371.5 cR from top of Chr17 lin | 3821 | WIAF\|WIAF-2582 |
| 17 | 445.5 cR from top of Chr17 lin | 2842 | WIAF\|WIAF-684 |
| 17 | 462.1 cR from top of Chr17 lin | 3570 | WIAF\|WIAF-2025 |
| 17 | | 4053 | SHGC/AFFYMETRIX\| |
| | | | SNPA-SHGC-31580 |
| 17 | | 4059 | SHGC/AFFYMETRIX\| |
| | | | SNPB-SHGC-31580 |
| 17 | | 4001 | SHGC/AFFYMETRIX\|SNP-SHGC-1216 |
| 17 | | 4006 | SHGC/AFFYMETRIX\|SNP-SHGC-1310 |
| 17 | | 4072 | SHGC/AFFYMETRIX\|SNP-SHGC-14793 |
| 17 | | 4092 | SHGC/AFFYMETRIX\|SNP-SHGC-17275 |
| 17 | | 4165 | SHGC/AFFYMETRIX\|SNP-SHGC-18143 |
| 17 | | 4095 | SHGC/AFFYMETRIX\|SNP-SHGC-18839 |
| 17 | | 4015 | SHGC/AFFYMETRIX\|SNP-SHGC-3939 |
| 17 | | 3120 | WIAF\|WIAF-1021 |
| 17 | | 3127 | WIAF\|WIAF-1028 |
| 17 | | 2550 | WIAF\|WIAF-171 |
| 17 | | 1335 | WIAF\|WIAF-2225 |
| 17 | | 3678 | WIAF\|WIAF-2406 |
| 17 | | 3777 | WIAF\|WIAF-2505 |
| 17 | | 3778 | WIAF\|WIAF-2506 |
| 17 | | 3800 | WIAF\|WIAF-2529 |
| 17 | | 2463 | WIAF\|WIAF-55 |
| 17 | | 3073 | WIAF\|WIAF-974 |
| 18 | 7.40 cR from top of Chr18 link | 2011 | WIAF\|WIAF-1505 |
| 18 | 7.40 cR from top of Chr18 link | 2012 | WIAF\|WIAF-1506 |
| 18 | 7.90 cR from top of Chr18 link | 1189 | WIAF\|WIAF-2076 |
| 18 | 19.5 cR from top of Chr18 link | 3834 | WIAF\|WIAF-2603 |
| 18 | 20.90 cR from top of Chr18 lin | 3226 | WIAF\|WIAF-1626 |
| 18 | 21.1 cR from top of Chr18 link | 2820 | WIAF\|WIAF-621 |
| 18 | 28.1 cR from top of Chr18 link | 3848 | WIAF\|WIAF-2631 |
| 18 | 32.1 cR from top of Chr18 link | 2819 | WIAF\|WIAF-620 |
| 18 | 35.0 cR from top of Chr18 link | 4584 | HU-CHINA\|18-525 |
| 18 | 35.0 cR from top of Chr18 link | 4557 | HU-CHINA\|18-525-2 |
| 18 | 35.0 cR from top of Chr18 link | 2163 | WIAF\|WIAF-525 |
| 18 | 35.0 cR from top of Chr18 link | 2164 | WIAF\|WIAF-526 |
| 18 | 36.20 cR from top of Chr18 lin | 3355 | WIAF\|WIAF-1808 |
| 18 | 36.20 cR from top of Chr18 lin | 3356 | WIAF\|WIAF-1809 |
| 18 | 43.10 cR from top of Chr18 lin | 1250 | WIAF\|WIAF-2137 |
| 18 | 45.4 cR from top of Chr18 link | 2587 | WIAF\|WIAF-222 |
| 18 | 45.6 cR from top of Chr18 link | 3101 | WIAF\|WIAF-1002 |
| 18 | 52.5 cR from top of Chr18 link | 3027 | WIAF\|WIAF-927 |
| 18 | 56.2 cR from top of Chr18 link | 3278 | WIAF\|WIAF-1731 |
| 18 | 56.2 cR from top of Chr18 link | 3279 | WIAF\|WIAF-1732 |
| 18 | 56.2 cR from top of Chr18 link | 3280 | WIAF\|WIAF-1733 |
| 18 | 57.00 cR from top of Chr18 lin | 1940 | WIAF\|WIAF-3879 |
| 18 | 58.1 cR from top of Chr18 link | 3100 | WIAF\|WIAF-1001 |
| 18 | 61.50 cR from top of Chr18 lin | 1127 | WIAF\|WIAF-1571 |
| 18 | 61.50 cR from top of Chr18 lin | 1128 | WIAF\|WIAF-1572 |
| 18 | 61.60 cR from top of Chr18 lin | 3164 | WIAF\|WIAF-1468 |
| 18 | 66.60 cR from top of Chr18 lin | 2486 | WIAF\|WIAF-84 |
| 18 | 66.70 cR from top of Chr18 lin | 1501 | WIAF\|WIAF-3440 |
| 18 | 66.70 cR from top of Chr18 lin | 1719 | WIAF\|WIAF-3658 |
| 18 | 68.20 cR from top of Chr18 lin | 2007 | WIAF\|WIAF-1471 |
| 18 | 72.30 cR from top of Chr18 lin | 3322 | WIAF\|WIAF-1775 |
| 18 | 80.30 cR from top of Chr18 lin | 1596 | WIAF\|WIAF-3535 |
| 18 | 81.60 cR from top of Chr18 lin | 1697 | WIAF\|WIAF-3636 |
| 18 | 81.60 cR from top of Chr18 lin | 1700 | WIAF\|WIAF-3639 |
| 18 | 109.00 cR from top of Chr18 li | 2918 | WIAF\|WIAF-817 |
| 18 | 202.8 cR from top of Chr18 lin | 2436 | WIAF\|WIAF-21 |
| 18 | 288.2 cR from top of Chr18 lin | 989 | WIAF\|WIAF-1432 |
| 18 | 288.2 cR from top of Chr18 lin | 1016 | WIAF\|WIAF-4064 |
| 18 | 321.0 cR from top of Chr18 lin | 3358 | WIAF\|WIAF-1811 |
| 18 | 323.9 cR from top of Chr18 lin | 2781 | WIAF\|WIAF-530 |
| 18 | 337.2 cR from top of Chr18 lin | 2093 | WIAF\|WIAF-112 |
| 18 | 355.2 cR from top of Chr18 lin | 910 | WIAF\|WIAF-1187 |
| 18 | 355.2 cR from top of Chr18 lin | 911 | WIAF\|WIAF-1188 |
| 18 | 394.1 cR from top of Chr18 lin | 2282 | WIAF\|WIAF-753 |
| 18 | 454.4 cR from top of Chr18 lin | 2109 | WIAF\|WIAF-210 |
| 18 | 455.8 cR from top of Chr18 lin | 2252 | WIAF\|WIAF-700 |
| 18 | 455.8 cR from top of Chr18 lin | 3029 | WIAF\|WIAF-929 |
| 18 | 500.2 cR from top of Chr18 lin | 2657 | WIAF\|WIAF-331 |
| 18 | 509.8 cR from top of Chr18 lin | 2903 | WIAF\|WIAF-801 |
| 18 | 541.3 cR from top of Chr18 lin | 2658 | WIAF\|WIAF-332 |
| 18 | 544.5 cR from top of Chr18 lin | 2373 | WIAF\|WIAF-2642 |
| 18 | | 4231 | MARSHFIELD\|MID-24 |
| 18 | | 4232 | MARSHFIELD\|MID-25 |
| 18 | | 4145 | SHGC/AFFYMETRIX\|SNP-SHGC-13976 |
| 18 | | 4161 | SHGC/AFFYMETRIX\|SNP-SHGC-14744 |
| 18 | | 4085 | SHGC/AFFYMETRIX\|SNP-SHGC-16937 |
| 18 | | 4105 | SHGC/AFFYMETRIX\|SNP-SHGC-31408 |
| 18 | | 3972 | SHGC/AFFYMETRIX\|SNP-SHGC-32292 |
| 18 | | 3364 | WIAF\|WIAF-1817 |
| 18 | | 3007 | WIAF\|WIAF-907 |
| 19 | 14.50 cR from top of Chr19 lin | 2735 | WIAF\|WIAF-446 |
| 19 | 16.30 cR from top of Chr19 lin | 4589 | HU-CHINA\|19-1653 |
| 19 | 16.30 cR from top of Chr19 lin | 3243 | WIAF\|WIAF-1653 |
| 19 | 16.30 cR from top of Chr19 lin | 3380 | WIAF\|WIAF-1833 |
| 19 | 17.50 cR from top of Chr19 lin | 1468 | WIAF\|WIAF-3403 |
| 19 | 20.80 cR from top of Chr19 lin | 1333 | WIAF\|WIAF-2223 |
| 19 | 27.90 cR from top of Chr19 lin | 1459 | WIAF\|WIAF-3393 |
| 19 | 27.90 cR from top of Chr19 lin | 1460 | WIAF\|WIAF-3394 |
| 19 | 35.50 cR from top of Chr19 lin | 1990 | WIAF\|WIAF-3929 |
| 19 | 36.30 cR from top of Chr19 lin | 1574 | WIAF\|WIAF-3513 |
| 19 | 36.50 cR from top of Chr19 lin | 1767 | WIAF\|WIAF-3706 |
| 19 | 37.50 cR from top of Chr19 lin | 2512 | WIAF\|WIAF-118 |
| 19 | 42.20 cR from top of Chr19 lin | 1297 | WIAF\|WIAF-2184 |
| 19 | 42.80 cR from top of Chr19 lin | 2520 | WIAF\|WIAF-128 |
| 19 | 47.90 cR from top of Chr19 lin | 1779 | WIAF\|WIAF-3718 |
| 19 | 47.90 cR from top of Chr19 lin | 1780 | WIAF\|WIAF-3719 |
| 19 | 50.00 cR from top of Chr19 lin | 1431 | WIAF\|WIAF-3359 |
| 19 | 55.60 cR from top of Chr19 lin | 2940 | WIAF\|WIAF-839 |
| 19 | 56.40 cR from top of Chr19 lin | 1106 | WIAF\|WIAF-1533 |
| 19 | 58.10 cR from top of Chr19 lin | 1483 | WIAF\|WIAF-3422 |
| 19 | 59.90 cR from top of Chr19 lin | 1836 | WIAF\|WIAF-3775 |
| 19 | 61.80 cR from top of Chr19 lin | 2041 | WIAF\|WIAF-1625 |
| 19 | 63.60 cR from top of Chr19 lin | 1316 | WIAF\|WIAF-2206 |
| 19 | 63.60 cR from top of Chr19 lin | 1321 | WIAF\|WIAF-2211 |
| 19 | 65.20 cR from top of Chr19 lin | 1957 | WIAF\|WIAF-3896 |
| 19 | 68.20 cR from top of Chr19 lin | 1656 | WIAF\|WIAF-3595 |
| 19 | 68.70 cR from top of Chr19 lin | 1934 | WIAF\|WIAF-3873 |
| 19 | 69.30 cR from top of Chr19 lin | 3205 | WIAF\|WIAF-1556 |
| 19 | 69.30 cR from top of Chr19 lin | 1607 | WIAF\|WIAF-3546 |
| 19 | 69.30 cR from top of Chr19 lin | 1609 | WIAF\|WIAF-3548 |
| 19 | 69.60 cR from top of Chr19 lin | 1593 | WIAF\|WIAF-3532 |
| 19 | 70.2 cR from top of Chr19 link | 1114 | WIAF\|WIAF-1549 |
| 19 | 84.00 cR from top of Chr19 lin | 1550 | WIAF\|WIAF-3489 |
| 19 | 84.00 cR from top of Chr19 lin | 1552 | WIAF\|WIAF-3491 |
| 19 | 88.00 cR from top of Chr19 lin | 1624 | WIAF\|WIAF-3563 |
| 19 | 88.00 cR from top of Chr19 lin | 1628 | WIAF\|WIAF-3567 |
| 19 | 89.8 cR from top of Chr19 link | 3039 | WIAF\|WIAF-940 |
| 19 | 95.40 cR from top of Chr19 lin | 1723 | WIAF\|WIAF-3662 |
| 19 | 97.60 cR from top of Chr19 lin | 1376 | WIAF\|WIAF-3280 |
| 19 | 97.90 cR from top of Chr19 lin | 1668 | WIAF\|WIAF-3607 |
| 19 | 99.80 cR from top of Chr19 lin | 1172 | WIAF\|WIAF-1689 |
| 19 | 100.30 cR from top of Chr19 li | 1214 | WIAF\|WIAF-2101 |
| 19 | 103.60 cR from top of Chr19 li | 1964 | WIAF\|WIAF-3903 |
| 19 | 104.90 cR from top of Chr19 li | 2427 | WIAF\|WIAF-7 |
| 19 | 106.70 cR from top of Chr19 li | 1584 | WIAF\|WIAF-3523 |
| 19 | 107.40 cR from top of Chr19 li | 1487 | WIAF\|WIAF-3426 |
| 19 | 109.90 cR from top of Chr19 li | 3534 | WIAF\|WIAF-1989 |
| 19 | 109.90 cR from top of Chr19 li | 1476 | WIAF\|WIAF-3414 |
| 19 | 256.4 cR from top of Chr19 lin | 2343 | WIAF\|WIAF-2574 |
| 19 | 280.9 cR from top of Chr19 lin | 3493 | WIAF\|WIAF-1948 |
| 19 | 285.1 cR from top of Chr19 lin | 2888 | WIAF\|WIAF-786 |
| 19 | 286.2 cR from top of Chr19 lin | 2617 | WIAF\|WIAF-264 |
| 19 | 290.4 cR from top of Chr19 lin | 2731 | WIAF\|WIAF-439 |
| 19 | 290.7 cR from top of Chr19 lin | 2717 | WIAF\|WIAF-416 |
| 19 | 324.0 cR from top of Chr19 lin | 3582 | WIAF\|WIAF-2037 |
| 19 | 324.0 cR from top of Chr19 lin | 3077 | WIAF\|WIAF-978 |
| 19 | 325.6 cR from top of Chr19 lin | 3854 | WIAF\|WIAF-2641 |
| 19 | 331.1 cR from top of Chr19 lin | 2423 | WIAF\|WIAF-1 |
| 19 | 331.2 cR from top of Chr19 lin | 2832 | WIAF\|WIAF-654 |
| 19 | 341.9 cR from top of Chr19 lin | 3045 | WIAF\|WIAF-946 |
| 19 | 349.3 cR from top of Chr19 lin | 4591 | HU-CHINA\|19-941 |
| 19 | 349.3 cR from top of Chr19 lin | 4592 | HU-CHINA\|19-941-2 |
| 19 | 349.3 cR from top of Chr19 lin | 3040 | WIAF\|WIAF-941 |
| 19 | 380.7 cR from top of Chr19 lin | 3867 | WIAF\|WIAF-2662 |
| 19 | 382.3 cR from top of Chr19 lin | 3372 | WIAF\|WIAF-1825 |
| 19 | 382.3 cR from top of Chr19 lin | 3373 | WIAF\|WIAF-1826 |
| 19 | 382.8 cR from top of Chr19 lin | 3851 | WIAF\|WIAF-2637 |
| 19 | 385.1 cR from top of Chr19 lin | 3485 | WIAF\|WIAF-1940 |
| 19 | | 4054 | SHGC/AFFYMETRIX\| |
| | | | SNPA-SHGC-35310 |
| 19 | | 4060 | SHGC/AFFYMETRIX\| |
| | | | SNPB-SHGC-35310 |
| 19 | | 3957 | SHGC/AFFYMETRIX\|SNPB-SHGC-9656 |
| 19 | | 3963 | SHGC/AFFYMETRIX\|SNP-SHGC-11607 |
| 19 | | 4068 | SHGC/AFFYMETRIX\|SNP-SHGC-13495 |
| 19 | | 3174 | WIAF\|WIAF-1488 |
| 19 | | 2038 | WIAF\|WIAF-1618 |
| 19 | | 3239 | WIAF\|WIAF-1649 |
| 19 | | 3253 | WIAF\|WIAF-1671 |
| 19 | | 3787 | WIAF\|WIAF-2515 |
| 19 | | 1740 | WIAF\|WIAF-3679 |
| 19 | | 2952 | WIAF\|WIAF-851 |
| 19 | | 2985 | WIAF\|WIAF-885 |
| 19 | | 2986 | WIAF\|WIAF-886 |
| 19 | | 2993 | WIAF\|WIAF-893 |
| 19 | | 2994 | WIAF\|WIAF-894 |
| 19 | | 3023 | WIAF\|WIAF-923 |
| 19 | | 3071 | WIAF\|WIAF-972 |
| 20 | 7.10 cR from top of Chr20 link | 1242 | WIAF\|WIAF-2129 |
| 20 | 8.20 cR from top of Chr20 link | 2057 | WIAF\|WIAF-1720 |
| 20 | 9.30 cR from top of Chr20 link | 1842 | WIAF\|WIAF-3781 |
| 20 | 9.40 cR from top of Chr20 link | 1232 | WIAF\|WIAF-2119 |
| 20 | 9.80 cR from top of Chr20 link | 1125 | WIAF\|WIAF-1568 |
| 20 | 9.80 cR from top of Chr20 link | 1126 | WIAF\|WIAF-1569 |
| 20 | 9.80 cR from top of Chr20 link | 3344 | WIAF\|WIAF-1797 |
| 20 | 10.1 cR from top of Chr20 link | 2856 | WIAF\|WIAF-749 |
| 20 | 10.10 cR from top of Chr20 lin | 2494 | WIAF\|WIAF-92 |
| 20 | 14.7 cR from top of Chr20 link | 2432 | WIAF\|WIAF-15 |
| 20 | 22.00 cR from top of Chr20 lin | 1880 | WIAF\|WIAF-3819 |
| 20 | 23.2 cR from top of Chr20 link | 3551 | WIAF\|WIAF-2006 |
| 20 | 24.7 cR from top of Chr20 link | 2745 | WIAF\|WIAF-464 |
| 20 | 24.7 cR from top of Chr20 link | 2746 | WIAF\|WIAF-465 |
| 20 | 25.6 cR from top of Chr20 link | 2851 | WIAF\|WIAF-730 |
| 20 | 30.60 cR from top of Chr20 lin | 3256 | WIAF\|WIAF-1684 |
| 20 | 32.60 cR from top of Chr20 lin | 1570 | WIAF\|WIAF-3509 |
| 20 | 32.60 cR from top of Chr20 lin | 1572 | WIAF\|WIAF-3511 |
| 20 | 35.10 cR from top of Chr20 lin | 1650 | WIAF\|WIAF-3589 |
| 20 | 36.80 cR from top of Chr20 lin | 2040 | WIAF\|WIAF-1621 |
| 20 | 39.90 cR from top of Chr20 lin | 1241 | WIAF\|WIAF-2128 |
| 20 | 41.20 cR from top of Chr20 lin | 2002 | WIAF\|WIAF-3941 |
| 20 | 41.40 cR from top of Chr20 lin | 2000 | WIAF\|WIAF-3939 |
| 20 | 41.60 cR from top of Chr20 lin | 1225 | WIAF\|WIAF-2112 |
| 20 | 41.60 cR from top of Chr20 lin | 2713 | WIAF\|WIAF-410 |
| 20 | 41.70 cR from top of Chr20 lin | 1786 | WIAF\|WIAF-3725 |
| 20 | 42.20 cR from top of Chr20 lin | 1988 | WIAF\|WIAF-3927 |
| 20 | 42.70 cR from top of Chr20 lin | 3013 | WIAF\|WIAF-913 |
| 20 | 47.80 cR from top of Chr20 lin | 2887 | WIAF\|WIAF-785 |
| 20 | 48.70 cR from top of Chr20 lin | 1490 | WIAF\|WIAF-3429 |
| 20 | 49 cM | 4325 | UWGC\|144 |
| 20 | 53.00 cR from top of Chr20 lin | 2896 | WIAF\|WIAF-794 |
| 20 | 55.00 cR from top of Chr20 lin | 1181 | WIAF\|WIAF-1711 |
| 20 | 55.00 cR from top of Chr20 lin | 2626 | WIAF\|WIAF-286 |
| 20 | 55.40 cR from top of Chr20 lin | 1758 | WIAF\|WIAF-3697 |
| 20 | 62.40 cR from top of Chr20 lin | 2009 | WIAF\|WIAF-1481 |
| 20 | 63.30 cR from top of Chr20 lin | 3564 | WIAF\|WIAF-2019 |
| 20 | 65.30 cR from top of Chr20 lin | 3398 | WIAF\|WIAF-1853 |
| 20 | 74.00 cR from top of Chr20 lin | 1099 | WIAF\|WIAF-1515 |
| 20 | 74.00 cR from top of Chr20 lin | 1875 | WIAF\|WIAF-3814 |
| 20 | 82.10 cR from top of Chr20 lin | 3514 | WIAF\|WIAF-1969 |
| 20 | 82.50 cR from top of Chr20 lin | 1537 | WIAF\|WIAF-3476 |
| 20 | 82.80 cR from top of Chr20 lin | 1978 | WIAF\|WIAF-3917 |
| 20 | 86.00 cR from top of Chr20 lin | 1745 | WIAF\|WIAF-3684 |
| 20 | 88.30 cR from top of Chr20 lin | 1507 | WIAF\|WIAF-3446 |
| 20 | 89.5 cR from top of Chr20 link | 2083 | WIAF\|WIAF-67 |
| 20 | 91.2 cR from top of Chr20 link | 2130 | WIAF\|WIAF-333 |
| 20 | 96.50 cR from top of Chr20 lin | 1998 | WIAF\|WIAF-3937 |
| 20 | 96.50 cR from top of Chr20 lin | 2971 | WIAF\|WIAF-871 |
| 20 | 106.6 cR from top of Chr20 lin | 2501 | WIAF\|WIAF-100 |
| 20 | 310.1 cR from top of Chr20 lin | 2283 | WIAF\|WIAF-756 |
| 20 | 316.2 cR from top of Chr20 lin | 2441 | WIAF\|WIAF-26 |
| 20 | 318.0 cR from top of Chr20 lin | 2628 | WIAF\|WIAF-288 |
| 20 | 318.9 cR from top of Chr20 lin | 3365 | WIAF\|WIAF-1818 |
| 20 | 319.9 cR from top of Chr20 lin | 3540 | WIAF\|WIAF-1995 |
| 20 | 320.2 cR from top of Chr20 lin | 2082 | WIAF\|WIAF-42 |
| 20 | 320.6 cR from top of Chr20 lin | 771 | WIAF\|WIAF-1402 |
| 20 | 334.7 cR from top of Chr20 lin | 4577 | HU-CHINA\|20-1357 |
| 20 | 334.7 cR from top of Chr20 lin | 763 | WIAF\|WIAF-1357 |
| 20 | 334.7 cR from top of Chr20 lin | 822 | WIAF\|WIAF-4221 |
| 20 | 343.6 cR from top of Chr20 lin | 2871 | WIAF\|WIAF-769 |
| 20 | 343.6 cR from top of Chr20 lin | 2872 | WIAF\|WIAF-770 |
| 20 | 343.6 cR from top of Chr20 lin | 2873 | WIAF\|WIAF-771 |
| 20 | 343.6 cR from top of Chr20 lin | 2874 | WIAF\|WIAF-772 |
| 20 | | 4086 | SHGC/AFFYMETRIX\|SNP-SHGC-16962 |
| 20 | | 4009 | SHGC/AFFYMETRIX\|SNP-SHGC-2774 |
| 20 | | 4010 | SHGC/AFFYMETRIX\|SNP-SHGC-2775 |
| 20 | | 4033 | SHGC/AFFYMETRIX\|SNP-SHGC-6179 |
| 20 | | 3238 | WIAF\|WIAF-1648 |
| 20 | | 3699 | WIAF\|WIAF-2427 |
| 20 | | 3052 | WIAF\|WIAF-953 |
| 21 | 11.30 cR from top of Chr21 lin | 1558 | WIAF\|WIAF-3497 |
| 21 | 11.30 cR from top of Chr21 lin | 1559 | WIAF\|WIAF-3498 |
| 21 | 17.60 cR from top of Chr21 lin | 1623 | WIAF\|WIAF-3562 |
| 21 | 30.50 cR from top of Chr21 lin | 1606 | WIAF\|WIAF-3545 |
| 21 | 31.8 cR from top of Chr21 link | 2243 | WIAF\|WIAF-683 |
| 21 | 32.50 cR from top of Chr21 lin | 1856 | WIAF\|WIAF-3795 |
| 21 | 35.10 cR from top of Chr21 lin | 1557 | WIAF\|WIAF-3496 |
| 21 | 35.30 cR from top of Chr21 lin | 1569 | WIAF\|WIAF-3508 |
| 21 | 38.20 cR from top of Chr21 lin | 3488 | WIAF\|WIAF-1943 |
| 21 | 38.30 cR from top of Chr21 lin | 3197 | WIAF\|WIAF-1534 |
| 21 | 39.30 cR from top of Chr21 lin | 1718 | WIAF\|WIAF-3657 |
| 21 | 45.00 cR from top of Chr21 lin | 1547 | WIAF\|WIAF-3486 |
| 21 | 57.40 cR from top of Chr21 lin | 3200 | WIAF\|WIAF-1537 |
| 21 | 57.40 cR from top of Chr21 lin | 3201 | WIAF\|WIAF-1538 |
| 21 | 58.30 cR from top of Chr21 lin | 1540 | WIAF\|WIAF-3479 |
| 21 | 58.30 cR from top of Chr21 lin | 1543 | WIAF\|WIAF-3482 |
| 21 | 59.30 cR from top of Chr21 lin | 1324 | WIAF\|WIAF-2214 |
| 21 | 59.30 cR from top of Chr21 lin | 1325 | WIAF\|WIAF-2215 |
| 21 | 59.60 cR from top of Chr21 lin | 4570 | HU-CHINA\|21-899 |
| 21 | 59.60 cR from top of Chr21 lin | 689 | WIAF\|WIAF-1199 |
| 21 | 59.60 cR from top of Chr21 lin | 1166 | WIAF\|WIAF-1678 |
| 21 | 59.60 cR from top of Chr21 lin | 1746 | WIAF\|WIAF-3685 |
| 21 | 59.60 cR from top of Chr21 lin | 1748 | WIAF\|WIAF-3687 |
| 21 | 59.60 cR from top of Chr21 lin | 1802 | WIAF\|WIAF-3741 |
| 21 | 59.60 cR from top of Chr21 lin | 2999 | WIAF\|WIAF-899 |
| 21 | 119.1 cR from top of Chr21 lin | 2206 | WIAF\|WIAF-624 |
| 21 | 153.3 cR from top of Chr21 lin | 3855 | WIAF\|WIAF-2643 |
| 21 | 153.3 cR from top of Chr21 lin | 3859 | WIAF\|WIAF-2648 |
| 21 | 157.0 cR from top of Chr21 lin | 2434 | WIAF\|WIAF-17 |
| 21 | 233.7 cR from top of Chr21 lin | 3438 | WIAF\|WIAF-1893 |
| 21 | | 3989 | SHGC/AFFYMETRIX\|SNPA-SHGC-9556 |
| 21 | | 4141 | SHGC/AFFYMETRIX\|SNP-SHGC-13352 |
| 21 | | 4011 | SHGC/AFFYMETRIX\|SNP-SHGC-2811 |
| 21 | | 4211 | SHGC/AFFYMETRIX\|SNP-SHGC-51813 |
| 21 | | 4212 | SHGC/AFFYMETRIX\|SNP-SHGC-51844 |
| 21 | | 4213 | SHGC/AFFYMETRIX\|SNP-SHGC-51849 |
| 21 | | 4214 | SHGC/AFFYMETRIX\|SNP-SHGC-51852 |
| 21 | | 3982 | SHGC/AFFYMETRIX\|SNP-SHGC-51888 |
| 21 | | 4215 | SHGC/AFFYMETRIX\|SNP-SHGC-51907 |
| 21 | | 4216 | SHGC/AFFYMETRIX\|SNP-SHGC-51925 |
| 21 | | 4030 | SHGC/AFFYMETRIX\|SNP-SHGC-51941 |
| 21 | | 4217 | SHGC/AFFYMETRIX\|SNP-SHGC-51944 |
| 21 | | 4031 | SHGC/AFFYMETRIX\|SNP-SHGC-51951 |
| 21 | | 3184 | WIAF\|WIAF-1500 |
| 21 | | 1170 | WIAF\|WIAF-1682 |
| 21 | | 1171 | WIAF\|WIAF-1683 |
| 21 | | 3402 | WIAF\|WIAF-1857 |
| 21 | | 3427 | WIAF\|WIAF-1882 |
| 21 | | 1212 | WIAF\|WIAF-2099 |
| X | 1.7 cR from top of ChrX linkag | 2384 | WIAF\|WIAF-2675 |
| X | 7.0 cR from top of ChrX linkag | 2188 | WIAF\|WIAF-576 |
| X | 7.4 cR from top of ChrX linkag | 2096 | WIAF\|WIAF-137 |
| X | 11.10 cR from top of ChrX link | 1293 | WIAF\|WIAF-2180 |
| X | 11.10 cR from top of ChrX link | 1294 | WIAF\|WIAF-2181 |
| X | 15.0 cR from top of ChrX linka | 3870 | WIAF\|WIAF-2669 |
| X | 15.0 cR from top of ChrX linka | 3075 | WIAF\|WIAF-976 |
| X | 15.7 cR from top of ChrX linka | 2148 | WIAF\|WIAF-440 |
| X | 23.5 cR from top of ChrX linka | 773 | WIAF\|WIAF-1404 |
| X | 28.10 cR from top of ChrX link | 2573 | WIAF\|WIAF-203 |
| X | 28.10 cR from top of ChrX link | 1244 | WIAF\|WIAF-2131 |
| X | 30.10 cR from top of ChrX link | 1833 | WIAF\|WIAF-3772 |
| X | 41.4 cR from top of ChrX linka | 3303 | WIAF\|WIAF-1756 |
| X | 43.4 cR from top of ChrX linka | 2803 | WIAF\|WIAF-572 |
| X | 43.4 cR from top of ChrX linka | 2804 | WIAF\|WIAF-573 |
| X | 59.80 cR from top of ChrX link | 3486 | WIAF\|WIAF-1941 |
| X | 91.6 cR from top of ChrX linka | 2608 | WIAF\|WIAF-254 |
| X | 91.6 cR from top of ChrX linka | 2609 | WIAF\|WIAF-255 |
| X | 91.8 cR from top of ChrX linka | 3408 | WIAF\|WIAF-1863 |
| X | 92.20 cR from top of ChrX link | 3417 | WIAF\|WIAF-1872 |
| X | 96.80 cR from top of ChrX link | 3548 | WIAF\|WIAF-2003 |
| X | 120.30 cR from top of ChrX lin | 705 | WIAF\|WIAF-1356 |
| X | 187.70 cR from top of ChrX lin | 3290 | WIAF\|WIAF-1743 |
| X | 187.70 cR from top of ChrX lin | 3291 | WIAF\|WIAF-1744 |
| X | 232.1 cR from top of ChrX link | 2257 | WIAF\|WIAF-707 |
| X | 282.4 cR from top of ChrX link | 761 | WIAF\|WIAF-1350 |
| X | 285.4 cR from top of ChrX link | 3030 | WIAF\|WIAF-930 |
| X | 290.1 cR from top of ChrX link | 2072 | WIAF\|WIAF-2 |
| X | 294.0 cR from top of ChrX link | 2778 | WIAF\|WIAF-522 |
| X | 301.2 cR from top of ChrX link | 2776 | WIAF WIAF-519 |
| X | 301.3 cR from top of ChrX link | 871 | WIAF\|WIAF-1098 |
| X | 301.3 cR from top of ChrX link | 872 | WIAF\|WIAF-1099 |
| X | 304.8 cR from top of ChrX link | 3824 | WIAF\|WIAF-2589 |
| X | 315.6 cR from top of ChrX link | 3599 | WIAF\|WIAF-2269 |
| X | 315.6 cR from top of ChrX link | 3600 | WIAF\|WIAF-2270 |
| X | 331.3 cR from top of ChrX link | 3104 | WIAF\|WIAF-1005 |
| X | Xp21.2, 32.550 Mb | 625 | KWOK\|Xp1226-1 |
| X | Xp21.2, 32.550 Mb | 624 | KWOK\|Xp1226-2 |
| X | Xq22, 105.900 Mb | 623 | KWOK\|Xq1136-1 |
| X | Xq22, 106.300 Mb | 661 | KWOK\|Xq544-1 |
| X | Xq22, 106.300 Mb | 660 | KWOK\|Xq544-2 |
| X | Xq24, 117.624 Mb | 641 | KWOK\|Xq3562-1 |
| X | Xq24, 117.685 Mb | 646 | KWOK\|Xq3655-2 |
| X | Xq24, 117.754 Mb | 648 | KWOK\|Xq3656-1 |
| X | Xq24, 117.754 Mb | 649 | KWOK\|Xq3656-2 |
| X | Xq24, 117.754 Mb | 647 | KWOK\|Xq3656-3 |
| X | Xq25 | 590 | KWOK\|Xq3855-1 |
| X | Xq25 | 591 | KWOK\|Xq3858-1 |
| X | Xq25, 122.820 Mb | 588 | KWOK\|Xq3847-1 |
| X | Xq25, 124.300 Mb | 593 | KWOK\|Xq3868-1 |
| X | Xq25, 124.362 Mb | 654 | KWOK\|Xq3773-1 |
| X | Xq25, 124.456 Mb | 655 | KWOK\|Xq3774-1 |
| X | Xq25, 124.456 Mb | 656 | KWOK\|Xq3774-2 |
| X | Xq25, 124.673 Mb | 592 | KWOK\|Xq3862-1 |
| X | Xq25, 124.860 Mb | 645 | KWOK\|Xq3570-1 |
| X | Xq25, 124.860 Mb | 644 | KWOK\|Xq3570-2 |
| X | Xq25, 124.860 Mb | 642 | KWOK\|Xq3570-3 |
| X | Xq25, 124.860 Mb | 643 | KWOK\|Xq3570-4 |
| X | Xq25, 125.110 | 621 | KWOK\|Xq3813-1 |
| X | Xq25, 125.110 | 622 | KWOK\|Xq3813-2 |
| X | Xq25, 126.091 Mb | 587 | KWOK\|Xq3846-1 |
| X | Xq25, 126.091 Mb | 586 | KWOK\|Xq3846-2 |
| X | Xq25, 126.257 | 617 | KWOK\|Xq3705-1 |
| X | Xq25, 126.257 | 618 | KWOK\|Xq3705-2 |
| X | Xq25, 126.296 Mb | 657 | KWOK\|Xq3804-1 |
| X | Xq25, 126.387 | 619 | KWOK\|Xq3812-1 |
| X | Xq25, 126.387 | 620 | KWOK\|Xq3812-2 |
| X | Xq25, 126.687 Mb | 589 | KWOK\|Xq3849-1 |
| X | Xq25, 126.846 Mb | 652 | KWOK\|Xq3699-1 |
| X | Xq25, 126.846 Mb | 653 | KWOK\|Xq3699-2 |
| X | Xq25, 126.954 Mb | 658 | KWOK\|Xq3811-1 |
| X | Xq25, 126.954 Mb | 659 | KWOK\|Xq3811-2 |
| X | Xq25, 127.053 Mb | 650 | KWOK\|Xq3698-1 |
| X | Xq25, 127.053 Mb | 651 | KWOK\|Xq3698-2 |
| X | Xq26 | 594 | KWOK\|Xq3871-1 |
| X | Xq26, 131.172 Mb | 597 | KWOK\|Xq3879-1 |
| X | Xq26, 131.990 Mb | 634 | KWOK\|Xq3070-1 |
| X | Xq26, 131.990 Mb | 635 | KWOK\|Xq3070-2 |
| X | Xq26, 136.541 Mb | 662 | KWOK\|Xq3874-1 |
| X | Xq26, 136.631 Mb | 595 | KWOK\|Xq3875-1 |
| X | Xq26, 136.631 Mb | 596 | KWOK\|Xq3875-2 |
| X | Xq26, 137.977 | 608 | KWOK\|Xq3695-1 |
| X | Xq26, 137.977 | 609 | KWOK\|Xq3695-2 |
| X | Xq26, 137.977 | 611 | KWOK\|Xq3695-3 |
| X | Xq26, 137.977 | 610 | KWOK\|Xq3695-4 |
| X | Xq26, 138.062 | 612 | KWOK\|Xq3696-1 |
| X | Xq26, 138.062 | 613 | KWOK\|Xq3696-2 |
| X | Xq26, 138.062 | 614 | KWOK\|Xq3696-3 |
| X | Xq26, 138.062 | 615 | KWOK\|Xq3696-4 |
| X | Xq26, 138.062 | 616 | KWOK\|Xq3696-5 |
| X | Xq27 | 598 | KWOK\|Xq3885-1 |
| X | Xq27 | 599 | KWOK\|Xq3885-2 |
| X | Xq27 | 600 | KWOK\|Xq3886-1 |
| X | Xq27 | 601 | KWOK\|Xq3886-2 |
| X | Xq27, 141.250 Mb | 631 | KWOK\|Xq2904-1 |
| X | Xq27, 141.250 Mb | 632 | KWOK\|Xq2904-2 |
| X | Xq27, 141.250 Mb | 633 | KWOK\|Xq2904-3 |
| X | Xq27, 141.499 Mb | 663 | KWOK\|Xq3887-1 |
| X | Xq27, 141.499 Mb | 664 | KWOK\|Xq3887-2 |
| X | Xq27, 141.580 Mb | 602 | KWOK\|Xq3888-1 |
| X | Xq28 | 603 | KWOK\|Xq3555-1 |
| X | Xq28 | 604 | KWOK\|Xq3555-2 |
| X | Xq28 | 605 | KWOK\|Xq3555-3 |
| X | Xq28 | 606 | KWOK\|Xq3555-4 |
| X | Xq28 | 607 | KWOK\|Xq3555-5 |
| X | Xq28, 157.074 Mb | 585 | KWOK\|Xq3841-1 |
| X | Xq28, 157.123 Mb | 583 | KWOK\|Xq3840-1 |
| X | Xq28, 157.123 Mb | 584 | KWOK\|Xq3840-2 |
| X | Xq28, 157.939 Mb | 640 | KWOK\|Xq3476-1 |
| X | Xq28, 157.939 Mb | 639 | KWOK\|Xq3476-2 |
| X | Xq28, 158.055 Mb | 637 | KWOK\|Xq3449-1 |
| X | Xq28, 158.059 Mb | 638 | KWOK\|Xq3471-1 |
| X | Xq28, 158.237 Mb | 630 | KWOK\|Xq2816-1 |
| X | Xq28, 158.265 Mb | 636 | KWOK\|Xq3274-1 |
| X | Xq28, 158.490 Mb | 626 | KWOK\|Xq1452-1 |
| X | Xq28, 158.490 Mb | 627 | KWOK\|Xq1452-2 |
| X | Xq28, 158.490 Mb | 628 | KWOK\|Xq1452-3 |
| X | Xq28, 158.490 Mb | 629 | KWOK\|Xq1452-4 |
| X | | 4099 | SHGC/AFFYMETRIX\|SNP-SHGC-18945 |
| X | | 2008 | WIAF\|WIAF-1472 |
| X | | 3271 | WIAF\|WIAF-1723 |
| X | | 3272 | WIAF\|WIAF-1724 |
| X | | 3469 | WIAF\|WIAF-1924 |
| X | | 3602 | WIAF\|WIAF-2274 |
| X | | 3869 | WIAF\|WIAF-2666 |
| X | | 3036 | WIAF\|WIAF-936 |
| Y | | 3930 | OEFNER\|M2 |
| Y | | 3930 | OEFNER\|M2 |
| Y | | 3931 | OEFNER\|M3 |
| Y | | 3931 | OEFNER\|M3 |
| Y | | 3932 | OEFNER\|M4 |
| Y | | 3933 | OEFNER\|M5 |
| Y | | 3933 | OEFNER\|M5 |
| Y | | 3934 | OEFNER\|M6 |
| Y | | 3935 | OEFNER\|M7 |
| Y | | 3936 | OEFNER\|M8 |
| Y | | 3937 | OEFNER\|M9 |
| Y | | 3938 | OEFNER\|M10 |
| Y | | 3939 | OEFNER\|M11 |
| Y | | 3940 | OEFNER\|M12 |
| Y | | 3941 | OEFNER\|M13 |
| Y | | 3942 | OEFNER\|M14 |
| Y | | 3943 | OEFNER\|M15 |
| Y | | 3944 | OEFNER\|M16 |
| Y | | 3944 | OEFNER\|M16 |
| Y | | 3945 | OEFNER\|M17 |
| Y | | 3946 | OEFNER\|M18 |
| Y | | 3947 | OEFNER\|M19 |
| Y | | 3948 | OEFNER\|M20 |
| Y | | 3949 | OEFNER\|M21 |
| Y | | 3950 | OEFNER\|M22 |

### E. Methods for Removing Nucleic Acid Duplex with Abnormal Base-Pairing

Provided herein is a method for removing a nucleic acid duplex containing one or more abnormal base-pairing in a population of nucleic acid duplexes, which method comprises: a) contacting a population of nucleic acid duplexes having or suspected of having a nucleic acid duplex containing one or more abnormal base-pairing with a mutant DNA repair enzyme or complex thereof, wherein the mutant DNA repair enzyme or complex thereof has binding affinity for the abnormal base-pairing in the duplex but has attenuated catalytic activity and whereby the nucleic acid duplex containing one or more abnormal base-pairing binds to the mutant DNA repair enzyme or complex thereof to form a binding complex; and b) removing the binding complex formed in step a) from the population of nucleic acid duplexes, thereby the nucleic acid duplex containing one or more abnormal base-pairing is removed from the population of nucleic acid duplexes.

In a specific embodiment, a population of nucleic acid duplexes comprise DNA:DNA, DNA:RNA and RNA:RNA duplexes. Preferably, the population comprises DNA:DNA duplexes.

In another specific embodiment, the nucleic acid duplex to be removed from the population comprise a base-pair mismatch, a base insertion, a base deletion or a pyrimidine dimer. Preferably, the base-pair mismatch is a base-pair mismatch.

In still another specific embodiment, the population of nucleic acid duplexes is produced by an enzymatic amplification. Preferably, the population of nucleic acid duplexes is produced by a polymerase chain reaction or a reaction utilizing reverse transcription and subsequent DNA amplification of one or more expressed RNA sequences.

The binding complex formed between the nucleic acid duplex containing one or more abnormal base-pairing and the mutant DNA repair enzyme or complex thereof can be removed from the population of nucleic acid duplexes by any methods known in the art. For example, the binding complex can be separated from the population by conventional separation methods such as electrophoresis, centrifugation, filtration and chromatograph. The separation can also be effected by affinity separation/purification, i.e., using moieties that bind proteins but not nucleic acids. For example, antibodies that bind proteins generally but not nucleic acids can be used, antibodies that specifically bind the mutant DNA repair enzyme or complex thereof can be used. In addition, the mutant DNA repair enzyme or complex thereof can be labelled and/or tagged and the separation can be effected through the labels or tags.

### F. Methods for Detecting and Localizing Abnormal Base-Pairing in Nucleic Acid Duplex

Also provided herein is a method for detecting and localizing an abnormal base-pairing in a nucleic acid duplex by contacting a nucleic acid duplex having or suspected of having an abnormal base-pairing with a mutant DNA repair enzyme or complex thereof, where the mutant DNA repair enzyme or complex thereof has binding affinity for the abnormal base-pairing in the duplex but has attenuated catalytic activity and whereby the nucleic acid duplex containing an abnormal base-pairing binds to the mutant DNA repair enzyme or complex thereof to form a binding complex; subjecting the nucleic acid duplex to hydrolysis with an exonuclease under conditions such that the binding complex formed in the first step blocks hydrolysis; and then determining the location within the nucleic acid duplex protected from the hydrolysis, thereby detecting and localizing the abnormal base-pairing in the nucleic acid duplex.

In a specific embodiment, the nucleic acid duplex to be assayed is a DNA:DNA, a DNA:RNA or a RNA:RNA duplex. Preferably, the nucleic acid duplex to be assayed is a DNA:DNA.

In another specific embodiment, the abnormal base-pairing to be detected and localized is a base-pair mismatch, a base insertion, a base deletion or a pyrimidine dimer. Preferably, the base-pair mismatch to be detected and localized is a single base-pair mismatch.

Any exonucleases can be used in the present methods. For example, the exonucleases with the following Genbank Accession Nos. can be used: AF194116 (Escherichia coli exonuclease X), AF191741 (Arabidopsis thaliana exonuclease RRP41 (RRP41)), AF013497 (Pyrococcus furiosus endo/exonuclease (fen-1)), AF058396 (Chlamydophila caviae strain GPIC ssDNA-specific exonuclease (recJ)), AF151105 (Homo sapiens 3′-5′ exonuclease TREX1 mRNA), AF151108 (Mus musculus 3′-5′ exonuclease TREX2), AF151107 (Homo sapiens 3′-5′ exonuclease TREX2 mRNA), AF151106 (Mus musculus 3′-5′ exonuclease TREX1), AF083915 (Chilo iridescent virus exonuclease II homolog (EX02)), AF140550 (Salmonella typhimurium exonuclease VII (xseA)), AF134570 (Xenopus laevis exonuclease Exo1 (EXOI)), AF084974 (Homo sapiens exonuclease I (EXOI)), AF030933 (Homo sapiens exonuclease homolog RAD1 (RAD1)), AF034258 (Caenorhabditis elegans exonuclease III homolog), AH006967 (Homo sapiens exonuclease I (EXOI) gene), AF091740 (Homo sapiens exonuclease 1a (EXO1 a), 5174 (Schizosaccharomyces pombe exonuclease I (exo1), AF084514 (Mus musculus DNA repair exonuclease (Rec1)), AF084513 (Homo sapiens DNA repair exonuclease (REC1)), AF084512 (Homo sapiens DNA repair exonuclease (REC1), AF060479 (Homo sapiens exonuclease I (EXO1), U76424 (Lactococcus lactis), U57401 (Choristoneura fumiferana alkaline exonuclease), U58147 (Haemophilus ducreyi), U86134 (Saccharomyces cerevisiae exonuclease 1 (EXO1), U57963 (Erwinia chrysanthemi single-stranded DNA exonuclease (recj) gene), M22592 (E.coli xth gene encoding exonuclease III), J02641 (E.coli sbcB gene encoding exonuclease I), L23927 (Escherichia coli exonuclease VIII (recE)

Preferably, exonucleases that specifically cleave double-stranded nucleic acids, but not single-stranded nucleic acids, are used in the present methods. Also preferably, nuclease BAL-31, exonuclease III, Mung Bean exonuclease or Lambda exonuclease is used.

### G. Labelling of Mutant DNA Repair Enzymes

Conjugates, such as fusion proteins and chemical conjugates, of the mutant DNA repair enzyme with a protein or peptide fragment (or plurality thereof) that functions, for example, to facilitate affinity isolation or purification of the mutant enzyme, attachment of the mutant enzyme to a surface, or detection of the mutant enzyme are provided. The conjugates can be produced by chemical conjugation, such as via thiol linkages, but are preferably produced by recombinant means as fusion proteins. In the fusion protein, the peptide or fragment thereof is linked to either the N-terminus or C-terminus of the mutant enzyme. In chemical conjugates the peptide or fragment thereof may be linked anywhere that conjugation can be effected, and there may be a plurality of such peptides or fragments linked to a single mutant enzyme or to a plurality thereof.

1. Conjugation

Conjugation can be effected by any method known to those of skill in the art. As described below, conjugation can be effected by chemical means, through covalent, ionic or any other suitable linkage.

a. Fusion Proteins

Fusion proteins are provided herein. A fusion protein contains: a) one or a plurality of mutant DNA repair enzymes and b) at least one protein or peptide fragment that facilitates, for example: i) affinity isolation or purification of the fusion protein; ii) attachment of the fusion protein to a surface; or iii) detection of the fusion protein, or any combination thereof.

The facilitating agent is selected to perform the desired purpose, such as (i)-(iii), and is linked a mutant DNA repair enzyme such that the resulting conjugate retains the mutant DNA repair enzyme property and also processes the property(ies) of the facilitating agent. For example, the facilitating agent can be a protein or a peptide fragment, such as a protein binding peptide, including but not limited to an epitope tag or an IgG binding protein, a nucleotide binding protein, such as a DNA or RNA binding protein, a lipid binding protein, a polysaccharide binding protein, and a metal binding protein or fragments thereof that possess the requisite desired facilitating activity.

Such facilitating agents can be designed, screened or selected according to the methods known in the art. The screening or selection process begins, for example, with nucleic acid encoding a particular protein or peptide to be used in the fusion protein, and screened or selected for its specific binding partner. Alternatively, the screening or selection process can start with a specific molecule that can be used in the subsequent isolation/purification, attachment or detection, and screen or select for a particular protein or peptide sequence to be used in the fusion protein that can specifically bind to the pre-selected molecule.

The conventional technique of random screening of natural products can be used in screening and selecting a protein or peptide sequence and its specific binding partner. In addition, numerous strategies can be used for preparing proteins having new binding specificities. These new approaches generally involve the synthetic production of large numbers of random molecules followed by some selection procedure to identify the molecule of interest. For example, epitope libraries have been developed using random polypeptides displayed on the surface of filamentous phage particles. The library is made by synthesizing a repertoire of random oligonucleotides to generate all combinations, followed by their insertion into a phage vector. Each of the sequences is separately cloned and expressed in phage, and the relevant expressed peptide can be selected by finding those phage that bind to the particular target. The phages recovered in this way can be amplified and the selection repeated. The sequence of the peptide is decoded by sequencing the DNA (See e.g., Cwirla et al., Proc. Natl. Acad. Sci., USA, 87:6378-6382 (1990); Scott et al., Science, 249:386-390 (1990); and Devlin et al., Science, 249:404-406 (1990).

Another approach involves large arrays of peptides that are synthesized in parallel and screened with acceptor molecules labelled with fluorescent or other reporter groups. The sequence of any effective peptide can be decoded from its address in the array (See e.g., Geysen et al., Proc. Natl. Acad. Sci., USA, 81:3998-4002 (1984); Maeji et al., J. Immunol. Met., 146:83-90 (1992); and Fodor et al., Science, 251:767-775 (1991).

Combinatorial approaches can also be employed. For example, in one exemplary approach, combinatorial libraries of peptides are synthesized on resin beads such that each resin bead contains about 20 pmoles of the same peptide. The beads are screened with labeled acceptor molecules and those with bound acceptor are searched for by visual inspection, physically removed, and the peptide identified by direct sequence analysis (Lam et al., Nature, 354:82-84 (1991)). Another useful combinatory method for identification of peptides of desired activity is that of Houghten et al. (see, e.g.,, Nature, 354:84-86 (1991)). For hexapeptides of the 20 natural amino acids, 400 separate libraries are synthesized, each with the first two amino acids fixed and the remaining four positions occupied by all possible combinations. An assay, based on competition for binding or other activity, is then used to find the library with an active peptide. Twenty new libraries are then synthesized and assayed to determine the effective amino acid in the third position, and the process is reiterated in this fashion until the active hexapeptide is defined.

b. Chemical Conjugation

To effect chemical conjugation herein, the targeting agent is linked via one or more selected linkers or directly to the targeted agent. Chemical conjugation must be used if the targeted agent is other than a peptide or protein, such a nucleic acid or a non-peptide drug. Any means known to those of skill in the art for chemically conjugating selected moieties may be used.

1) Heterobifunctional Cross-Linking Reagents

Numerous heterobifunctional cross-linking reagents that are used to form covalent bonds between amino groups and thiol groups and to introduce thiol groups into proteins, are known to those of skill in this art (see, e.g., the PIERCE CATALOG, Immuno Technology Catalog & Handbook, 1992-1993, which describes the preparation of and use of such reagents and provides a commercial source for such reagents; see, also, e.g., Cumber et al. (1992) Bioconjugate Chem. 3′:397-401; Thorpe et al. (1987) Cancer Res. 47:5924-5931; Gordon et al. (1987) Proc. Natl Acad Sci. 84:308-312; Walden et al. (1986) J. Mol. Cell Immunol. 2:191-197; Carlsson et al (1978) Biochem. J. 173: 723-737; Mahan et al. (1987) Anal. Biochem. 162:163-170; Wawryznaczak et al. (1992) Br. J. Cancer 66:361-366; Fattom et al., (1992) Infection & Immun. 60:584-589). These reagents may be used to form covalent bonds between the mutant analyte binding enzyme and the facilitating agent. These reagents include, but are not limited to: N-succinimidyl-3-(2-pyridyldithio)propionate (SPDP; disulfide linker); sulfosuccinimidyl 6-[3-(2-pyridyldithio)propionamido]hexanoate (sulfo-LC-SPDP); succinimidyloxycarbonyl-á-methyl benzyl thiosulfate (SMBT, hindered disulfate linker); succinimidyl 6-[3-(2-pyridyldithio) propionamido]hexanoate (LC-SPDP); sulfosuccinimidyl 4-(N-maleimidomethyl)cyclohexane-1-carboxylate (sulfo-SMCC); succinimidyl 3-(2-pyridyldithio)butyrate (SPDB; hindered disulfide bond linker); sulfosuccinimidyl 2-(7-azido-4-methylcoumarin-3-acetamide) ethyl-1,3′-dithiopropionate (SAED); sulfo-succinimidyl 7-azido-4-methylcoumarin-3-acetate (SAMCA); sulfosuccinimidyl 6-[alpha-methyl-alpha-(2-pyridyldithio)toluamido]hexanoate (sulfo-LC-SMPT); 1,4-di-[3′-(2′-pyridyldithio)propionamido]butane (DPDPB); 4-succinimidyloxycarbonyl-á-methyl-á-(2-pyridylthio)toluene (SMPT, hindered disulfate linker);sulfosuccinimidyl6[á-methyl-á-(2-pyridyldithio)toluamido]hexanoate (sulfo-LC-SMPT); m-maleimidobenzoyl-N-hydroxysuccinimide ester (MBS); m-maleimidobenzoyl-N-hydroxysulfosuccinimide ester (sulfo-MBS); N-succinimidyl(4-iodoacetyl)aminobenzoate (SIAB; thioether linker); sulfosuccinimidyl(4-iodoacetyl)amino benzoate (sulfo-SIAB); succinimidyl4(p-maleimidophenyl)butyrate (SMPB); sulfosuccinimidyl4-(p-maleimidophenyl)butyrate (sulfo-SMPB); azidobenzoyl hydrazide (ABH).

Other heterobifunctional cleavable cross-linkers include, N-succinimidyl (4-iodoacetyl)-aminobenzoate; sulfosuccinimydil (4-iodoacetyl)-aminobenzoate; 4-succinimidyl-oxycarbonyl-a-(2-pyridyldithio)toluene; sulfosuccinimidyl-6-[a-methyl-a-(pyridyldithiol)-toluamido] hexanoate; N-succinimidyl-3-(-2-pyridyldithio)-proprionate; succinimidyl 6[3(-(-2-pyridyldithio)-proprionamido] hexanoate; sulfosuccinimidyl 6[3(-(-2-pyridyldithio)-propionamido] hexanoate; 3-(2-pyridyldithio)-propionyl hydrazide, Ellman's reagent, dichlorotriazinic acid, S-(2-thiopyridyl)-L-cysteine. Further exemplary bifunctional linking compounds are disclosed in U.S. Pat. Nos. 5,349,066, 5,618,528, 4,569,789, 4,952,394, and 5,137,877.

2) Exemplary Linkers

Any linker known to those of skill in the art for preparation of conjugates may be used herein. These linkers are typically used in the preparation of chemical conjugates; peptide linkers may be incorporated into fusion proteins.

Linkers can be any moiety suitable to associate the mutant DNA repair enzyme and the facilitating agent. Such linkers and linkages include, but are not limited to, peptidic linkages, amino acid and peptide linkages, typically containing between one and about 60 amino acids, more generally between about 10 and 30 amino acids, chemical linkers, such as heterobi-functional cleavable cross-linkers, including but are not limited to, N-succinimidyl (4-iodoacetyl)-aminobenzoate, sulfosuccinimydil (4-iodoacetyl)-aminobenzoate, 4-succinimidyl-oxycarbonyl-a-(2-pyridyldithio)toluene, sulfosuccinimidyl-6-[a-methyl-a-(pyridyldithiol)-toluamido] hexanoate, N-succinimidyl-3-(-2-pyridyldithio)-proprionate, succinimidyl 6[3(-(-2-pyridyldithio)-proprionamido] hexanoate, sulfosuccinimidyl 6[3(-(-2-pyridyldithio)-propionamido] hexanoate, 3-(2-pyridyldithio)-propionyl hydrazide, Ellman's reagent, dichlorotriazinic acid, and S-(2-thiopyridyl)-L-cysteine. Other linkers include, but are not limited to peptides and other moieties that reduce stearic hindrance between the mutant analyte binding enzyme and the facilitating agent, intracellular enzyme substrates, linkers that increase the flexibility of the conjugate, linkers that increase the solubility of the conjugate, linkers that increase the serum stability of the conjugate, photocleavable linkers and acid cleavable linkers.

Other exemplary linkers and linkages that are suitable for chemically linked conjugates include, but are not limited to, disulfide bonds, thioether bonds, hindered disulfide bonds, and covalent bonds between free reactive groups, such as amine and thiol groups. These bonds are produced using heterobifunctional reagents to produce reactive thiol groups on one or both of the polypeptides and then reacting the thiol groups on one polypeptide with reactive thiol groups or amine groups to which reactive maleimido groups or thiol groups can be attached on the other. Other linkers include, acid cleavable linkers, such as bismaleimideothoxy propane, acid labile-transferrin conjugates and adipic acid diihydrazide, that would be cleaved in more acidic intracellular compartments; cross linkers that are cleaved upon exposure to UV or visible light and linkers, such as the various domains, such as C_{H}1, C_{H}2, and C_{H}3, from the constant region of human IgG₁ (see, Batra et al. (1993) Molecular Immunol. 30:379-386). In some embodiments, several linkers may be included in order to take advantage of desired properties of each linker.

Chemical linkers and peptide linkers may be inserted by covalently coupling the linker to the mutant DNA repair enzyme and the facilitating agent. The heterobifunctional agents, described below, may be used to effect such covalent coupling. Peptide linkers may also be linked by expressing DNA encoding the linker and TA, linker and targeted agent, or linker, targeted agent and TA as a fusion protein. Flexible linkers and linkers that increase solubility of the conjugates are contemplated for use, either alone or with other linkers are also contemplated herein.

a) Acid Cleavable, Photocleavable and Heat Sensitive Linkers

Acid cleavable linkers, photocleavable and heat sensitive linkers may also be used, particularly where it may be necessary to cleave the targeted agent to permit it to be more readily accessible to reaction. Acid cleavable linkers include, but are not limited to, bismaleimideothoxy propane; and adipic acid dihydrazide linkers (see, e.g., Fattom et al. (1992) Infection & Immun. 60:584-589) and acid labile transferrin conjugates that contain a sufficient portion of transferrin to permit entry into the intracellular transferrin cycling pathway (see, e.g., Welhöner et al. (1991) J. Biol. Chem. 266:4309-4314).

Photocleavable linkers are linkers that are cleaved upon exposure to light (see, e.g., Goldmacher et al. (1992) Bioconj. Chem. 3:104-107, which linkers are herein incorporated by reference), thereby releasing the targeted agent upon exposure to light. Photocleavable linkers that are cleaved upon exposure to light are known (see, e.g., Hazum et al. (1981) in Pept., Proc. Eur. Pept. Symp., 16th, Brunfeldt, K (Ed), pp. 105-110, which describes the use of a nitrobenzyl group as a photocleavable protective group for cysteine; Yen et al. (1989) Makromol. Chem 190:69-82, which describes water soluble photocleavable copolymers, including hydroxypropylmethacrylamide copolymer, glycine copolymer, fluorescein copolymer and methylrhodamine copolymer; Goldmacher et al. (1992) Bioconj. Chem. 3:104-107, which describes a cross-linker and reagent that undergoes photolytic degradation upon exposure to near UV light (350 nm); and Senter et al. (1985) Photochem. Photobiol 42:231-237, which describes nitrobenzyloxycarbonyl chloride cross linking reagents that produce photocleavable linkages), thereby releasing the targeted agent upon exposure to light. Such linkers would have particular use in treating dermatological or ophthalmic conditions that can be exposed to light using fiber optics. After administration of the conjugate, the eye or skin or other body part can be exposed to light, resulting in release of the targeted moiety from the conjugate. Such photocleavable linkers are useful in connection with diagnostic protocols in which it is desirable to remove the targeting agent to permit rapid clearance from the body of the animal.

b) Other Linkers for Chemical Conjugation

Other linkers, include trityl linkers, particularly, derivatized trityl groups to generate a genus of conjugates that provide for release of therapeutic agents at various degrees of acidity or alkalinity. The flexibility thus afforded by the ability to preselect the pH range at which the therapeutic agent will be released allows selection of a linker based on the known physiological differences between tissues in need of delivery of a therapeutic agent (see, e.g., U.S. Pat. No. 5,612,474). For example, the acidity of tumor tissues appears to be lower than that of normal tissues.

c) Peptide Linkers

The linker moieties can be peptides. Peptide linkers can be employed in fusion proteins and also in chemically linked conjugates. The peptide typically a has from about 2 to about 60 amino acid residues, for example from about 5 to about 40, or from about 10 to about 30 amino acid residues. The length selected will depend upon factors, such as the use for which the linker is included.

The proteinaceous ligand binds with specificity to a receptor(s) on one or more of the target cell(s) and is taken up by the target cell(s). In order to facilitate passage of the chimeric ligand-toxin into the target cell, it is presently preferred that the size of the chimeric ligand-toxin be no larger than can be taken up by the target cell of interest. Generally, the size of the chimeric ligand-toxin will depend upon its composition. In the case where the chimeric ligand toxin contains a chemical linker and a chemical toxin (i.e., rather than proteinaceous one), the size of the ligand toxin is generally smaller than when the chimeric ligand-toxin is a fusion protein. Peptidic linkers can conveniently be encoded by nucleic acid and incorporated in fusion proteins upon expression in a host cell, such as E. coli.

Peptide linkers are advantageous when the facilitating agent is proteinaceous. For example, the linker moiety can be a flexible spacer amino acid sequence, such as those known in single-chain antibody research. Examples of such known linker moieties include, but are not limited to, peptides, such as (GlyₘSer)ₙ and (SerₘGly)ₙ, in which n is 1 to 6, preferably 1 to 4, more preferably 2 to 4, and m is 1 to 6, preferably 1 to 4, more preferably 2 to 4, enzyme cleavable linkers and others.

Additional linking moieties are described, for example, in Huston et al., Proc. Natl. Acad. Sci. U.S.A. 85:5879-5883, 1988; Whitlow, M., et al., Protein Engineering 6:989-995, 1993; Newton et al., Biochemistry 35:545-553, 1996; A. J. Cumber et al., Bioconj. Chem. 3:397-401, 1992; Ladurner et al., J. Mol. Biol. 273:330-337, 1997; and U.S. Pat. No. 4,894,443. In some embodiments, several linkers may be included in order to take advantage of desired properties of each linker.

2. Selection of Facilitating Agents

Any agent that facilitates detection, immobilization, or purification of the conjugate is contemplated for use herein. For chemical conjugates any moiety that has such properties is contemplated; for fusion proteins, the facilitating agent is a protein, peptide or fragment thereof that is sufficient to effect the facilitating activity.

a. Protein Binding Moieties

The conjugate contains a protein binding moiety, particularly a protein binding protein, peptide or effective fragment thereof. Its specific binding partner can be proteins or peptides generally, a set of proteins or peptides or mixtures thereof, or a particular protein or peptide. Any protein-protein interaction pair known to those of skill in the art is contemplated. For example, the protein-protein interaction pair can be enzyme/protein or peptide substrate, antibody/protein or peptide antigen, receptor/protein or peptide ligand, etc. Any protein-protein interaction pair can be designed, screened or selected according to the methods known in the art (See generally, Current Protocols in Molecular Biology (1998) § 20, John Wiley & Sons, Inc.). Examples of such methods for identifying protein-protein interactions include the interaction trap/two-hybrid system and the phage-based expression cloning.

1) Interaction Trap/Two-Hybrid System

Interacting proteins can be identified by a selection or screen in which proteins that specifically interact with a target protein of interest are isolated from a library. One particular approach to detect interacting proteins is the two-hybrid system or interaction trap (See generally, Current Protocols in Molecular Biology (1998) § 20.1.-20.2., John Wiley & Sons, Inc.), which uses yeast as a “test tube” and transcriptional activation of a reporter system to identify associating proteins.

In the two-hybrid system, a yeast vector such as the plasmid pEG202 or a related vector can be used to express the probe or “bait” protein as a fusion to the heterologous DNA-binding protein LexA. Many proteins, including transcription factors, kinases, and phosphatases, can be used as bait proteins. The major requirements for the bait protein are that it should not be actively excluded from the yeast nucleus, and it should not possess an intrinsic ability to strongly activate transcription. The plasmid expressing the LexA-fused bait protein can be used to transform yeast possessing a dual reporter system responsive to transcriptional activation through the LexA operator.

In one such example, the yeast strain EGY48 containing the reporter plasmid pSH18-34 can be used. In this case, binding sites for LexA are located upstream of two reporter genes. In the EGY48 strain, the upstream activating sequences of the chromosomal LEU2 gene, which is required in the biosynthetic pathway for leucine (Leu), are replaced with LexA operators (DNA binding sites). PSH18-34 contains a LexA operator-lacZ fusion gene. These two reporters allow selection for transcriptional activation by permitting selection for viability when cells are plated on medium lacking Leu, and discrimination based on color when the yeast is grown on medium containing Xga1.

The EGY48/PSH18-34 transformed with a bait is first characterized for its ability to express protein, growth on medium lacking Leu, and for the level of transcriptional activation of lacZ. A number of alternative strains, plasmids, and strategies can be employed if a bait proves to have an unacceptably high level of background transcriptional activation.

In an interactor hunt, the stain EGY48/PSH18-34 containing the bait expression plasmid is transformed, preferably along with carrier DNA, with a conditionally expressed library made in a suitable vector such as the vector pJG4-5. This library uses the inducible yeast GAL1 promoter to express proteins as fusions to an acidic domain (“acid blob”) that functions as a portable transcriptional activation motif (act) and to other useful moieties. Expression of library-encoded proteins is induced by plating transformants on medium containing galactose (Gal), so yeast cells containing library proteins that do not interact specifically with the bait protein will fail to grow in the absence of Leu. Yeast cells containing library proteins that interact with the bait protein will form colonies within 2 to 5 days, and the colonies will turn blue when the cells are streaked on medium containing Xga1 . The DNA from interaction trap positive colonies can be analyzed by polymerase chain reaction (PCR) to streamline screening and detect redundant clones in cases where many positives are obtained in screening. The plasmids can be isolated and characterized by a series of tests to confirm specificity of the interaction with the initial bait protein.

An alternative way of conducting an interactor hunt is to mate a strain that expresses the bait protein with a strain that has been pretransformed with the library DNA, and screen the resulting diploid cells for interactors (Bendixen et al., Nucl. Acids. Res., 22:1778-1779 (1994); and Finley and Brent, Proc. Natl. Sci. U.S.A., 91:12980-12984 (1994)). This “interaction mating” approach can be used for any interactor hunt, and is particularly useful in three special cases. The first case is when more than one bait will be used to screen a single library. Interaction mating allows several interactor hunts with different baits to be conducted using a single high-efficiency yeast transformation with library DNA. This can be a considerable savings, since the library transformation is one of the most challenging tasks in an interactor hunt. The second case is when a constitutively expressed bait interferes with yeast viability. For such baits, performing a hunt by interaction mating avoids the difficulty associated with achieving a high-efficiency library transformation of a strain expressing a toxic bait. Moreover, the actual selection for interactors will be conducted in diploid yeast, which are more vigorous than haploid yeast and can better tolerate expression of toxic proteins. The third case is when a bait cannot be used in a traditional interactor hunt using haploid yeast stains because it activates transcription of even the least sensitive reporters. In diploids the reporters are less sensitive to transcription activation than they are in haploids. Thus, the interaction mating hunt provides an additional method to reduce background from transactivating baits.

The interaction trap/two-hybrid system and the identified protein-protein interaction pairs have been successfully used (see, e.g., Bartel et al., Using the two-hybrid system to detect protein-protein interactions, In Cellular Interactions in Development: A Practical Approach, (D. A. Hartley, ed.) pp. 153-179, Oxford University Press, Oxford (1993); Bartel et al., A protein linkage map of Escherichia coli bacteriophage T7, Nature Genet., 12:72-77 (1996); Bendixen et al., A yeast mating-selection scheme for detection of protein-protein interactions, Nucl. Acids. Res., 22:1778-1779 (1994); Breeden and Nasmyth, Regulation of the yeast HO gene., Cold spring Harbor Symp. Quant. Biol, 50:643-650 (1985); Brent and Ptashne, A bacterial repressor protein or a yeast transcriptional terminator can block upstream activation of a yeast gene, Nature, 312:612-615 (1984); Brent et al., A eukaryotic transcriptional activator bearing the DNA specificity of a prokaryotic repressor, Cell, 43:729-736 (1985); Chien et al., The two-hybrid system: A method to identify and clone genes for proteins that interact with a protein of interest, Proc. Natl. Acad. Sci. U.S.A., 88:9578-9582 (1991); Chiu et al., RAPT1, a mammalian homolog of yeast Tor, interacts with the FKBP12/rapamycin complex, Proc. Nat. Acad. Sci., U.S.A., 91:12574-12578 (1994); Colas et al., Genetic selection of peptide aptamers that recognize and inhibit cyclin-dependent kinase 2., Nature, 380:548-550 (1996); Durfee et al, The retinoblastoma protein associates with the protein phosphatase type 1 catalytic subunit, Genes & Dev., 7:555-569 (1993); Estojak et al, Correlation of two-hybrid affinity data with in vitro measurements, Mol. Cell. Biol., 15:5820-5829 (1995); Fearon et al., Karyoplasmic interaction selection strategy: A general strategy to detect protein-protein interaction in mammalian cells, Proc. Nat., Acad. Sci. U.S.A., 89:7958-7962 (1992); Fields and Song, A novel genetic system to detect protein-protein interaction, Nature, 340:245-246 (1989); Finley and Brent, Interaction mating revels binary and ternary connections between Drosophila cell cycle regulators, Proc. Natl. Sci. U.S.A., 91:12980-12984 (1994); Gietz et al., Improved method for high-efficiency transformation of intact yeast cells, Nucl. Acids. Res., 20:1425 (1992); Golemis and Brent, Fused protein domains inhibit DNA biding by LexA, Mol. Cell Biol., 12:3006-3014 (1992); Gyuris et al., Cdi1, a human G1and S-phase protein phosphatase that associates with Cdk1, Cell, 75:791-803 (1993); Kaiser et al., A., Methods in Yeast Genetics, a Cold Spring Harbor Laboratory Course Manual, pp. 135-136. Cold Spring Harbor Laboratory Press, Cold Spring Harbor, N.Y. (1994); Kolonin and Finley, Jr., Targeting cyclin-dependent kinases in Drosophila with peptide aptamers, Proc. Natl. Acad. Sci. U.S.A., In press (1998); Licitra and Liu, A three-hybrid system for detecting small ligand-protein receptor interactions, Proc. Nat. Acad. Sci. U.S.A., 93:12817-12821 (1996); Ma and Ptashne, A new class of yeast transcriptional activators, Cell, 51:113-119 (1987); Ma and Ptashne, Converting an eukaryotic transcriptional inhibitor into an activator, Cell, 55:443-446 (1988); Osborne et al., The yeast tribrid system: Genetic detection of transphosphorylated ITAM-SH2 interactions, Bio/Technology, 13:1474-1478 (1995); Ruden et al., Generating yeast transcriptional activators containing no yeast protein sequences, Nature, 350:426-430 (1991); Samson et al, Gene activation and DNA binding by Drosophila Ubx and abd-A proteins, Cell, 57:1045-1052 (1989); Stagljar et al., Use of the two-hybrid system and random sonicated DNA to identify the interaction domain of a protein, BioTechniques, 21:430-432 (1996); Vasavada et al, A contingent replication assay for the detection of protein-protein interactions in animal cells, Proc. Nat. Acad. Sci. U.S.A., 88:10686-10690 (1991); Vojtex et al., Mammalian Ras interacts directly with the serine/threonine kinase Raf, Cell, 74:205-214 (1993); Watson et al., Vectors encoding alternative antibiotic resistance for use in the yeast two-hybrid system, BioTechniques, 21:255-259 (1996); West et al., Saccharomyces cerevisiae GAL10 divergent promoter region: Location and function of the upstream activator sequence UASG, Mol. Cell Biol., 4:2467-2478 (1984); and Yang et al., Protein-peptide interactions analyzed with the yeast two-hybrid system, Nucl. Acids Res., 23:1152-1156 (1995)) and can be used in the present system.

2) Phage-Based Expression Cloning

Interaction cloning (also known as expression cloning) is a technique to identify and clone genes that encode proteins that interact with a protein of interest, or “bait” protein. Phage-based interaction cloning requires a gene encoding the bait protein and an appropriate expression library constructed in a bacteriophage expression vector, such as ëgt11 (See generally, Current Protocols in Molecular Biology (1998) § 20.3, John Wiley & Sons, Inc.). The gene encoding the bait protein is used to produce recombinant fusion protein in E. coli. The cDNA is radioactively labeled with ³²p. A recognition site for a protein kinase such as the cyclic adenosine 3′,5′-phosphate (cAMP)—dependent protein kinase (Protein kinase A; PKA) is introduced into the recombinant fusion protein to allow its enzymatic phosphorylation by the kinase and [ë-³²P]ATP.

In one example, the procedure involves a fusion protein containing bait protein and glutathione-S-transferase (GST) with a PKA site at the junction between them. The labeled protein is subsequently used as a probe to screen a ë bacteriophage-derived cDNA expression library, which expresses â-galactosidase fusion proteins that contain in-frame gene fusions. The phages lyse cells, form plaques, and release fusion proteins that are adsorbed onto nitrocellulose membrane filters. The filters are blocked with excess nonspecific protein to eliminate nonspecific binding and probed with the radiolabeled bait protein. This procedure leads directly to the isolation of genes encoding the interacting protein, bypassing the need for purification and microsequencing or for antibody production.

The phage-based interaction cloning system and the identified protein-protein interaction pairs have been successfully employed (Blanar et al., Interaction cloning: Identification of a helix-loop-helix zipper protein that interacts with c-Fos, Science, 256:1014-1018 (1992); Carr and Scott, Blotting and band-shifting: Techniques for studying protein-protein interactions, Trends Biochem. Sci., 17:246-249 (1992); Chapline et al., Interaction cloning of protein kinase C substrates, J. Biol. Chem., 268:6858-6861 (1993); Hoeffler et al., Identification of multiple nuclear factors that interact with cyclic AMP response element-binding protein and activation transcription factor-2 by protein interactions, Mol. Endocrinol., 5:256-266 (1991); Kaelin et al., Expression cloning of a cDNA encoding a retinoblastoma-binding protein with E2F-like properties, Cell, 70:351-364 (1992); Lester et al., Cloning and characterization of a novel A-kinase anchoring protein: AKAP220, association with testicular peroxisomes, J. Biol. Chem., 271:9460-9465 (1996); Lowenstein et al., The SH2 and SH2 domain-containing protein GRB2 links receptor tyrosine kinase to ras signaling, Cell, 70:431-442 (1992); Margolis et al., High-efficiency expression/cloning of epidermal growth factor-receptor-binding proteins with src homology 2 domains, Proc. Natl. Acad. Sci. U.S.A., 89:8894-8898 (1992); Skolnik et al., Cloning of P13 kinase-associated p85 utilizing a novel method of expression/cloning of target proteins for receptor tyrosine kinases, Cell, 65:83-90 (1991); and Stone et al., Interaction of a protein phosphatase with an Arabidopsis serine-threonine receptor kinase, Science, 266:793-795 (1994)) and can be used in the present system.

3) Detection of Protein-Protein Interactions

Surface plasmon resonance (SPR) can be used to verify the protein-protein interactions identified by other systems such as the interaction trap/two-hybrid system and the phage-based expression cloning systems (See generally, Current Protocols in Molecular Biology (1998) § 20.4, John Wiley & Sons, Inc.). This is an in vitro technique based on an optical phenomenon, called SPR, that can simultaneously detect interactions between unmodified proteins and directly measure kinetic parameters of the interaction.

SPR devices are commercially available. The BIAcore instrument (BIAcore) is presently preferred herein. This instrument contains sensing optics, an automated sample delivery system, and a computer for instrument control, data collection, and data processing. Experiments are performed on disposable chips. In practice, a ligand protein is immobilized on the chip while buffer continuously flows over the surface. The sensing apparatus monitors changes in the angle of minimum reflectance from the interface that result when a target protein associates with the ligand protein. Molecular interactions can be directly visualized (on the computer monitor) in real time as the optical response is plotted against time. This response is measured in resonance units (RUs, where 1000 RUs=1 ng protein/mm²).

The SPR system has been successfully used (see, e.g., BioSupplyNet Source Book, BioSupplyNet, Plainview, N.Y., and Cold Spring Harbor Laboratory Press, Cold Spring Harbor, N.Y. (1999); Feng et al., Functional binding between Gâ and the LIM domain of Ste5 is required to activate the MEKK Ste11, Cur. Biol., 8:267-278 (1998); Field et al., Purification of RAS-responsive adenylyl cyclase complex from Sacchariomyces cerevisiae by use of an epitope addition method, Mol. Cell. Biol., 8:2159-2165 (1988); Phizicky and Fields, Protein-protein interactions: Methods for detection and analysis, Microbiol. Rev., 59:94-123 (1995); Tyers et al., Comparison of the Saccharomyces cerevisiae G1 cyclins: Cln3 may be an upstream activator of Cln1, Cln2, and other cyclins, EMBO J., 11:1773-1784 (1993)) and the identified protein-protein interaction pairs can be used in the present system.

b. Epitope Tags

The facilitating agent can be any moiety, particularly a protein, peptide or effective fragment thereof that is specifically recognized by an antibody. In these embodiments, the conjugate contains an epitope tag that is specifically recognized by a set of antibodies or by a particular antibody. Any epitope/antibody pair can be used in the present system (See generally, Current Protocols in Molecular Biology (1998) 10.15, John Wiley & Sons, Inc.). The following Table 3 provides exemplary epitope tags and illustrates certain properties of several commonly used epitope tag systems.

TABLE 3
Exemplary epitope tag systems

| Epitope | Peptide | SEQ ID | Antibody | Reference |
|---|---|---|---|---|
| FLAG | AspTyrLysAspAspAspLys | 1 | 4E11 | Prickett¹ |
| HA | TyrProTyrAspValPRoAspTyrAla | 2 | 12Ca5 | Xie² |
| HA1 | CysGlnAspLeuProGlyAsnAspAsnSerThr | 3 | mouse MAb | Nagelkerken³ |
| c-Myc | GluGlnLysLeuIleSerGluGluAspLeu | 4 | 9E10 | Xie² |
| 6-His | HisHisHisHisHisHis | 5 | BAbCO* | |
| AU1 | AspThrTyrArgTyrIle | 6 | BAbCO | |
| FE | GluTyrMetProMetGlu | 7 | anti-EE | Tolbert⁴ |
| T7 | AlaSerMetThrGlyGlyGlnGlnMetGlyArg | 8 | Invitrogen | Chen⁵ |
| | | | | Tseng⁶ |
| 4A6 | SerPheProGlnPheLysProGlnGluIle | 9 | 4A6 | Rudiger⁷ |
| å | LysGlyPheSerTyrPheGlyGluAspLeuMetPro | 10 | anti-PKCå | Olah⁸ |
| B | GlnTyrProAlaLeuThr | 11 | D11, F10 | Wang⁹ |
| gE | GlnArgGlnTyrGlyAspValPheLysGlyAsp | 12 | 3B3 | Grose¹⁰ |
| Ty1 | GluValHisThrAsnGlnAspProLeuAsp | 13 | BB2, TYG5 | Bastin¹¹ |

1. Prickett et al., Bio Techniques, 7(6):580-584 (1989)
2. Xie et al., Endocrinology, 139(11):4563-4567 (1998)
3. Nagelkerke et al., Electrophoresis, 18:2694-2698 (1997)
4. Tolbert and Lameh, J. Neurochem., 70:113-119 (1998)
5. Chen and Katz, Bio Techniques, 25(1):22-24 (1998)
6. Tseng and Verma, Gene, 169:287-288 (1996)
7. Rudiger et al., Bio Techniques, 23(1):96-97 (1997)
8. Olah et al., Biochem., 221:94-102 (1994)
9. Wang et al., Gene, 169(1):53-58 (1996)
10. Grose, U.S. Pat. No. 5,710,248
11. Bastin etal., Mol. Biochem. Parasitology, 77:235-239 (1996)
* Invitrogen, Sigma, Santa Cruz Biotech

For example, in one embodiment, the selected epitope tag is the 6-His tag. Vectors for constructing a fusion protein containing the 6-His tag and reagents for isolating or purifying such fusion proteins are commercially available. For example, the Poly-His gene fusion vector from Invitrogen, Inc. (Carlsbad, Calif.) includes the following features: 1) high-level regulated transcription for the trc promotor; 2) enhanced translation efficiency of eukaryotic genes in E. coli; 3) the LacO operator and the Lac^{q} repressor gene for transcriptional regulation in any E. coli system; N-terminal Xpress epitope for easy detection with an Anti-Xpress antibody; and 4) enterokinase cleaving site for removal of the fusion tag. The fusion protein can be purified by nickel-chelating agarose resin, and the purified fusion protein can be coated onto a microtiter plate pre-coated with nickel (e.g., Reacti-Binding meta chelate polystyrene plates, Pierce) for diagnostic usage.

In addition, the fusion protein containing the 6-His tag can be isolated or purified using the His MicroSpin Purification Module or HisTrap Kit from Amersham Pharmacia Biotech, Inc. The His MicroSpin Purification Module provides fifty MicroSpin columns prepacked with nickel-charged Chelating Sepharose Fast Flow. The module enables the simple and rapid screening of large numbers of small-scale bacterial lysates for the analysis of putative clones and optimization of expression and purification conditions. Each column contains 50 ìl bed volume, enough to purify>100 ìg His-tagged fusion protein, from up to 400 ìl of his tagged fusion protein sample, e.g., crude lysate and purification intermediates. The HisTrap Kit is designed for rapid, mild affinity purification of histidine-tagged fusion proteins in a single step. The high dynamic capacity of HiTrap Chelating enables milligrams of protein to be purified in less than 15 minutes at flow rates of up to 240 column volumes per hour. The high capacity is maintained after repeated use ensuring cost-effective, reproducible purifications. The Kit includes three HiTrap Chelating columns and buffer concentrates to perform F 10-12 purifications with a syringe. The anti-His antibody from Amersham Pharmacia Biotech, Inc. is an IgG₂ subclass of monoclonal antibody directed against 6 Histidine residues. The antibody is unconjugated to offer the flexibility of detection with a secondary antibody conjugated with either horseradish peroxidase or alkaline phosphatase. The antibody provides high sensitivity with low background.

Further examples of epitope tagging can be found in Kolodziej and Young, Epitope tagging and protein surveillance, Methods Enzymol., 194:508-519 (1991). Methods for preparing and using other such tags and other such tags similarly can be used in the methods and products provided herein.

c. IgG Binding Proteins

In other embodiments, the conjugate contains an IgG binding protein, which, for example provides a means for selective binding of the conjugate. Any IgG binding protein/IgG pair can be used in the present system. Protein A and Protein G are suitable facilitating. Any Protein A or Protein G can be used in the present system.

For example, the following nucleotide sequences can be used for amplifying and constructing Protein A or Protein G fusion proteins: E04365 (Primer for amplifying IgG binding domain AB of protein A); E04364 (Primer for amplifying IgG binding domain AB of protein A); E01756 (DNA sequence encoding subunit which can bind IgG of protein A like substance); M74187 (Cloning vector pKP497 (cloning, screening, fusion vector) encoding an IgG-binding fusion protein from protein A analogue (ZZ) and beta-Gal′ (lacZ) genes). In addition, several Protein A gene fusion vectors such as pEZZ 18 and pRIT2T are commercially available (Amersham Pharmacia Biotech, Inc.).

1) pEZZ 18 Protein A Gene Fusion Vector

pEZZ 18 Protein A gene fusion vector can be used for rapid expression of secreted fusion proteins and their one-step purification using IgG Sepharose 6FF. The phagemid pEZZ 18 contains the proteins A signal sequence and two synthetic “Z” domains based on the “B” IgG binding domain of Protein A (Löwenadler., et al., Gene, 58:87 (1987); and Nilsson., et al., Prot. Engineering, 1:107 (1987)). Proteins are expressed as fusions with the “ZZ” peptide and secreted into the aqueous culture medium under the direction of the protein A signal sequence. They are easily purified using IgG Sepharose 6FF to which the “ZZ” domain binds tightly. Because of its unique folding properties, the 14 kDa “ZZ” peptide has little effect on folding of the fusion partner into a native conformation.

Expression

Expression is controlled by the lacUV5 and protein A promoters and is not inducible. Elements of the protein A gene provide the ATG and ribosome-binding sites. Stop codons must be provided by the insert.

Sequencing

The M13 Universal Sequencing Primer is used for double-stranded and single-stranded sequencing. A protocol for production of single-stranded DNA is provided with the vector

Cloning

Inserts containing a stop codon will yield white colonies when grown on media containing X-gal.

Host(s)

E. coli strains carrying a lac deletion but capable of á-complementation of lacZ′.

Selectable Marker(s)

Plasmid confers resistance to ampicillin.

Amplification

Amplification, though not necessarily required can be included.

2) pRIT2T Protein A Gene Fusion Vector

The pRIT2T Protein A gene fusion vector (available from Pharmacia) can be used for high-level expression of intracellular fusion proteins. pRIT2T, a derivative of pRIT2 (Nilsson., et al., EMBO J., 4:1075 (1985)), contains the IgG-binding domains of staphylococcal protein A which permits rapid affinity purification of fusion proteins on IgG Sepharose 6 FF. Thermo-inducible expression of the fusion protein is achieved in a suitable E. coli host strain which carries the temperature-sensitive repressor cI857 (N4830-1) (Zabeau and Stanley, EMBO J., 1:1217 (1982)).

Induction

The ëP_{R} promoter is induced by shifting the growth temperature from 30?C. to 42?C. for 90 minutes.

Expression

Genes inserted into the MCS are expressed from the ë right promoter (P_{R}) as fusions with the IgG-binding domains of staphylococcal protein A. A portion of the ë cro gene, fused to the IgG-binding domain, supplies the ATG start codon. Since no signal sequence is provided, the protein remains intracellular. Protein A gene transcription and translation termination signals are provided. Fusion protein can be purified on IgG Sepharose 6FF (17-0969-01). The protein A carrier protein is ˜30 kDa.

Host(s)

E. Coli N4830-1/N99cl⁺. Supplied with E. Coli N4830-1 which contains the temperature-sensitive cI857 repressor.

Selectable Marker(s)

Plasmid confers resistance to ampicillin.

3) The IgG Sepharose 6 Fast Flow System

The Protein A and Protein G fusion protein can be isolated or purified by affinity binding with IgG, such as the IgG Sepharose 6 Fast Flow System (Amersham Pharmacia Biotech, Inc.). The IgG Sepharose 6 Fast Flow System includes IgG coupled to the highly cross-linked 6% agarose matrix Sepharose 6 Fast Flow, and is designed for the rapid purification of Protein A and Protein A fusion conjugates. The system binds at least 2 mg Protein A/ml drained gel with flow possible rates of 300 cm/hr at 1 bar (14.5 psi, 0.1 MPa) in an XK 50/30 column (Lundstrëm et al., Biotechnology and Bioengineering, 36:1056 (1990)).

d. â-Galactosidase Fusion Proteins

The pMC1871 fusion vector (commercially available from Pharmacia, see, also Shapira et al. Gene 25:71 (1983); Casadaban et al. Methods Enzymol. 100:293 (1983)) for production of enzymatically active â-galactosidase hybrid proteins for gene expression or functional studies. Vector pMC1871 is derived from pBR322 and contains a promoterless lacZ gene, which also lacks a ribosome-binding site and the first eight non-essential N-terminal amino acid codons. Its unique Sma I site allows fusions to the N-terminal part of the â-galactosidase gene. Insertion of a gene into the E. coil lacZ gene results in the production of a hybrid protein, whose presence can be readily detected by following its â galactosidase activity (Miller, J. H., in Experiments in Molecular Gener. (Cold Spring Harbor, N.Y.) (1972); Nielsen et al. Proc. Natl. Acad. Sci. U.S.A., 80:5198 (1983)). Hybrid proteins can then be easily purified by affinity chromatography (Germino et al. Proc. Natl. Acad. Sci. U.S.A., 81: 4692 (1984)). Multiple cloning sites flanking the lacZ gene permit its excision as a BamH I, Sa1 I, Pst I or EcoR I gene cassette. If lacZ is excised as an EcoRI cassette, a portion of its 3′-end will be deleted. The resulting â-galactosidase protein (à-donor) will be functional if the C-terminus of the â-galactosidase protein (à-acceptor) is available through intercistronic complementation.

Expression

Inserts cloned into the unique Sma I site give fusion proteins with the N-terminal part of â-galactosidase. Insert must contain a promoter, ATG and ribosome-binding site.

Host(s)

E. coil Strains Carrying a 1ac deletion.

Selectable Marker(s)

Plasmid confers resistance to 15 ìg/ml tetracycline.

GenBank Accession Number L08936.

e. Nucleic Acid Binding Moieties

In another embodiment, the conjugate includes a nucleotide binding protein, peptide or effective fragment thereof as a facilitating agent. The specific binding partner can be nucleotide sequences generally, a set of nucleotide sequences or a particular nucleotide sequence. Any protein-nucleotide interaction pair can be used in the present system. For example, the protein-nucleotide interaction pair can be protein/DNA or protein/RNA pairs, or a combination thereof. Protein-nucleotide interaction pairs can be designed, screened or selected according to the methods known in the art (See generally, Current Protocols in Molecular Biology (1998) § 12, John Wiley & Sons, Inc.). Examples of such methods for identifying protein-nucleotide interactions include the gel mobility shift assay, methylation and uracil interference assay, DNase I footprint analysis, ëgt11 expression library screening and rapid separation of protein-bound DNA from free DNA using nitrocellulose filters.

1) DNA Binding Proteins

The conjugate can contain a DNA binding protein and its specific binding partner can be DNA molecules generally, a set of DNA molecules or a particular sequence of nucleotides. Any DNA binding protein can be used in the present system. For example, the DNA binding protein can bind to a single-stranded or double-stranded DNA sequence, or to an A-, B- or Z-form DNA sequence. The DNA binding sequence can also bind to a DNA sequence that is involved in replication, transcription, DNA repair, recombination, transposition or DNA structure maintenance. The DNA binding sequence can further be derived from a DNA binding enzyme such as a DNA polymerase, a DNA-dependent RNA polymerase, a DNAase, a DNA ligase, a DNA topoisomerase, a transposase, a DNA kinase, or a restriction enzyme.

Any DNA binding sequence/DNA sequence pair can be designed, screened or selected according to the methods known in the art including methods described in Section L.2. above.

The following Table 4 illustrates certain properties of several DNA binding sequence/DNA sequence pair systems.

TABLE 4
Examples of DNA binding sequence/DNA sequence binding pairs

| DNA binding | DNA binding | | Reference |
|---|---|---|---|
| sequence | sequence motif | DNA sequence | (U.S. Pat. No.) |
| NF-ATₚ | T lymphocyte | GCCCAAAGAGGAAA | 5,656,452 |
| (SEQ ID NO. 14) | DNA-binding protein | ATTTGTTTCATACAG | |
| | | (SEQ ID NO. 15) | |
| Max | helix-loop-helix | CACGTG | 5,693,487 |
| (SEQ ID NO. 16) | zipper protein | | |
| Chicken Lung 140 Kd | | Z-DNA | 5,726,050 |
| Protein | | | |
| EGR1, EGR2, GLI, | Zinc finger proteins | GACC, GCAC | 5,789,538 |
| Wilm's tumor gene, | | | |
| Sp1, Hunchback, | | | |
| Kruppel, ADR1 and | | | |
| BrLA | | | |
| LIL-Stat protein | Stat | TTNCNNAGA, | 5,821,053 |
| | family of | TTCCTGAGA | |
| | transcription factors | | |
| Egr | zinc finger protein | CGCCCCCGC | 5,866,325 |
| (SEQ ID NO. 17) | | | |
| S1-3 protein (SEQ ID | zinc finger protein | CATRRWWG | 5,905,146 |
| NO. 18) | | | |

2) RNA Binding Proteins

In another preferred embodiment, the conjugate can contain an RNA binding protein and its specific binding partner can be RNA generally, a set of RNA molecules or a particular sequence of ribonucleotides. Any RNA binding protein can be used in the present system. For example, the RNA binding protein can bind to a single-stranded or double-stranded RNA, or to rRNA, mRNA or tRNA. The RNA binding protein may specifically bind to a RNA that is involved in reverse transcription, transcription, RNA editing, RNA splicing, translation, RNA stabilization, RNA destabilization, or RNA localization. The RNA binding protein can be derived from or be an RNA binding enzyme such as a RNA-dependent DNA polymerase, a RNA-dependent RNA polymerase, a RNase, a RNA ligase, a RNA maturase, or a ribosome.

Other RNA recognition sequence or binding motifs that can be used in the present system include the zinc-finger motif, the Y-box, the KH motif, AUUUA, histone, RNP motif (U1), arginine-rich motif (ARM or PRE), double-stranded RNA binding motifs (IRE) and RGG box (APP) (U.S. Pat. Nos. 5,834,184, 5,859,227 and 5,858,675). The RNP motif is a 90-100 amino acid sequence that is present in one or more copies in proteins that bind pre mRNA, mRNA, pre-ribosomal RNA and snRNA. The consensus sequence and the sequences of several exemplary proteins containing the RNP motif are provided in Burd and Dreyfuss, Science, 265:615-621 (1994); Swanson et al., Trends Biochem. Sci., 13:86 (1988); Bandziulis et al., Genes Dev., 3:431 (1989); and Kenan et al., Trends Biochem. Sci., 16:214 (1991). The RNP consensus motif contains two short consensus sequences RNP-1and RNP-2. Some RNP proteins bind specific RNA sequences with high affinities (dissociation constant in the range of 10⁻⁸-10⁻¹¹ M). Such proteins often function in RNA processing reactions. Other RNP proteins have less stringent sequence requirements and bind less strongly (dissociation constant about 10⁻⁶-10⁻⁷ M) (Burd & Dreyfuss, EMBO J., 13:1197 (1994)).

A second characteristic RNA binding motif found in viral, phage and ribosomal proteins is an arginine-rich motif (ARM) of about 10-20 amino acids. RNA binding proteins having this motif include the HIV Tat and Rev proteins. Rev binds with high affinity disassociation constant (10⁻⁹ M) to an RNA sequence termed RRE, which is found in all HIV mRNAs (Zapp et al., Nature, 342:714 (1989); and Dayton et al., Science, 246:1625 (1989)). Tat binds to an RNA sequence termed TAR with a dissociation constant of 5×10⁻⁹ M (Churcher et al., J. Mol. Biol., 230:90 (1993)). For Tat and Rev proteins, a fragment containing the arginine-rich motif binds as strongly as the intact protein. In other RNA binding proteins with ARM motifs, residues outside the ARM also contribute to binding.

The double-stranded RNA-binding domain (dsRBD) exclusively binds double-stranded RNA or RNA-DNA. A dsRBD motif includes a region of approximately 70 amino acids which includes basic residues and contains a conserved core sequence with a predicted a-helical structure. The dsRBD motif is found in at least 20 known or putative RNA-binding proteins from different organisms. There are two types of dsRBDs; Type A, which is homologous along its entire length with the defined consensus sequence, and Type B, which is more highly conserved at its C terminus than its N terminus. These domains have been functionally delineated in specific proteins by deletion analysis and RNA binding assays (St Johnston, et al., Proc. Natl. Acad. Sci., 89:10979-10983 (1992)).

Any RNA binding sequence/RNA sequence pair can be designed, screened or selected according to the methods known in the art including the methods described in Section L.2. above and the methods, such as those described in U.S. Pat. Nos. 5,834,184 and 5,859,227, and in SenGupta et al., A three-hybrid system to detect RNA-protein interactions in vivo, Proc. Nat. Acad. Sci. U.S.A., 93:8496-8501 (1996)).

For example, U.S. Pat. No. 5,834,184 describes a method of screening a plurality of polypeptides for RNA binding activity. The method includes the steps of: (1) culturing a library of procaryotic cells that constitute a library, and (2) detecting expression of the reporter gene in a cell from the library, the expression indicating that the cell comprises a polypeptide having RNA binding activity. The cells contain at least one vector that contains a first DNA segment that encodes a fusion protein of a prokaryotic anti-terminator protein having anti-terminator activity linked in-frame to the test polypeptide, which varies among the cells in the library, that is operably linked to a second DNA segment. The second DNA segment contains a promoter, an RNA recognition sequence foreign to the anti-terminator protein, a transcription termination site and a reporter gene. The termination site blocks transcription of the reporter gene in the absence of a protein with anti-termination activity and affinity for the RNA recognition sequence. If the test polypeptide has specific affinity for the recognition sequence, it binds via the polypeptide to the RNA recognition sequence of a transcript from the second DNA segment thereby inducing transcription of the second DNA segment to proceed through the termination site to the reporter gene resulting in expression of the reporter gene.

U.S. Pat. No. 5,859,227 describes methods for identifying possible binding sites for RNA binding proteins in nucleic acid molecules, and confirming the identity of such prospective binding sites by detection of interaction between the prospective binding site and RNA binding proteins. These methods involve identification of possible binding sites for RNA binding proteins, by either searching databases for untranslated regions of gene sequences or cloning untranslated sequences using a single specific primer and an universal primer, followed by confirmation that the untranslated regions in fact interact with RNA binding proteins using the RNA/RBP detection assay. Genomic nucleic acid can further be screened for putative binding site motifs in the nucleic acid sequences. Information about binding sites that are confirmed in the assay then can be used to redefine or redirect the nucleic acid sequence search criteria, for example, by establishing or refining a consensus sequence for a given binding site motif.

SenGupta et al., Proc. Nat. Acad. Sci. U.S.A., 93:8496-8501 (1996) describes a yeast genetic method to detect and analyze RNA-protein interactions in which the binding of a bifunctional RNA to each of two hybrid proteins activates transcription of a reporter gene in vivo (see also Wang et al., Genes & Dev., 10:3028-3040 (1996)). SenGupta et al. demonstrate that this three-hybrid system enables the rapid, phenotypic detection of specific RNA-protein interactions. As examples, SenGupta et al. use the binding of the iron regulatory protein 1 (IRP 1) to the iron response element (IRE), and of HIV trans-activator protein (Tat) to the HIV trans-activation response element (TAR) RNA sequence. The three-hybrid assay relies only on the physical properties of the RNA and protein, and not on their natural biological activities; as a result, it may have broad application in the identification of RNA-binding proteins and RNAs, as well as in the detailed analysis of their interactions.

The following Table 5 illustrates certain properties of several RNA binding sequence/RNA sequence pair systems.

TABLE 5
Examples of RNA binding sequence/RNA sequence pairs

| | RNA binding sequence | | Reference (U.S. Pat. |
|---|---|---|---|
| RNA binding sequence | motif | RNA sequence | No.) |
| BINDR | double-stranded | double-stranded RNA | 5,858,675 |
| | RNA-binding | poly(rI) and poly (rC) | |
| Protein extract from SH- | 5′ untranslated region | UTR of Glut1 (SEQ ID | 5,859,227 |
| SY5Y cells | (UTR) | NO. 19); 5′ UTR of | |
| | | (HMG,CoA Red) (SEQ | |
| | | ID NO. 20); 5′ UTR of | |
| | | human C4b-binding á | |
| | | chain (SEQ ID NO. 21); | |
| | | 5′ UTR of human CD45 | |
| | | (SEQ ID NO. 22) | |

3) Preparation of Nucleic Acid Binding Proteins

Extracts prepared from the isolated nuclei of cultured cells are functional in accurate in vitro transcription and mRNA processing (See generally, Current Protocols in Molecular Biology (1998) § 12.1., John Wiley & Sons, Inc.). Thus, such extracts can be used directly for functional studies and as the starting material for purification of the proteins involved in these processes. To prepare nuclear extracts, tissue culture cells are collected, washed, and suspended in hypotonic buffer. The swollen cells are homogenized and nuclei are pelleted. The cytoplasmic fraction is removed and saved, and nuclei are resuspended in a low-salt buffer. Gentle dropwise addition of a high-salt buffer then releases soluble proteins from the nuclei (without lysing the nuclei). Following extraction, the nuclei are removed by centrifugation, the nuclear extract supernatant is dialyzed into a moderate salt solution, and any precipitated protein is removed by centrifugation.

The nuclear and cytoplasmic extraction procedure (see, e.g., Dignam et al., 1983, Nucl. Acids. Res. 11:1475-1489 (Accurate transcription initiation by RNA polymerase II in a soluble extract from isolated mammalian nuclei); Dignam, et al., 1983, Methods Enzymol. 101:582-598 (Eukaryotic gene transcription with purified components); Krainer, et al., 1984, Cell 36:993-1005 (Normal and mutant human â-globin pre-mRNAs are faithfully and efficiently spliced in vitro); Lue, et al, 1987, Proc. Natl. Acad. Sci. U.S.A. 84:8839-8843 (Accurate initiation at RNA polymerase II promoters in extracts from Saccharomyces cerevisiae); Manley, et al., 1980, Proc. Natl. Acad. Sci. U.S.A. 77:3855-3859 (DNA-dependent transcription of adenovirus genes in a soluble whole-cell extract); Weil, et al., 1979, J. Biol. Chem. 254:6163-6173 (Faithful transcription of eukaryotic genes by RNA polymerase III in systems reconstituted with purified DNA templates); and Weil, et al., 1979, Cell 18:469-484 (Selective and accurate initiation of transcription at the Ad2 major late promotor in a soluble system dependent on purified RNA polymerase II and DNA)) and the identified protein-DNA interaction pairs can be used in the present system.

4) Assays for Identifying Nucleic Acid Binding Proteins

a) Mobility shift DNA-Binding Assay

The DNA-binding assay using nondenaturing polyacrylamide gel electrophoresis (PAGE) provides a simple, rapid, and extremely sensitive method for detecting sequence-specific DNA-binding proteins (See generally, Current Protocols in Molecular Biology (1998) § 12.2., John Wiley & Sons, Inc.). Proteins that bind specifically to an end-labeled DNA fragment retard the mobility of the fragment during electrophoresis, resulting in discrete bands corresponding to the individual protein-DNA complexes. The assay can be used to test binding of purified proteins or of uncharacterized factors found in crude extracts. This assay also permits quantitative determination of the affinity, abundance, association rate constants, dissociation rate constants, and binding specificity of DNA-binding proteins.

b) Basic Mobility Shift Assay Procedure

The basic mobility shift assay procedure includes 4 steps: (1) preparation of a radioactively labeled DNA probe containing a particular protein binding site; (2) preparation of a nondenaturing gel; (3) a binding reaction in which a protein mixture is bound to the DNA probe; and (4) electrophoresis of protein-DNA complexes through the gel, which is then dried and autoradiographed. The mobility of the DNA-bound protein is retarded while that of the non-bound protein is not retarded.

c) Competition Mobility Shift Assay

One important aspect of the mobility shift DNA-binding assay is the ease of assessing the sequence specificity of protein-DNA interactions using a competition binding assay. This is necessary because most protein preparations will contain specific and nonspecific DNA binding proteins. For a specific competitor, the same DNA fragment (unlabeled) as the probe can be used. The nonspecific competitor can be essentially any fragment with an unrelated sequence, but it is useful to roughly match the probe and specific competitor for size and configuration of the ends. For example, some proteins bind blunt DNA ends nonspecifically. These would not be competed by circular plasmid or a fragment with overhands, leading to the false conclusion that the protein-DNA complex represented specific binding. Perhaps the best control competitor is a DNA fragment that is identical to the probe fragment except for a mutation(s) in the binding site that is known to disrupt function (and presumably binding).

d) Antibody Supershift Assay

Another useful variation of the mobility shift DNA-binding assay is to use antibodies to identify proteins present in the protein-DNA complex. Addition of a specific antibody to a binding reaction can have one of several effects. If the protein recognized by the antibody is not involved in complex formation, addition of the antibody should have no effect. If the protein that forms the complex is recognized by the antibody, the antibody can either block complex formation, or it can form an antibody-protein-DNA ternary complex and thereby specifically result in a further reduction in the mobility of the protein-DNA complex (supershift). Results may be different depending upon whether the antibody is added before or after the protein binds DNA (particularly if there are epitopes on the DNA-binding surface of the protein).

The mobility shift DNA-binding assay has been successfully employed (see, e.g., Carthew, et al., 1985, Cell 43:439-448 (An RNA polymerase II transcription factor binds to an upstream element in the adenovirus major late promoter); Chodosh, et al., 1986, Mol. Cell. Biol. 6:4723-4733 (A single polypeptide possesses the binding and activities of the adenovirus major late transcription factor); Fried, et al., 1981, Nucl. Acids. Res., 9:6505-6525 (Equilibria and kinetics of lac repressor-operator interactions by polyacrylamide gel electrophoresis); Fried, et al., 1984, J. Mol. Biol. 172:241-262 (Kinetics and mechanism in the reaction of gene regulatory proteins with DNA); Fried, et al., 1984, J. Mol. Biol. 172:263-282 (Equilibrium studies of the cyclic AMP receptor protein-DNA interaction); Garner, et al., 1981, Nucl. Acids Res. 9:3047-3060 (A gel electrophoresis method for quantifying the binding of proteins to specific DNA regions: Application to components of the Escherichia coli lactose operon regulatory system); Hendrickson, et al., 1984, J. Mol. Biol. 174:611-628 (Regulation of the Escherichia coli L-arabinose operon studied by gel electrophoresis DNA binding assay); Kristie, et al., 1986, Proc. Natl. Acad. Sci. U.S.A. 83:3218-3222 (The major regulatory protein of herpes simplex virus type 1, is stably and specifically associated with promoter-regulatory domains of a genes and/or selected viral genes); Lieberman, et al., 1994, Genes & Dev. 8:995-1006 (A mechanism for TAFs in transcriptional activation: Activation domain enhancement of TFIID-TFIIA-promoter DNA complex formation); Riggs, et al., 1970, J. Mol. Biol. 48:67-83 (Lac repressor-operator interactions: I. Equilibrium studies); Singh, et al., 1986, Nature 319:154-158 (A nuclear factor that binds to a conserved sequence motif in transcriptional control elements of immunoglobulin genes); Staudt, et al., 1986, Nature 323:640-643 (A lymphoid-specific protein binding to the octamer motif of immunoglobulin genes); Strauss, et al., 1984, Cell 37:889-901 (A protein binds to a satellite DNA repeat at three specific sites that would be brought into mutual proximity by DNA folding in the nucleosome); and Zinkel, et al., 1987, Nature 328:178-181 (DNA bend direction by phase-sensitive detection)) and the identified protein-DNA interaction pairs can be used in the present system.

e) Methylation and Uracil Interference Assay

Interference assays identify specific residues in the DNA binding site that, when modified, interfere with binding of the protein (See generally, Current Protocols in Molecular Biology (1998) § 12.3., John Wiley & Sons, Inc.). These protocols use end-labeled DNA probes that are modified at an average of one site per molecule of probe. These probes are incubated with the protein of interests, and protein-DNA complexes are separated from free probe by the mobility shift assay. A DNA probe that is modified at a position that interferes with binding will not be retarded in this assay; thus, the specific protein-DNA complex is depleted for DNA that contains modifications on bases important for binding. After gel purification the bound and unbound DNA are specifically cleaved at the modified residues and the resulting products analyzed by electrophoresis on polyacrylamide sequencing gels and autoradiography. These procedures provide complementary information about the nucleotides involved in protein-DNA interactions.

1) Methylation Interference Assays

In methylation interference, probes are generated by methylating guanines (at the N-7 position) and adenines (at the N-3 position) with DMS; these methylated bases are cleaved specifically by piperidine. Methylation interference identifies guanines and adenines in the DNA binding site that, when methylated, interfere with binding of the protein. The protocol uses a single end-labeled DNA probe that is methylated at an average of one site per molecule of probe. The labeled probe is a substrate for a protein-binding reaction. DNA-protein complexes are separated from the free probe by the mobility shift DNA-binding assay. A DNA probe that is methylated at a position that interferes with binding will not be retarded in this assay. Therefore, the specific DNA-protein complex is depleted for DNA that contains methyl groups on purines important for binding. After gel purification, DNA is cleaved with piperidine. Finally, these fragments are electrophoresed on polyacrylamide sequencing gels and autoradiographed. Guanines and adenines that interfere with binding are revealed by their absence in the retarded complex relative to a lane containing piperidine-cleaved free probe. This procedure offers a rapid and highly analytical means of characterizing DNA-protein interactions.

2) Uracil Interference Assay

In uracil interference, probes are generated by PCR amplification in the presence of a mixture of TTP and dUTP, thereby producing products in which thymine residues are replaced by deoxyuracil residues (which contains hydrogen in place of the thymine 5-methyl group). Uracil bases are specifically cleaved by uracil-N-glycosylase to generate apyrimidinic sites that are susceptible to piperidine. Uracil interference identifies thymines in a DNA binding site that, when modified, interfere with binding of the protein. Probes generated by PCR amplification in the presence of TTP and dUTP incorporate deoxyuracil in place of thymine residues. PCR products are incubated with the binding protein and resulting complexes are separated from unbound DNA. The DNA recovered from the protein-DNA complex is treated with uracil-N-glycosylase and piperidine, and the products are then electrophoresed on a denaturing polyacrylamide gel.

The methylation and uracil interference assays have been successfully used (see, e.g., Baldwin, et al., 1988, Proc. Natl. Acad. Sci. U.S.A. 85:723-727 (Two transcription factors, H2TF 1 and NF-kB, interact with a single regulatory sequence in the class I MHC promoter); Brunelle, et al., 1987, Proc. Natl. Acad. Sci. U.S.A. 84:6673-6676 (Missing contact probing of DNA-protein interactions); Goeddel, et al., 1978, Proc. Natl. Acad. Sci. U.S.A. 75:3579-3582 (How lac repressor recognizes lac operator); Ivarie, et al., 1987, Nucl. Acids Res. 15:9975-9983 (Thymine methyls and DNA-protein interactions); Maxam, et al., 1980, Methods Enzymol 65:499-560 (Sequencing end-labeled DNA with base-specific chemical cleavages); Pu, et al., 1992, Nucl. Acids Res. 20:771-775 (Uracil interference, a rapid and general method for defining protein-DNA interactions involving the 5-methyl group of thymines: The GCN4-DNA complex); Siebenlist, et al., 1980, Proc. Natl Acad. Sci. U.S.A. 77:122-126 (Contacts between E. coli RNA polymerase and an early promoter of phase T7); and Hendrickson, et al., 1985, Proc. Natl. Acad. Sci. U.S.A. 82:3129-3133 (A dimer of AraC protein contacts three adjacent major groove regions at the Ara I DNA site)) and the identified protein-DNA interaction pairs can be used in the present system.

3) DNase I Footprint Analysis

Deoxyribonuclease I (DNase I) protection mapping, or footprinting, is a valuable technique for locating the specific binding sites of proteins on DNA (See generally, Current Protocols in Molecular Biology (1998) § 12.4., John Wiley & Sons, Inc.). The basis of this assay is that bound protein protects that phosphodiester backbone of DNA from DNase I catalyzed hydrolysis. Binding sites are visualized by autoradiography of the DNA fragments that result form hydrolysis, following separation by electrophoresis on denaturing DNA sequencing gels. Footprinting has been developed further as a quantitative technique to determine separate binding curves for each individual protein-binding site on the DNA. For each binding site, the total energy of binding is determined directly from that site's binding curve. For sites that interact cooperatively, simultaneous numerical analysis of all the binding curves can be used to resolve the intrinsic binding and cooperative components of these energies.

DNase I footprint analysis has been successfully employed (see, e.g., Ackers, et al., 1982, Proc. Natl. Acad. Sci. U.S.A. 79:1129-1133 (Quantitative model for gene regulation by lambda phage repressor); Ackers, et al., 1983, J. Mol. Biol. 170:223-242 (Free energy coupling within macromolecules: The chemical work of ligand binding at the individual sites in cooperative systems); Brenowitz, et al., 1986, Proc. Natl. Acad. Sci. U.S.A. 83:8462-8466 (Footprint titrations yield valid thermodynamic isotherms.); Brenowitz, et al., 1986, Meth. Enzymol. 130:132-181 (Quantitative DNase I footprint titration: A method for studying protein-DNA interactions); Dabrowiak, et al., 1989, In Chemistry and Physics of DNA-Ligand Interactions (N. R. Kallenback, ed.) Adenine Press. (Quantitative footprinting analysis of drug-DNA interactions); Galas, et al., 1978, Nucl. Acids Res. 5:3157-3170 (DNase footprinting: A simple method for the detection of protein-DNA binding specificity); Hertzberg, et al., 1982, J. Am. Chem. Soc. 104:313-315 (Cleavage of double helical DNA by (methidiumpropyl-EDTA) iron (II)); Johnson, et al., 1979, Proc. Natl. Acad. Sci. U.S.A. 76:5061-5065 (Interactions between DNA-bound repressors govern regulation by the lambda phage repressor); Johnson, et al., 1985, Meth. Enzymol. 117:301-342 (Nonlinear least-squares analysis); Senear, et al., 1986, Biochemistry 25:7344-7354 (Energetics of cooperative protein-DNA interactions: Comparison between quantitative DNase I footprint titration and filter binding); and Tullius, et al., 1987, Meth. Enzymol. 155:537-558 (Hydroxyl radical footprinting: A high resolution method for mapping protein-DNA contacts), and the identified protein-DNA interaction pairs can be used in the present system.

4) Screening a ëgt11 Expression Library with Recognition-Site DNA

A clone encoding a sequence-specific protein can be detected in a ëgt11 library because its recombinant protein binds specifically to a radiolabeled recognition-site DNA (See generally, Current Protocols in Molecular Biology (1998) § 12.7., John Wiley & Sons, Inc.). Bacteriophage from a cDNA library constructed in the vector ëgt11 are plated under lytic growth conditions. After plaques appear, expression of the â-galactosidase fusion proteins encoded by the recombinant phage is induced by placing nitrocellulose filters impregnated with IPTG onto the plate. Phage growth is continued and is accompanied by the immobilization of proteins, from lysed cells, onto the nitrocellulose filters. The filters are lifted after this incubation, blocked with protein, then reacted with a radiolabeled recognition-site DNA (containing one or more binding sites for the relevant sequence-specific protein) in the presence of an excess of nonspecific competitor DNA. After the binding reaction, the filters are washed to remove nonspecifically bound probe and processed for autoradiography. Potentially positive clones detected in the primary screen are rescreened after a round of plaque purification. Recombinants which screen positively after enrichment and whose detection specifically requires the recognition-site probe (non detected with control probes lacking the recognition site for the relevant protein) are then isolated by further rounds of plaque purification.

The ëgt11 expression screening methods have been successfully used (see, e.g., Androphy, et al., 1987, Nature (Lond.) 325:70-73 (Bovine papillomavirus E2 trans-activating gene product binds to specific sites in papillomavirus DNA); Arndt, et al., 1986, Proc. Natl. Acad. Sci. U.S.A. 83:8516-8520 (GCN4 protein, a positive transcription factor in yeast, binds general control promoters at 5′TGACTC3′ sequences); Chodosh, et al., 1988, Cell 53:25-35 (A yeast and a human CCAAT-binding protein have heterologous subunits that are functionally interchangeable); Desplan, et al., 1985, Nature (Lond.) 318:630-635 (The Drosophila developmental gene, engrailed, encodes a sequence-specific DNA binding activity); Hoeffler, et al., 1988, Science 242:1430-1433 (Cyclic AMP-responsive DNA-binding protein: Structure based on a cloned placental cDNA); Hsiou-Chi, et al., 1988, Science 242:69-71 (Distinct cloned class II MHC DNA binding proteins recognize the X box transcription element); Ingraham, et al., 1988, Cell 55:519-529 (A tissue-specific transcription factor containing a homeo domain specifies a pituitary phenotype); Kadonaga, et al., 1987, Cell 51:1079-1090 (Isolation of cDNA encoding transcription factor Sp1 an functional. analysis of the DNA binding domain); Keegan, et al., 1986, Science 231:699-704 (Separation of DNA binding from the transcription-activating function of a eukaryotic regulatory protein); Miyamoto, et al., 1988, Cell 54:903-913 (Regulated expression of a gene encoding a nucleic factor, IRF-1, that specifically binds to IFN-â gene regulatory elements); Murre, et al., 1989, Cell 56:777-783 (A new DNA binding and dimerization motif in immunoglobulin enhancer binding, daughterless, MyoD and myc proteins); Müller, et al., 1988, Nature (Lond.) 336:544-551 (A cloned octamer transcription factor stimulates transcription from lymphoid specific promoters in non-B cells); Rawlins, et al., 1985, Cell 42:859-868 (Sequence-specific DNA binding of the Epstein-Barr viral nuclear antigen (EBNA-1) to clustered sites in the plasmid maintenance region); Reith, et al., 1989, Proc. Natl. Acad. Sci. U.S.A. 86:4200-4204 (Cloning of the major histocompatibility complex class II promoter affected in a hereditary defect in class II gene regulation); Singh, et al., 1988, Cell 52:415-423 (Molecular cloning of an enhancer binding protein: Isolation by screening of an expression library with a recognition site); Staudt, et al., 1988, Science 241:577-580 (Molecular cloning of a lymphoid-specific cDNA encoding a protein that binds to the regulatory octamer DNA motif); Sturm, et al., 1988, Genes & Dev. 2:1582-1599 (The ubiquitous octamer protein Oct-1contains a Pou domain with a homeo subdomain); Vinson, et al., 1988, Genes & Dev. 2:801-806 (In situ detection of sequence-specific DNA binding activity specified by a recombinant bacteriophage); Weinberger, et al., 1985, Science 228:740-742 (Identification of human glucocorticoid receptor complementary DNA clones by epitope selection); and Young, et al., 1983, Science 222:778-782 (Yeast RNA polymerase II genes: Isolation with antibody probes)) and the identified protein-DNA interaction pairs can be used in the present system.

5) Rapid Separation of Protein-Bound DNA from Free DNA

This method relies on the ability of nitrocellulose to bind proteins but not double-stranded DNA (See generally, Current Protocols in Molecular Biology (1998) § 12.8., John Wiley & Sons, Inc.). Use of radioactively labeled double-stranded DNA fragments allows quantitation of DNA bound to the protein at various times and under various conditions, permitting kinetic and equilibrium studies of DNA-binding interactions. Purified protein is mixed with double-stranded DNA in an appropriate buffer to allow interaction. After incubation, the mixture is suction filtered through nitrocellulose, allowing unbound DNA to pass through the filter while the protein (and any DNA interacting with it) is retained.

Nitrocellulose filter methods have been successfully used (see, e.g., Barkley, et al., 1975, Biochemistry 14:1700-1712 (Interaction of effecting ligands with lac repressor and repressor-operator complex); Fried, et al., 1981, Nucl. Acids Res. 9:6505-6525 (Equilibria and kinetics of lac repressor-operator interactions by polyacrylamide gel electrophoresis); Hinkle, et al., 1972, J. Mol. Biol. 70:157-185 (Studies of the binding of Escherichia coli RNA polymerase to DNA I. The role of sigma subunit in site selection); Hinkle, et al., 1972, J. Mol. Biol. 70:187-195 (Studies of the binding of Escherichia coli RNA polymerase to DNA II. The kinetics of the binding reaction); Hinkle, et al., 1972, J. Mol. Biol. 70:197-207 (Studies of the binding of Escherichia coli RNA polymerase to DNA III. Tight binding of RNA polymerase holoenzyme to single-strand breaks in T7 DNA); Jones, et al., 1966, J. Mol. Biol. 22:199-209 (Studies on the binding of RNA polymerase to polynucleotides); Lin, et al., 1972, J. Mol. Biol. 72:671-690 (Lac repressor binding to non-operator DNA: Detailed studies and a comparison of equilibrium and rate competition methods); Lin, et al., 1975, Cell 4:107-111 (The general affinity of lac repressor for E. Coli DNA: Implications for gene regulation in procaryotes and eucaryotes); Nirenberg, et al., 1964, Science 145:1399-1407 (RNA codewords and protein synthesis: The effect of trinucleotides upon the binding of sRNA to ribosomes); Ptashne, et al., 1987, A Genetic Switch: Gene Control and Phage ëpp. 80-83 and 109-118. Cell Press, Cambridge, Mass. and Blackwell Scientific, Boston, Mass.; Riggs, et al., 1970, J. Mol Biol. 48:67-83 (Lac repressor-operator interactions: I. Equilibrium studies); Strauss, et al., 1980, Biochemistry 19:3496-3504 (Binding of Escherichia coli ribonucleic acid polymerase holoenzyme to a bacteriophage T7 promoter-containing fragment: Selectivity exists over a wide range of solution conditions); Strauss, et al., 1980, Biochemistry 19:3504-3515 (Binding of Escherichia coli ribonucleic acid polymerase holoenzyme to a bacteriophage T7 promoter-containing fragment: Evaluation of promoter binding constants as a function of solution conditions); and Strauss, et al., 1981, Gene 13:75-87 (Variables affecting the selectivity and efficiency of retention of DNA fragments by E. coli RNA polymerase in the nitrocellulose-filter binding assay)) and the identified protein-DNA interaction pairs can be used in the present system.

f. Lipid Binding Moieties

The conjugate can also contain a lipid binding protein, peptide or effective fragment thereof. Its specific binding partner can be lipids generally, a set of lipids or a particular lipid. Any lipid binding moiety, particularly proteins, peptides or effective fragments thereof can be used in the present system. For example, the lipid binding protein can bind to a triacylglycerol, a wax, a phosphoglyceride, a sphingolipid, a sterol and a sterol fatty acid ester. More preferably, the lipid binding sequence comprises a C2 motif or an amphipathic á-helix motif.

Any lipid binding sequence/lipid pair can be designed, screened or selected according to the methods known in the art (see, e.g., Kane et al., Anal. Biochem., 233(2):197-204 (1996); Arnold et al., Biochim. Biophys. Acta, 1233(2):198-204 (1995); Miller and Cistola, Mol. Cell. Biochem., 123(1-2):29-37 (1993); and Teegarden et al., Anal Biochem., 199(2):293-9 (1991).

For example, Kane et al., Anal. Biochem., 233(2):197-204 (1996) describes that the fluorescent probe 1-anilinonapthalene 8-sulfonic acid (1,8-ANS) has been used to characterize a general assay for members of the intracellular lipid-binding protein (iLBP) multigene family. The adipocyte lipid-binding protein (ALBP), the keratinocyte lipid-binding protein (KLBP), the cellular retinol-binding protein (CRBP), and the cellular retinoic acid-binding protein I (CRABPI) have been characterized as to their ligand binding activities using 1,8-ANS. ALBP and KLBP exhibited the highest affinity probe binding with apparent dissociation constants (Kd) of 410 and 530 nM, respectively, while CRBP and CRABPI bound 1,8-ANS with apparent dissociation constants of 7.7 and 25 microM, respectively. In order to quantitate the fatty acid and retinoid binding specificity and affinity of ALBP, KLBP, and CRBP, a competition assay was developed to monitor the ability of various lipid molecules to displace bound 1,8-ANS from the binding cavity. Oleic acid and arachidonic acid displaced bound 1,8-ANS from ALBP, with apparent inhibitor constants (Ki) of 134 nM, while all-trans-retinoic acid exhibited a seven-fold lower Ki (870 nM). The short chain fatty acid octanoic acid and all-trans-retinol did not displace the fluorophore from ALBP to any measurable extent. In comparison, the displacement assay revealed that KLBP bound oleic acid and arachidonic acid with high affinity (Ki=420 and 400 nM, respectively) but bound all-trans-retinoic acid with a markedly reduced affinity (Ki=3.6 microM). Like that for ALBP, neither octanoic acid nor all-trans-retinol were bound by KLBP. Displacement of 1,8-ANS from CRBP by all-trans-retinal and all-trans-retinoic acid yielded Ki values of 1.7 and 5.3 microM, respectively. These results indicate the utility of the assay for characterizing the ligand binding characteristics of members of the iLBP family and suggests that this technique may be used to characterize the ligand binding properties of other hydrophobic ligand binding proteins.

Arnold et al., Biochim. Biophys. Acta, 1233(2):198-204 (1995) describes an assay for analyzing the specific binding of proteins to lipid ligands contained within vesicles or micelles. This assay, referred to as the electrophoretic migration shift assay, was developed using a model system composed of cholera toxin and of its physiological receptor, monosialoganglioside GM1. Using polyacrylamide gel electrophoresis in non-denaturing conditions, the migration of toxin components known to interact with GM1 was retarded when GM1 was present in either lipid vesicles or micelles. This effect was specific, as the migration of proteins not interacting with GM1 was not modified. The localization of retarded proteins and of lipids on gels was further determined by autoradiography. The stoichiometry of binding between cholera toxin and GM1 was determined, giving a value of five GM1per one pentameric assembly of cholera toxin B-subunits, in agreement with previous studies. The general applicability of this assay was further established using streptavidin and annexin V together with specific lipid ligands. This assay is fast, simple, quantitative, and requires only microgram quantities of protein.

Miller and Cistola, Mol. Cell. Biochem., 123(1-2):29-37 (1993) teaches that titration calorimetry can be used as a method for obtaining binding constants and thermodynamic parameters for the cytosolic fatty acid- and lipid-binding proteins. A feature of this method is its ability to accurately determine binding constants in a non-perturbing manner. This is achieved because the assay does not require separation of bound and free ligand to obtain binding parameters. Also, the structure of the lipid-protein complex was not perturbed, since native ligands were used rather than non-native analogues. As illustrated for liver fatty acid-binding protein, the method distinguished affinity classes whose dissociation constants differed by an order of magnitude or less. It also distinguished endothermic from exothermic binding reactions, as illustrated for the binding of two closely related bile salts to ileal lipid-binding protein. The main limitations of the method are its relatively low sensitivity and the difficulty working with highly insoluble ligands, such as cholesterol or saturated long-chain fatty acids. The signal-to-noise ratio was improved by manipulating the buffer conditions, as illustrated for oleate binding to rat intestinal fatty acid binding protein.

Teegarden et al., Anal. Biochem., 199(2):293-9 (1991) describes an assay for measurement of the affinity of serum vitamin D binding protein for 25-hydroxyvitamin D3, 1,25-dihydroxyvitamin D3, and vitamin D3, using uniform diameter (6.4 microns) polystyrene beads coated with phosphatidylcholine and vitamin D metabolites as the vitamin D donor. The lipid metabolite coated beads have a solid core, and thus all of the vitamin D metabolites are on the bead surface from which transfer to protein occurs. After incubating these beads in neutral buffer for 3 h, essentially no ³H-labeled vitamin D metabolites desorb from this surface. Phosphatidylcholine/vitamin D metabolite-coated beads (1 microM vitamin D metabolite) were incubated with varying concentrations of serum vitamin D binding protein under conditions in which the bead surfaces were saturated with protein, but most of the protein was free in solution. After incubation, beads were rapidly centrifuged without disturbing the equilibrium of binding and vitamin D metabolite bound to sDBP in solution was assayed in the supernatant. All three vitamin D metabolites became bound to serum vitamin D binding protein, and after 10 min of incubation the transfer of the metabolites to serum vitamin D binding protein was time independent. The transfer followed a Langmuir isotherm, and the Kd for each metabolite binding to serum vitamin D binding protein was derived by nonlinear least-squares fit analysis. From this analysis the following values for the Kd were obtained: 5.59×10⁻⁶ M, 25-hydroxyvitamin D; 9.45×10⁻⁶ M, 1,25-dihydroxyvitamin D; and 9.17×10⁻⁵ M, vitamin D. The method disclosed herein avoids problems encountered in previous assays and allows the precise and convenient determination of binding affinities of vitamin D metabolites and serum vitamin D binding protein.

In addition, known protein/lipid binding pairs can be used in the methods and with the products provided herein (see, e.g., Hinderliter et al., Biochim. Biophys. Acta, 1448(2):227-35 (1998) (C2 motif binds phospholipid in a manner that is modulated by Ca2+ and confers membrane-binding ability on a wide variety of proteins, primarily proteins involved in signal transduction and membrane trafficking events); Campagna et al., J. Diary Sci., 81(12):3139-48 (1998) (an amphipathic helical lipid-binding motif of a glycosylated phosphoprotein, component PP3 in bovine milk); Chae et al., J. Biol. Chem., 273(40):25659-63 (1998) (The C2A domain of synaptotagmin I, which binds Ca2+ and anionic phospholipids); Johnson et al., Biochemistry, 37(26):9509-19 (1998) (the membrane binding domain of phosphocholine cytidylyltransferase (CT) includes a continuous amphipathic alpha-helix between residues approximately 240-295 anionic lipids); Kiyosue et al., Plant Mol. Biol., 35(6):969-72 (1997) (Ca2+-dependent lipid-binding domains of cytosolic phospholipase A2, protein kinase C, Rabphilin-3A, and Synaptotagmin 1 of animals); Welters et al., Proc. Natl. Acad. Sci. USA, 91(24):11398-402 (1994) (calcium-dependent lipid-binding domain is near the N terminus of phosphatidylinositol (PI) 3-kinase cloned from Arabidopsis thaliana); and Filoteo et al., J. Biol. Chem., 267(17):11800-5 (1992) (Peptide G25: LysLysAlaValLysValProLysLysGluLysSerValLeuGlnGlyLysLeuThrArgLeuAlaValGlnIle (SEQ ID No. 23) representing the putative lipid-binding region (G region) of the erythrocyte Ca2+ pump interacted with acidic lipids, as shown by the increase in size of phosphatidylserine liposomes in its presence)).

Polysaccharide Binding Moieties

The conjugate can include a polysaccharide binding protein, peptide or effective fragment thereof. Its specific binding partner can be polysaccharides generally, a set of polysaccharides or a particular polysaccharide. Any polysaccharide binding moiety, such as a protein, can be used in the present system and include but are not limited to a polysaccharide binding sequence that binds to starch, glycogen, cellulose or hyaluronic acid.

Any polysaccharide binding protein/polysaccharide pair can be designed, screened or selected according to the methods known in the art including the methods disclosed in Kuo et al., J. Immunol. Methods, 43(1):35-47 (1981); and Brandt et al., J. Immunol., 108(4):913-20 (1972) (a radioactive antigen-binding assay for Neisseria meningitidis polysaccharide antibody). Kuo et al., J. Immunol. Methods, 43(1):35-47 (1981) provides a polyethylene glycol (PEG) radioimmunoprecipitation assay for the detection of antibody to Haemophilus influenza b capsular polysaccharide, polyribosylribitol phosphate (PRP). The radioactive antigen, [³ H]PRP, with a high specific activity, was produced by growing the organism in the presence of [3H]ribose and was purified by hydroxylapatite and Sepharose™ 4B column chromatography. In the assay, PEG (12.5%) was used to separate antibody-bound [³H]PRP from free [³H]PRP. The assay covered the range of 0.5 and 20 ng antibody/assay at a maximum sensitivity of 0.5 approximately 1.0 ng antibody/assay. With various dilutions (1-20 ng antibody/assay) of S. Klein reference antiserum, the within-run coefficient of variation (CV) of 10 replicates ranged from 3.5 to 8.5%. Average CVs of 8.9% and 11.0% were obtained in the between-run and day-to-day reproducibility studies. The binding of [3 H]PRP to S. Klein reference antiserum was severely inhibited by a minute amount of non-radioactive PRP; however, no significant interference was found in the presence of high concentrations of polysaccharides from Escherichia coli K100 and Streptococcus pneumoniae indicating that the RIA was highly specific for antibody to H. influenza b PRP.

In addition, known protein/polysaccharide binding pairs can be used in the methods and with the products provided herein (see, e.g., Yamaguchi, et al., Oral Microbiol. Immunol., 13(6):348-54 (1998) (capsule-like serotype-specific polysaccharide antigen lipopolysaccharide from Actinobacillus actinomycetemcomitans/human complement-derived opsonins); Lucas, et al., J. Immunol., 161(7):3776-80 (1998) (kappa II-A2 light chain CDR-3 junctional residues in human antibody/Haemophilus influenza type b polysaccharide); Miller, et al., Carbohydr. Res., 309(3):219-26 (1998) (fragments of the Shigella dysenteriae type 1 O-specific polysaccharide/monoclonal IgM 3707 E9); Prehm, et al., Protein Expr. Purif, 7(4):343-6 (1996) (digitonin/hyaluronate synthase); Jiang, et al., Infect. Immun., 63(7):2537-40 (1995) (mannose-binding protein/Klebsiella O3 lipopolysaccharide); Pelkonen, et al., J. Bacteriol., 174(23):7757-61 (1992) (bacteriophage depolymerase/bacterial polysaccharide); Morishita, et al., Biochem. Biophys. Res. Commun., 176(3):949-57 (1991) (Microbial polysaccharide, HS-142-1/guanylyl cyclase-containing receptor); Ohtomo, et al., Can. J. Microbiol., 36(3):206-10 (1990) (staphylococcal cell surface polysaccharide/human fibrinogen); Yamagishi, et al., FEBS Lett., 225(1-2):109-12 (1987) (heparin or dermatan sulfate/thrombin); DeAngelis, et al., J. Biol. Chem., 262(29):13946-52 (1987) (sulfated fucans/bindin, the adhesive protein from sea urchin sperm); Volanakis, et al., Mol. Immunol., 20(11):1201-7 (1983) (human C4/C-reactive protein-pneumococcal C-polysaccharide complexes); Naruse, et al., J. Biochem. (Tokyo), 90(3):581-7 (1981) (a polysaccharide from the cortex of sea urchin egg/microtubule-associated proteins); Levy, et al., J. Exp. Med., 153(4):883-96 (1981) (agaropectin and heparin/human IgG proteins); Hu, et al., Biochemistry, 14(10):2224-30 (1975) (glycogen phosphorylase A/a series of semisynthetic, branched saccharides); Fagerstrom, Microbiology, 140(9):2399-407 (1994) (raw-starch-binding consensus amino acids in the C-terminal part of glucoamylase P); Murata, et al., J. Vet. Med. Sci., 57(3):419-25 (1995) (C-polysaccharide/C-reactive protein (CRP)); Reason, et al., Infect. Immun., 67(2):994-7 (1999) (Antibodies having light (L) chains encoded by the kappaII-A2 variable region/Haemophilus influenza type b polysaccharide (Hib PS)).

h. Metal Binding Moieties

The conjugate can contain a metal binding moiety, such as a metal binding protein, peptide or effective fragment thereof. The specific binding partner can be metal ions generally, a set of metal ions or a particular metal ion. Any metal binding moiety is contemplated. For example, the metal binding sequence can bind to a sodium, a potassium, a magnesium, a calcium, a chlorine, an iron, a copper, a zinc, a manganese, a cobalt, an iodine, a molybdenum, a vanadium, a nickel, a chromium, a fluorine, a silicon, a tin, a boron or an arsenic ion.

Any metal binding moiety/metal ion pair can be designed, screened or selected according to the methods known in the art including the methods disclosed in U.S. Pat. No. 5,679,548; Kang et al., Virus Res., 49(2):147-54 (1997); Dealwis et al., Biochemistry, 34(43):13967-73 (1995); and Hutchens et al., J. Chromatogr., 604(1):125-32 (1992).

U.S. Pat. No. 5,679,548 discloses a method for producing a metal binding site in a polypeptide capable of binding a preselected metal ion-containing molecule, the step of inducing mutagenesis of a complementarity determining region (CDR) of an immunoglobulin heavy or light chain gene, where mutagenesis introduces a metal binding site, by amplifying the CDR of the gene by a primer extension reaction using a primer oligonucleotide, the oligonucleotide comprising: a) a 3′ terminus and a 5′ terminus comprising; b) a nucleotide sequence at the 3′ terminus complementary to a first framework region of the heavy or light chain immunoglobulin gene; c) a nucleotide sequence at the 5′ terminus complementary to a second framework region of the heavy or light chain immunoglobulin gene; and d) a nucleotide sequence between the 3′ terminus and 5′ terminus according to the formula; [NNS]ₐ, wherein N is independently any nucleotide, S is G or C, and a is from 3 to about 50, and the 3′ and 5′ terminal nucleotide sequences having a length of about 6 to 50 nucleotides, and sequences complementary thereto.

U.S. Pat. No. 5,679,548 also describes a method for producing a metal binding site in a polypeptide capable of binding a preselected metal ion-containing molecule, the step of inducing mutagenesis of a complementarity determining region (CDR) of an immunoglobulin heavy or light chain gene by amplifying the CDR of the gene by a primer extension reaction using a primer oligonucleotide, the oligonucleotide comprising: a) a 3′ terminus and a 5′ terminus; b) a nucleotide sequence at the 3′ terminus complementary to a first framework region of the heavy or light chain immunoglobulin gene; c) a nucleotide sequence at the 5′ terminus complementary to a second framework region of the heavy or light chain immunoglobulin gene; and d) a nucleotide sequence between 3′ terminus and 5′ terminus according to the formula: —X—[NNK]ₐ—X—[NNK]—X, wherein N is independently any nucleotide, K is G or T, X is a trinucleotide encoding a native amino acid residue coded by the immunoglobulin gene and a is from 3 to about 50, and the 3′ and 5′ terminal nucleotide sequences having a length of about 6 to 50 nucleotides, and sequences complementary thereto. Preferably, the immunoglobulin to be mutagenized is a human immunoglobulin, the CDR is CDR3, the mutagenizing oligonucleotide has the formula: 5′-GTGTATTATTGTGCGAGA[NNS]ₐTGGGGCCAAGGGACCACG-3′ (SEQ ID No. 24), and the preselected metal ion-containing molecule is magnetite, copper(II), zinc(II), lead(II), cerium(III), or iron(III).

Kang et al., Virus Res., 49(2):147-54 (1997) isolated human papillomavirus (HPV) type 18 E7 gene by polymerase chain reaction (PCR) amplification from tissues of Korean cervical cancer patients and cloned into a plasmid vector, pET-3a, for the expression of recombinant E7 protein (rE7) in Escherichia coli. The rE7 protein was purified to the homogeneity and its purity was confirmed by HPLC. The purified protein was analyzed for the metal-binding properties by UV spectroscopy and it was shown that two Cd²⁺ or Zn²⁺ ions bind to one E7 protein by the metal-sulfur ligand formation via two Cys-X-X-Cys motifs in E7 protein. When the change of intrinsic fluorescence of tryptophan residue was analyzed for rE7-Zn complex, the blue shift of emission wavelength and the decrease in maximum intensity of emission were observed compared with rE7. These results suggest that Zn²⁺-bound rE7 has undergone conformational change, in which a tryptophan residue located in the second Cys-X-X-Cys motif was moved into solvent-inaccessible or hydrophobic environment.

Dealwis et al., Biochemistry, 34(43):13967-73 (1995) present the refined crystal structures of three different conformational states of the Asp153-->Gly mutant (D153G) of alkaline phosphatase (AP), a metalloenzyme from Escherichia coli. The apo state is induced in the crystal over a 3 month period by metal depletion of the holoenzyme crystals. Subsequently, the metals are reintroduced in the crystalline state in a time-dependent reversible manner without physically damaging the crystals. Two structural intermediates of the holo form based on data from a 2 week (intermediate I) and a 2 month soak (intermediate II) of the apo crystals with Mg²⁺ and Zn²+ have been identified. The three-dimensional crystal structures of the apo (R=18.1%), intermediate I (R=19.5%), and intermediate II (R=19.9%) of the D153G enzyme have been refined and the corresponding structures analyzed and compared. Large conformational changes that extend from the mutant active site to surface loops, located 20 A away, are observed in the apo structure with respect to the holo structure. The structure of intermediate I shows the recovery of the entire enzyme to an almost native-like conformation, with the exception of residues Asp 51 and Asp 369 in the active site and the surface loop (406-410) which remains partially disordered. In the three-dimensional structure of intermediate II, Asp 51 and Asp 369 are essentially in a native-like conformation, but the main chain of residues 406-408 within the loop is still not fully ordered. The D153G mutant protein exhibits weak, reversible, time dependent metal binding in solution and in the crystalline state.

Hutchens et al., J. Chromatogr., 604(1):125-32 (1992) prepared synthetic peptides representing metal-binding protein surface domains from the human plasma metal transport protein known as histidine-rich glycoprotein (HRG) to evaluate biologically relevant peptide-metal ion interactions. Three synthetic peptides, representing multiples of a 5-residue repeat sequence (Gly-His-His-Pro-His) (SEQ ID No. 25) from within the histidine- and proline-rich region of the C-terminal domain were prepared. Prior to immobilization, the synthetic peptides were evaluated for identity and sample homogeneity by matrix-assisted UV laser desorption time-of-flight mass spectrometry (LDTOF-MS). Peptides with bound sodium and potassium ions were observed; however, these signal intensities were reduced by immersion of the sample probe tip in water. Mixtures of the three different synthetic peptides were also evaluated by LDTOF-MS after their elution through a special immobilized peptide-metal ion column designed to investigate metal ion transfer. It was found that LDTOF-MS to be a useful new method to verify the presence of peptide-bound metal ions.

In addition, the protein/metal binding pairs, which are known (see, e.g., DiDonato, et al., Adv. Exp. Med. Biol., 448:165-73 (1999) (copper/copper binding domain from the Wilson disease copper transporting ATPase (ATP7B)); Buchko, et al., Biochem Biophis. Res. Commun., 254(1):109-13 (1999) (Zn²⁺/Xenopus laevis nucleotide excision repair protein XPA); Lai, et al., Biochemistry, 37(48):7005-15 (1998) (Zn²⁺/hdm2 RING finder domain); Mitterauer, et al., Biochemistry, 37(46):16183-91 (1998) (The C2 catalytic domain of adenylyl cyclase contains the second metal ion (Mn2+) binding site); Hess, et al., Protein Sci., 7(9):1970-5 (1998) (Zn²⁺/Human nucleotide excision repair protein XPA); Goedken, et al., Proteins, 33(1):135-43 (1998) (Mg²⁺ and Mn²⁺/ribonuclease H domain of Moloney murine leukemia virus reverse transcriptase); Chang, et al., Protein Eng., 11(1):41-6 (1998) (beta-domain of metallothionein); Champeil, et al., J. Biol. Chem., 273(12):6619-31 (1998) (cytosolic portion of sarcoplasmic reticulum Ca2+-ATPase); Bavoso, et al., Biochem. Biophys. Res. Commun., 242(2):385-9 (1998) (zinc finger peptide containing the Cys-X2-Cys-X4-His-X4-Cys domain encoded by the Drosophila Fw-element); Gitschier, et al., Nat. Struct. Biol., 5(1):47-54 (1998) (metal-binding domain from the Menkes copper-transporting ATPase); Gadhavi, FEBS Lett., 417(1):145-9 (1997) (Zn²⁺/ion binding site in the DNA binding domain of the yeast transcriptional activator GAL4); Roehm, et al., Biochemistry, 36(33):10240-5 (1997) (Zn²⁺/RING finger domain of BRCA1); Dalton, et al., Mol. Cell Biol, 17(5):2781-9 (1997) (metal response element-binding transcription factor 1 DNA binding involves zinc interaction with the zinc finger domain); Essen, et al., Biochemistry, 36(10):2753-62 (1997) (Ca²⁺/A ternary metal binding site in the C2 domain of phosphoinositide-specific phospholipase C-delta1); Curtis, et al., EMBO J., 16(4):834:43 (1997) (Zn²⁺/CCHC metal-binding domain in Nanos); Worthington, et al., Proc. Natl. Acad. Sci. USA, 93(24):13754-9 (1996) (zinc-binding domain of Nup475); Mahadevan, et al., Biochemistry, 34(7):2095-106 (1995) (Ba²⁺, Ca²⁺, Mg²⁺, Mn²⁺, Ni²⁺, Zn²⁺/A divalent metal ion binding site in the kinase insert domain of the alpha-platelet-derived growth factor receptor); Pan, et al., Biochem. Biophys. Res. Commun., 202(1):621-8 (1994) (alpha and beta domains of mammalian metallothionein); Borden, et al., FEBS Lett., 335(2):255-60 (1993) (Cu²⁺, Zn²+/cysteine/histidine-rich metal binding domain from Xenopus nuclear factor XNF7); Chauhan, et al., J. Bacteriol., 175(22):7222-7 (1993) (Mg²⁺/Bradyrhizobium japonicum delta-aminolevulinic acid dehydratase is metal-binding domain); Knegtel, et al, Biochem. Biophys. Res. Commun., 192(2):492-8 (1993) (Zn²⁺/metal coordination in the human retinoic acid receptor-beta DNA binding domain); Spencer, et al., Biochem. J., 290(1):279-87 (1993) (Co²⁺, Mg²⁺, Zn²⁺/5-aminolaevulinic acid dehydratase from Escherichia coli reactive thiols at the metal-binding domain); Mau, et al., Protein Sci., 1(11):1403-12 (1992) (Zn²⁺/GAL4 DNA-binding domain); Vaughan, et al., Virology, 189(1):377-84 (1992) (Zn²⁺/The herpes simplex virus immediate early protein ICP27 metal binding domain); Boese, et al., J. Biol. Chem., 266(26):17060-6 (1991) (Mg²⁺/Aminolevulinic acid dehydratase in pea metal-binding domain); Hutchens, et al, J. Biol. Chem., 264(29):17206-12 (1989) (Cu²⁺, Ni²⁺, Zn²⁺/DNA-binding estrogen receptor); Stillman, et al., Biochem. J., 262(1):181-8 (1989) (Cd²⁺and Zn²⁺/rabbit liver metallothionein 2); Freedman, et al., Nature, 334(6182):543-6 (1988) (Cd²⁺ and Zn²⁺/metal coordination sites within the glucocorticoid receptor DNA binding domain); Stillman, et al., J. Biol. Chem., 263(13):6128-33 (1988) (Cd²⁺ and Zn²⁺/metallothionein); and Corson, et al., Biochemistry, 25(7):1817-26 (1986) (Ca²⁺/calcium-binding proteins C-terminal alpha-helix of a helix-loop-helix metal-binding domain)) can be used in the present system.

Among the preferred pairs, are the following metal binding sequence/metal ion pairs (see, U.S. Pat. No. 5,679,548) set forth in the following table.

TABLE 6

Examples of Metal Ion Binding Sequence/Metal Ion Pairs

| | | |
|---|---|---|
| Metal Ion | Metal Ion Binding Sequence | SEQ ID NO. |

| | | |
|---|---|---|
| Mg(II) | SerArgArgSerArgHisHisProArgMetTrpAsnGlyLeuAspVal | 26 |
| | GlyArgPheLysArgValArgAspArgTrpValValIlePheAspPhe | 27 |
| | GlyValAlaArgSerLysLysMetArgGlyLeuTrpArgLeuAspVal | 28 |
| | GlyLeuAlaValArgSerLysArgGlyArgPhePheLeupheAspVal | 29 |
| Cu(II) | GlyArgValHisHisHisSerLeuAspVal | 30 |
| | SerTrpLysHisHisAlaHisTrpAspVal | 31 |
| | GlySerTrpAspHisArgGlyCysAspGly | 32 |
| | GlyHisHisMetTyrGlyGlyTrpAspHis | 33 |
| | GlyHisTrpGlyArgHisSerLeuAspThr | 34 |
| | GlyHisIleLeuHisHisGlnLeuAspLeu | 35 |
| | SerSerGlnArgLeuMetLeuGlyAspAsn | 36 |
| | SerHisHisGlyHisHisTyrLeuAsnHis | 37 |
| | GlyLysLeuMetMetSerTrpCysArgAspThrGluGlyCysAspHis | 38 |
| | GlyAspThrHisArgGlyHisLeuArgHisHisLeuProHisAspTrp | 39 |
| | GlyTrpGlyLeuTrpMetLysProPheValTrpArgAlaTrpAspMet | 40 |
| Zn(II) | GlyArgValHisHisHisSerLeuAspVal | 41 |
| | SerHisThrHisAlaLeuProLeuAspPhe | 42 |
| | GlyGlnSerSerGlyGlyAspThrAspAsp | 43 |
| | GlyGlnTrpThrProArgGlyAspAspPhe | 44 |
| | GlyArgCysCysProSerSerCysAspGlu | 45 |
| | GlyProAlaLysHisArgHisArgHisValGlyGlnMetHisAspSer | 46 |
| Pb(III) | GlyAsnLeuArgArgLysThrSerAspIle | 47 |
| | GLyGluSerAspSerLysArgGluAspGly | 48 |
| | GlyGlyProSerLeuAlaValGlyAspTrp | 49 |
| | GlyProLeuGlnHisThrTyrProAspTyr | 50 |
| | GlyTrpLysValThrAlaGluAspSerThrGluGlyLeuPheAspLeu | 51 |
| | GlyThrArgValTrpArgValCysGlnTrpAsnHisGluGluAspGly | 52 |
| | GlyGluTrpTrpCysSerPheAlaMetCysProAlaArgTrpAspPhe | 53 |
| | GlyAspThrIlePheGlyValThrMetGlyTyrTyrAlaMetAspVal | 54 |
| Ce(III) | GlyGlnValMetGlnGluLeuGlyAspAla | 55 |
| | GlyLeuThrGluGlnGlnLeuGlnAspGly | 56 |
| | GlyTyrSerTyrSerValSerProAspAla | 57 |
| | GlyArgLeuGlyLeuValMetThrAspGlu | 58 |
| | SerThrTrpProGlyArgGlnArgLeuGlyGlnAlaLeuSerAspSer | 59 |
| | GlyTyrGluLeuSerTrpGlyValAspGlnGlnGluTrpTrpAspIle | 60 |
| | GlyProValArgGlyLeuAspGlnSerLysGlyValArgTyrAspAsn | 61 |
| | GlyLeuSerGlnHisIleValSerGluThrGlnSerSerGlyAspLeu | 62 |
| | GlyLeuGluSerLeuLysValLeuGlyValGlnLeuGlyGlyAspLeu | 63 |
| | GlyAsnMetIleLeuGlyGlyProGlyCysTrpSerSerAlaAspIle | 64 |
| | GlyCysTrpAsnValGlnArgLeuValValTyrHisProProAspGly | 65 |
| | GlyPheGluValThrCysSerTrpPheGlyHisTrpGlyArgAspSer | 66 |
| Fe(III) | SerAlaSerMetArgSerAlaIleGlyLeuTrpArgThrMetAspTyr | 67 |
| | GlyAspArgGluIlePheHisMetGlnTrpProLeuArgValAspVal | 68 |
| | SerGlnAsnProGlnGlnValCysGlyValArgCysGlyGlnAspLys | 69 |
| | GlyAsnArgLeuSerSerGlyHisLeuLeuLysGlnGlyGlnAspGly | 70 |
| | GlyGlySerAspTrpGlnIleGlyAlaCysCysArgGluAspAspLeu | 71 |
| | GlyMetValSerMetMetGlyGlnSerArgProThrGlnCysAspCys | 72 |
| | GlyValIleLysTrpIleArgArgTrpValArgThrAlaArgAspVal | 73 |
| | GlyTrpPheTrpArgLeuLeuProThrProArgAlaProSerAspVal | 74 |

Facilitating agents can be derived from an enzyme, a transport protein, a nutrient or storage protein, a contractile or motile protein, a structural protein, a defense protein, a regulatory protein, or a fluorescent protein. Exemplary of such other fragments are those derived from an enzyme such as a peroxidase, a urease, an alkaline phosphatase, a luciferase and a glutathione S-transferase.

1) Peroxidase

Any peroxidase can be used in the present system. More preferably, a horseradish peroxidase is used. For example, the horseradish peroxidases with the following GenBank accession Nos. can be used: E01651; D90116 (prxC3 gene); D90115 (prxC2 gene); J05552 (Synthetic isoenzyme C(HRP-C)); S14268 (neutral); OPRHC (C1 precursor); S00627 (C1C precursor); JH0150 (C3 precursor); S00626 (C1B precursor); JH0149 (C2 precursor); CAA00083 (Armoracia rusticana); and AAA72223 (synthetic horseradish perioxidase isoenzyme C (HRP-C)).

2) Urease

Any urease can be used in the present system. For example, the ureases with the following GenBank accession Nos. can be used: AF085729 (Ureaplasma urealyticum serovar); AF056321 (Actinomyces naeslundii); AF095636 (Yersinia pestis); AF006062 (Filobasidiella neoformans var. neoformans (URE1)); U81509 (Coccidioides immitis urease); AF000579 (Bordetella bronchiseptica); U352248 (Streptococcus salivarius); U33011 (Mycobacterium tuberculosis); U89957 (Actinobacillus pleuropneumoniae urease operon (ureABCXEFGD); D14439 (Thermophilic Bacillus); L40490 (Ureaplasma urealyticum T960 urease); L40489 (Ureaplasma urealyticum strain 7); U40842 (Yersinia pseudotuberculosis); M65260 (Canavalia ensiformis); U29368 (Bacillus pasteurii urease operon); L25079 (Heliobacter heilmannii urease); L24101 (Yersinia enterocolitica); M31834 (P.mirabilis urease operon); M36068 (K.aerogenes); L07039 (Kiebsiella pneumoniae); M60398 (H.pylori); L03308 (E.coli urease gene cluster); L03307 (E.coli urease gene cluster).

3) Alkaline Phosphatase

Any alkaline phosphatase can be used in the present system. For example, the alkaline phosphatases encoded by nucleic acids with the following GenBank accession Nos. can be used: AB013386 (Bombyx mori s-Alp soluble alkaline phosphatase); AF154110 (Enterococcus faecalis (phoZ); M13077 (Human placental); AF052227 (Bos taurus intestinal); AF052226 (Bos taurus intestinal); AF079878 (Thermus sp. (TAP)); AF047381 (Pseudomonas aeruginosa (phoA)); U49060 (Bacillus subtilis (phoD)); J03930 (Human intestinal (ALPI)); J03252 (Human alkaline (ALPP)); U19108 (Gallus tissue-nonspecific); M13345 (E. coli); U31569 (Felis catus (alp1)); L36230 (Zymomonas mobilis (phoD)); M19159 (Human placental heat-stable (PLAP-1)); M12551 (Human placental (PLAP)); M31008 (Human intestinal); J04948 (Human (ALP-1); J03572 (Rat); M61705 (Mouse intestinal (IAP); M61704 (Mouse embryonic); M61706 (Mouse (AP) pseudogene); M21134 (S.cerevisiae (rALPase)); L07733 (Cow intestinal (IAP)); M18443 (Bovine); M77507 (Synechococcus sp. atypical); M33965 (S.marcescens (phoA)); M33966 (E.fergusonii (phoA)); M29670 (E.coli (phoA)); M29669 (E.coli (phoA)); M29668 (E.coli (phoA)); M29667 (E.coli (phoA)); M29666 (E.coli (phoA)); M29665 (E.coli (phoA)); M29664 (E.coli (phoA)); M29663 (E.coli (phoA)); M23549 (Bacillus subtilis (phop gene, 3′ end and phoR gene); M16775 (B.subtilis phoP); M33634 (B.subtilis (phoAIII); L27993 (Neurospora crassa); U02550 (Bacillus subtilis (phoA)).

4) Luciferase

Any luciferase can be used in the present system. Numerous luciferases are available and have been cloned. For example, the luciferases encoded by nucleic acids with the following GenBank accession Nos. can be used: AH007711 (Streptomyces clavuligerus (cvm5)); AF124929 (cvm5); U43958 (Cloning vector pRcCMV-luc luciferase gene); M90092 (Xenorhabdus luminescens (luxA)); AF093688 (MMTV-luciferase reporter vector pHH Luc *SA *PS); AF093687 (MMTV-luciferase reporter vector PHH Luc *SA); AF093686 (MMTV-luciferase reporter vector pHH Luc); AF093685 (Luciferase reporter vector pXP2 *SA *PS); AF093684 (Luciferase reporter vector pXP2 *SA); AF093683 (Luciferase reporter vector pXP1); AF093682 (Luciferase reporter vector pXP2); U40374 (Luciferase reporter gene shuttle vector pMH30); AF003893 (Gonyaulax polyedra luciferase); L39928 (Pyrocoelia miyako (clone pB-PmL41); L39929 (Hotaria parvula (clone pB-Hp); AF085332 (Gonyaulax polyedra); U89490 (Vargula hilgendorfii); AF027129 (Eukaryotic luciferase expression vector pCMVtkLUC+); AF027128 (Eukaryotic luciferase expression vector ptkLUC+); AF027127 (Eukaryotic luciferase expression vector pTATALUC+); AF027126 (Eukaryotic luciferase expression vector pLUC+); U31240 (Photuris pennsylvanica); D25416 (Firefly clone pPFL7); D25415 (Firefly clone pPFL19); U84006 (Expression vector pBSII-LUCINT firefly luciferase (LUCINT); U55819 (Plasmid pRL765 with transposon Tn5 and luciferase (luxA and luxB) genes); U55385 (Plasmid pRL1063a with transposon Tn5 and luciferase (luxA and luxB) genes); U51019 (Luciola lateralis); U49182 (Luciola lateralis); U49181 (Luciola lateralis); M36597 (K. alfredi symbiont); U47298 (Cloning vector pGL-3-Promoter firefly luciferase (luc+) gene); U47297 (Cloning vector pGL3-Enhancer firefly luciferase (luc+) gene); U47296 (Cloning vector pGL3-Control firefly luciferase (luc+) gene); U47295 (Cloning vector pGL3-Basic firefly luciferase (luc+) gene); U47123 (Cloning vector pSP-luc+NF, luciferase cassette fusion vector); U47122 (Cloning vector pSP-luc+, Luciferase cassette vector); M10961 (V.harveyi (luxA and luxB); M65067 (Photobacterium phosphoreum (luxA and luxB); M62917 (Xenorhabdus luminescens (luxA, luxB, luxC, and luxD); M25666 (V.hilgendorfii); M63501 (Renilla reniformis); M15077 (P.pyralis (firefly)); M26194 (Luciola cruciata); M55977 (X.luminescens (luxA and luxB)); M90093 (Xenorhabdus luminescens (luxA) and (luxB) (luxE)); U03687 (Photinus pyralis modified luciferase gene).

5) Glutathione S-Transferase

A glutathione S-transferase (GST), more preferably a Schistosoma japonicum glutathione S-transferase, can be included in the conjugate. GST occurs naturally as a 26 kDa protein which can be expressed in E. Coli with full enzymatic activity. Conjugates that contain the full length GST also demonstrate GST enzymatic activity and can undergo dimerization as observed in nature (Parker et al., J. Mol. Biol., 213:221 (1990); Ji, et al., Biochemistry, 31:10169 (1992); and Maru et al., J. Biol. Chem., 271:15353 (1996)). The crystal structure of recombinant Schistosoma japonicum GST from pGEX vectors has been determined (McTigue et al., J, Mol. Biol., 246:21 (1995)) and matches that of the native protein. Conjugates that contain a GST can be readily purified.

For example, fusion proteins are easily purified from bacterial lysates by affinity chromatography using Glutathione Sepharose 4B contained in the GST Purification Modules (Amersham Pharmacia Biotech, Inc.). Cleavage of the desired protein from GST is achieved using a site-specific protease whose recognition sequence is located immediately upstream from the multiple cloning site on the pGEX plasmids. Fusion proteins can be detected using a colorimetric assay or immunoassay provided in the GST Detection Module, or by Western blotting with anti-GST antibody. The system has been used successfully in many applications such as molecular immunology (Toye et al., Infect. Immun., 58:3909 (1990)), the production of vaccines (Fikrig et al, Science, 250:553 (1990); and Johnson et al., Nature, 338:585 (1989)) and studies involving protein-protein (Kaelin et al., Cell, 64:521 (1991)) and DNA-protein (Kaelin et al., Cell, 65:1073 (1991)) interactions.

Any glutathione S-transferase is contemplated. For example, the glutathione S-transferase encoded by nucleic acid with the following GenBank accession Nos. can be used: [AF112567], Fasciola gigantica; [M77682], Fasciola hepatica; [AB016426], Cavia porcellus; [AF144382], Arabidopsis thaliana; [AF133251], Gallus; [AB021655], Issatchenkia orientalis; [AF133268], Manduca sexta; [AF125273], Homo sapiens tissue-type skeletal muscle; [AF125271], Homo sapiens tissue-type pancreas; [AB026292], Sphingomonas paucimobilis; [AB026119], Oncorhynchus nerka; [U49179], Bos taurus; [AF106661], Rattus norvegicus (GstYb4); [L15387], Gallus class-alpha; [AF051318], Clonorchis sinensis; [AF101269], Echinococcus granulosus; [AF077609], Boophilus microplus; [AA956087], Homo sapiens microsomal; [AF004358], Aegilops squarrosa; [AF109714], Triticum aestivum; [U86635], Rattus norvegicus glutathione; [AF111428], Drosophila melanogaster microsomal; [AF111426], Drosophila melanogaster microsomal; [AF071163], Anopheles gambiae; [AF071162], Anopheles gambiae; [AF071161], Anopheles gambiae; [AF071160], Anopheles gambiae; [D10524], Nicotiana tabacum; [AF062403], Oryza sativa; [U77604], Homo sapiens microsomal (MGST2); [U30897], Human (P1b); [U62589], Human (GSTp1c); [U42463], Coccomyxa sp. PA; [AF001779], Sphingomonas paucimobilis strain epa505; [U51165], Cycloclasticus oligotrophus (XYLK); [AF025887], Homo sapiens (GSTA4); [U66342], Plutella xylostella; [AF051238], Picea mariana (Sb52); [AF051214], Picea mariana (Sb18); [AF079511], Mesembryanthemum crystallinum clone R6-R37; [D10026], Rattus norvegicus Yrs-Yrs; [AF048978], Glycine max 2,4-D inducible (GSTa); [AF043105], Homo sapiens (GSTM3); [AF057172], Homo sapiens (GSTT2P); [U21689], Human; [AH006027], Homo sapiens (GSTT2); [AF057176], Homo sapiens (GSTT2); [AF050102], Oryza sativa (GST1); [AF044411], Schistosoma japonicum; [U87958], Culicoides variipennis (CVGST1); [AF026977], Homo sapiens microsomal (MGST3); [AF027740], Homo sapiens microsomal (MGST1L1); [AF005928], Echinococcus granulosus; [AF001103], Pseudomonas (phnC); [AF010241], Caenorhabditis elegans (CeGST3); [AF010240], Caenorhabditis elegans (CeGST2); [AF010239], Caenorhabditis elegans (CeGST1); [AF002692], Solanum commersonii (GST1); [L38503], Homo sapiens (GSTT2); [M97937], E. coli/S. japonicum; [L29427], Rat GST-P gene; [M14654], Schistosoma japonicum Sj26 antigen; [AB000884], Sus scrofa; [D44465], Arabidopsis thaliana; [D17673], Arabidopsis thaliana; [D17672], Arabidopsis thaliana; [U78784], Anopheles dirus; [U71213], Human microsomal; [U70672], Arabidopsis thaliana; [U24428], Mus musculus; [U43126], Naegleria fowleri; [X14233], D.melanogaster (GST); [L32092], Manduca sexta; [L32091], Manduca sexta; [U30489], Arabidopsis thaliana; [M24889], Artificial maize; [L05915], Dianthus caryophyllus; [M15872], Human; [L23766], Oryctolagus cuniculus; [J03679], Solanum tuberosum; [U12472], Human (GST phi); [U15654], Mus musculus; [M24485], Homo sapiens (GSTP1); [L28771], Onchocerca volvulus; [M14777], Human; [M16594], Human; [M21758], Human; [J03914], Rat; [K01932], Rat liver; [J02810], Rat prostate; [M25891], Rat; [M11719], Rat liver; [M28241], Rat; [J03752], Rat; [M73483], Mouse (GST Yc); [J04696], Mouse (GST5-5); [J04632], Mouse (GST1-1); [M59772], M.auratus; [L20466], Chinese hamster; [M25627], Human liver; [J03746], Human (SEQ ID No. 75); [M16901], Maize; [M64268], Dianthus caryophyllus; [L11601], Arabidopsis thaliana; [L07589], Arabidopsis thaliana; [M74529], Oryctolagus cuniculus; [M74528], Oryctolagus cuniculus; [M98271], Schistosoma mansoni 28 kDa; [L23126], Lucilia cuprina; [M95198], Drosophila melanogaster; [L26544], Methylophilus sp.; [U14753], Dirofilaria immitus; [U12679], Zea mays; [L02321], Human (GSTM5); [L15386], Chicken.

In addition, commercially available Glutathione S-transferase (GST) gene fusion system can be used. For example, the Glutathione S-transferase (GST) Gene Fusion System (Amersham Pharmacia Biotech, Inc.) can be used. The system from Amersham Pharmacia Biotech, Inc. is an integrated system for the expression, purification and detection of fusion proteins produced in E. coli. The system includes three primary components: pGEX plasmid vectors, various options for GST purification and a variety of GST detection products. A series of site-specific proteases complements the system. The pGEX plasmids are designed for inducible, high-level intracellular expression of genes or gene fragments as fusions with Schistosoma japonicum GST (Smith and Johnson, Gene, 67:31 (1988)). All pGEX Vectors (GST Gene fusion) offer: 1) A tac promoter for chemically inducible, high-level expression; 2) an internal lac I^{q} gene for use in any E. Coli host; 3) very mild elution conditions for release of fusion proteins form the affinity matrix, thus minimizing effects on antigenicity and functional activity; and 4) PreScission, thrombin or factor Xa protease recognition sites for cleaving the desired protein from the fusion product.

The GST Detection Module from Amersham Pharmacia Biotech, Inc. can be used for identification of GST fusion proteins using either a biochemical or immunological assay. In the biochemical assay, glutathione and 1-chloro-2-4-dinitrobenzene (CDNB) serve as substrates for GST to yield a yellow product detectable at 340 nm (Habig et al., J. Biol. Chem., 249:7130 (1974)). An affinity-purified goat anti-GST polyclonal antibody suitable for Western blots is used in the immunoassay.

The GST 96-Well Detection Module from Amersham Pharmacia Biotech, Inc. contains five microtitre strip plates, horseradish peroxidase (HRP) conjugated anti-GST antibody and recombinant GST protein. The wells of each plate are coated with purified anti-GST antibody to capture GST fusion proteins and are preblocked to provide a low background. HRP conjugated antibody enables sensitive detection of GST proteins.

The anti-GST antibody supplied in the system from Amersham Pharmacia Biotech, Inc. is a polyclonal antibody purified from the sera of goats immunized with purified schistosomal glutathione S-transferase (GST). Because of its polyclonal nature, it can recognize more than one epitope on GST, thereby improving its capacity for recognizing GST fusion proteins even if some binding sites are masked due to recombinant protein folding.

Factor Xa can be used for site-specific separation of the GST affinity tag from proteins expressed using pGEX X vectors. Factor Xa enables the site-specific cleavage of fusion proteins containing an accessible Factor Xa recognition sequence. It can be used either following affinity purification or while fusion proteins are bound to Glutathione Sepharose 4B. Factor Xa, purified from bovine plasma, is used to digest fusion proteins prepared from pGEX vectors containing the recognition sequence for factor Xa (pGEX-3X, pGEX-5X-1, pGEX-5X-2 and pGEX-5X-3). It specifically cleaves following the tetrapeptide Ile-Glu-Gly-Arg (SEQ ID No. 77) (Nagai and Thgersen, Nature, 309:810 (1984); and Nagai and Thgersen, Methods Enzymol., 153:461 (1987)). In the system from Amersham Pharmacia Biotech, Inc., one unit of Factor Xa cleaves≧90% of 100 ìg of a test GST fusion protein when incubated in 1 mM CaCl₂, 100 mM NaCl and 50 mM Tris-HCl (pH 8.0) at 22?C. for 16 hours.

PreScission protease can be used for site-specific separation of the GST affinity tag from proteins expressed using pGEX-6P vectors. It enables the low-temperature cleavage of fusion proteins containing the PreScission Protease recognition sequence. It can be used either following affinity purification or while fusion proteins are bound to Glutathione Sepharose 4B. PreScission Protease is a genetically engineered fusion protein containing human rhinovirus 3C protease and GST (Walker et al., Bio/Technology, 12:601 (1994)). This protease was specifically designed to facilitate removal of the protease by allowing simultaneous protease immobilization and cleavage of GST fusion proteins produced from pGEX-6P vectors (pGEX-6P-1, pGEX-6P-2, and pGEX-6P-3). PreScission Protease specifically cleaves between the Gln and Gly residues of the recognition sequence of LeuGluValLeuPheGln/GlyPro (SEQ ID No. 78) (Cordingley et al., J. Bio. Chem., 265:9062 (1990)). In the system from Amersham Pharmacia Biotech, Inc., one unit of PreScission protease will cleave≧90% of 100 ìg of a test GST-fusion protein in 50 mM Tris-HCl, 150 mM NaCl, 1 mM EDTA, 1 mM DTT, pH 7.0 at 5?C. for 16 hours.

Thrombin can be used for site-specific separation of the GST affinity tag from proteins expressed using pGEX T vectors. It enables the site-specific cleavage of fusion proteins containing an accessible thrombin recognition sequence. It is purified from bovine plasma; functionally free of other clotting factors, plasminogen and plasmin. It can be used either following affinity purification or while fusion proteins are bound to Glutathione Sepharose 4B. Thrombin is used to digest fusion proteins prepared from pGEX vectors containing the recognition sequence for thrombin (pGEX-1ëT, pGEX-2T, pGEX-2TK, pGEX-4T-1, pGEX-4T2 and pGEX-4T-3). In the system from Amersham Pharmacia Biotech, Inc., one unit of Thrombin cleaves≧90% of 100 ìg of a test GST fusion protein when incubated in 1×PBS at 22?C. for 16 hours.

6) Defense Proteins

The conjugates can contain defense protein, such as an antibody. Any antibody, including polyclonal, monoclonal, single chain or Fab fragments, can be used.

7) Fluorescent Moieties

The conjugates can contain a fluorescent moiety, such as a green, a blue or a red fluorescent protein. Any green, blue or red fluorescent protein can be used in the present system. For instance, the green fluorescent proteins encoded by nucleic acids with the following GenBank accession Nos. can be used: U47949 (AGP1); U43284; AF007834 (GFPuv); U89686 (Saccharomyces cerevisiae synthetic green fluorescent protein (cox3::GFPm-3) gene); U89685 (Saccharomyces cerevisiae synthetic green fluorescent protein (cox3::GFPm) gene); U87974 (Synthetic construct modified green fluorescent protein GFP5-ER (mgfp5-ER)); U87973 (Synthetic construct modified green fluorescent protein GFP5 (mgfp5)); U87625 (Synthetic construct modified green fluorescent protein GFP-ER (mfgp4-ER)); U87624 (Synthetic construct green fluorescent protein (mgfp4) mRNA)); U73901 (Aequorea victoria mutant 3); U50963 (Synthetic); U70495 (soluble-modified green fluorescent protein (smGFP)); U57609 (enhanced green fluorescent protein gene); U57608 (enhanced green fluorescent protein gene); U57607 (enhanced green fluorescent protein gene); U57606 (enhanced green fluorescent protein gene); U55763 (enhanced green fluorescent protein (egfp); U55762 (enhanced green fluorescent protein (egfp); U55761 (enhanced green fluorescent protein (egfp); U54830 (Synthetic E. coli Tn3-derived transposon green fluorescent protein (GF); U36202; U36201; U19282; U19279; U19277; U19276; U19281; U19280; U19278; L29345 (Aequorea victoria); M62654 (Aequorea victoria); M62653 (Aequorea victoria); AAB47853 ((U87625) synthetic construct modified green fluorescent protein (GFP-ER)); AAB47852 ((U87624) synthetic construct green fluorescent protein).

Similarly, the blue fluorescent proteins encoded by nucleic acids with the following GenBank accession Nos. can be used: U70497 (soluble-modified blue fluorescent protein (smBFP); 1BFP (blue variant of green fluorescent protein); AAB16959 (soluble-modified blue fluorescent protein).

Also similarly, the red fluorescent proteins encoded by nucleic acids with the following GenBank accession Nos. can be used: U70496 (soluble-modified red-shifted green fluorescent protein (smRSGFP); AAB16958 ((U70496) soluble-modified red-shifted green fluorescent protein).

### H. Immobilization of Mutant DNA Repair Enzymes and Nucleic Acids

In the methods for detecting abnormal base-pairings, mutations, and polymorphisms, and the methods for localizing and removing abnormal base-pairings described in Sections B-F, the target nucleic acid strand to be assayed, the reference nucleic acid strand, the target nucleic acid duplex to be assayed, the nucleic acid duplex formed via hybridization of the target strand and the reference strand, or the mutant DNA repair enzyme or complex thereof can be immobilized on the surface of a support, either directly via a linker. Preferably, the support used is an insoluble support such as a silicon chip. Non-limiting examples of the geometry of the support include beads, pellets, disks, capillaries, hollow fibers, needles, solid fibers, random shapes, thin films, membranes and chips. Also more preferably, the nucleic acid strand, the nucleic acid duplex or the mutant DNA repair enzyme or complex thereof is immobilized in an array or a well format on the surface.

1. Immobilization of the Mutant DNA Repair Enzymes

In certain embodiments, where the facilitating agents are designed for linkage to surfaces, recovered, isolated or purified conjugates, such as fusion proteins can be attached to a surface of a matrix material. Immobilization may be effected directly or via a linker. The conjugates may be immobilized on any suitable support, including, but are not limited to, silicon chips, and other supports described herein and known to those of skill in the art. A plurality of conjugates, which may contain the same or different or a variety of mutant DNA repair enzymes (abnormal base-pairing trapping enzymes) may be attached to a support, such as an array (i.e., a pattern of two or more) of conjugates on the surface of a silicon chip or other chip for use in high throughput protocols and formats.

It is also noted that the mutant DNA repair enzymes can be linked directly to the surface or via a linker without a facilitating agent linked thereto. Hence, chips containing arrays of mutant DNA repair enzymes are contemplated.

For example, an isolated or purified fusion protein can be attached to the surface as the intact fusion proteins. Alternatively, the protein or peptide fragment portion can be cleaved off and the mutant DNA repair enzyme be attached to the surface. The fusion protein can be cleaved by any methods known in the art such as chemical or enzymatic means. The cleavage means must be compatible with the linking sequence between the protein or peptide fragment portion and the mutant DNA repair enzyme so that the cleavage is linker sequence specific and the cleaved mutant enzyme is functional, i.e., can be used as a abnormal base-pairing-trapping enzyme. Those skilled in the art can readily determine, if necessary, with empirical studies, which cleavage/linker sequence pair to be used. Many cleavage/linker sequence pairs are well known in the art. For example, Factor Xa can be used for site-specific separation of the GST affinity tag from proteins expressed using pGEX X vectors; PreScission protease can be used for site-specific separation of the GST affinity tag from proteins expressed using pGEX-6P vectors; and Thrombin can be used for site-specific separation of the GST affinity tag from proteins expressed using pGEX T vectors.

The matrix material substrates contemplated herein are generally insoluble materials used to immobilize ligands and other molecules, and are those that are used in many chemical syntheses and separations. Such substrates, also called matrices, are used, for example, in affinity chromatography, in the immobilization of biologically active materials, and during chemical syntheses of biomolecules, including proteins, amino acids and other organic molecules and polymers. The preparation of and use of matrices is well known to those of skill in this art; there are many such materials and preparations thereof known. For example, naturally-occurring matrix materials, such as agarose and cellulose, may be isolated from their respective sources, and processed according to known protocols, and synthetic materials may be prepared in accord with known protocols.

The substrate matrices are typically insoluble materials that are solid, porous, deformable, or hard, and have any required structure and geometry, including, but not limited to: beads, pellets, disks, capillaries, hollow fibers, needles, solid fibers, random shapes, thin films and membranes. Thus, the item may be fabricated from the matrix material or combined with it, such as by coating all or part of the surface or impregnating particles.

Typically, when the matrix is particulate, the particles are at least about 10-2000 ìM, but may be smaller or larger, depending upon the selected application. Selection of the matrices will be governed, at least in part, by their physical and chemical properties, such as solubility, functional groups, mechanical stability, surface area swelling propensity, hydrophobic or hydrophilic properties and intended use.

If necessary, the support matrix material can be treated to contain an appropriate reactive moiety. In some cases, the support matrix material already containing the reactive moiety may be obtained commercially. The support matrix material containing the reactive moiety may thereby serve as the matrix support upon which molecules are linked. Materials containing reactive surface moieties such as amino silane linkages, hydroxyl linkages or carboxysilane linkages may be produced by well established surface chemistry techniques involving silanization reactions, or the like. Examples of these materials are those having surface silicon oxide moieties, covalently linked to gamma-aminopropylsilane, and other organic moieties; N-[3-(triethyoxysilyl)propyl]phthelamic acid; and bis-(2-hydroxyethyl)amino-propyltriethoxysilane. Exemplary of readily available materials containing amino group reactive functionalities, include, but are not limited to, para-aminophenyltriethyoxysilane. Also derivatized polystyrenes and other such polymers are well known and readily available to those of skill in this art (e.g., the Tentagel® Resins are available with a multitude of functional groups, and are sold by Rapp Polymere, Tubingen, Germany; see, U.S. Pat. Nos. 4,908,405 and 5,292,814; see, also Butz et al., Peptide Res., 7:20-23 (1994); and Kleine et al., Immunobiol., 190:53-66 (1994)).

These matrix materials include any material that can act as a support matrix for attachment of the molecules of interest. Such materials are known to those of skill in this art, and include those that are used as a support matrix. These materials include, but are not limited to, inorganics, natural polymers, and synthetic polymers, including, but are not limited to: cellulose, cellulose derivatives, acrylic resins, glass, silica gels, polystyrene, gelatin, polyvinyl pyrrolidone, co-polymers of vinyl and acrylamide, polystyrene cross-linked with divinylbenzene and others (see, Merrifield, Biochemistry, 3:1385-1390 (1964)), polyacrylamides, latex gels, polystyrene, dextran, polyacrylamides, rubber, silicon, plastics, nitrocellulose, celluloses, natural sponges. Of particular interest herein, are highly porous glasses (see, e.g., U.S. Pat. No. 4,244,721) and others prepared by mixing a borosilicate, alcohol and water.

Synthetic matrices include, but are not limited to: acrylamides, dextran-derivatives and dextran co-polymers, agarose-polyacrylamide blends, other polymers and co-polymers with various functional groups, methacrylate derivatives and co-polymers, polystyrene and polystyrene copolymers (see, e.g., Merrifield, Biochemistry, 3:1385-1390 (1964); Berg et al., in Innovation Perspect. Solid Phase Synth. Collect. Pap., Int. Symp., 1 st, Epton, Roger (Ed), pp. 453-459 (1990); Berg et al., Pept., Proc. Eur. Pept. Symp., 20th, Jung, G. et al. (Eds), pp. 196-198 (1989); Berg et al., J. Am. Chem. Soc., 111:8024-8026 (1989); Kent et al., Isr. J. Chem., 17:243-247 (1979); Kent et al., J. Org. Chem., 43:2845-2852 (1978); Mitchell et al., Tetrahedron Lett., 42:3795-3798 (1976); U.S. Pat. Nos. 4,507,230; 4,006,117; and 5,389,449). Methods for preparation of such matrices are well-known to those of skill in this art.

Synthetic matrices include those made from polymers and co-polymers such as polyvinylalcohols, acrylates and acrylic acids such as polyethylene-co-acrylic acid, polyethylene-co-methacrylic acid, polyethylene-co-ethylacrylate, polyethylene-co-methyl acrylate, polypropylene-co-acrylic acid, polypropylene-co-methyl-acrylic acid, polypropylene-co-ethylacrylate, polypropylene-co-methyl acrylate, polyethylene-co-vinyl acetate, poly-propylene-co-vinyl acetate, and those containing acid anhydride groups such as polyethylene-co-maleic anhydride, polypropylene-co-maleic anhydride and the like. Liposomes have also been used as solid supports for affinity purifications (Powell et al. Biotechnol. Bioeng., 33:173 (1989)).

For example, U.S. Pat. No. 5,403,750, describes the preparation of polyurethane-based polymers. U.S. Pat. No. 4,241,537 describes a plant growth medium containing a hydrophilic polyurethane gel composition prepared from chain-extended polyols; random copolymerization is preferred with up to 50% propylene oxide units so that the prepolymer will be a liquid at room temperature. U.S. Pat. No. 3,939,123 describes lightly crosslinked polyurethane polymers of isocyanate terminated prepolymers containing poly(ethyleneoxy) glycols with up to 35% of a poly(propyleneoxy) glycol or a poly(butyleneoxy) glycol. In producing these polymers, an organic polyamine is used as a crosslinking agent. Other matrices and preparation thereof are described in U.S. Pat. Nos. 4,177,038, 4,175,183, 4,439,585, 4,485,227, 4,569,981, 5,092,992, 5,334,640, 5,328,603.

U.S. Pat. No. 4,162,355 describes a polymer suitable for use in affinity chromatography, which is a polymer of an aminimide and a vinyl compound having at least one pendant halo-methyl group. An amine ligand, which affords sites for binding in affinity chromatography is coupled to the polymer by reaction with a portion of the pendant halo-methyl groups and the remainder of the pendant halo-methyl groups are reacted with an amine containing a pendant hydrophilic group. A method of coating a substrate with this polymer is also described. An exemplary aminimide is 1,1-dimethyl-1-(2-hydroxyoctyl)amine methacrylimide and vinyl compound is a chloromethyl styrene.

U.S. Pat. No. 4,171,412 describes specific matrices based on hydrophilic polymeric gels, preferably of a macroporous character, which carry covalently bonded D-amino acids or peptides that contain D-amino acid units. The basic support is prepared by copolymerization of hydroxyalkyl esters or hydroxyalkylamides of acrylic and methacrylic acid with crosslinking acrylate or methacrylate comonomers are modified by the reaction with diamines, aminoacids or dicarboxylic acids and the resulting carboxyterminal or aminoterminal groups are condensed with D-analogs of aminoacids or peptides. The peptide containing D-aminoacids also can be synthesized stepwise on the surface of the carrier. For example, U.S. Pat. No. 4,178,439 describes a cationic ion exchanger and a method for preparation thereof U.S. Pat. No. 4,180,524 describes chemical syntheses on a silica support.

The fusion protein can be attached to the surface of the matrix material by methods known in the art. Numerous methods have been developed for the immobilization of proteins and other biomolecules onto solid or liquid supports (see, e.g., Mosbach, Methods in Enzymology, 44 (1976); Weetall, Immobilized Enzymes, Antigens, Antibodies, and Peptides, (1975); Kennedy et al., Solid Phase Biochemistry, Analytical and Synthetic Aspects, Scouten, ed., pp. 253-391 (1983); see, generally, Affinity Techniques. Enzyme Purification: Part B. Methods in Enzymology, Vol. 34, ed. W. B. Jakoby, M. Wilchek, Acad. Press, N.Y. (1974); and Immobilized Biochemicals and Affinity Chromatography, Advances in Experimental Medicine and Biology, vol. 42, ed. R. Dunlap, Plenum Press, N.Y. (1974)).

Among the most commonly used methods are absorption and adsorption or covalent binding to the support, either directly or via a linker, such as the numerous disulfide linkages, thioether bonds, hindered disulfide bonds, and covalent bonds between free reactive groups, such as amine and thiol groups, known to those of skill in art (see, e.g., the PIERCE CATALOG, Immuno Technology Catalog & Handbook, 1992-1993, which describes the preparation of and use of such reagents and provides a commercial source for such reagents; Wong, Chemistry of Protein Conjugation and Cross Linking, CRC Press (1993); see also DeWitt et al., Proc. Natl. Acad. Sci. U.S.A., 90:6909 (1993); Zuckermann et al., J. Am. Chem. Soc., 14:10646 (1992); Kurth et al., J. Am. Chem. Soc., 116:2661 (1994); Ellman et al., Proc. Natl. Acad. Sci. U.S.A., 91:4708 (1994); Sucholeiki, Tetrahedron Lttrs., 35:7307 (1994); Su-Sun Wang, J. Org. Chem., 41:3258 (1976); Padwa et al., J. Org. Chem., 41:3550 (1971); and Vedejs et al., J. Org. Chem., 49:575 (1984), which describe photosensitive linkers).

To effect immobilization, a composition containing the protein or other biomolecule is contacted with a support material such as alumina, carbon, an ion-exchange resin, cellulose, glass or a ceramic. Fluorocarbon polymers have been used as supports to which biomolecules have been attached by adsorption (see, U.S. Pat. No. 3,843,443; Published International PCT Application WO 86/03840).

A large variety of methods are known for attaching biological molecules, including proteins and nucleic acids, molecules to solid supports (see e.g., U.S. Pat. No. 5451683). For example, U.S. Pat. No. 4,681,870 describes a method for introducing free amino or carboxyl groups onto a silica matrix. These groups may subsequently be covalently linked to other groups, such as a protein or other anti-ligand, in the presence of a carbodiimide. Alternatively, a silica matrix may be activated by treatment with a cyanogen halide under alkaline conditions. The anti-ligand is covalently attached to the surface upon addition to the activated surface. Another method involves modification of a polymer surface through the successive application of multiple layers of biotin, avidin and extenders (see e.g., U.S. Pat. No. 4,282,287). Other methods involve photoactivation in which a polypeptide chain is attached to a solid substrate by incorporating a light-sensitive unnatural amino acid group into the polypeptide chain and exposing the product to low-energy ultraviolet light (see e.g., U.S. Pat. No. 4,762,881). Oligonucleotides have also been attached using a photochemically active reagent, such as a psoralen compound, and a coupling agent, which attaches the photoreagent to the substrate (see e.g., U.S. Pat. Nos. 4,542,102 and 4,562,157). Photoactivation of the photoreagent binds a nucleic acid molecule to the substrate to give a surface-bound probe.

Covalent binding of the protein or other biomolecule or organic molecule or biological particle to chemically activated solid matrix supports such as glass, synthetic polymers, and cross-linked polysaccharides is a more frequently used immobilization technique. The molecule or biological particle may be directly linked to the matrix support or linked via linker, such as a metal (see, e.g., U.S. Pat. No. 4,179,402; and Smith et al., Methods: A Companion to Methods in Enz., 4:73-78 (1992)). An example of this method is the cyanogen bromide activation of polysaccharide supports, such as agarose. The use of perfluorocarbon polymer-based supports for enzyme immobilization and affinity chromatography is described in U.S. Pat. No. 4,885,250. In this method the biomolecule is first modified by reaction with a per-fluoroalkylating agent such as perfluorooctylpropylisocyanate described in U.S. Pat. No. 4,954,444. Then, the modified protein is adsorbed onto the fluorocarbon support to effect immobilization.

The activation and use of matrices are well known and may be effected by any such known methods (see, e.g., Hermanson et al., Immobilized Affinity Ligand Techniques, Academic Press, Inc., San Diego (1992)). For example, the coupling of the amino acids may be accomplished by techniques familiar to those in the art and provided, for example, in Stewart and Young, Solid Phase Synthesis, Second Edition, Pierce Chemical Co., Rockford (1984).

Other suitable methods for linking molecules to solid supports are well known to those of skill in this art (see, e.g., U.S. Pat. No. 5,416,193). These include linkers that are suitable for chemically linking molecules, such as proteins, to supports and include, but are not limited to, disulfide bonds, thioether bonds, hindered disulfide bonds, and covalent bonds between free reactive groups, such as amine and thiol groups. These bonds can be produced using heterobifunctional reagents to produce reactive thiol groups on one or both of the moieties and then reacting the thiol groups on one moiety with reactive thiol groups or amine groups to which reactive maleimido groups or thiol groups can be attached on the other.

Other linkers include, acid cleavable linkers, such as bismaleimideothoxy propane, acid labile-transferrin conjugates and adipic acid diihydrazide, that would be cleaved in more acidic intracellular compartments; cross linkers that are cleaved upon exposure to UV or visible light and linkers, such as the various domains, such as C_{H}1, C_{H}2, and C_{H}3, from the constant region of human IgG₁, (Batra et al., Molecular Immunol., 30:379-386 (1993)). Presently preferred linkages are direct linkages effected by adsorbing the molecule to the surface of the matrix.

Other linkages are photocleavable linkages that can be activated by exposure to light (see, e.g., Goldmacher et al., Bioconj. Chem., 3:104-107 (1992)). The photocleavable linker is selected such that the cleaving wavelength does not damage linked moieties. Photocleavable linkers are linkers that are cleaved upon exposure to light (see, e.g., Hazum et al., Pept., Proc. Eur. Pept. Symp., 16th, Brunfeldt, K (Ed), pp. 105-110 (1981), which describes the use of a nitrobenzyl group as a photocleavable protective group for cysteine; Yen et al., Makromol. Chem., 190:69-82 (1989), which describes water soluble photocleavable copolymers, including hydroxypropylmethacrylamide copolymer, glycine copolymer, fluorescein copolymer and methylrhodamine copolymer; Goldmacher et al., Bioconj. Chem., 3:104-107 (1992), which describes a cross-linker and reagent that undergoes photolytic degradation upon exposure to near UV light (350 nm); and Senter et al., Photochem. Photobiol., 42:231-237 (1985), which describes nitrobenzyloxycarbonyl chloride cross linking reagents that produce photocleavable linkages). The selected linker will depend upon the particular application and, if needed, may be empirically selected.

In a preferred embodiment, the recovered fusion protein is attached to the surface through affinity binding between the protein or peptide fragment of the fusion protein and an affinity binding moiety on the surface.

2. Immobilization of Nucleic Acids

The target nucleic acid strand to be assayed, the reference nucleic acid strand, the target nucleic acid duplex to be assayed, the nucleic acid duplex formed via hybridization of the target strand and the reference strand can be immobilized by any methods known in the art. For example, the immobilization procedures disclosed in the following literatures can be used: Bresser et al., DNA, 2(3):243-54 (1983); Hirayama et al., Nucleic Acids Res., 24(20):4098-9 (1996); Kremsky et al., Nucleic Acids Res., 15(7):2891-909 (1987); Macdougall et al., Biochem. J., 191(3):855-8 (1980); Mykoniatis, J. Biochem. Biophys. Methods, 10(5-6):321-8 (1985); Nagasawa et al., J. Appl. Biochem., 7(4-5 :296-302 (1985); Nikiforov and Rogers, Anal. Biochem., 227(l):201-9 (1995); Proudnikov et al., Anal. Biochem., 259(1):34-41 (1998); Rasmussen et al., Anal. Biochem., 198(1):138-42 (1991); and Rogers et al., Anal. Biochem., 266(1):23-30 (1999).

Bresser et al., DNA, 2(3):243-54 (1983) discloses a method for selectively immobilizing either mRNA or DNA on nitrocellulose. Essential elements of the procedure for immobilizing DNA include tissue lysis, proteinase K treatment, solubilization of nucleic acids in hot 12.2 molal NaI, passage through a nitrocellulose filter, and acetylation of residual protein with acetic anhydride. Advantages include speed, quantitative recovery, low background, and elimination of the usual baking step. Essential elements of the procedure for selectively immobilizing mRNA include dissolving cells in Brij-35 and desoxycholate, proteinase K treatment, solubilizing nucleic acids in room temperature 12.2 molal NaI, filtration through nitrocellulose, and acetylation of residual protein. Advantages include selective immobilization of mRNA but not tRNA, rRNA, or DNA, and the maintenance of biological activity of the immobilized mRNA.

Hirayama et al., Nucleic Acids Res., 24(20):4098-9 (1996) discloses an improved and simplified protocol for DNA immobilization to enhance DNA-DNA hybridization on microwell plates. Target DNA was immobilized by simple dry-adsorption. Efficiencies of DNA immobilization and retention were enhanced 1.4-6.5 times and 4.2-19.6 times, respectively, compared with a conventional method. The overall hybridization efficiency was increased 3.1-5.2 times. This simple new protocol can reduce the consumption of scarce DNA samples.

Kremsky et al., Nucleic Acids Res., 15(7):2891-909 (1987) discloses a general method for the immobilization of DNA through its 5′-end has. A synthetic oligonucleotide, modified at its 5′-end with an aldehyde or carboxylic acid, was attached to latex microspheres containing hydrazide residues. Using T4 polynucleotide ligase and an oligonucleotide splint, a single stranded 98mer was efficiently joined to the immobilized synthetic fragment. After impregnation of the latex microspheres with the fluorescent dye, Nile Red and attachment of an aldehyde 16mer, 5×10⁵ bead-DNA conjugates could be detected with a conventional fluorimeter.

Macdougall et al., Biochem. J., 191(3):855-8 (1980) discloses a method in which double-stranded DNA is alkylated with 4-bis-(2-chloroethyl)amino-L-phenylalanine and the product immobilized on an insoluble support via the primary amino group of the phenylalanine moiety. The DNA is irreversibly bound to the matrix by both strands at a limited number of points.

Mykoniatis, J. Biochem. Biophys. Methods, 10(5-6):321-8 (1985) discloses a method for the immobilization of DNA on Sephadex G200 in the presence of water soluble carbodiimide. An increase in the extent of binding was observed when the incubation temperature of the DNA-Sephadex mixture was changed. It was found that native DNA immobilized to Sephadex with higher efficiency than denatured DNA. The stability of native DNA-Sephadex complex was about the same as that of denatured DNA-Sephadex. The size of DNA released by DNA-Sephadex after incubation of a suspension of the complex was the same as that of the DNA used for immobilization.

Nagasawa et al., J. Appl. Biochem., 7(4-5 :296-302 (1985) discloses a method in which DNA was immobilized covalently to Sepharose by several methods using epichlorohydrin, cyanogen bromide, carbodiimide, hydroxysuccinimide, carbonyldiimidazole, trichlorotriazine, and diazonium salt. These immobilizing methods were compared from the standpoint of the preparation of immunosorbent for anti-DNA antibodies. Among these methods, that involving epichlorohydrin was the most suitable because of large coupling capacity, stability of bound DNA, and nonadsorption of anti-DNA by the support itself.

Nikiforov and Rogers, Anal. Biochem., 227(1):201-9 (1995) discloses 3 methods for the immobilization of relatively short (12-30 mer) oligonucleotide probes to 96-well polystyrene plates for use in DNA hybridization-based assays. Two of the methods are modifications of previously published procedures, requiring the use of modified oligonucleotides and/or modified plates. These were compared to a newly developed method, whereby passive immobilization occurs by incubation in the presence of salt or a cationic detergent. While all methods resulted in the productive binding of the DNA probes and could therefore be used for hybridization, only the passive immobilization approach met strict performance criteria for use in DNA genotyping.

Proudnikov et al., Anal. Biochem., 259(1):34-41 (1998) discloses immobilization of DNA in polyacrylamide gel for the manufacture of DNA and DNA-oligonucleotide microchips. Activated DNA was immobilized in aldehyde-containing polyacrylamide gel for use in manufacturing the MAGIChip (microarrays of gel-immobilized compounds on a chip). First, abasic sites were generated in DNA by partial acidic depurination. Amino groups were then introduced into the abasic sites by reaction with ethylenediamine and reduction of the aldimine bonds formed. It was found that DNA could be fragmented at the site of amino group incorporation or preserved mostly unfragmented. In similar reactions, amino-DNA and amino-oligonucleotides were attached through their amines to polyacrylamide gel derivatized with aldehyde groups. Single- and double-stranded DNA of 40 to 972 nucleotides or base pairs were immobilized on the gel pads to manufacture a DNA microchip. The microchip was hybridized with fluorescently labeled DNA-specific oligonucleotide probes. This procedure for immobilization of amino compounds was used to manufacture MAGIChips containing DNA and oligonucleotides.

Rasmussen et al., Anal. Biochem., 198(l):138-42 (1991) discloses covalent immobilization of DNA onto polystyrene microwells via the DNA's 5′ end. DNA is bound onto the microwells by formation of a phosphoramidate bond between the 5′ terminal phosphate group and the microwells. Immobilization of 25 to 30 ng DNA per well is obtained. DNA molecules bound covalently at only the 5′ end are, ideally, perfect for hybridization.

Rogers et al., Anal. Biochem., 266(1):23-30 (1999) discloses immobilization of oligonucleotides onto a glass support via disulfide bonds in preparation of DNA microarrays. This method provides an efficient and specific covalent attachment chemistry for immobilization of DNA probes onto a solid support. Glass slides were derivatized with 3-mercaptopropyl silane for attachment of 5-prime disulfide-modified oligonucleotides via disulfide bonds. An attachment density of approximately 3×10⁵ oligonucleotides/micron² was observed. Oligonucleotides attached by this method provided a highly efficient substrate for nucleic acid hybridization and primer extension assays. In addition, patterning of multiple DNA probes on a glass surface utilizing this attachment chemistry has been demonstrated, which allows for array densities of at least 20,000 spots/cm².

### I. High-Throughput Assay Format

Although the methods for detecting abnormal base-pairing, mutations or polymorphisms, or methods for removing or localizing such abnormal base-pairing described in Sections B-F can be used wherein a single sample is assayed in one assay, the assay is preferably conducted in a high throughput mode, i.e., a plurality of the abnormal base-pairing, mutations or polymorphisms are detected, localized and/or removed simultaneously (See generally, High Throughput Screening: The Discovery of Bioactive Substances (Devlin, Ed.) Marcel Dekker, 1997; Sittampalam et al., Curr. Opin. Chem. Biol., 1(3):384-91 (1997); and Silverman et al., Curr. Opin. Chem. Biol., 2(3):397-403 (1998)). For example, the assay can be conducted in a multi-well (e.g., 24-, 48-, 96-, or 384-well), chip or array format.

Current state-of-the-art high-throughput assay operations are highly automated and computerized to handle sample preparation, assay procedures and the subsequent processing of large volumes of data. Each one of these steps requires careful optimization to operate efficiently and can assay 100-300,000 samples in a 2-6 month period. Hence, a modern high-throughput assay operation is a multidisciplinary field involving analytical chemistry, biology, biochemistry, synthesis chemistry, molecular biology, automation engineering and computer science (Fernandes, J. Biomol. Screening, 2:1 (1997)).

1. High-Throughput Assay Instrumentation and Capabilities

In general, the instrumentation used in high-throughput assays should be accurate, reliable and easily amenable to automation. Analytical methods should be robust and reproducible, with stable reagents and signal responses. Signal-to-noise (S/N) ratios should be large enough to generate signal windows (Sittampalam et al., J. Biomol. Screening, 2:159-169 (1997)) that allow reliable detection of “hits”.

2. Detection Technologies

Detection technologies employed in high-throughput screens depend on the type of biochemical pathway being investigated (Sittampalam et al., Curr. Opin. Chem. Biol., 1(3):384-91 (1997)).

a. Radiochemical Methods

Although filtration-based receptor binding assays have been used extensively in the past (to separate the bound and free radiolabeled ligand), the scintillation proximity assay (SPA) has become the standard assay in many HTS operations, mainly because it does not require a separation step, and can be easily automated (Braunwalder et al., J. Biomol. Screening, 1:23-26 (1996); Cole, Methods Enzymol., 275:310-328 (1996); Cook, Drug Discov. Tech., 1:287-294 (1996); Kahl et al., J. Biomol. Screening, 2:33-40 (1997); Lemer et al., J. Biomol. Screening, 1:135-143 (1996); Baker et al., Anal. Biochem., 239:20-24 (1996); Baum et al., Anal. Biochem., 237:129-134 (1996); Sullivan et al., J. Biomol. Screening, 2:19-23 (1997); De Serres et al., Anal. Biochem., 233:228-233 (1996); Sonatore et al., Anal. Biochem., 240:289-297 (1996); Chen et al., J. Biol. Chem., 271:25308-25315 (1996); Patel et al., Biochem. Biophys. Res. Commun., 221:821-825 (1996); and Fox, Pharm. Forum, 6:1-3 (1996)). SPA can also be easily adapted to a variety of enzyme assays (Lemer et al., J. Biomol. Screening, 1: 135-143 (1996); Baker et al., Anal. Biochem., 239:20-24 (1996); Baum et al., Anal. Biochem., 237:129-134 (1996); and Sullivan et al., J. Biomol. Screening, 2:19-23 (1997)) and protein-protein interaction assays (Braunwalder et al., J. Biomol. Screening, 1:23-26 (1996); Sonatore et al., Anal. Biochem., 240:289-297 (1996); and Chen et al., J. Biol. Chem., 271:25308-25315 (1996)).

One version of SPA utilizes polyvinyltoluene (PVT) microspheres or beads (˜5 ìm diameter, density˜1.05 g/cm³) into which a scintillant has been incorporated (Hook, Drug Discov. Tech., 1:287-294 (1996)). When a radiolabeled ligand is captured on the surface of the bead, the radioactive decay occurs in close proximity to the bead, and effectively transfers energy to the scintillant, which results in light emission. When the radiolabel is displaced or inhibited from binding to the bead, it remains free in solution and is too distant from the scintillant for efficient energy transfer. Energy from radioactive decay is dissipated into the solution, which results in no light emission from the beads. Hence, the bound and free radiolabel can be detected without the physical separation required in filtration assays.

The ideal isotopes for labeling ligands used in SPA assays are ³H and ¹²⁵I. This is because the â particles from ³H have a relatively short pathlength, about 1.5 ìM, which easily fulfills the distance requirement for SPA. The Auger electrons emitted by ¹²⁵I, which travel between approximately 1 ìm and 17.6 ìm in aqueous media, also satisfy this distance requirement.

SPA can also be carried out in scintillating microplates (Braunwalder et al., J. Biomol. Screening, 1:23-26 (1996); Fox, Pharm. Forum, 6:1-3 (1996); and Harris et al., Anal. Biochem., 243:249-256 (1996)), in which the scintillant is directly incorporated into the plastic, or is coated on the inner surface of the wells. These plates are commercially available. For example, Flashplate® is from NEN™ Life Science Products (Boston, Mass.) in which the scintillant is coated on the inner surface of the wells. The Scinitstrip® plate is from WallacOy (Turku, Finland) which is made by incorporating the scintillant into the entire plastic. A more recent development is the Cytostar-T™ (Amerisham Life Sciences, Cardiff, Wales) scintillating microplates (Fox, Pharm. Forum, 6:1-3 (1996) which were specially designed for cell-based proximity assays. Scintillant is incorporated into the base plate of microtiter plates and can also detect additional isotopes such as ¹⁴C, ⁴⁵Ca, ³⁵S, and ³³P.

b. Non-Isotopic Detection Methods

1) Colorimetry and Luminescence

Colorimetric and luminescence detection methods have significant advantages for HTS laboratories, particularly in light of the cost, safety and disposal issues associates with radiochemical methods. Since luminescence methods can be as sensitive as radioactive methods, with low detection limits, these techniques are being used increasingly in HTS assays (Brown et al., Curr. Opin. Biotechnol., 8:45-49 (1997); Glazer, BioRadiations, 98:4-8 (1997); Czamik, Chem. Biol., 2:423-428 (1995); Wang et al., Tetrahedron Lett., 31:6493-6496 (1991); Mathis, Clin. Chem., 41:1391-1397 (1995); Kolb et al., J. Biomol. Screening, 1:203-210 (1996); Gonzalez et al., Biophys. J., 69:1272-1280 (1995); Schroeder et al., J. Biomol. Screening, 1:75-80 (1996); Waggoner et al., Hum. Pathol., 27:494-502 (1996); Jameson et al., Methods Enzymol., 246:283-300 (1995); Lundblad et al., Mol. Endocrinol., 10:607-612 (1996); Checovich et al., Nature, 375:254-256 (1995); Levine et al., Anal. Biochem., 247:83-88 (1997); Jolley, J. Biomol. Screening, 1:33-38 (1996); Schade et al., Anal. Biochem., 243:1-7 (1996); Lynch et al., Anal. Biochem., 247:77-82 (1997); Sterrer et al., J. Recept. Signal Transduct Res., 17:511-520 (1997); Rigler, J. Biotechnol., 41:177-186 (1995); Rauer et al., Biophys. Chem., 58:3-12 (1996); Sarubbi et al., Anal. Biochem., 237:70-75 (1996); Rose et al., Network Science, 2(9):1-12 (1996); Dhundale et al., J. Biomol. Screening, 1:115-118 (1996); Suto et al., J. Biomol. Screening, 2:7-9 (1997); Bronstein et al., Anal. Biochem., 219:169-181 (1994); Hastings, Gene, 173:5-11 (1996); Lehel et al., Anal. Biochem., 244:340-346 (1997); Kolb et al., J. Biomol. Screening, 1:85-88 (1996); Bran et al., J. Biomol. Screening, 1:43-45 (1996); Rizzuto et al., Curr. Biol, 6:183-188 (1996)). Glazer (Glazer, BioRadiations, 98:4-8 (1997)) and Czamik (Czamik, Chem. Biol, 2:423-428 (1995)) and the Fluorescent Chemosensors and Biosensors Database on the World Wide Web URL; http://biomednet.com/fluoro/ have reviewed the utility and need for fluorescence-based techniques for biological applications, which can be easily extended to HTS assays.

3) Resonance Energy Transfer

Resonance energy transfer (RET) between a fluorophore and chromophore was one of the earliest methods developed for HTS. For example, a peptide substrate for an HIV protease was synthesized with EDANS (as the amino terminus) as the donor fluorophore, and DABCYL (at the carboxyl terminus) as the acceptor chromophore (Wang et al., Tetrahedron Lett., 31:6493-6496 (1991)). Energy transfer from EDANS to DABCYL in the intact peptide resulted in quenching of EDANS fluorescence.

4) Time-Resolved Fluorescence

A new homogeneous time-resolved fluorescence (HTRF) technology has been described (Mathis, Clin. Chem., 41:1391-1397 (1995)). The assay utilizes fluorescence energy transfer between two fluorophores (an europium cryptate and a 105 kDa phycobiliprotein, allophycocyanin) as labels. The Eu-trisbipyridine cryptate (TBP-EU³⁺, ëₑₓ=337 nm) has two bipyridyl groups that harvest light and channel it to the caged Eu³⁺. It has a long fluorescence, lifetime and nonradioactively transfers the energy to allophycocyanin when the two labels are in close proximity (>50% transfer efficiency at a donor-acceptor distance of 9.5 nm). The resulting fluorescence of allophycocyanin ëₑₘ=665 nm) retains the long lifetime of the donor TBP)-EU³⁺, allowing time-resolved measurement. These labels and their spectroscopic characteristics are very stable in biological media.

5) Cell-Based Fluorescence Assays

An interesting fluorescence resonance energy transfer (FRET) procedure for sensing voltage across cell membranes has been described recently (Gonzalez et al., Biophys. J., 69:1272-1280 (1995)). The technique uses membrane permeable, anionic oxonols which rapidly locate on the inner or outer membrane surface depending on polarization state of the membrane. FRET occurs between fluorescein-labeled WGA and the oxonols bound to the other surface of the membrane at a resting negative potential. As a positive potential, the oxonols are relocated to the inner membrane surface, and the FRET is greatly reduced.

Many fluorescence intensity measurements, including FRET, can be configured on a instruments specifically designed for cell-based HTS assays in 96-well or higher density plates called FLIPr (Schroeder et al., J. Biomol. Screening, 1:75-80 (1996)]. FLIPR utilizes a water-cooled argon ion laser (5 watt) or a xenon are lamp and a semiconfocal optical system with a charge-coupled device (CCD) camera to illuminate and image the entire plate. The spatial resolution of the optics is ˜200 ìm at the cell plane. The plate chamber temperature can be controlled precisely, and a 96-well pipettor head is integrated into the instrument. These features allow accurate measurements of cellular biochemistry in confluent layers of cells at the bottom of plates. FLIPR software can rapidly quantify transient fluorescence signals in intact cells that are growing attached to the bottom of the well. HTS assays involving intracellular calcium, pH and membrane potential measurements have been designed using this instrument (Waggoner et al., Hum. Pathol., 27:494-502 (1996)).

6) Fluorescence Polarization

Another technique that has gained popularity recently is fluorescence polarization or anisotropy (Jameson et al., Methods Enzymol., 246:283-300 (1995); Lundblad et al., Mol. Endocrinol., 10:607-612 (1996); Checovich et al., Nature, 375:254-256 (1995); Levine et al., Anal. Biochem., 247:83-88 (1997); Jolley, J. Biomol. Screening, 1:33-38 (1996); Schade et al., Anal. Biochem., 243:1-7 (1996); Lynch et al., Anal. Biochem., 247:77-82 (1997)). When fluorescently labeled molecules in solution are illuminated with plane-polarized light, the emitted fluorescence will be in the same plane provided the molecules remain stationary. Since all molecules tumble as a result of collisional motion, depolarization phenomenon is proportional to the rotational relaxation time (ì) of the molecule, which is defined by the expression 3çV/RT. At constant viscosity (ç) and temperature (T) of the solution, polarization is directly proportional to the molecular volume (V) (R is the universal gas constant). Hence, changes in molecular volume or molecular weight due to binding interactions can be detected as a change in polarization. For example, the binding of a fluorescently labeled ligand to its receptor will result in significant changes in measured fluorescence polarization values for the ligand. Once again, the measurements can be made in a “mix and measure” mode without physical separation of the bound and free ligands. The polarization measurements are relatively insensitive to fluctuations in fluorescence intensity when working in solutions with moderate optical intensity.

7) Fluorescence Correlation Spectroscopy

Fluorescence correlation spectroscopy (FCS) has been recently described for HTS applications (Sterrer et al., J. Recept. Signal Transduct Res., 17:511-520 (1997); Rigler, J. Biotechnol., 41:177-186 (1995); Rauer et al., Biophys. Chem., 58:3-12 (1996)). FCS measures time-dependent and spontaneous fluctuations in fluorescence intensities in very small volumes (nanoliters). These fluctuations usually result from Brownian motion associated with chemical reactions, diffusion or the flow of fluorescently labeled molecules. The average fluctuation is proportional to the square foot of N, where N is the average number of molecules in the volume. Since Brownian diffusion is directly affected by molecular interactions, FCS is an excellent tool to measure binding interactions (Brown et al., Curr. Opin. Biotechnol., 8:45-49 (1997)). Using powerful lasers and autocorrelation techniques, sensitive measurements (at concentrations of ˜10-¹²M) can be made in solution and in cellular compartments.

3. Miniaturization

Several factors are fueling efforts to increase the speed of HTS and decrease the volume of individual reactions within an HTS format (Silverman et al., Curr. Opin. Chem. Biol., 2(3):397-403 (1998)). Split-bead synthesis, or other similar approaches to combinatorial chemistry, dramatically increases the number of compounds that can be produced in a library but do so at the cost of quantity of material.

One approach involves reducing the well size and increasing the density of the assay plate but retaining the overall assay format used in current 96-well based HTS. Densities of 6,500 assays in a 10 cm array have been reported to cell-free enzyme based assays (Schullek et al., Anal. Biochem., 246:20-29 (1997)) and for ligand binding in cell based assays (You et al., Chem. Biol., 4:969-975 (1997)). This approach of miniaturizing existing formats significantly increases the number of assays per plate and the overall throughput of the screen but is intrinsically limited by the physical constraints of delivering small volumes to wells, and of detecting responses in a sensitive and timely manner. Another approach uses glass chips containing microchannels in which reagents, target proteins and compounds are herded by electrokinetic flow controlled by electric potentials applied at the ends of the channels (Hadd et al., Anal. Chem., 69:3407-3412 (1997)). A related approach attains high-throughput of chemical synthesis and activity assessment by parallel arrays of three-dimensional channels in which flow is controlled by miniature hydrostatic actuators (Rogers, Drug Discov. Today, 2:306 (1997)). These approaches provide significant reduction in the volume of assays and a corresponding savings in reagent costs over conventional HTS. In addition, with further development in parallel processing in multiple chips, the number of assays performed in a given period of time can increase dramatically.

In a specific embodiment, the HTS methods disclosed in the following literatures can be used, with or without modification, in the present methods for detecting, localizing and/or removing abnormal base-pairing, mutations and polymorphisms: Janzen et al., The 384-well plate: pros and cons, J. Biomol. Screening, 1:63-64 (1996); Lutz, et al., Experimental design for high-throughput screening, Drug Discov. Tech., 1:277-286 (1996); Klein, et al., Recombinant microorganisms as tools for high throughput screening for non antibiotic compounds, J. Biomol. Screening, 2:41-49 (1997); Webb, et al., Transcription-specific assay for quantifying mRNA: A potential replacement for reporter gene assays, J. Biomol. Screening, 1:119-121 (1996); Charych, et al., Direct calorimetric detection of receptor-ligand interaction by a polymerized bilayer assembly, Science, 261:585-588 (1993); Charych, et al., A ‘litmus test’ for molecular recognition using artificial membranes, Chem. Biol., 3:113-120 (1996); Spevak, et al., Carbohydrates in an acidic multivalent assembly: nanomolar P-selectin inhibitors, J. Med. Chem., 38:1018-1020 (1996); Allen, et al., Atomic force microscopy in analytical biotechnology, Trends Biotechnol., 15:101-105 (1997); Troy, et al., Scanning force microscopy helps in the design of cancer drugs, Biophoton Int., 9/10:52-53 (1996); Paborsky, et al., A nickel chelate microtiter plate assay for six histidine-containing proteins, Anal. Biochem., 234:60-65 (1996); Weiss-Wichert, et al., A new analytical device based on gated ion channels: A peptide channel biosensor, J. Biomol. Screening, 2:11-18 (1997); Brecht, et al., Transducer-based approaches for parallel binding assays in HTS, J. Biomol. Screening, 1:191-201 (1996); Tyagi, et al., Molecular beacons: probes that fluoresce upon hybridization, Nat. Biotechnol., 14:303-308 (1996); Heller, et al., Discovery and analysis of inflammatory disease-related genes using cDNA microarrays, Proc. Natl. Acad. Sci. USA, 94:2150-2155 (1997); Nicolaou, et al., Radiofrequency encoded combinatorial chemistry, Angew Chem. Int. Ed., 34:2289-2291 (1995); Fitzgerald, et al., Direct characterization of solid phase resin-bound molecules by mass spectrometry, Bioorg. Med. Chem. Lett., 6:979-982 (1996); Chu, et al., Affinity capillary electrophoresis-mass spectrometry for screening combinatorial libraries, J. Am. Chem. Soc., 118:7827-7835 (1996); and Evans, et al., Affinity-based screening of combinatorial libraries using automated, serial-column chromatography, Nat. Biotechnol., 14:504-507 (1996).

### J. Sample Collection

Any sample can be assayed for detecting, localizing and/or removing abnormal base-pairing, mutations or polymorphisms using the methods described in the above Sections B-F. In one embodiment, the sample being assayed is a biological sample from a mammal, particularly a human, such as a biological fluid or a biological tissue. Biological fluids, include, but are not limited to, urine, blood, plasma, serum, saliva, semen, stool, sputum, hair and other keratinous samples, cerebral spinal fluid, tears, mucus and amniotic fluid. Biological tissues contemplated include, but are not limited to, aggregates of cells, usually of a particular kind together with their intercellular substance that form one of the structural materials of a human, animal, plant, bacterial, fungal or viral structure, including connective, epithelium, muscle and nerve tissues, organs, tumors, lymph nodes, arteries and individual cell(s). In one specific embodiment, the body fluid to be assayed is urine. In another specific embodiment, the body fluid to be assayed is blood. Preferably, the blood sample is further separated into a plasma or sera fraction.

Serum or plasma can be recovered from the collected blood by any methods known in the art. In one specific embodiment, the serum or plasma is recovered from the collected blood by centrifugation. Preferably, the centrifugation is conducted in the presence of a sealant having a specific gravity greater than that of the serum or plasma and less than that of the blood corpuscles which will form the lower, whereby upon centrifugation, the sealant forms a separator between the upper serum or plasma layer and the lower blood corpuscle layer. The sealants that can be used in the processes include, but are not limited to, styrene resin powders (Japanese Patent Publication No. 38841/1973), pellets or plates of a hydrogel of a crosslinked polymer of 2-hydroxyethyl methacrylate or acrylamide (U.S. Pat. No. 3,647,070), beads of polystyrene bearing an antithrombus agent or a wetting agent on the surfaces (U.S. Pat. No. 3,464,890) and a silicone fluid (U.S. Pat. Nos. 3,852,194 and 3,780,935). In a preferred embodiment, the sealant is a polymer of unsubstituted alkyl acrylates and/or unsubstituted alkyl methacrylates, the alkyl moiety having not more than 18 carbon atoms, the polymer material having a specific gravity of about 1.03 to 1.08 and a viscosity of about 5,000 to 1,000,000 cps at a shearing speed of about 1 second⁻¹ when measured at about 25° C. (U.S. Pat. No. 4,140,631).

In another specific embodiment, the serum or plasma is recovered from the collected blood by filtration. Preferably, the blood is filtered through a layer of glass fibers with an average diameter of about 0.2 to 5 ì and a density of about 0.1 to 0.5 g./cm³, the total volume of the plasma or serum to be separated being at most about 50% of the absorption volume of the glass fiber layer; and collecting the run-through from the glass fiber layer which is plasma or serum (U.S. Pat. No. 4,477,575). Also preferably, the blood is filtered through a layer of glass fibers having an average diameter 0.5 to 2.5 ì impregnated with a polyacrylic ester derivative and polyethylene glycol (U.S. Pat. No. 5,364,533). More preferably, the polyacrylic ester derivative is poly(butyl acrylate), poly(methyl acrylate) or poly(ethyl acrylate), and (a) poly(butyl acrylate), (b) poly(methyl acrylate) or poly(ethyl acrylate) and (c) polyethylene glycol are used in admixture at a ratio of (10-12):(1-4):(1-4). In still another specific embodiment, the serum or plasma is recovered from the collected blood by treating the blood with a coagulant containing a lignan skelton having oxygen-containing side chains or rings (U.S. Pat. No. 4,803,153). Preferably, the coagulant contains a lignan skelton having oxygen-containing side chains or rings, e.g., d-sesamin, l-sesamin, paulownin, d-asarinin, l-asarinin, 2á-paulownin, 6á-paulownin, pinoresinol, d-eudesmin, l-pinoresinol β-D-glucoside, l-pinoresinol, l-pinoresinol monomethyl ether β-D-glucoside, epimagnolin, lirioresinol-B, syringaresinol (dl), lirioresinonB-dimethyl ether, phillyrin, magnolin, lirioresinol-A, 2á, 6á-d-sesamin, d-diaeudesmin, lirioresinol-C dimethyl ether (d-diayangambin) and sesamolin. More preferably, the coagulant is used in an amount ranging from about 0.01 to 50 g per 1 l of the blood.

### K. Combinations, Kits and Articles of Manufacture

Combinations, kits and articles of manufacture for detecting abnormal base-pairings, mutations, polymorphisms, and for localizing and/or removing abnormal base-pairings are provided herein.

In a specific embodiment, a combination for detecting abnormal base-pairing in a nucleic acid duplex is provided herein, which combination comprises: a) a mutant DNA repair enzyme or complex thereof; and b) reagents for detecting binding between abnormal base-pairing in a nucleic acid duplex and the mutant DNA repair enzyme or complex thereof. A kit comprising the above combination is also provided. An article of manufacture is further provide herein, which article of manufacture comprises: a) packaging material; b) the above-described combination; and c) a label indicating that the article is for use in detecting abnormal base-pairing in a nucleic acid duplex.

In another specific embodiment, a combination for detecting a mutation in a nucleic acid duplex is provided herein, which combination comprises: a) a strand of a wild-type nucleic acid complementary to a nucleic acid having or suspected of having a mutation; b) a mutant DNA repair enzyme or complex thereof; and c) reagents for detecting binding between abnormal base-pairing in a nucleic acid duplex and the mutant DNA repair enzyme or complex thereof. A kit comprising the above combination is also provided. An article of manufacture is further provided, comprising: a) packaging material; b) the above combination; and c) a label indicating that the article is for use in detecting a mutation in a nucleic acid duplex.

In still another specific embodiment, a combination for detecting a polymorphism in a locus is provided herein, which combination comprises: a) a complementary reference strand of a nucleic acid comprising a known allele of a locus; b) a mutant DNA repair enzyme or complex thereof; and c) reagents for detecting binding between abnormal base-pairing in a nucleic acid duplex and the mutant DNA repair enzyme or complex thereof. A kit comprising the above combination is also provided. An article of manufacture is further provided, comprising: a) packaging material; b) the above combination; and c) a label indicating that the article is for use in detecting a polymorphism in a locus.

In yet another specific embodiment, a combination for removing a nucleic acid duplex containing one or more abnormal base-pairing in a population of nucleic acid duplexes is provided herein, which combination comprises: a) a mutant DNA repair enzyme or complex thereof; and b) reagents for removing a binding complex formed between a nucleic acid duplex containing one or more abnormal base-pairing and the mutant DNA repair enzyme or complex thereof. A kit comprising the above combination is also provided. An article of manufacture is further provided, comprising: a) packaging material; b) the above combination; and c) a label indicating that the article is for use in removing a nucleic acid duplex containing one or more abnormal base-pairing in a population of nucleic acid duplexes.

In yet another specific embodiment, a combination for detecting and localizing an abnormal base-pairing in a nucleic acid duplex is provided herein, which combination comprises: a) a mutant DNA repair enzyme or complex thereof; and b) an exonuclease. A kit comprising the above combination is also provided. An article of manufacture is further provided, comprising: a) packaging material; b) the above combination; and c) a label indicating that the article is for use in for detecting and localizing an abnormal base-pairing in a nucleic acid duplex.

### L. Example

In order to select an appropriate single nucleotide polymorphism (SNP) substrate trapping enzyme (STE), mismatch repair wild-type enzyme MutS (LeClerc et al., Science, 274(5290):1208-11 (1996)) was cloned by the following steps. First, a mismatch repair enzyme gene from Escherichia coli bacterial genomic DNA was amplified by polymerase chain reaction (PCR). Second, the PCR product generated with polynucleotide DNA primers 5′-ATG AGT GCA ATA GAA AAT TTC GAC-3′ (SEQ ID NO:79) and 5′-CCC ACC AGA CTC TTC AAG CGA TAA ATC C-3′ (SEQ ID NO:80) was cloned into a commercially available gene expression vector downstream of an inducible promoter (pBAD/ThioE, Invitrogen Corporation, Carlsbad, Calif.). The SNP-STE wild-type enzyme, thus cloned and sequenced (Diazyme-SNP-STE-WT), bears an epitope tag (V5-His) at the C-terminal end (Invitrogen Corporation, Carlsbad, Calif.). Third, the cloned wild-type enzyme was subsequently mutagenized based on published structure and function information of the enzyme (Wu and Marinus, J. Bacteriol., 176(17):5393-400 (1994); Das Gupta and Kolodner, Nat. Genet., 24(1):53-6 (2000); Lamers et al., Nature, 407(6805):711-7 (2000); and Obmolova et al., Nature, 407(6805):703-10 (2000)).

Two exemplary mutant MutS enzymes, E673K (5′-CCT TTA TGG TGA AGA TGA CTG AAA-3′) (SEQ ID NO:81) and H728A (5′-CGT TAT TTG CTA CCG CCT ATT TCG AGC TG-3′) (SEQ ID NO:82), were generated using the above described methods. The mutant mismatch repair enzymes were produced by system manufacturer's recommended protocol (catalog no. ET100-10C, Invitrogen Corporation, Carlsbad, Calif.) and purified by standardized nickel-affinity chromatography as per system manufacturer's instructions (catalog no. 30210, Qiagen, Valencia, Calif.) for subsequent evaluation of DNA binding abilities by a plate-based assay described below.

The assay for selection of candidate SNP-STE is described below.

1) Complementary polynucleotide DNA substrates 5′-GCA CCT GAC TCC TGX GGA GAA GTC TGC CGT-3′ (SEQ ID NO:83) and 5′-ACG GCA GAC TTC TCC XCA GGA GTC AGG TGC-3′ (SEQ ID NO:84) were commercially synthesized based on a published study (Wagner et al., Nucleic Acids Res., 23(19):3944-8 (1995)). The polynucleotide pairs were identical 30 mers in size with the exception of a centrally located designated test nucleotide marked X. The centrally located nucleotide served as the correct pairing or mispairing site when complementary polynucleotide substrates were annealed in all possible combinations (G, A, T, C). The forward or top oligo in the complementary pair contained a biotin moiety conjugated at the 5′ end.

2) The polynucleotide DNA substrates were heated in a suitable annealing buffer (25 mM HEPES, 1 mM DTT, 2 mM MgCl₂, 15% glycerol) to 94° C. for 15 minutes in an automated thermal cycler and then cooled slowly for hybridization.

3) The polynucleotide duplex substrates prepared as described above contain a 5′ biotin label on the top (forward) strand. An optimized amount of DNA substrates, suitably buffered (25 mM HEPES, 1 mM DTT, 2 mM MgCl₂, 15% glycerol), was transferred to multiwell microplates that were pre-coated with neutravidin and blocked as per manufacturer's instructions (Pierce Chemical Co., Rockford, Ill.). The DNA microplates were thus prepared with different types of correct pairing or mispairing DNA substrates. The buffers were further optimized for highest signal-to-noise discrimination by use of various competitors.

4) Mutant SNP-STEs were prepared as described and tested with the microplate DNA substrates for binding efficiencies as follows. Protein was added to each well and the mixture was incubated at ambient temperature for 30 minutes.

5) After washing out unbound protein with a suitable solution (25 mM Tris, 150 mM NaCl, 0.05% Tween, 0.1 % BSA), buffered antibody conjugated horse radish peroxidase enzyme (V5-HRP; Invitrogen Corp., Carlsbad, Calif.) was added to the wells. The antibody specifically recognizes the V5 epitope present at the C-terminus on the SNP-STE proteins.

6) After another 30 minute incubation step, excess antibody was washed out of each well with a suitable buffer and substrate for HRP enzyme (TMB; Pierce Chemical Co., Rockford, Ill.) was added for calorimetric detection of bound protein as per manufacturer's instruction.

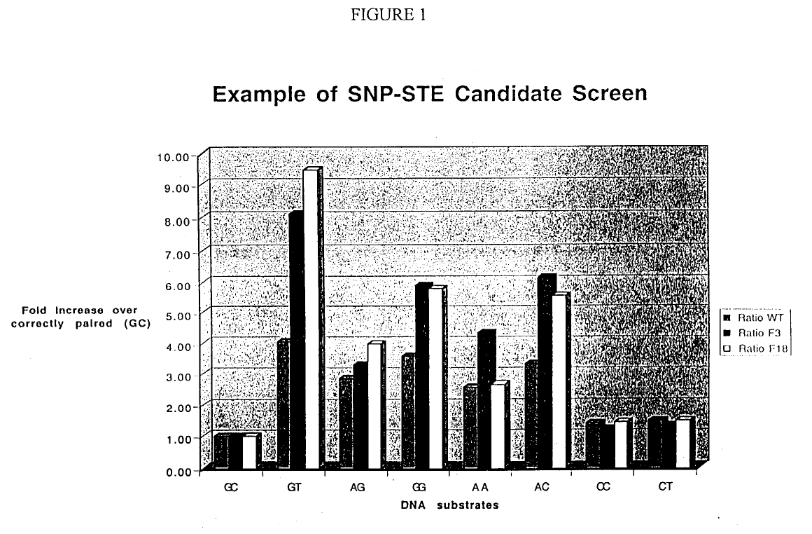

FIG. 1 shows a representative graph of mismatch binding abilities of SNP-STE-F3 (E673K) and SNP-STE-F18 (H728A), respectively, compared to the SNP-STE-WT enzyme. SNP-STE-WT is the unmodified original enzyme with native protein sequence. Under the optimized assay conditions, which include various competitors for DNA-binding proteins, the candidate SNP-STE proteins showed an significantly increased affinity towards a majority of mismatched DNA mispairs in comparison with the wild-type enzyme.

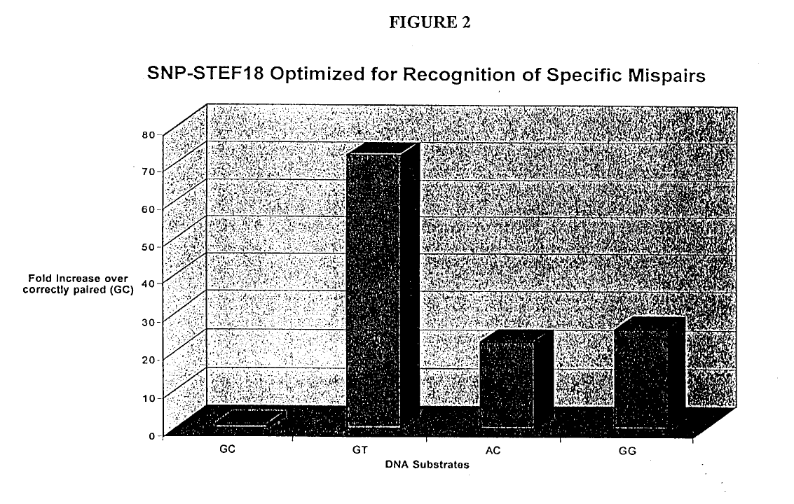

Under one optimized assay condition for specific mismatches, the SNP-STE-F 18 tested in our experiments has more than 60-fold higher binding ability toward GT, and more than 20-fold higher binding ability toward AC and GG mispairs in comparison with correctly paired control DNA (FIG. 2).

Since modifications will be apparent to those of skill in this art, it is intended that this invention be limited only by the scope of the appended claims.

What is claimed:

1. A method for detecting abnormal base-pairing in a nucleic acid duplex, which method comprises:
a) contacting a nucleic acid duplex having or suspected of having an abnormal base-pairing with a mutant nucleic acid repair enzyme or complex thereof, wherein the mutant nucleic acid repair enzyme or complex thereof has binding affinity for the abnormal base-pairing in the duplex but has attenuated catalytic activity compared to the wild-type enzyme; and
b) detecting binding between the nucleic acid duplex and the mutant nucleic acid repair enzyme or complex thereof, whereby the presence or quantity of the abnormal base-pairing in the duplex is assessed.

2. The method of claim 1, wherein the nucleic acid duplex is selected from the group consisting of a DNA:DNA, a DNA:RNA and an RNA:RNA duplex.

3. The method of claim 2, wherein the nucleic acid duplex is a DNA:DNA duplex.

4. The method of claim 1, wherein the abnormal base-pairing is selected from the group consisting of a base-pair mismatch, a base insertion, a base deletion and a pyrimidine dimer.

5. The method of claim 4, wherein the base-pair mismatch is a single base-pair mismatch.

6. The method of claim 1, wherein the mutant nucleic acid repair enzyme or enzyme complex is selected from the group consisting of a mutant mutH, a mutant mutL, a mutant mutM, a mutant mutS, a mutant mutY, a mutant uvrD, a mutant dam, a mutant thymidine DNA glycosylase (TDG), a mutant mismatch-specific DNA glycosylase (MUG), a mutant AlkA, a mutant MLH1, a mutant MSH2, a mutant MSH3, a mutant MSH6, a mutant Exonuclease I, a mutant T4 endonuclease V, a mutant FEN1 (RAD27), a mutant DNA polymerase ä, a mutant DNA polymerase \dot{a}, a mutant RPA, a mutant PCNA, a mutant RFC, a mutant Exonuclease V, a mutant DNA polymerase III holoenzyme, a mutant DNA helicase, a mutant RecJ exonuclease and combinations thereof.

7. The method of claim 1, wherein the nucleic acid duplex is formed by hybridizing a single strands of nucleic acid that contain a known sequence with a nucleic acids from a test sample, whereby binding of the mutant enzyme to any duplexes indicates that presence of a sequence difference in the nucleic acid from the sample from that of the nucleic acid containing the known sequence.

8. The method of claim 1, wherein the single strands of nucleic acid fragments with known sequences are immobilized on a solid support.

9. The method of claim 8, wherein the fragments are arranged in an array.

10. The method of claim 8 that is automated.

11. A method for detecting a mutation in a nucleic acid, comprising:
a) hybridizing a strand of a nucleic acid having or suspected of having a mutation with a complementary strand of a nucleic acid fragment having a wild type sequence, whereby the mutation results in an abnormal base-pairing in the formed nucleic acid duplex;
b) contacting the nucleic acid duplex formed in step a) with a mutant nucleic acid repair enzyme or complex thereof, wherein the mutant nucleic acid repair enzyme or complex thereof has binding affinity for the abnormal base-pairing in the duplex but has attenuated catalytic activity; and
c) detecting binding between the nucleic acid duplex and the mutant nucleic acid repair enzyme or complex thereof, whereby the presence or quantity of the mutation is assessed.

12. The method of claim 11, wherein the nucleic acid strand to be tested and the complementary wild-type nucleic acid strand are NA strands.

13. The method of claim 11, wherein the mutation is associated with a disease or disorder, or infection by a pathological agent, and the method is used for prognosis or diagnosis of the presence or severity of the disease, disorder or infection.

14. The method of claim 13, wherein the disease or disorder is selected from the group consisting of a cancer, an immune system disease or disorder, a metabolism disease or disorder, a muscle and bone disease or disorder, a nervous system disease or disorder, a signal disease or disorder and a transporter disease or disorder.

15. The method of claim 13, wherein the a plurality of mutations are identified by hybridizing nucleic acid single stands to a plurality of different fragments comprising loci encompassing different mutations.

16. The method of claim 15 that is automated.

17. A method for detecting polymorphism in a gene locus, comprising:
a) hybridizing a target strand of a nucleic acid comprising a locus to be tested with a complementary reference strand of a nucleic acid comprising a known allele of the locus, whereby the allelic identity between the target and the reference strands results in the formation of a nucleic acid duplex without an abnormal base-pairing and the allelic difference between the target and the reference strands results in the formation of a nucleic acid duplex with an abnormal base-pairing;
b) contacting the nucleic acid duplex formed in step a) with a mutant nucleic acid repair enzyme or complex thereof, wherein the mutant nucleic acid repair enzyme or complex thereof has binding affinity for the abnormal base-pairing in the duplex but has attenuated catalytic activity; and
c) detecting binding between the nucleic acid duplex and the mutant nucleic acid repair enzyme or complex thereof, whereby the polymorphism in the locus is assessed.

18. The method of claim 17, wherein a plurality of reference strands are hybridized.

19. The method of claim 18, wherein the reference strands are immobilized on a solid support.

20. The method of claim 19, wherein the reference strands are immobilized in an array.

21. The method of claim 17, wherein the polymorphism to be detected is a variable nucleotide type polymorphism (“VNTR”).

22. The method of claim 17, wherein the polymorphism to be detected is a single nucleotide polymorphism (SNP).

23. The method of claim 22, wherein the SNP is a human genome SNP.

24. The method of claim 23, wherein the hybridization between the target strand of a nucleic acid comprising a locus to be tested and the complementary reference strand of a nucleic acid comprising a known allele of the locus is facilitated by a recombinase.

25. The method of claim 18 that is automated.

26. A method for purifying or separating nucleic acid duplex containing one or more abnormal base-pairing from a population of nucleic acid duplexes, which method comprises:
a) contacting a population of nucleic acid duplexes having or suspected of having a nucleic acid duplex containing one or more abnormal base-pairing with a mutant nucleic acid repair enzyme or complex thereof, wherein the mutant nucleic acid repair enzyme or complex thereof has binding affinity for the abnormal base-pairing in the duplex but has attenuated catalytic activity and whereby the nucleic acid duplex containing one or more abnormal base-pairing binds to the mutant nucleic acid repair enzyme or complex thereof to form a binding complex; and
b) removing nucleic acid duplexes that contain the binding complex formed in step a) from the population of nucleic acid duplexes.

27. The method of claim 1, wherein the abnormal base-pairing is selected from the group consisting of a base-pair mismatch, a base insertion, a base deletion and a pyrimidine dimer.

28. The method of claim 11, wherein the abnormal base-pairing is selected from the group consisting of a base-pair mismatch, a base insertion, a base deletion and a pyrimidine dimer.

29. The method of claim 26, wherein the abnormal base-pairing is selected from the group consisting of a base-pair mismatch, a base insertion, a base deletion and a pyrimidine dimer.

30. The method of claim 26, wherein the population of nucleic acid duplexes is produced by an enzymatic amplification.

31. A method for detecting and localizing an abnormal base-pairing in a nucleic acid duplex, which method comprises:
a) contacting a nucleic acid duplex having or suspected of having an abnormal base-pairing with a mutant nucleic acid repair enzyme or complex thereof, wherein the mutant nucleic acid repair enzyme or complex thereof has binding affinity for the abnormal base-pairing in the duplex but has attenuated catalytic activity and whereby the nucleic acid duplex containing an abnormal base-pairing binds to the mutant nucleic acid repair enzyme or complex thereof to form a binding complex;
b) subjecting the nucleic acid duplex to hydrolysis with an exonuclease under conditions such that the binding complex formed in step a) blocks hydrolysis; and
c) determining the location within the nucleic acid duplex protected from the hydrolysis, thereby detecting and localizing the abnormal base-pairing in the nucleic acid duplex.

32. The method of claim 31, wherein the nucleic acid duplex is selected from the group consisting of a DNA:DNA, a DNA:RNA and a RNA:RNA duplex.

33. The method of claim 31, wherein the abnormal base-pairing is selected from the group consisting of a base-pair mismatch, a base insertion, a base deletion and a pyrimidine dimer.

34. The method of claim 31, wherein the exonuclease is selected from the group consisting of nuclease BAL-31, exonuclease III, Mung Bean exonuclease and Lambda exonuclease.

35. The method of claim 1, wherein the mutant nucleic acid repair enzyme or complex thereof is labeled with a detectable label.

36. The method of claim 35, wherein the mutant nucleic acid repair enzyme or complex thereof is labeled with biotin.

37. The method of claim 36, wherein the binding between the abnormal base-pairing and the biotin-labeled mutant nucleic acid repair enzyme or complex thereof is detected with a streptavidin labeled enzyme.

38. The method of claim 37, wherein the streptavidin labeled enzyme is selected from the group consisting of a peroxidase, a urease, an alkaline phosphatase, a luciferase and a glutathione S-transferase.

39. The method of claim 31, wherein the mutant nucleic acid repair enzyme or complex thereof is labeled.

40. The method of claim 11, wherein the mutant nucleic acid repair enzyme or complex thereof is labeled with a detectable label.

41. The method of claim 17, wherein the mutant nucleic acid repair enzyme or complex thereof is labeled with a detectable label.

42. The method of claim 26, wherein the mutant nucleic acid repair enzyme or complex thereof is labeled with a detectable label.

43. The method of claim 1, wherein the nucleic acid duplex or the mutant nucleic acid repair enzyme or complex thereof is immobilized on the surface of a support.

44. The method of claim 43, wherein the nucleic acid duplex or the mutant nucleic acid repair enzyme or complex thereof is immobilized directly on the surface or is immobilized on the surface via a linker.

45. The method of claim 43, wherein the insoluble support is a silicon chip.

46. The method of claim 45, wherein the geometry of the support is selected from the group consisting of beads, pellets, disks, capillaries, hollow fibers, needles, solid fibers, random shapes, thin films, membranes and chips.

47. The method of claim 44, wherein the nucleic acid duplex or the mutant nucleic acid repair enzyme or complex thereof is immobilized in an array or a well format on the surface.

48. The method of claim 11, wherein the strand of a nucleic acid having or suspected of having a mutation, the complementary strand of a wild-type nucleic acid, or the mutant nucleic acid repair enzyme or complex thereof is immobilized on the surface of a support.

49. The method of claim 17, wherein the target strand of a nucleic acid comprising a locus to be tested, the complementary reference strand of a nucleic acid comprising a known allele of the locus, or the mutant nucleic acid repair enzyme or complex thereof is immobilized on the surface of a support.

50. The method of claim 26, wherein the mutant nucleic acid repair enzyme or complex thereof is immobilized on the surface of a support.

51. The method of claim 31, wherein the nucleic acid duplex having or suspected of having an abnormal base-pairing or the mutant nucleic acid repair enzyme or complex thereof is immobilized on the surface of a support.

52. The method of claim 1, wherein the nucleic acid duplex having or suspected of having an abnormal base-pairing is isolated from a sample.

53. The method of claim 52, wherein the sample is a body fluid or a biological tissue.

54. The method of claim 53, wherein the body fluid is selected from the group consisting of urine, blood, plasma, serum, saliva, semen, stool, sputum, cerebral spinal fluid, tears, mucus and amniotic fluid.

55. The method of claim 53, wherein the biological tissue is selected from the group consisting of connective tissue, epithelium tissue, muscle tissue, nerve tissue, organs, tumors, lymph nodes, arteries and individual cell(s).

56. The method of claim 11, wherein the strand of a nucleic acid having or suspected of having a mutation is isolated from a sample.

57. The method of claim 17, wherein the strand of a nucleic acid comprising a locus to be tested is isolated from a sample.

58. The method of claim 26, wherein the population of nucleic acid duplexes is isolated from a sample.

59. The method of claim 31, wherein the nucleic acid duplex having or suspected of having an abnormal base-pairing is isolated from a sample.

60. The method of claim 1, wherein abnormal base-pairings in a plurality of the nucleic acid duplexes are detected simultaneously.

61. The method of claim 11, wherein mutations in a plurality of the nucleic acids are detected simultaneously.

62. The method of claim 17, wherein polymorphisms in a plurality of the loci are detected simultaneously.

63. The method of claim 26, wherein a plurality of nucleic acid duplexes containing one or more abnormal base-pairing are removed simultaneously.

64. The method of claim 31, wherein a plurality of the abnormal base-pairings are detected and localized simultaneously.

65. A combination for detecting abnormal base-pairing in a nucleic acid duplex, which combination comprises:
a) a mutant nucleic acid repair enzyme or complex thereof; and
b) a reagent for detecting binding between abnormal base-pairing in a nucleic acid duplex and the mutant nucleic acid repair enzyme or complex thereof.

66. A kit comprising the combination of claim 65 and instructions for binding the mutant repair enzyme to nucleic acid duplexes to detect a mutation in a nucleic acid duplex, or to detect a polymorphism in a locus, or to diagnose a disease or disorder or plurality thereof, or for gene mapping or identification by detecting a plurality of polymorphisms or mutations.

67. An isolated substantially pure mutant nucleic acid repair enzyme that further comprises a detectable label, wherein the mutant enzyme has attenuated catalytic activity compared to the wild type but retains binding affinity for a nucleic acid duplex containing an abnormal base pairing.

68. The mutant enzyme of claim 67 that comprises a fusion protein or conjugate of the mutant enzyme and an enzyme label.

69. An isolated substantially pure biotinylated mutant nucleic acid repair enzyme.

70. An article of manufacture, comprising:
a) packaging material;
b) a mutant nucleic acid repair enzyme that has attenuated catalytic activity compared to the wild type but retains binding affinity for a nucleic acid duplex containing an abnormal base pairing; and
c) a label indicating that the article is for use in detecting abnormal base-pairing in a nucleic acid duplex.

71. A combination for detecting and localizing an abnormal base-pairing in a nucleic acid duplex, comprising
a) a mutant nucleic acid repair enzyme or complex thereof, wherein the mutant enzyme that has attenuated catalytic activity compared to the wild type but retains binding affinity for a nucleic acid duplex containing an abnormal base pairing; and
b) an exonuclease.

72. A kit, comprising the combination of claim 71 and instructions for performing an assay for detecting and localizing an abnormal base-pairing an a nucleic acid duplex.

73. A method for detecting polymorphism in a gene locus, comprising:
a) hybridizing a target strand of a nucleic acid comprising a locus to be tested with a complementary reference strand of a nucleic acid comprising a known allele of the locus, wherein the reference strand is so chosen that a first nucleic acid duplex formed between the reference strand and the target strand that contains an allele identical to the known allele in the reference strand has a first binding affinity with a nucleic acid repair enzyme or complex thereof and a second nucleic acid duplex formed between the reference strand and the target strand that contains an allele different from the known allele in the reference strand has a second binding affinity with the nucleic acid repair enzyme or complex thereof, whereby the difference between the first and second binding affinities is detectable;
b) contacting the first and second nucleic acid duplexes formed in step a) with the nucleic acid repair enzyme or complex thereof; and
c) detecting the difference between the first and second binding affinities, whereby the polymorphism in the locus is assessed.

74. The method of claim 73, wherein the allelic sequence in the reference strand is changed to increase the difference between the first and second binding affinities.

75. The method of claim 73, wherein the nucleic acid repair enzyme or complex thereof is mutated to increase the difference between the first and second binding affinities.

76. The method of claim 75, wherein the nucleic acid repair enzyme or complex thereof has increased binding affinity to the first and/or the second nucleic acid duplexes.

77. The method of claim 75, wherein the nucleic acid repair enzyme or complex thereof has attenuated catalytic activity compared to the wild type enzyme but retains binding affinity to the first and/or the second nucleic acid duplexes.

78. The method of claim 73, wherein the first binding affinity is higher than the second binding affinity.

79. The method of claim 73, wherein the first binding affinity is lower than the second binding affinity.

80. The method of claim 73, wherein the first and/or the second nucleic acid duplex is a DNA:DNA, a DNA:RNA or an RNA:RNA duplex.

81. The method of claim 73, wherein the first and the second nucleic acid duplex are DNA:DNA duplexes.

82. The method of claim 73, wherein the nucleic acid repair enzyme or enzyme complex is selected from the group consisting of a mutH, a mutL, a mutM, a mutS, a mutY, a uvrD, a dam, a thymidine DNA glycosylase (TDG), a mismatch specific DNA glycosylase (MUG), an AlkA, a MLH1, a MSH2, a MSH3, a MSH6, an Exonuclease I, a T4 endonuclease V, a FEN1 (RAD27), a DNA polymerase â, a DNA polymerase a, a RPA, a PCNA, a RFC, an Exonuclease V, a DNA polymerase III holoenzyme, a DNA helicase, a RecJ exonuclease and combinations thereof.

83. The method of claim 73, wherein the reference strand, the first and/or the second nucleic acid duplex is immobilized on a solid support.

84. The method of claim 83, wherein the reference strand, the first and/or the second nucleic acid duplex are arranged in an array.

85. The method of claim 73 that is automated.

86. The method of claim 73, wherein the polymorphism is associated with a disease or disorder, or infection by a pathological agent, and the method is used for prognosis or diagnosis of the presence or severity of the disease, disorder or infection.

87. The method of claim 86, wherein the disease or disorder is selected from the group consisting of a cancer, an immune system disease or disorder, a metabolism disease or disorder, a muscle and bone disease or disorder, a nervous system disease or disorder, a signal disease or disorder and a transporter disease or disorder.

88. The method of claim 73, wherein a plurality of reference strands are hybridized.

89. The method of claim 88, wherein the reference strands are immobilized on a solid support.

90. The method of claim 89, wherein the reference strands are immobilized in an array.

91. The method of claim 73, wherein the polymorphism to be detected is a variable nucleotide type polymorphism (“VNTR”).

92. The method of claim 73, wherein the polymorphism to be detected is a single nucleotide polymorphism (SNP).

93. The method of claim 92, wherein the SNP is a human genome SNP.

94. The method of claim 73, wherein the hybridization between the target strand of a nucleic acid comprising a locus to be tested and the complementary reference strand of a nucleic acid comprising a known allele of the locus is facilitated by a recombinase.

95. The method of claim 73, wherein the nucleic acid repair enzyme or complex thereof is labeled with a detectable label.

96. The method of claim 95, wherein the detectable label is an enzyme selected from the group consisting of a peroxidase, a urease, an alkaline phosphatase, a luciferase and a glutathione S-transferase.

97. The method of claim 83, wherein the solid support is a silicon chip.

98. The method of claim 83, wherein the geometry of the support is selected from the group consisting of beads, pellets, disks, capillaries, hollow fibers, needles, solid fibers, random shapes, thin films, membranes, wells and chips.

99. The method of claim 73, wherein the target strand is isolated from a sample.

100. The method of claim 99, wherein the sample is a body fluid or a biological tissue.

101. The method of claim 100, wherein the body fluid is selected from the group consisting of urine, blood, plasma, serum, saliva, semen, stool, sputum, cerebral spinal fluid, tears, mucus and amniotic fluid.

102. The method of claim 100, wherein the biological tissue is selected from the group consisting of connective tissue, epithelium tissue, muscle tissue, nerve tissue, organs, tumors, lymph nodes, arteries and individual cell(s).

103. The method of claim 73, wherein polymorphisms in a plurality of the loci are detected simultaneously.

104. A combination for detecting polymorphism in a gene locus, which combination comprises:
a) a reference strand of a nucleic acid comprising a known allele of the locus complementary to a locus to be tested, wherein the reference strand is so chosen that a first nucleic acid duplex formed between the reference strand and the target strand that contains an allele identical to the known allele in the reference strand has a first binding affinity with a nucleic acid repair enzyme or complex thereof and a second nucleic acid duplex formed between the reference strand and the target strand that contains an allele different from the known allele in the reference strand has a second binding affinity with the nucleic acid repair enzyme or complex thereof, whereby the difference between the first and second binding affinities is detectable;
b) a nucleic acid repair enzyme or complex thereof; and
c) a reagent for detecting the difference between the first and second binding affinities is detectable.

105. A kit comprising the combination of claim 104 and instructions for detecting the difference between the first and second binding affinities to detect a polymorphism in a locus, or to diagnose a disease or disorder or plurality thereof, or for gene mapping or identification by detecting a plurality of polymorphisms.
